SEQUENCE LISTING (34 sequences)

SEQ ID NO: 1 - DNA, 18 bp, Artificial Sequence
  42bs_42bs
gacgctgctgacgctgct

SEQ ID NO: 2 - DNA, 22 bp, Artificial Sequence
  37bs_37bs
tgaggacgtgttgaggacgtgt

SEQ ID NO: 3 - DNA, 18 bp, Artificial Sequence
  BCRbs_BCRbs
gcagaagccgcagaagcc

SEQ ID NO: 4 - DNA, 18 bp, Artificial Sequence
  ErbB2bs_ErbB2bs
gccgcagtggccgcagtg

SEQ ID NO: 5 - protein, 107 aa, Artificial Sequence
  FKBP12F36V
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 6 - protein, 87 aa, Artificial Sequence
  ErbB2ZFWT
ERPFQCRICMRNFSRSDVLANHTRTHTGEKPFQCRICMRNFSQSSTLTRHLRTHTGEKPFQCRICMRNFSERQGLKRHLKTHTGEKG

SEQ ID NO: 7 - protein, 87 aa, Artificial Sequence
  ErbB2ZFR39A
ERPFQCRICMRNFSRSDVLANHTRTHTGEKPFQCRICMANFSQSSTLTRHLRTHTGEKPFQCRICMRNFSERQGLKRHLKTHTGEKG

SEQ ID NO: 8 - protein, 87 aa, Artificial Sequence
  ErbB2ZFR2AR39A
EAPFQCRICMRNFSRSDVLANHTRTHTGEKPFQCRICMANFSQSSTLTRHLRTHTGEKPFQCRICMRNFSERQGLKRHLKTHTGEKG

SEQ ID NO: 9 - protein, 87 aa, Artificial Sequence
  ErbB2ZFR2AR39AR67A
EAPFQCRICMRNFSRSDVLANHTRTHTGEKPFQCRICMANFSQSSTLTRHLRTHTGEKPFQCRICMANFSERQGLKRHLKTHTGEKG

SEQ ID NO: 10 - protein, 86 aa, Artificial Sequence
  37ZFWT
ERPFQCRICMRNFSRNFILQRHIRTHTGEKPFQCRICMRNFSDRANLRRHIRTHTGEKPFQCRICMRNFSRHDQLTRHIRTHTGLR

SEQ ID NO: 11 - protein, 86 aa, Artificial Sequence
  37ZFR39A
ERPFQCRICMRNFSRNFILQRHIRTHTGEKPFQCRICMANFSDRANLRRHIRTHTGEKPFQCRICMRNFSRHDQLTRHIRTHTGLR

SEQ ID NO: 12 - protein, 86 aa, Artificial Sequence
  37ZFR2AR39A
EAPFQCRICMRNFSRNFILQRHIRTHTGEKPFQCRICMANFSDRANLRRHIRTHTGEKPFQCRICMRNFSRHDQLTRHIRTHTGLR

SEQ ID NO: 13 - protein, 86 aa, Artificial Sequence
  37ZFR2AR39AR67A
EAPFQCRICMRNFSRNFILQRHIRTHTGEKPFQCRICMANFSDRANLRRHIRTHTGEKPFQCRICMANFSRHDQLTRHIRTHTGLR

SEQ ID NO: 14 - protein, 86 aa, Artificial Sequence
  42ZFR2AR39AR67A
EAPFQCRICMRNFSTGQILDRHIRTHTGEKPFQCRICMANFSVAHSLKRHIRTHTGEKPFQCRICMANFSDPSNLRRHIRTHTGLR

SEQ ID NO: 15 - protein, 86 aa, Artificial Sequence
  92ZFWT
ERPFQCRICMRNFSDSPTLRRHIRTHTGEKPFQCRICMRNFSQRSSLVRHIRTHTGEKPFQCRICMRNFSERGNLTRHIRTHTGLR

SEQ ID NO: 16 - protein, 86 aa, Artificial Sequence
  92ZFR39A
ERPFQCRICMRNFSDSPTLRRHIRTHTGEKPFQCRICMANFSQRSSLVRHIRTHTGEKPFQCRICMRNFSERGNLTRHIRTHTGLR

SEQ ID NO: 17 - protein, 86 aa, Artificial Sequence
  92ZFR2AR39A
EAPFQCRICMRNFSDSPTLRRHIRTHTGEKPFQCRICMANFSQRSSLVRHIRTHTGEKPFQCRICMRNFSERGNLTRHIRTHTGLR

SEQ ID NO: 18 - protein, 87 aa, Artificial Sequence
  92ZFR2AR39AR67A
EAPFQCRICMRNFSDSPTLRRHTRTHTGEKPFQCRICMANFSQRSSLVRHLRTHTGEKPFQCRICMANFSERGNLTRHLKTHTGEKG

SEQ ID NO: 19 - protein, 86 aa, Artificial Sequence
  97ZFWT
ERPFQCRICMRNFSRQSNLSRHIRTHTGEKPFQCRICMRNFSRNEHLVLHIRTHTGEKPFQCRICMRNFSQKTGLRVHIRTHTGLR

SEQ ID NO: 20 - protein, 86 aa, Artificial Sequence
  97ZFR39A
ERPFQCRICMRNFSRQSNLSRHIRTHTGEKPFQCRICMANFSRNEHLVLHIRTHTGEKPFQCRICMRNFSQKTGLRVHIRTHTGLR

SEQ ID NO: 21 - protein, 86 aa, Artificial Sequence
  97ZFR2AR39A
EAPFQCRICMRNFSRQSNLSRHIRTHTGEKPFQCRICMANFSRNEHLVLHIRTHTGEKPFQCRICMRNFSQKTGLRVHIRTHTGLR

SEQ ID NO: 22 - protein, 87 aa, Artificial Sequence
  BCRZF
ERPFQCRICMRNFSDSPTLRRHTRTHTGEKPFQCRICMRNFSQGANLRRHLRTHTGEKPFQCRICMRNFSQANTLQRHLKTHTGEKG

SEQ ID NO: 23 - protein, 87 aa, Artificial Sequence
  BCRZFR39A
ERPFQCRICMRNFSDSPTLRRHTRTHTGEKPFQCRICMANFSQGANLRRHLRTHTGEKPFQCRICMRNFSQANTLQRHLKTHTGEKG

SEQ ID NO: 24 - protein, 87 aa, Artificial Sequence
  HIV1ZFWT
ERPFQCRICMRNFSLRTDLDRHTRTHTGEKPFQCRICMRNFSLSQTLRRHLRTHTGEKPFQCRICMRNFSLRSNLGRHLKTHTGEKG

SEQ ID NO: 25 - protein, 87 aa, Artificial Sequence
  HIV1ZFR39A
ERPFQCRICMRNFSLRTDLDRHTRTHTGEKPFQCRICMANFSLSQTLRRHLRTHTGEKPFQCRICMRNFSLRSNLGRHLKTHTGEKG

SEQ ID NO: 26 - protein, 87 aa, Artificial Sequence
  HIV1ZFR2AR39A
EAPFQCRICMRNFSLRTDLDRHTRTHTGEKPFQCRICMANFSLSQTLRRHLRTHTGEKPFQCRICMRNFSLRSNLGRHLKTHTGEKG

SEQ ID NO: 27 - protein, 87 aa, Artificial Sequence
  HIV1ZFR2AR39AR67A
EAPFQCRICMRNFSLRTDLDRHTRTHTGEKPFQCRICMANFSLSQTLRRHLRTHTGEKPFQCRICMANFSLRSNLGRHLKTHTGEKG

SEQ ID NO: 28 - protein, 87 aa, Artificial Sequence
  HIV2ZFWT
ERPFQCRICMRNFSNNAMLVRHTRTHTGEKPFQCRICMRNFSLSQTLQRHLRTHTGEKPFQCRICMRNFSMQGNLSRHLKTHTGEKG

SEQ ID NO: 29 - protein, 87 aa, Artificial Sequence
  HIV2ZFR39A
ERPFQCRICMRNFSNNAMLVRHTRTHTGEKPFQCRICMANFSLSQTLQRHLRTHTGEKPFQCRICMRNFSMQGNLSRHLKTHTGEKG

SEQ ID NO: 30 - protein, 87 aa, Artificial Sequence
  HIV2ZFR2AR39A
EAPFQCRICMRNFSNNAMLVRHTRTHTGEKPFQCRICMANFSLSQTLQRHLRTHTGEKPFQCRICMRNFSMQGNLSRHLKTHTGEKG

SEQ ID NO: 31 - protein, 87 aa, Artificial Sequence
  HIV2ZFR2AR39AR67A
EAPFQCRICMRNFSNNAMLVRHTRTHTGEKPFQCRICMANFSLSQTLQRHLRTHTGEKPFQCRICMANFSMQGNLSRHLKTHTGEKG

SEQ ID NO: 32 - protein, 467 aa, Artificial Sequence
  NLS-FKBP12F36V-37ZFR2AR11AR39AR67A-VP16-NLS- DHFR
PKKKRKVSGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGSEAPFQCRICMANFSRNFILQRHTRTHTGEKPFQCRICMANFSDRANLRRHLRTHTGEKPFQCRICMANFSRHDQLTRHLKTHTGEKGGGSSGAPPTDVSLGDELHLDGEDVAMAHADALDDFDLDMLGDGDSPGPGFTPHDSAPYGALDMADFEFEQMFTDALGIDEYGGGSPKKKRKVGSSDYKDDDDKSSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 33 - protein, 467 aa, Artificial Sequence
  NLS-FKBP12F36V-BCRZFR39A-VP16-NLS-DHFR
PKKKRKVSGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGSERPFQCRICMRNFSDSPTLRRHTRTHTGEKPFQCRICMANFSQGANLRRHLRTHTGEKPFQCRICMRNFSQANTLQRHLKTHTGEKGGGSSGAPPTDVSLGDELHLDGEDVAMAHADALDDFDLDMLGDGDSPGPGFTPHDSAPYGALDMADFEFEQMFTDALGIDEYGGGSPKKKRKVGSSDYKDDDDKSSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 34 - protein, 467 aa, Artificial Sequence
  NLS-FKBP12F36V-ErbB2ZFR2AR39A-VP16-NLS-DHFR
PKKKRKVSGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGSEAPFQCRICMRNFSRSDVLANHTRTHTGEKPFQCRICMANFSQSSTLTRHLRTHTGEKPFQCRICMRNFSERQGLKRHLKTHTGEKGGGSSGAPPTDVSLGDELHLDGEDVAMAHADALDDFDLDMLGDGDSPGPGFTPHDSAPYGALDMADFEFEQMFTDALGIDEYGGGSPKKKRKVGSSDYKDDDDKSSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

(19) United States
(12) Patent Application Publication
(10) Pub. No.: US 2022/0177911 A1
(43) Pub. Date: Jun. 9, 2022

(54) SYNTHETIC CIRCUIT FOR CELLULAR MULTISTABILITY

(71) Applicant: California Institute of Technology, Pasadena, CA (US)

(72) Inventors: Ronghui Zhu, Pasadena, CA (US); Michael B. Elowitz, Pasadena, CA (US)

(21) Appl. No.: 17/544,865

(22) Filed: Dec. 7, 2021

Related U.S. Application Data
(60) Provisional application No. 63/122,850, filed on Dec. 8, 2020.

(51) Int. Cl. C12N 15/85; C07K 14/47

(52) U.S. Cl. CPC C12N 15/85; C07K 14/4705

(57) ABSTRACT

Disclosed herein include circuits, compositions, nucleic acids, populations, systems, and methods enabling single circuits to generate multiple molecularly and functionally distinct states that are each stable across multiple cell division cycles. Synthetic circuits provided herein can stably exist in multiple distinct states characterized by differences in the concentrations and expression levels of its components. In the absence of changes to the external environment, each of these states can be stable. In some embodiments, transcription factors provided herein activate when dimerized, and show much weaker activity as monomers. In some embodiments, each transcription factor homodimer activates expression of its own gene. In some embodiments, transcription factors can form mixed heterodimers with one another that do not strongly activate any genes in the circuit. Different embodiments of the synthetic circuits provided herein can use different numbers of transcription factors to produce a growing number of stable states.

### RELATED APPLICATIONS

This application claims the benefit under 35 U.S.C. § 119(e) of U.S. Provisional Patent Application Ser. No. 63/122,850, filed Dec. 8, 2020, the content of this related application is incorporated herein by reference in its entirety for all purposes.

### STATEMENT REGARDING FEDERALLY SPONSORED R&D

This invention was made with government support under Grant No. HR0011-17-2-008 awarded by DARPA. The government has certain rights in the invention.

### REFERENCE TO SEQUENCE LISTING

The present application is being filed along with a Sequence Listing in electronic format. The Sequence Listing is provided as a file entitled 30KJ_302437_US_Sequence Listing, created Dec. 6, 2021, which is 36 kilobytes in size. The information in the electronic format of the Sequence Listing is incorporated herein by reference in its entirety.

### BACKGROUND

### Field

The present disclosure relates generally to the field of synthetic biology.

### Description of the Related Art

Synthetic biology involves the engineering of biological circuits that can generate useful new functions in living cells. There is a need for single circuits that can generate multiple molecularly and functionally distinct states that are each stable across multiple cell division cycles. The key property required for this is termed multistability, defined as the ability of the circuit to stably exist in multiple distinct states characterized by differences in the concentrations and expression levels of its components. In the absence of changes to the external environment, each of these states should ideally be stable. There is a need for compositions, methods, systems, and kits for the generation of multistable synthetic circuits.

### SUMMARY

Disclosed herein include nucleic acid compositions. In some embodiments, the nucleic acid composition comprises: a first promoter operably linked to a first polynucleotide encoding a first transcription factor (TF) and to a second polynucleotide encoding one or more first payloads. In some embodiments, the first promoter comprises one or more pairs of first TF binding sites. In some embodiments, the first TF comprises a first DNA-binding domain capable of binding a first TF binding site. In some embodiments, the first TF comprises a dimerization domain. In some embodiments, the dimerization domain of two first TF are capable of associating to generate a first TF homodimer. In some embodiments, a first TF homodimer is capable of binding the pair of first TF binding sites. In some embodiments, the dimerization domain of each of two first TF are capable of associating to generate the first TF homodimer in the presence of a dimerization ligand. In some embodiments, the dimerization ligand is a dimeric ligand. In some embodiments, the dimerization domain of each of two first TF are incapable of associating to generate the first TF homodimer in the absence of the dimerization ligand.

In some embodiments, the first TF comprises a transactivation domain. In some embodiments, the first TF further comprises a degron capable of binding a degron stabilizing molecule. In some embodiments, the first TF changes from a destabilized state to a stabilized state when the degron binds to the degron stabilizing molecule. In some embodiments, the one or more first payloads comprise one or more first payload proteins and/or one or more first payload RNA agents. In some embodiments, upon the first TF homodimer binding a pair of first TF binding sites, the first promoter is capable of inducing transcription of the first polynucleotide and the second polynucleotide to generate a first polycistronic transcript. In some embodiments, the first polynucleotide and the second polynucleotide are operably linked to a tandem gene expression element. In some embodiments, the tandem gene expression element is an internal ribosomal entry site (IRES). In some embodiments, the first polycistronic transcript is capable of being translated to generate the first TF and the one or more first payloads. In some embodiments, the first promoter further comprises one or more copies of a transactivator recognition sequence that a transactivator is capable of binding. In some embodiments, in the presence of the transactivator and a transactivator-binding compound, the first promoter is capable of inducing transcription of the first polynucleotide and the second polynucleotide to generate the first polycistronic transcript. In some embodiments, the first promoter further comprises one or more copies of a basal expression motif capable of inducing transcription of the first polynucleotide and the second polynucleotide to generate the first polycistronic transcript. In some embodiments, the basal expression motif comprises (GACGCTGCT). In some embodiments, the first promoter further comprises one or more first input elements capable of inducing or repressing transcription of the first polynucleotide and the second polynucleotide upon a first input reaching a threshold first input level.

Disclosed herein include nucleic acid compositions. In some embodiments, the nucleic acid composition comprises: a second promoter operably linked to a third polynucleotide encoding a second transcription factor (TF) and to a fourth polynucleotide encoding one or more second payloads. In some embodiments, the second promoter comprises one or more pairs of second TF binding sites. In some embodiments, the second TF comprises a second DNA-binding domain capable of binding a second TF binding site. In some embodiments, the second TF comprises a dimerization domain. In some embodiments, the dimerization domain of two second TF are capable of associating to generate a second TF homodimer. In some embodiments, a second TF homodimer is capable of binding the pair of second TF binding sites. In some embodiments, the dimerization domain of each of two second TF are capable of associating to generate the second TF homodimer in the presence of a dimerization ligand. In some embodiments, the dimerization ligand is a dimeric ligand. In some embodiments, the dimerization domain of each of two second TF are incapable of associating to generate the second TF homodimer in the absence of the dimerization ligand.

In some embodiments, the second TF comprises a transactivation domain. In some embodiments, the second TF further comprises a degron capable of binding a degron stabilizing molecule. In some embodiments, the second TF changes from a destabilized state to a stabilized state when the degron binds to the degron stabilizing molecule. In some embodiments, the one or more second payloads comprise one or more second payload proteins and/or one or more second payload RNA agents. In some embodiments, upon the second TF homodimer binding a pair of second TF binding sites, the second promoter is capable of inducing transcription of the third polynucleotide and the fourth polynucleotide to generate a second polycistronic transcript. In some embodiments, the third polynucleotide and the fourth polynucleotide are operably linked to a tandem gene expression element. In some embodiments, the tandem gene expression element is an internal ribosomal entry site (IRES). In some embodiments, the second polycistronic transcript is capable of being translated to generate the second TF and the one or more second payloads. In some embodiments, the second promoter further comprises one or more copies of a transactivator recognition sequence that a transactivator is capable of binding. In some embodiments, in the presence of the transactivator and a transactivator-binding compound, the second promoter is capable of inducing transcription of the third polynucleotide and the fourth polynucleotide to generate the second polycistronic transcript. In some embodiments, the second promoter further comprises one or more copies of a basal expression motif capable of inducing transcription of the third polynucleotide and the fourth polynucleotide to generate the second polycistronic transcript. In some embodiments, the basal expression motif comprises (GACGCTGCT). In some embodiments, the second promoter further comprises one or more second input elements capable of inducing or repressing transcription of the third polynucleotide and the fourth polynucleotide upon a second input reaching a threshold second input level.

Disclosed herein include nucleic acid compositions. In some embodiments, the nucleic acid composition comprises: a third promoter operably linked to a fifth polynucleotide encoding a third transcription factor (TF) and to a sixth polynucleotide encoding one or more third payloads. In some embodiments, the third promoter comprises one or more pairs of third TF binding sites. In some embodiments, the third TF comprises a third DNA-binding domain capable of binding a third TF binding site. In some embodiments, the third TF comprises a dimerization domain. In some embodiments, the dimerization domain of two third TF are capable of associating to generate a third TF homodimer. In some embodiments, a third TF homodimer is capable of binding the pair of third TF binding sites. In some embodiments, the dimerization domain of each of two third TF are capable of associating to generate the third TF homodimer in the presence of a dimerization ligand. In some embodiments, the dimerization ligand is a dimeric ligand. In some embodiments, the dimerization domain of each of two third TF are incapable of associating to generate the third TF homodimer in the absence of the dimerization ligand.

In some embodiments, the third TF comprises a transactivation domain. In some embodiments, the third TF further comprises a degron capable of binding a degron stabilizing molecule. In some embodiments, the third TF changes from a destabilized state to a stabilized state when the degron binds to the degron stabilizing molecule. In some embodiments, the one or more third payloads comprise one or more third payload proteins and/or one or more third payload RNA agents. In some embodiments, upon the third TF homodimer binding a pair of third TF binding sites, the third promoter is capable of inducing transcription of the fifth polynucleotide and the sixth polynucleotide to generate a third polycistronic transcript. In some embodiments, the fifth polynucleotide and the sixth polynucleotide are operably linked to a tandem gene expression element. In some embodiments, the tandem gene expression element is an internal ribosomal entry site (IRES). In some embodiments, the third polycistronic transcript is capable of being translated to generate the third TF and the one or more third payloads. In some embodiments, the third promoter further comprises one or more copies of a transactivator recognition sequence that a transactivator is capable of binding. In some embodiments, in the presence of the transactivator and a transactivator-binding compound, the third promoter is capable of inducing transcription of the fifth polynucleotide and the sixth polynucleotide to generate the third polycistronic transcript. In some embodiments, the third promoter further comprises one or more copies of a basal expression motif capable of inducing transcription of the fifth polynucleotide and the sixth polynucleotide to generate the third polycistronic transcript. In some embodiments, the basal expression motif comprises (GACGCTGCT). In some embodiments, the third promoter further comprises one or more third input elements capable of inducing or repressing transcription of the fifth polynucleotide and the sixth polynucleotide upon a third input reaching a threshold third input level.

Disclosed herein include nucleic acid compositions. In some embodiments, the nucleic acid composition comprises: n supplemental promoters each operably linked to a nth supplemental polynucleotide encoding an nth supplemental transcription factor (sTF) and to a (n+1)th supplemental polynucleotide encoding one or more nth supplemental payloads. In some embodiments, n is 1, 2, 3, 4, 5, or 6. In some embodiments, the nth supplemental promoter comprises one or more pairs of nth supplemental TF binding sites. In some embodiments, the nth supplemental TF comprises a nth supplemental DNA-binding domain capable of binding a nth supplemental TF binding site. In some embodiments, the nth supplemental TF comprises a dimerization domain. In some embodiments, the dimerization domain of two nth supplemental TF are capable of associating to generate a nth supplemental TF homodimer. In some embodiments, a nth supplemental TF homodimer is capable of binding the pair of nth supplemental TF binding sites. In some embodiments, the dimerization domain of each of two nth supplemental TF are capable of associating to generate the nth supplemental TF homodimer in the presence of a dimerization ligand. In some embodiments, the dimerization ligand is a dimeric ligand. In some embodiments, the dimerization domain of each of two nth supplemental TF are incapable of associating to generate the nth supplemental TF homodimer in the absence of the dimerization ligand.

In some embodiments, the nth supplemental TF comprises a transactivation domain. In some embodiments, the nth supplemental TF further comprises a degron capable of binding a degron stabilizing molecule. In some embodiments, the nth supplemental TF changes from a destabilized state to a stabilized state when the degron binds to the degron stabilizing molecule. In some embodiments, the one or more nth supplemental payloads comprise one or more nth supplemental payload proteins and/or one or more nth supplemental payload RNA agents. In some embodiments, upon the nth supplemental TF homodimer binding a pair of nth supplemental TF binding sites, the nth supplemental promoter is capable of inducing transcription of the nth supplemental polynucleotide and the (n+1)th supplemental polynucleotide to generate a nth supplemental polycistronic transcript. In some embodiments, the nth supplemental polynucleotide and the (n+1)th supplemental polynucleotide are operably linked to a tandem gene expression element. In some embodiments, the tandem gene expression element is an internal ribosomal entry site (IRES). In some embodiments, the nth supplemental polycistronic transcript is capable of being translated to generate the nth supplemental TF and the one or more nth supplemental payloads. In some embodiments, the nth supplemental promoter further comprises one or more copies of a transactivator recognition sequence that a transactivator is capable of binding. In some embodiments, in the presence of the transactivator and a transactivator-binding compound, the nth supplemental promoter is capable of inducing transcription of the nth supplemental polynucleotide and the (n+1)th supplemental polynucleotide to generate the nth supplemental polycistronic transcript. In some embodiments, the nth supplemental promoter further comprises one or more copies of a basal expression motif capable of inducing transcription of the nth supplemental polynucleotide and the (n+1)th supplemental polynucleotide to generate the nth supplemental polycistronic transcript. In some embodiments, the basal expression motif comprises (GACGCTGCT). In some embodiments, the nth supplemental promoter further comprises one or more nth supplemental input elements capable of inducing or repressing transcription of the nth supplemental polynucleotide and the (n+1)th supplemental polynucleotide upon a nth supplemental input reaching a threshold nth supplemental input level.

Disclosed herein include nucleic acid compositions. In some embodiments, the nucleic acid composition comprises: two or more of the nucleic acid compositions disclosed herein.

In some embodiments, the dimerization domain comprises or is derived from GCN4, FKBP, cyclophilin, steroid binding protein, estrogen binding protein, glucocorticoid binding protein, vitamin D binding protein, tetracycline binding protein, extracellular domain of a cytokine receptor, a receptor tyrosine kinase, a TNFR-family receptor, an immune co-receptor, or any combination thereof. In some embodiments, the dimerization domain comprises an amino acid sequence at least 50, 60, 70, 75, 80, 85, 90, 95, 96, 97, 98, or 99 percent identical to FKBP12F36V (SEQ ID NO: 5). In some embodiments, the dimerization domain comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the dimerization domain is a homodimerization domain or a multimerization domain (e.g., a homo- or hetero-dimerizing or multimerizing leucine zipper, a PDZ domains, a SH3 domain, aGBD domain, or any combination thereof). In some embodiments, the dimerization ligand comprises or is derived from AP1903, AP20187, dimeric FK506, a dimeric FK506-like analog, derivatives thereof, or any combination thereof. In some embodiments, the dimerization domain enables dose-dependent control of TF activation by the dimerization ligand. In some embodiments, the dimerization domain of the first TF, the second TF, the third TF, and/or nth sTF are the same. In some embodiments, the dimerization domain of the first TF, the second TF, the third TF, and/or nth sTF are different. In some embodiments, the dimerization domains of (i) a first TF and a second TF, (ii) a first TF and a third TF, (iii) a first TF and an nth sTF; (iv) a second TF and a third TF, (v) a second TF and a nth sTF, and/or (vi) a third TF and a nth sTF, are capable of associating to generate a TF heterodimer, In some embodiments, the dimerization domains of (i) a first TF and a second TF, (ii) a first TF and a third TF, (iii) a first TF and an nth sTF; (iv) a second TF and a third TF, (v) a second TF and a nth sTF, and/or (vi) a third TF and a nth sTF, are capable of associating to generate a TF heterodimer in the presence of a dimerization ligand. In some embodiments, the dimerization ligand is a dimeric ligand.

In some embodiments, a TF heterodimer has at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, or 100-fold, less binding affinity for a pair of TF binding sites as compared to a TF homodimer. In some embodiments, a TF heterodimer is not capable of binding a pair of TF binding sites. In some embodiments, a first promoter, second promoter, third promoter, and/or nth supplemental promoter induces transcription at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, or 100-fold, less in the presence of a TF heterodimer as compared to a TF homodimer. In some embodiments, a TF heterodimer is incapable of causing a first promoter, second promoter, third promoter, and/or nth supplemental promoter to induce transcription. In some embodiments, a TF monomer has at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, or 100-fold, less binding affinity for a pair of TF binding sites as compared to a TF homodimer. In some embodiments, a first promoter, second promoter, third promoter, and/or nth supplemental promoter induces transcription at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, or 100-fold, less in the presence of a TF monomer as compared to a TF homodimer. In some embodiments, TF homodimerization and heterodimerization occur with a substantially equal dissociation constant (K_{d}).

In some embodiments, a DNA-binding domain comprises or is derived from: a TALE DNA binding domain 2, catalytically dead CRISPR/Cas9 (dCas9) 3-5, Gal4, hypoxia inducible factor (HIF), HIF1a, cyclic AMP response element binding (CREB) protein, LexA, rtTA, an endonuclease, a zinc finger (ZF) binding domain, a transcription factor, portions thereof, or any combination thereof. In some embodiments, the DNA-binding domain is a synthetic DNA-binding domain configured to decrease monomeric TF activity without reducing TF homodimer activity. In some embodiments, a DNA-binding domain comprises or is derived from a zinc finger DNA-binding domain. In some embodiments, the zinc finger (ZF) DNA-binding domain comprises or is derived from ErbB2 ZF, BCRZF, HIV1ZF, HIV2ZF, 37ZF (37-12 array), 42ZF (42-10 array), 43ZF (43-8 array), 92ZF (92-1 array), and/or 97ZF (97-4 array). In some embodiments, the ZF DNA-binding domain comprises one or more arginine-to-alanine mutations. In some embodiments, the ZF DNA-binding domain comprises three fingers that bind weakly as monomers to 9 bp target sites and bind at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, or 100-fold, more strongly as homodimers to 18 bp tandem binding site pairs. In some embodiments, a DNA-binding domain comprises an amino acid sequence at least 50, 60, 70, 75, 80, 85, 90, 95, 96, 97, 98, or 99 percent identical to ErbB2ZFWT (SEQ ID NO: 6), ErbB2ZFR39A (SEQ ID NO: 7), ErbB2ZFR2AR39A (SEQ ID NO: 8), ErbB2ZFR2AR39AR67A (SEQ ID NO: 9), 37ZFWT (SEQ ID NO: 10), 37ZFR39A (SEQ ID NO: 11), 37ZFR2AR39A (SEQ ID NO: 12), 37ZFR2AR39AR67A (SEQ ID NO: 13), 42ZFR2AR39AR67A (SEQ ID NO: 14), 92ZFWT (SEQ ID NO: 15), 92ZFR39A (SEQ ID NO: 16), 92ZFR2AR39A (SEQ ID NO: 17), 92ZFR2AR39AR67A (SEQ ID NO: 18), 97ZFWT (SEQ ID NO: 19), 97ZFR39A (SEQ ID NO: 20), 97ZFR2AR39A (SEQ ID NO: 21), BCRZF (SEQ ID NO: 22), BCRZFR39A (SEQ ID NO: 23), HIV1ZFWT (SEQ ID NO: 24), HIV1ZFR39A (SEQ ID NO: 25), HIV1ZFR2AR39A (SEQ ID NO: 26), HIV1ZFR2AR39AR67A (SEQ ID NO: 27), HIV2ZFWT (SEQ ID NO: 28), HIV2ZFR39A (SEQ ID NO: 29), HIV2ZFR2AR39A (SEQ ID NO: 30), and/or HIV2ZFR2AR39AR67A (SEQ ID NO: 31). In some embodiments, the first TF, the second TF, the third TF, and/or nth sTF share substantially identical biochemical parameters and differ only in their DNA binding site specificity. In some embodiments, the first TF, the second TF, the third TF, and/or nth sTF have orthogonal DNA-binding specificities. In some embodiments, the pair of first TF binding sites, the pair of second TF binding sites, the pair of third TF binding sites, and/or the pair of nth supplemental TF binding sites is at least 50, 60, 70, 75, 80, 85, 90, 95, 96, 97, 98, or 99 percent identical to 42bs_42bs (SEQ ID NO: 1), 37bs_37bs (SEQ ID NO: 2), BCRbs_BCRbs (SEQ ID NO: 3), ErbB2bs_ErbB2bs (SEQ ID NO: 4), portions thereof, or any combination thereof.

In some embodiments, a transactivation domain comprises or is derived from VP16, TA2, VP64 (a tetrameric repeat of the minimal activation domain of VP16), VP48 (a trimeric repeat of the minimal activation domain of VP16), signal transducer and activator of transcription 6 (STAT6), reticuloendotheliosis virus A oncogene (relA), TATA binding protein associated factor-1 (TAF-1), TATA binding protein associated factor-2 (TAF-2), glucocorticoid receptor TAU-1, or glucocorticoid receptor TAU-2, a steroid/thyroid hormone nuclear receptor transactivation domain, a polyglutamine transactivation domain, a basic or acidic amino acid transactivation domain, a GAL4 transactivation domain, an NF-κB transactivation domain, a p65 transactivation domain, a BP42 transactivation domain, HSF1, VP16, VP64, p65, MyoD1, RTA, SET7/9, VPR, histone acetyltransferase p300, an hydroxylase catalytic domain of a TET family protein (e.g., TETl hydroxylase catalytic domain), LSD1, CIB1, AD2, CR3, EKLF1, GATA4, PRVIE, p53, SP1, MEF2C, TAX, and PPARγ, Gal4, Gcn4, MLL, Rtg3, Gln3, Oaf1, Pip2, Pdr1, Pdr3, Pho4, Leu3, portions thereof having transcription activating activity, or any combination thereof. In some embodiments, the transactivation domain of the first TF, the second TF, the third TF, and/or nth sTF are the same. In some embodiments, the transactivation domain of the first TF, the second TF, the third TF, and/or nth sTF are different.

In some embodiments, the nucleic acid composition comprises: one or more polynucleotides encoding a transactivator. In some embodiments, the one or more polynucleotides encoding a transactivator are under the control of a ubiquitous promoter. In some embodiments, the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

In some embodiments, a transactivator recognition sequence comprises a Tet3G binding site (TRE3G) or a ERT2-Gal4 binding site (UAS). In some embodiments, the transactivator-binding compound comprises 4-hydroxy-tamoxifen (4-OHT), Dox, derivatives thereof, or any combination thereof. In some embodiments, in the presence of the transactivator and a transactivator-binding compound, the first promoter is capable of inducing transcription up to, but not substantially beyond, the level produced by a TF homodimer binding a pair of TF binding sites. In some embodiments, a transactivator recognition sequence comprises an element of an inducible promoter. In some embodiments, the inducible promoter is a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, or an RU-486 responsive promoter.

In some embodiments, the degron comprises a dihydrofolate reductase (DHFR) degron, a FKB protein (FKBP) degron, derivatives thereof, or any combination thereof. In some embodiments, the degron stabilizing molecule comprises trimethoprim (TMP), Shield-1, derivatives thereof, or any combination thereof.

In some embodiments, the first TF, the second TF, the third TF, and/or nth sTF is capable of self-activating and sustaining its own expression. In some embodiments, the first TF, the second TF, the third TF, and/or nth sTF comprises an amino acid sequence at least 50, 60, 70, 75, 80, 85, 90, 95, 96, 97, 98, or 99 percent identical to NLS-FKBP12F36V-37ZFR2AR11AR39AR67A-VP16-NLS-DHFR (SEQ ID NO: 32), NLS-FKBP12F36V-BCRZFR39A-VP16-NLS-DHFR (SEQ ID NO: 33), or NLS-FKBP12F36V-ErbB2ZFR2AR39A-VP16-NLS-DHFR (SEQ ID NO: 34). In some embodiments, one or more of the first TF, the second TF, the third TF, and/or nth sTF are configured to homodimerize and to not heterodimerize with another TF. In some embodiments, one or more of the first TF, the second TF, the third TF, and/or nth sTF are configured to homodimerize and to heterodimerize with a subset of TFs.

In some embodiments, a payload comprises a component of a synthetic protein circuit. In some embodiments, a payload protein comprises fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. In some embodiments, a payload protein comprises nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. In some embodiments, a payload protein comprises a CRE recombinase, GCaMP, a cell therapy component, a knock-down gene therapy component, a cell-surface exposed epitope, or any combination thereof. In some embodiments, a payload protein comprises a diagnostic agent. In some embodiments, the diagnostic agent comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCitrine, mCherry, mruby3, rsCherry, rsCherryRev, derivatives thereof, or any combination thereof. In some embodiments, a payload encodes a siRNA, a shRNA, an antisense RNA oligonucleotide, an antisense miRNA, a trans-splicing RNA, a guide RNA, single-guide RNA, crRNA, a tracrRNA, a trans-splicing RNA, a pre-mRNA, a mRNA, or any combination thereof. In some embodiments, a payload protein comprises a synthetic protein circuit component. In some embodiments, a payload comprises a bispecific T cell engager (BiTE). In some embodiments, a payload protein comprises a cytokine. In some embodiments, the cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof. In some embodiments, a payload protein comprises a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. In some embodiments, a payload protein comprises a member of the TNF family of cytokines selected from the group consisting of TNF-alpha, TNF-beta, LT-beta, CD40 ligand, Fas ligand, CD 27 ligand, CD 30 ligand, and 4-1 BBL. In some embodiments, a payload protein comprises a member of the immunoglobulin superfamily of cytokines selected from the group consisting of B7.1 (CD80) and B7.2 (B70). In some embodiments, a payload protein comprises an interferon. In some embodiments, the interferon is selected from interferon alpha, interferon beta, or interferon gamma. In some embodiments, a payload protein comprises a chemokine. In some embodiments, the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. In some embodiments, a payload protein comprises an interleukin. In some embodiments, the interleukin is selected from IL-10 IL-12, IL-1, IL-6, IL-7, IL-15, IL-2, IL-18 or IL-21. In some embodiments, a payload protein comprises a tumor necrosis factor (TNF). In some embodiments, the TNF is selected from TNF-alpha, TNF-beta, TNF-gamma, CD252, CD154, CD178, CD70, CD153, or 4-1BBL. In some embodiments, a payload protein comprises a factor locally down-regulating the activity of endogenous immune cells. In some embodiments, a payload protein is capable of remodeling a tumor microenvironment and/or reducing immunosuppression at a target site of a subject.

In some embodiments, a payload protein comprises a chimeric antigen receptor (CAR) or T-cell receptor (TCR). In some embodiments, the CAR and/or TCR comprises one or more of an antigen binding domain, a transmembrane domain, and an intracellular signaling domain. In some embodiments, the intracellular signaling domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCERIG), FcR beta (Fc Epsilon Rib), CD79a, CD79b, Fcgamma RIIa, DAP10, and DAP12, or a functional variant thereof. In some embodiments, the costimulatory domain comprises a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

In some embodiments, the antigen binding domain binds a tumor antigen. In some embodiments, the tumor antigen is a solid tumor antigen. In some embodiments, the tumor antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). In some embodiments, the tumor antigen is selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, 02-Microglobulin, Fc Receptor-like 5 (FcRL5), or molecules expressed by HIV, HCV, HBV, or other pathogens.

In some embodiments, the antigen binding domain comprises an antibody, an antibody fragment, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. In some embodiments, the antigen binding domain is connected to the transmembrane domain by a hinge region. In some embodiments, the transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Rα, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. In some embodiments, the CAR or TCR further comprises a leader peptide. In some embodiments, the TCR further comprises a constant region and/or CDR4.

In some embodiments, a payload protein is an activity regulator. In some embodiments, the activity regulator is capable of reducing T cell activity. In some embodiments, the activity regulator comprises a ubiquitin ligase involved in TCR/CAR signal transduction selected from the group comprising c-CBL, CBL-B, ITCH, R F125, R F128, WWP2, or any combination thereof. In some embodiments, the activity regulator comprises a negative regulatory enzyme selected from the group comprising SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof. In some embodiments, the activity regulator is a negative regulatory scaffold/adapter protein selected from the group comprising PAG, LIME, NTAL, LAX31, SIT, GAB2, GRAP, ALX, SLAP, SLAP2, DOK1, DOK2, or any combination thereof. In some embodiments, the activity regulator is a dominant negative version of an activating TCR signaling component selected from the group comprising ZAP70, LCK, FYN, NCK, VAV1, SLP76, ITK, ADAP, GADS, PLCgamma1, LAT, p85, SOS, GRB2, NFAT, p50, p65, API, RAPI, CRKII, C3G, WAVE2, ARP2/3, ABL, ADAP, RIAM, SKAP55, or any combination thereof. In some embodiments, the activity regulator comprises the cytoplasmic tail of a negative co-regulatory receptor selected from the group comprising CD5, PD1, CTLA4, BTLA, LAG3, B7-H1, B7-1, CD160, TFM3, 2B4, TIGIT, or any combination thereof. In some embodiments, the activity regulator is targeted to the plasma membrane with a targeting sequence derived from LAT, PAG, LCK, FYN, LAX, CD2, CD3, CD4, CD5, CD7, CD8a, PD1, SRC, LYN, or any combination thereof. In some embodiments, the activity regulator reduces or abrogates a pathway and/or a function selected from the group comprising Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, or any combination thereof.

In some embodiments, a payload protein is capable of modulating the concentration, localization, stability, and/or activity of the one or more targets. In some embodiments, a payload protein is capable of repressing the transcription of the one or more targets. In some embodiments, a target transcript is capable of being translated to generate a target protein. In some embodiments, a payload protein is capable of reducing the concentration, localization, stability, and/or activity of the target protein. In some embodiments, the concentration, localization, stability, and/or activity of the target protein is inversely related to the concentration, localization, stability, and/or activity of a payload protein. In some embodiments, a payload protein comprises a protease. In some embodiments, the target protein comprises a degron and a cut site the protease is capable of cutting to expose the degron. In some embodiments, the degron of the target protein being exposed changes the target protein to a target protein destabilized state. In some embodiments, the protease comprises tobacco etch virus (TEV) protease, tobacco vein mottling virus (TVMV) protease, hepatitis C virus protease (HCVP), derivatives thereof, or any combination thereof. In some embodiments, the target protein comprises a cage polypeptide, wherein the cage polypeptide comprises: (a) a helical bundle, comprising between 2 and 7 alpha-helices, wherein the helical bundle comprises: (i) a structural region; and (ii) a latch region, wherein the latch region comprises a degron located within the latch region, wherein the structural region interacts with the latch region to prevent activity of the degron; and (b) amino acid linkers connecting each alpha helix. In some embodiments, a payload protein comprises a key polypeptide capable of binding to the cage polypeptide structural region, thereby displacing the latch region and activating the degron.

In some embodiments, a payload protein comprises a programmable nuclease. In some embodiments, the programmable nuclease is selected from the group comprising: SpCas9 or a derivative thereof, VRER, VQR, EQR SpCas9; xCas9-3.7; eSpCas9; Cas9-H1F1; HypaCas9; evoCas9; HiFi Cas9; ScCas9; StCas9; NmCas9; SaCas9; CjCas9; CasX; Cas9 H940A nickase; Cas12 and derivatives thereof, dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. In some embodiments, the programmable nuclease comprises a zinc finger nuclease (ZFN) and/or transcription activator-like effector nuclease (TALEN). In some embodiments, the programmable nuclease comprises Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. In some embodiments, the nucleic acid composition comprises: a polynucleotide encoding (i) a targeting molecule and/or (ii) a donor nucleic acid. In some embodiments, a payload comprises (i) a targeting molecule and/or (ii) a donor nucleic acid. In some embodiments, the targeting molecule is capable of associating with the programmable nuclease. In some embodiments, the targeting molecule comprises single strand DNA or single strand RNA. In some embodiments, the targeting molecule comprises a single guide RNA (sgRNA).

In some embodiments, the payload comprises a pro-death protein is capable of halting cell growth and/or inducing cell death. In some embodiments, the pro-death protein comprises cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. In some embodiments, the pro-death protein is capable of halting cell growth and/or inducing cell death in the presence of a pro-death agent. In some embodiments, the pro-death protein comprises Caspase-9 and the pro-death agent comprises AP1903; the pro-death protein comprises HSV thymidine kinase (TK) and the pro-death agent Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT); the pro-death protein comprises Cytosine Deaminase (CD) and the pro-death agent comprises 5-fluorocytosine (5-FC); the pro-death protein comprises Purine nucleoside phosphorylase (PNP) and the pro-death agent comprises 6-methylpurine deoxyriboside (MEP) and/or fludarabine (FAMP); the pro-death protein comprises a Cytochrome p450 enzyme (CYP) and the pro-death agent comprises Cyclophosphamide (CPA), Ifosfamide (IFO), and/or 4-ipomeanol (4-IM); the pro-death protein comprises a Carboxypeptidase (CP) and the pro-death agent comprises 4-[(2-chloroethyl)(2-mesyloxyethyl)amino]benzoyl-L-glutamic acid (CMDA), Hydroxy- and amino-aniline mustards, Anthracycline glutamates, and/or Methotrexate α-peptides (MTX-Phe); the pro-death protein comprises Carboxylesterase (CE) and the pro-death agent comprises Irinotecan (IRT), and/or Anthracycline acetals; the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones; the pro-death protein comprises Horse radish peroxidase (HRP) and the pro-death agent comprises Indole-3-acetic acid (IAA) and/or 5-Fluoroindole-3-acetic acid (FIAA); the pro-death protein comprises Guanine Ribosyltransferase (XGRTP) and the pro-death agent comprises 6-Thioxanthine (6-TX); the pro-death protein comprises a glycosidase enzyme and the pro-death agent comprises HM1826 and/or Anthracycline acetals; the pro-death protein comprises Methionine-α,γ-lyase (MET) and the pro-death agent comprises Selenomethionine (SeMET); and/or the pro-death protein comprises thymidine phosphorylase (TP) and the pro-death agent comprises 5′-Deoxy-5-fluorouridine (5′-DFU).

In some embodiments, a payload comprises one or more receptors and/or a targeting moiety configured to bind a component of a target site of a subject. In some embodiments, the one or more receptors and/or the one or more targeting moieties are selected from the group comprising mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. In some embodiments, the one or more targeting moieties and/or one or more receptors comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof. In some embodiments, the antibody or antigen-binding fragment thereof comprises a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the one or more targeting moieties and/or one or more receptors are configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11(C—C motif chemokine 11), CCR4 (C—C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B 1, CYP1B 1, Cytomegalovirus, Cytomegalovirus glycoprotein B, Dabigatran, DLL4 (delta-like—ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor a-chain, Growth differentiation factor 8, GP100, GPNMB (Transmembrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-7, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αᵤβ₃, ανβ3, α₄β₇, α5β1, α6β4, α7β7, α11β3, α5β5, ανβ5), Interferon gamma-induced protein, ITGA2, ITGB2, KIR2D, LCK, Le, Legumain, Lewis-Y antigen, LFA-1(Lymphocyte function-associated antigen 1, CD11a), LHRH, LINGO-1, Lipoteichoic acid, LIVIA, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

In some embodiments, a payload protein is associated with an agricultural trait of interest selected from the group consisting of increased yield, increased abiotic stress tolerance, increased drought tolerance, increased flood tolerance, increased heat tolerance, increased cold and frost tolerance, increased salt tolerance, increased heavy metal tolerance, increased low-nitrogen tolerance, increased disease resistance, increased pest resistance, increased herbicide resistance, increased biomass production, male sterility, or any combination thereof. In some embodiments, a payload protein is associated with a biological manufacturing process selected from the group comprising fermentation, distillation, biofuel production, production of a compound, production of a polypeptide, or any combination thereof.

In some embodiments, a payload encodes a cellular reprogramming factor capable of converting an at least partially differentiated cell to a less differentiated cell, such as, for example, Oct-3, Oct-4, Sox2, c-Myc, Klf4, Nanog, Lin28, ASCL1, MYT1L, TBX3b, SV40 large T, hTERT, miR-291, miR-294, miR-295, or any combinations thereof. In some embodiments, a payload encodes a cellular reprogramming factor capable of differentiating a given cell into a desired differentiated state, such as, for example, nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof.

In some embodiments, an input element comprises a heterologous promoter element and/or an endogenous promoter element. In some embodiments, the heterologous promoter element is capable of being bound by a component of a synthetic protein circuit. In some embodiments, the endogenous promoter element comprises a tissue-specific promoter and/or a lineage-specific promoter. In some embodiments, the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. In some embodiments, the tissue specific promoter is a neuron-specific promoter. In some embodiments, the neuron-specific promoter comprises a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Naᵥ1.7 promoter, a Naᵥ1.8 promoter, a Naᵥ1.9 promoter, or an Advillin promoter. In some embodiments, the tissue specific promoter is a muscle-specific promoter. In some embodiments, the muscle-specific promoter comprises a creatine kinase (MCK) promoter. In some embodiments, the nucleic acid composition comprises: one more polynucleotides encoding at least one synthetic protein circuit component. In some embodiments, a synthetic protein circuit component modulates the expression and/or activity of one or more TFs and/or one or more payloads. In some embodiments, a Synthetic Notch (SynNotch) receptor, a Modular Extracellular Sensor Architecture (MESA) receptor, Tango, dCas9-synR, or any combination thereof, is capable of modulating the expression and/or activity of one or more TFs and/or one or more payloads.

In some embodiments, a first promoter, second promoter, third promoter, and/or nth supplemental promoter comprises a minimal promoter (e.g., TATA, miniCMV, and/or miniPromo). In some embodiments, a TF, a payload, and/or a transactivator comprises a constitutive signal peptide for protein degradation (e.g., PEST). In some embodiments, a TF, a payload, and/or a transactivator comprises a nuclear localization signal (NLS) or a nuclear export signal (NES). In some embodiments, the first polynucleotide, the second polynucleotide, the third polynucleotide, the fourth polynucleotide, the fifth polynucleotide, the sixth polynucleotide, the nth supplemental polynucleotide, and/or (n+1)th supplemental polynucleotide are operably linked to a tandem gene expression element. In some embodiments, the tandem gene expression element is an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. In some embodiments, the first polynucleotide, the second polynucleotide, the third polynucleotide, the fourth polynucleotide, the fifth polynucleotide, the sixth polynucleotide, the nth supplemental polynucleotide, and/or (n+1)th supplemental polynucleotide further comprises a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. In some embodiments, the first polynucleotide, the second polynucleotide, the third polynucleotide, the fourth polynucleotide, the fifth polynucleotide, the sixth polynucleotide, the nth supplemental polynucleotide, and/or (n+1)th supplemental polynucleotide is evolutionarily stable for at least about 10 days, about 20 days, about 40 days, about 80 days, about 80 days, or about 100 days, of serial passaging.

In some embodiments, the nucleic acid composition comprises one or more vectors. In some embodiments, at least one of the one or more vectors is a viral vector, a plasmid, a transposable element, a naked DNA vector, a lipid nanoparticle, or any combination thereof. In some embodiments, the viral vector is an AAV vector, a lentivirus vector, a retrovirus vector, an integration-deficient lentivirus (IDLV) vector. In some embodiments, the transposable element is piggybac transposon or sleeping beauty transposon.

Disclosed herein include compositions. In some embodiments, the composition comprises: one or more nucleic acid compositions provided herein. In some embodiments, the composition comprises one or more vectors, a ribonucleoprotein (RNP) complex, a liposome, a nanoparticle, an exosome, a microvesicle, or any combination thereof. In some embodiments, the vector is a viral vector, a plasmid, a transposable element, a naked DNA vector, a lipid nanoparticle, or any combination thereof. In some embodiments, the transposable element is piggybac transposon or sleeping beauty transposon. In some embodiments, the viral vector is an AAV vector, a lentivirus vector, a retrovirus vector, an integration-deficient lentivirus (IDLV) vector. In some embodiments, the AAV vector comprises single-stranded AAV (ssAAV) vector or a self-complementary AAV (scAAV) vector.

Disclosed herein include cells. In some embodiments, the cell comprises: one or more of the nucleic acid compositions provided herein.

Disclosed herein include cell populations. In some embodiments, the cell population comprises a plurality of cells. In some embodiments, each cell comprises one or more of the nucleic acid compositions provided herein.

In some embodiments, the cell population comprises a plurality of monoclonal cells. In some embodiments, the cell population comprises one or more subpopulations of cells. In some embodiments, subpopulations are metabolically or functionally distinct subpopulations. In some embodiments, each subpopulation of cells is characterized by differences in the concentration and/or expression level of one or more TFs and one or more payloads. In some embodiments, each subpopulation of cells is characterized by a distinct expression state. In some embodiments, the expression state is mitotically heritable. In some embodiments, an expression state is stable across multiple cell division cycles. In some embodiments, the expression state is robust to biological gene expression noise. In some embodiments, less than about 10% of cells within a subpopulation transition to another expression state due to intrinsic noise.

In some embodiments, one or more subpopulations comprise: a first subpopulation of cells characterized by a first expression state. In some embodiments, the first expression state comprises: tuned expression levels of the first TF and first payload(s), and depleted expression levels of the second TF, second payload(s), third TF, and/or third payload(s). In some embodiments, one or more subpopulations comprise: a second subpopulation of cells characterized by a second expression state. In some embodiments, the second expression state comprises: tuned expression levels of the second TF and second payload(s), and depleted expression levels of the first TF, first payload(s), third TF, and/or third payload(s). In some embodiments, one or more subpopulations comprise: a third subpopulation of cells characterized by a third expression state. In some embodiments, the third expression state comprises: tuned expression levels of the third TF and third payload(s), and depleted expression levels of the first TF, first payload(s), second TF, and/or second payload(s). In some embodiments, one or more subpopulations comprise: a fourth subpopulation of cells characterized by a fourth expression state. In some embodiments, the fourth expression state comprises: tuned expression levels of the first TF, first payload(s), second TF, and second payload(s), and. In some embodiments, the fourth expression state comprises: depleted expression levels of the third TF, and/or third payload(s). In some embodiments, one or more subpopulations comprise: a fifth subpopulation of cells characterized by a fifth expression state. In some embodiments, the fifth expression state comprises: tuned expression levels of the first TF, first payload(s), third TF, and third payload(s), and depleted expression levels of the second TF, and/or second payload(s). In some embodiments, one or more subpopulations comprise: a sixth subpopulation of cells characterized by a sixth expression state. In some embodiments, the sixth expression state comprises: tuned expression levels of the second TF, second payload(s), third TF, and third payload(s), and depleted expression levels of the first TF, and/or first payload(s). In some embodiments, one or more subpopulations comprise: a seventh subpopulation of cells characterized by a seventh expression state. In some embodiments, the seventh expression state comprises: tuned expression levels of the first TF, first payload(s), second TF, second payload(s), third TF, and third payload(s).

In some embodiments, tuned expression levels range between a lower tuned threshold and an upper tuned threshold of a tuned expression range. In some embodiments, the tuned expression range is capable of being tuned by modulating one or more of dimerization domain affinity, TF protein stability, transactivation domain strength, DNA-binding domain, or any combination of thereof. In some embodiments, the difference between the lower untuned threshold and the upper untuned threshold of the tuned expression range is greater than about one order of magnitude. In some embodiments, the difference between the lower untuned threshold and the upper untuned threshold of the tuned expression range is less than about one order of magnitude. In some embodiments, depleted expression levels comprise basal expression levels. In some embodiments, depleted expression levels comprise absent expression. In some embodiments, tuned expression levels are at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, or 100-fold, greater than depleted expression levels. In some embodiments, expression levels comprise transcript levels and/or protein levels.

In some embodiments, transient induction of expression of one or more TFs is capable of transitioning cells from one expression state to another expression state. In some embodiments, transient induction of one or more TFs is capable of irreversibly transitioning cells from one expression state to another expression state. In some embodiments, a transactivator-binding compound causes transient induction of expression of the one or more TFs. In some embodiments, one or more subpopulations comprise: an eighth subpopulation of cells characterized by an off expression state. In some embodiments, the off expression state comprises: depleted expression levels of the first TF, first payload(s), second TF, second payload(s), third TF, and/or third payload(s). In some embodiments, some or all cells of the cell population are capable of transitioning to the off state in the absence of the degron stabilizing molecule and the dimerization ligand. In some embodiments, some or all cells of the cell population are capable of transitioning from the off state to the first expression state, second expression state, third expression state, fourth expression state, fifth expression state, sixth expression state, and/or seventh expression state, in the presence of a first threshold level of the degron stabilizing molecule and the dimerization ligand. In some embodiments, the number of expression states increases monotonically with the number of distinct TF species in the cell population. In some embodiments, the number of robust expression states decreases monotonically with TF protein stability. In some embodiments, the number of robust expression states decreases monotonically with the concentration of the degron stabilizing molecule. In some embodiments, reducing TF stability is capable of transitioning cells from one expression state to another expression state. In some embodiments, reducing TF stability is capable of irreversibly transitioning cells from one expression state to another expression state. In some embodiments, restoring TF stability is not capable of causing cells to return to previously destabilized states. In some embodiments, restoring TF stability comprises increasing the concentration of the degron stabilizing molecule. In some embodiments, below a second threshold level of the degron stabilizing molecule, the seventh expression state is destabilized. In some embodiments, below a second threshold level of the degron stabilizing molecule, the seventh expression state is destabilized irreversibly. In some embodiments, below a third threshold level of the degron stabilizing molecule, the fourth expression state, the fifth expression state, and/or the sixth expression state, is destabilized. In some embodiments, below a third threshold level of the degron stabilizing molecule, the fourth expression state, the fifth expression state, and/or the sixth expression state, is destabilized irreversibly.

In some embodiments, tuned expression levels, the number of subpopulations, the types of subpopulations, the relative number of cells within each subpopulation, and/or the expression state of one or more cells is configured to be responsive to changes in: the local concentration of a degron stabilizing molecule, a transactivator-binding compound, a dimerization ligand, or any combination thereof, cell environment (e.g., location relative to a target site of a subject and/or changes in the presence and/or absence of target cell(s) comprising target-specific antigen(s)); one or more signal transduction pathways regulating cell survival, cell growth, cell proliferation, cell adhesion, cell migration, cell metabolism, cell morphology, cell differentiation, apoptosis, or any combination thereof, input(s) of a synthetic cell-cell communication system (e.g., Synthetic Notch (SynNotch) receptor, a Modular Extracellular Sensor Architecture (MESA) receptor, a synthekine, engineered GFP, and/or auxin); and/or T cell activity (e.g., T cell simulation, T cell activation, cytokine secretion, T cell survival, T cell proliferation, CTL activity, T cell degranulation, and T cell differentiation). In some embodiments, a synthetic protein circuit component is capable of modulating the expression and/or activity of a TF. In some embodiments, the expression and/or activity of a TF is configured to be responsive to immune cell stimulation. In some embodiments, immune cell stimulation comprises signal transduction induced by binding of a stimulatory molecule with its cognate ligand on the surface of an immune cell. In some embodiments, the cognate ligand is a CAR or a TCR. In some embodiments, one or more of the expression states is configured to activate a state-specific program. In some embodiments, the state-specific program is a therapeutic program. In some embodiments, the population of cells is configured to generate mixture of subpopulations at defined ratios. In some embodiments, the defined ratio is selected to generate synergy between the state-specific programs of said subpopulations. In some embodiments, the one or more subpopulations comprise and/or are capable of differentiating into two or more cell types. In some embodiments, the two or more cell types are capable of providing different overall functions and/or different components of a single function. In some embodiments, the two or more cell types are found within the same tissue. In some embodiments, the population of cells is configured to respond to the inputs of a synthetic cell-cell communication system. In some embodiments, the tuned expression levels and/or the expression state of one or more cells is configured to be responsive to changes in one or more inputs. In some embodiments, a threshold input level. In some embodiments, the input level is sensed by an engineered biosensor. In some embodiments, the tuned expression levels and/or the expression state of one or subpopulations is capable of being modulated by one or more of a Synthetic Notch (SynNotch) receptor, a Modular Extracellular Sensor Architecture (MESA) receptor, Tango, dCas9-synR, or any combination thereof. In some embodiments, one or more cells of the population of cells is configured to activate a therapeutic program in the presence of an input threshold. In some embodiments, a local input threshold at a target site. In some embodiments, the therapeutic program comprises expression of one or more payloads. In some embodiments, one or more cells of the population of cells are immune cells is configured to switch from an immune cell inactivated state to an immune cell activated state in the presence of an input threshold (e.g., a local input threshold at a target site). In some embodiments, one or more cells of the population of cells is configured to differentiate into one or more cell types in the presence of an input threshold (e.g., a local input threshold at a target site). In some embodiments, the population of cells are capable of being employed in synthetic organogenesis and/or tissue repair.

In some embodiments, the cell comprises a eukaryotic cell. In some embodiments, the eukaryotic cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof. In some embodiments, the cell is a bacterial cell, a yeast cell, a fungal cell, a mammalian cell, a human cell, a stem cell, a progenitor cell, an induced pluripotent stem cell, a human induced pluripotent stem cell, a plant cell or an animal cell. In some embodiments, one or more subpopulations are configured to express one or more targeting moieties configured to bind a component of a target site of a subject.

Disclosed herein include method of treating a disease or disorder in a subject. In some embodiments, the method comprises: introducing into one or more cells one or more of the nucleic acid compositions provided herein or one or more of the compositions provided herein; and administering to the subject an effective amount of the one or more cells, or a cell population derived therefrom. In some embodiments, the method comprises: isolating the one or more cells from the subject prior to the introducing step. In some embodiments, the introducing step is performed in vivo, in vitro, and/or ex vivo. In some embodiments, the introducing step comprises calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof.

Disclosed herein include method of treating a disease or disorder in a subject. In some embodiments, the method comprises: administering to the subject an effective amount of cell(s) or cell population(s) provided herein.

In some embodiments, the method comprises: administering to the subject an effective amount of a degron stabilizing molecule, a transactivator-binding compound, a dimerization ligand, or any combination thereof. In some embodiments, a target site of a subject comprises a site of disease or disorder or is proximate to a site of a disease or disorder, In some embodiments, the target site comprises a tissue. In some embodiments, the tissue is inflamed tissue, cancerous tissue, and/or infected tissue. In some embodiments, the tissue comprises adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. In some embodiments, the tissue comprises: (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue.

In some embodiments, the disease is associated with expression of a tumor antigen. In some embodiments, the disease associated with expression of a tumor antigen is selected from the group consisting of a proliferative disease, a precancerous condition, a cancer, and a non-cancer related indication associated with expression of the tumor antigen. In some embodiments, the disease or disorder is a blood disease, an immune disease, a neurological disease or disorder, a cancer, a solid tumor, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof. In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, the method comprises: administering one or more additional agents to the subject. In some embodiments, the one or more additional agents comprise a protein phosphatase inhibitor, a kinase inhibitor, a cytokine, an inhibitor of an immune inhibitory molecule, and/or or an agent that decreases the level or activity of a T_{REG} cell. In some embodiments, the one or more additional agents comprise an immune modulator, an anti-metastatic, a chemotherapeutic, a hormone or a growth factor antagonist, an alkylating agent, a TLR agonist, a cytokine antagonist, a cytokine antagonist, or any combination thereof. In some embodiments, the one or more additional agents comprise an agonistic or antagonistic antibody specific to a checkpoint inhibitor or checkpoint stimulator molecule such as PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, TIM-3. In some embodiments, the one or more additional agents are selected from the group consisting of alkylating agents (nitrogen mustards, ethylenimine derivatives, alkyl sulfonates, nitrosoureas and triazenes); uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®); bendamustine (Treakisym®, Ribomustin®, Treanda®); chlormethine (Mustargen®); cyclophosphamide (Cytoxan®, Neosar®, Clafen®, Endoxan®, Procytox®, Revimmune™); ifosfamide (Mitoxana®); melphalan (Alkeran®); Chlorambucil (Leukeran®); pipobroman (Amedel®, Vercyte®); triethylenemelamine (Hemel®, Hexylen®, Hexastat®); triethylenethiophosphoramine; Temozolomide (Temodar®); thiotepa (Thioplex®); busulfan (Busilvex®, Myleran®); carmustine (BiCNU®); lomustine (CeeNU®); streptozocin (Zanosar®); estramustine (Emcyt®, Estracit®); fotemustine; irofulven; mannosulfan; mitobronitol; nimustine; procarbazine; ranimustine; semustine; triaziquone; treosulfan; and Dacarbazine (DTIC-Dome®); anti-EGFR antibodies (e.g., cetuximab (Erbitux®), panitumumab (Vectibix®), and gefitinib (Iressa®)); anti-Her-2 antibodies (e.g., trastuzumab (Herceptin®) and other antibodies from Genentech); antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), carmofur, cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®), decitabine (Dacogen®), enocitabine (Sunrabin®), sapacitabine, tegafur-uracil, tiazofurine, tioguanine, trofosfamide, and gemcitabine (Gemzar®); vinca alkaloids: vinblastine (Velban®, Velsar®), vincristine (Vincasar®, Oncovin®), vindesine (Eldisine®), vinorelbine (Navelbine®), vinflunine (Javlor®); platinum-based agents: carboplatin (Paraplat®, Paraplatin®), cisplatin (Platinol®), oxaliplatin (Eloxatin®), nedaplatin, satraplatin, and triplatin; anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®), aclarubicin, amrubicin, liposomal doxorubicin, liposomal daunorubicin, pirarubicin, pixantrone, and zorubicin; topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101), belotecan, and rubitecan; taxanes: paclitaxel (Taxol®), docetaxel (Taxotere®), larotaxel, cabazitaxel, ortataxel, and tesetaxel; antibiotics: actinomycin (Cosmegen®), bleomycin (Blenoxane®), hydroxyurea (Droxia®, Hydrea®), mitomycin (Mitozytrex®, Mutamycin®); immunomodulators: lenalidomide (Revlimid®), thalidomide (Thalomid®); immune cell antibodies: alemtuzamab (Campath®), gemtuzumab (Myelotarg®), rituximab (Rituxan®), tositumomab (Bexxar®); interferons (e.g., IFN-alpha (Alferon®, Roferon-A®, Intron®-A) or IFN-gamma (Actimmune®)); interleukins: IL-1, IL-2 (Proleukin®), IL-24, IL-6 (Sigosix®), IL-12; HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”); anti-androgens which include, without limitation nilutamide (Nilandron®) and bicalutamide (Caxodex®); antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride; anti-hypercalcaemia agents which include without limitation gallium (III) nitrate hydrate (Ganite®) and pamidronate disodium (Aredia®); apoptosis inducers which include without limitation ethanol, 2-[[3-(2,3-dichlorophenoxy)propyl]amino]-(9Cl), gambogic acid, elesclomol, embelin and arsenic trioxide (Trisenox®); Aurora kinase inhibitors which include without limitation binucleine 2; Bruton's tyrosine kinase inhibitors which include without limitation terreic acid; calcineurin inhibitors which include without limitation cypermethrin, deltamethrin, fenvalerate and tyrphostin 8; CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide; CD45 tyrosine phosphatase inhibitors which include without limitation phosphonic acid; CDC25 phosphatase inhibitors which include without limitation 1,4-naphthalene dione, 2,3-bis[(2-hydroxyethyl)thio]-(9Cl); CHK kinase inhibitors which include without limitation debromohymenialdisine; cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid); cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl); cyclin dependent kinase inhibitors which include without limitation olomoucine and its derivatives, purvalanol B, roascovitine (Seliciclib®), indirubin, kenpaullone, purvalanol A and indirubin-3′-monooxime; cysteine protease inhibitors which include without limitation 4-morpholinecarboxamide, N-[(1S)-3-fluoro-2-oxo-1-(2-phenylethyl)propyl]amino]-2-oxo-1-(phenylmeth-yl)ethyl]-(9Cl); DNA intercalators which include without limitation plicamycin (Mithracin®) and daptomycin (Cubicin®); DNA strand breakers which include without limitation bleomycin (Blenoxane®); E3 ligase inhibitors which include without limitation N-((3,3,3-trifluoro-2-trifluoromethyl)propionyl)sulfanilamide; EGF Pathway Inhibitors which include, without limitation tyrphostin 46, EKB-569, erlotinib (Tarceva®), gefitinib (Iressa®), lapatinib (Tykerb®) and analogues; farnesyltransferase inhibitors which include without limitation ahydroxyfarnesylphosphonic acid, butanoic acid, 2-[(2S)-2-[[(2S,3S)-2-[[(2R)-2-amino-3-mercaptopropyl]amino]-3-methylpent-yl]oxy]-1-oxo-3-phenylpropyl]amino]-4-(methylsulfonyl)-1-methylethylester (2S)-(9Cl), tipifarnib (Zarnestra®), and manumycin A; Flk-1 kinase inhibitors which include without limitation 2-propenamide, 2-cyano-3-[4-hydroxy-3,5-bis(1-methylethyl)phenyl]-N-(3-phenylpropyl)-(2E-)-(9Cl); glycogen synthase kinase-3 (GSK3) inhibitors which include without limitation indirubin-3′-monooxime; histone deacetylase (HDAC) inhibitors which include without limitation suberoylanilide hydroxamic acid (SAHA), [4-(2-amino-phenylcarbamoyl)-benzyl]carbamic acid pyridine-3-ylmethylester and its derivatives, butyric acid, pyroxamide, trichostatin A, oxamflatin, apicidin, depsipeptide, depudecin, trapoxin, vorinostat (Zolinza®), and compounds disclosed in WO 02/22577; I-kappa B-alpha kinase inhibitors (IKK) which include without limitation 2-propenenitrile, 3-[(4-methylphenyl)sulfonyl]-(2E)-(9Cl); imidazotetrazinones which include without limitation temozolomide (Methazolastone®, Temodar® and its derivatives (e.g., as disclosed generically and specifically in U.S. Pat. No. 5,260,291) and Mitozolomide; insulin tyrosine kinase inhibitors which include without limitation hydroxyl-2-naphthalenylmethylphosphonic acid; c-Jun-N-terminal kinase (INK) inhibitors which include without limitation pyrazoleanthrone and epigallocatechin gallate; mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hy-droxyethyl)-4-methoxy-(9Cl); MDM2 inhibitors which include without limitation trans-4-iodo, 4′-boranyl-chalcone; MEK inhibitors which include without limitation butanedinitrile, bis[amino[2-aminophenyl)thio]methylene]-(9Cl); MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996; mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD; NGFR tyrosine kinase inhibitors which include without limitation tyrphostin AG 879; p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl); p56 tyrosine kinase inhibitors which include without limitation damnacanthal and tyrphostin 46; PDGF pathway inhibitors which include without limitation tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854; phosphatidylinositol 3-kinase inhibitors which include without limitation wortmannin, and quercetin dihydrate; phosphatase inhibitors which include without limitation cantharidic acid, cantharidin, and L-leucinamide; protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid; PKC inhibitors which include without limitation 1-H-pyrollo-2,5-dione, 3-[1-3-(dimethylamino)propyl]-1H-indol-3-yl]-4-(1H-indol-3-yl)-(9Cl), Bisindolylmaleimide IX, Sphinogosine, staurosporine, and Hypericin; PKC delta kinase inhibitors which include without limitation rottlerin; polyamine synthesis inhibitors which include without limitation DMFO; PTP1B inhibitors which include without limitation L-leucinamide; protein tyrosine kinase inhibitors which include, without limitation tyrphostin Ag 216, tyrphostin Ag 1288, tyrphostin Ag 1295, geldanamycin, genistein and 7H-pyrrolo[2,3-d]pyrimidine derivatives as generically and specifically described in PCT Publication No.: WO 03/013541 and U.S. Publication No.: 2008/0139587; SRC family tyrosine kinase inhibitors which include without limitation PP1 and PP2; Syk tyrosine kinase inhibitors which include without limitation piceatannol; Janus (JAK-2 and/or JAK-3) tyrosine kinase inhibitors which include without limitation tyrphostin AG 490 and 2-naphthyl vinyl ketone; retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®); RNA polymerase H elongation inhibitors which include without limitation 5,6-dichloro-1-beta-D-ribofuranosylbenzimidazole; serine/Threonine kinase inhibitors which include without limitation 2-aminopurine; sterol biosynthesis inhibitors which include without limitation squalene epoxidase and CYP2D6; VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

In some embodiments, administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

### BRIEF DESCRIPTION OF THE DRAWINGS

FIG. 1A-FIG. 1D depict non-limiting exemplary embodiments showing the MultiFate architecture provided herein can generate diverse types of multistability in the model. FIG. 1A depicts a non-limiting synthetic multistable circuit represented by cell cartoons (top) and attractors in a transcription factor phase space (bottom, TF A-C on coordinate axes represent transcription factor concentrations). In some embodiments, an ideal synthetic multistable circuit should generate multiple stable states, support control of state-switching (left) and state stability (middle), and allow easy expansion of states by addition of more transcription factors (right). FIG. 1B depicts non-limiting examples of competitive protein-protein interactions and autoregulatory feedback prevalent in natural multistable circuits that control myogenesis (left) and endodermal differentiation (right), as shown by these diagrams. Blue arrows indicate competitive protein-protein interactions, which can involve higher order multimerization. Orange dashed arrows indicate direct or indirect positive transcriptional feedback. FIG. 1C-FIG. 1D depict non-limiting exemplary models of the MultiFate-2 circuit and MultiFate-3 circuit (See, Example 1 below) that generate diverse types of multistability in different parameter regimes (indicated above plots). In the model of the MultiFate-3 circuit, low protein stability generates 4 stable states (type I quadrastability), but the state in which all transcription factors are lowly expressed is unstable in the presence of biological noise (FIG. 28A-FIG. 28C), consistent with experimental results in FIG. 5B, Low TMP columns. Complete lists of multistability regimes are shown in FIG. 6A-FIG. 6B and FIG. 7A-FIG. 7B. All models used here are symmetric and non-dimensionalized, with rescaled dimerization dissociation constant K_{d}=1 and Hill coefficient n=1.5 (See, Example 1 below). Each axis represents the dimensionless total concentration of each transcription factor. Note that in the non-dimensionalized model, changing protein stability is equivalent to multiplying α and β with the same factor (See, Example 1 below).

FIG. 2A-FIG. 2D depict non-limiting exemplary embodiments showing engineered transcription factors enable homodimer-dependent autoregulation and heterodimerization-based inhibition. FIG. 2A shows non-limiting exemplary data depicting ZF transcription factors enable homodimer-dependent activation. (Left) Design of test constructs, in which ErbB2ZF (red circle) fused to VP48 (AD) and in some cases GCN4 (blue squiggle) domains bind to target sites (red pads) to activate Citrine expression. Activators were expressed from a constitutive CAG promoter. (Right) R-to-A mutations in ZF modulated reporter activation by ZF-GCN4-AD and ZF-AD. The R2AR39A variant was selected due to high ZF-GCN4-AD activation and minimal ZF-AD activation. Fold activation is defined in FIG. 9A. WT, wild-type variant. FIG. 2B depicts non-limiting exemplary data showing that using FKBP12F36V (FKBP) as the dimerization domain (light cyan partial box) allows dose-dependent control of activation by AP1903 (cyan circle). Red circle, BCRZFR39A. FIG. 2C depicts non-limiting exemplary embodiments showing that transcription factor self-activation can be controlled by TMP and AP1903. Cartoon depicts the design of the controllable self-activation circuit. IRES, internal ribosome entry site; PEST, constitutive degradation tag; (Top graph) Stable polyclonal cells showed bimodal mCitrine distribution upon circuit activation. An empirical threshold at mCitrine=104 separates the distribution into two fractions, and the normalized mCitrine+ fraction was used to quantify the self-activation strength (See, Example 1 below). (Bottom graph) Colored arrows indicate data from the top graph. AP1903+ samples had 100 nM AP1903. FIG. 2D depicts non-limiting exemplary data showing self-activation was inhibited by proteins with a different ZF and matching dimerization domains. Two monoclonal stable lines could spontaneously self-activate in media containing AP1903 and TMP (FIG. 10B-FIG. 10C). Each perturbation construct is introduced by stable integration (See, Example 1 below). The integrated construct in the “None” group did not express any perturbation protein. Dark circle, 42ZFR2AR39AR67A; Light circle, BCRZFR39A. In all panels, each dot represents one biological replicate, and each red line or bar indicates the mean of replicates. Lists of constructs and cell lines are in Table 2 and Table 3.

FIG. 3A-FIG. 3D depict non-limiting exemplary embodiments showing MultiFate-2 generates multiple stable states. FIG. 3A shows a non-limiting cartoon of the experimental MultiFate-2 design using two self-activation cassettes differing only in their ZF DNA-binding domains and binding sites, and fluorescent proteins. Each cassette expresses FKBP-ZF-VP16-DHFR-IRES-FP-PEST, where ZF represents either BCRZFR39A or 37ZFR2AR11AR39AR67A and FP represents either mCherry or mCitrine, for A and B, respectively. Details of constructs and differences among MultiFate-2 lines are shown in Table 2 and Table 3. FIG. 3B depicts non-limiting exemplary data showing MultiFate-2.1 cells spontaneously activated A, B or both cassettes upon addition of 100 nM AP1903 and 10 μM TMP. Cell percentages in OFF, A-only, B-only and A+B states were quantified and plotted as a square with four shaded circles. FIG. 3C depicts non-limiting exemplary data showing that three MultiFate-2 lines all exhibited type II tristability in the High TMP condition, and bistability in the Low TMP condition. In all conditions, 100 nM AP1903 was added. Exact concentrations of TMP are shown in FIG. 14A-FIG. 16B. Unstable states, defined by states having more than 10% cells escaping their initial states after 18 days, were marked in pink rectangles. Each square represents the mean fractions of three biological replicates. Initial A-only, B-only and A+B cells were sorted from a population of cells in different states, while initial OFF cells came from cells in regular CHO media without any inducers. FIG. 3D depicts non-limiting exemplary data showing that A-only, B-only and A+B states were each stable during growth from single MultiFate-2.3 cells into colonies over 5 days under a time-lapse microscope. (Top) Mixed MultiFate-2.3 cell populations were first sorted to separate cells in 3 different states. Then cells in these three states were seeded at equal ratio in the same well and time-lapse imaging was performed (See, Example 1 below). (Bottom) Scale bar: 500 μm for the wide field image, 100 μm for zoomed in images. “High TMP”, 100 nM AP1903+10 μM TMP.

FIG. 4A-FIG. 4B depict non-limiting exemplary embodiments showing MultiFate-2 supports modulation of state stability and allows state-switching. FIG. 4A depicts non-limiting exemplary data showing escape from the destabilized A+B state was irreversible, as shown by both modeling, and experiment using MultiFate-2.1 cells. (Top) The model used here is symmetric and non-dimensionalized, with rescaled dimerization dissociation constant K_{d}=1 and Hill coefficient n=1.5 (See, Example 1 below). The x and y axes are total dimensionless concentrations of TF A and TF B, respectively. Simulated cells on phase portraits were calculated using the Gillespie algorithm (See, Example 1 below). Note that in the non-dimensionalized model, changing protein stability is equivalent to multiplying α and β with the same factor (See, Example 1 below). (Bottom) Throughout the experiment, 100 nM AP1903 was added. Exact concentrations of TMP are shown in FIG. 14A-FIG. 14C. FIG. 4B shows non-limiting exemplary data related to the finding that MultiFate-2.3 cells can be switched between states by transient 4-OHT or Dox treatment. In all conditions, 100 nM AP1903 was added. Exact concentrations of TMP are shown in FIG. 21A-FIG. 21B. 4-OHT, 25 nM, Dox, 500 ng/ml. In all panels, initial A-only, B-only and A+B cells were sorted from a population of cells in different states. Each square represents the mean fractions of three biological replicates.

FIG. 5A-FIG. 5D depict non-limiting exemplary embodiments showing that MultiFate architecture is expandable to include three or more transcription factors. FIG. 5A shows a cartoon of a non-limiting exemplary experimental MultiFate-3 design using three self-activation cassettes differing only in their ZF DNA-binding domains and binding sites, and fluorescent proteins. Each cassette expresses FKBP-ZF-VP16-DHFR-IRES-FP-PEST, where ZF represents either BCRZFR39A, 37ZFR2AR11AR39AR67A or ErbB2ZFR2AR39A, and FP represents either mCherry, mCitrine or mTurquoise2 fluorescent protein, for A, B and C, respectively (Table 2). FIG. 5B depicts non-limiting exemplary data showing that the MultiFate-3 line exhibited type II septastability, hexastability and tristability in three different TMP conditions. State percentages in each octant were quantified and plotted as eight shaded circles (See, Example 1 below). High TMP condition, 100 nM AP1903+100 nM TMP; Intermediate TMP condition, 100 nM AP1903+40 nM TMP; Low TMP condition, 100 nM AP1903+10 nM TMP. Except for OFF state cells, cells in different initial states were sorted from a mixed population of cells in the High TMP condition. Initial OFF cells came from cells in regular CHO media without any inducers. Each plot represents the mean percentages of three biological replicates. FIG. 5C shows non-limiting exemplary data that cells in each of the seven states were stable during growth from single cells into colonies over 6 days under a time-lapse microscope. Cells were sorted and an equal ratio of cells in 7 states were seeded using the same method for FIG. 3D. Scale bar: 500 μm for the wide field image (top), 100 μm for zoomed in images (bottom). FIG. 5D depicts non-limiting exemplary embodiments showing that MultiFate is expandable (model). The number of robust stable fixed points grows monotonically with the number of transcription factors species (N) in the model. A robust stable fixed point is defined as a stable fixed point that has fewer than 10% cells escaping at the end of stochastic simulations (See, Example 1 below). The parameter set provided above the plot (with K_{d}=1 and n=1.5) is the same non-dimensionalized parameter set used in MultiFate-2 and MultiFate-3 models under high protein stability.

FIG. 6A-FIG. 6B depict non-limiting exemplary embodiments showing the MultiFate-2 model generates diverse types of multistability. FIG. 6A depicts non-limiting exemplary data showing that in different symmetric parameter regimes (in which parameters for the two transcription factors are identical), MultiFate-2 can generate two types of monostability, bistability, two types of tristability, and quadrastability. For each regime, non-dimensionalized parameters α and β are provided above the plot, and K_{d}=1 and n=1.5. The x and y axes are dimensionless TF A and TF B, respectively. FIG. 6B depicts non-limiting exemplary data showing that a parameter screen reveals how each of the non-dimensionalized parameters individually affects the global structure of the system. Each row and column in the grid of plots represents a titration of one parameter value, indicated at left and bottom. Within each plot, different shading represent different stability regimes, as in FIG. 6A, determined by numerically solving for steady state values and their linear stability at each point in each parameter space (See, Example 1 below). In the non-dimensionalized model, changing protein stability is equivalent to multiplying α and β with the same factor (See, Example 1 below), and is shown as “protein stability factor—X”, with higher values representing greater protein stability. Higher leaky transcription (high α) allows transcription factors to spontaneously self-activate, destabilizing the OFF state (α column). Very high α values push the system towards monostability where only the state in which both transcription factors are highly expressed is stable. Stronger self-activation (higher values of β) is more likely to produce type II tristability and quadrastability (β column). Strong dimerization (low K_{d}) is essential for type II tristability (K_{d} row). A broad range of Hill coefficients n≥1 are compatible with different types of multistability (n row). While higher values of n reduce sensitivity to other parameters and allow the system generate type II tristability even with higher values of α, they also stabilize the OFF state to favor quadrastability.

FIG. 7A-FIG. 7B show non-limiting exemplary embodiments depicting the MultiFate-3 model generates more diverse types of multistability. FIG. 7A depicts exemplary models showing that in different parameter regimes, symmetric MultiFate-3 can generate a diverse repertoire of different types of stability. The x, y and z axes are total dimensionless concentrations of TF A, B and C, respectively. For each regime, non-dimensionalized parameters α and β are provided above the plot, and K_{d}=1 and n=1.5. FIG. 7B depicts non-limiting exemplary data showing that a parameter screen reveals how each of the non-dimensionalized parameters individually affects the global structure of the system. As in FIG. 6B, changing protein stability in the non-dimensionalized model is equivalent to multiplying α and β with protein stability factor (X). Each plot in the grid shows a titration of two parameter values (left and bottom). Higher leaky transcription (high α) allows transcription factors to spontaneously self-activate, destabilizing the OFF state. Very high α pushes the system towards monostability where only the state which all transcription factors are highly expressed is stable (α column). Stronger self-activation (higher values of β) generally favors higher levels of multistability, such as septastability and octostability (β column). Strong dimerization (low K_{d}) is essential for type II septastability (K_{d} column). A broad range of Hill coefficients n≥1 are compatible with different types of multistability. Higher values of n reduce sensitivity to other parameters, and allow the system to generate type II septastability even at higher values of α. However, they also stabilize the OFF state to favor octostability (n column). Note that the MultiFate-3 parameter screen graph structure resembles that of MultiFate-2 (FIG. 6B), in which monostability, octostability and type II septastability of MultiFate-3 appear at similar positions as monostability, quadrastability and type II tristability of MultiFate-2. As in FIG. 6B, each plot is calculated by numerical solution of the MultiFate-3 model for steady state values and their linear stability (See, Example 1 below).

FIG. 8A-FIG. 8B depict exemplary modeling of state-switching dynamics. Gillespie simulations were used to simulate the effects of transient perturbations on switching of cells among different states in both bistable regime (FIG. 8A) and type II tristable regimes (FIG. 8B). A modified MultiFate-2 model was used incorporating external inducers (See, Example 1 below). In this model, the strength of inducers is represented by the parameter indA or indB. Initially (left plot), cells (red dots) are in A-only, B-only or A+B state. Increasing indA or indB destabilizes the initial state (central two plots), allowing cells to transition to the target state. Terminating inducer treatment restores indA=indB=0. However, at this point, cells are already stabilized in the target state. The α, β, K_{d}, n used for bistable regime and type II tristable regime are the same as FIG. 1C. For each case, four representative timepoints are shown.

FIG. 9A-FIG. 9C depict non-limiting exemplary embodiments related to engineering dimer-dependent transcriptional regulation. In FIG. 9A, to characterize the activation strength of different zinc finger transcription factor variants, each of them was co-transfected with a reporter construct (Citrine) and a co-transfection marker (mTagBFP2). Highly transfected cells were gated with co-transfected BFP>3×105 (dashed box) to extract their individual histograms (bottom left). From each histogram, median Citrine fluorescence intensities of gated cells was used to calculate fold activation (bottom right) (See, Example 1 below). Thin black arrow indicates the original zinc finger sequence from the bottom left panel (WT). The R2AR39A variant (red box) was selected for its high ZF-GCN4-AD activation and minimal ZF-AD activation (AD denotes the VP48 activation domain). FIG. 9B shows non-limiting exemplary data depicting Zinc finger mutation variants with minimal activation by ZF-AD and strong activation by ZF-GCN4-AD (red boxes) were selected for use in MultiFate circuits (Table 5). Each dot represents one biological replicate, and each bar indicates the mean of all replicates. 37ZF, 42ZF, 43ZF, 92ZF, 97ZF were taken from A. S. Khalil, T. K. Lu, C. J. Bashor, C. L. Ramirez, N. C. Pyenson, J. K. Joung, J. J. Collins, A synthetic biology framework for programming eukaryotic transcription functions. Cell. 150, 647-658 (2012). BCRZF, HIV1ZF, HIV2ZF, and ErbB2ZF are from J. J. Lohmueller, T. Z. Armel, P. A. Silver, A tunable zinc finger-based framework for Boolean logic computation in mammalian cells. Nucleic Acids Research. 40 (2012), pp. 5180-5187. BCR denotes the BCR_ABL domain. FIG. 9C depicts a heat map showing the four selected zinc finger transcription factors exhibit orthogonal trans-activation. Each row represents a transcription factor with abbreviated labels for figure layout. Full transcription factor descriptions are, from top to bottom, 37ZFR2AR11AR39AR67A-GCN4-VP48; 42ZFR2AR39AR67A-GCN4-VP48; BCRZFR39A-GCN4-VP48; and ErbB2ZFR2AR39A-GCN4-VP48 (Table 5). Each target (column) is the same Citrine fluorescent reporter used in FIG. 9A, with 2 repeats of 18 bp tandem binding site pairs, denoted “ZFbs_ZFbs” for each type, at the promoter (Table 5). Each square in the matrix is the mean of two biological replicates.

FIG. 10A-FIG. 10C depict non-limiting exemplary embodiments showing engineered dimer-dependent transcription factors enable transcriptional positive autoregulation and mutual inhibition through competitive dimerization. FIG. 10A depicts non-limiting exemplary data showing positive autoregulation can be controlled by TMP and AP1903. Here, normalized mCitrine+ fractions were used to quantify self-activation strength, as in FIG. 2C (See, Example 1 below for quantification methods) for 6 additional ZFs. FIG. 10B-FIG. 10C depict non-limiting exemplary data showing positive autoregulation was inhibited by competing proteins with matching dimerization domains. Two monoclonal stable lines (plot subtitles) could spontaneously self-activate in media containing 100 nM AP1903 and 10 μM TMP (histograms). Potentially competing proteins were expressed from plasmids and stably integrated into each monoclonal line (See, Example 1 below). Note that in 42ZFR2AR39AR67A-GCN4-VP48-DFHR self-activation cells (FIG. 10B), the GCN4 domain by itself did not have inhibitory effects, but could efficiently inhibit self-activation when fused with BCRZFR39A. In FKBP-42ZFR2AR39AR67A-VP48-DHFR self-activation cells (FIG. 10C), the FKBP domain by itself can partially inhibit self-activation. The mCitrine threshold is 2×104 for 42ZFR2AR39AR67A-GCN4-VP48-DHFR self-activating cells, and 5×104 for FKBP-42ZFR2AR39AR67A-VP48-DHFR cells. In all panels, each dot represents one biological replicate, and each red line or bar indicates the mean of replicates.

FIG. 11A-FIG. 11D depict exemplary modeling of the relationship between TF concentrations and fluorescence readout. FIG. 11A shows non-limiting exemplary data depicting that a self-activation module creates a threshold-like behavior: in some embodiments, when transcription factor concentration is higher than the threshold, the module is highly active and expresses a high level of fluorescent proteins, resulting in a ‘high’ state; when transcription factor concentration is lower than the threshold, the module is inactive and express a minimal level of fluorescent proteins, resulting in a ‘low’ state. (Bottom) The threshold is the TF concentration that produces a homodimer concentration of 1. (Top) In the ‘high’ state, transcription factor concentrations are sensitive to protein half-life, while fluorescence readouts (right) are not sensitive and almost overlap with each other. FIG. 11B depicts non-limiting exemplary modeling showing that MultiFate-2 fluorescence readouts are well separated into distinct clusters, and each cluster can be unambiguously assigned to its corresponding state defined by transcription factor concentrations. (Top) Transcription factor concentrations of simulated cells cluster around stable fixed points. For A-only state or B-only state, transcription factor concentrations differ by more than 2 folds between type II tristable regime and bistable regime, as shown on the ‘Overlap’ plot. (Bottom) By contrast, fluorescence readouts for A-only state or B-only state almost overlap with each other between type II tristable regime and bistable regime, consistent with experimental observation in FIG. 14A-FIG. 16B. In this panel, the MultiFate-2 parameters related to transcription factor dynamics (α, β, K_{d}, n) are the same as those used in FIG. 1C. Parameters related to fluorescent protein dynamics are in Table 1. FIG. 11C-FIG. 11D show non-limiting exemplary data related to fluorescence readouts having a time delay compared with transcription factor concentrations during cell state transition. The same modified MultiFate-2 model incorporating external input as that used in FIG. 8A-FIG. 8B, with α=0.4, β=10, K_{d}=1, n=1.5 was used. FIG. 11C depicts non-limiting exemplary simulated single cell dynamics of transcription factor concentrations (top) and fluorescence readouts (bottom) during cell state transition from A-only state to B-only state. Selected timepoints are: t1 is when the cell is in A-only state at the start of simulation; t2 is when transcription factor concentrations cross the state boundary (i.e. [TF A]=[TF B]); t3 is when fluorescence readouts cross the state boundary (i.e. [Mature FP A]=[Mature FP B]); t4 is when the cell is in B-only state at the end of simulation. For this simulation, maturation time of both fluorescence proteins is 8 hours, and fluorescent protein half-life is 10.125 hours. FIG. 11D shows an exemplary heat map depicting both longer maturation time and longer fluorescent protein half-life increase the time delay of fluorescence readouts. The color of each block represents the mean delay time of 200 cells.

FIG. 12A-FIG. 12B depict exemplary schematics of MultiFate-2 and MultiFate-3 clone selection processes. FIG. 12A shows an exemplary selection process wherein MultiFate-2 monoclones were selected from a population of cells that can maintain a stable double-positive state for at least 72 hours. FIG. 12B depicts an exemplary selection process wherein MultiFate-3 monoclones were selected from a population of cells that can maintain a stable triple-positive state for at least 72 hours.

FIG. 13A-FIG. 13E show non-limiting exemplary data related to doubling time of MultiFate cells. For each MultiFate line, most differences in doubling time among cells in different states are not significant. Welch's t-test (threshold p=0.05) was used, since it is suitable for pairwise comparison without assuming equal variance. FIG. 13D shows MultiFate-3 cells in A+B state grow slower than cells in C-only state. The differences in doubling time among different MultiFate lines are statistically significant. Without being bound by any particular theory, this may be due to clonal differences. Each dot represents one biological replicate, and each red line indicates the mean of all replicates. Doubling time of the same cell line from different states (from first four plots) are combined to generate FIG. 13E. *: 1e-2<p≤5e-2; **: 1e-3<p≤1e-2; ***: 1e-4<p≤1e-3; ****: p≤1e-4.

FIG. 14A-FIG. 14C depict non-limiting exemplary data related to raw flow cytometry analysis of the MultiFate-2.1 line. Each plot represents one of three biological replicates at the indicated time point (cf. FIG. 3C, MultiFate-2.1 columns, and FIG. 4A). Initial A-only, B-only and A+B cells (rows) were sorted under the media conditions indicated on the top, and initial OFF cells came directly from cells in regular CHO media without any inducers. Each 2-dimensional flow cytometry plot was divided at mCherry=104 and mCitrine=2×104 into four quadrants, representing four states. For each plot, the percentage of cells in each of the four states is labeled on the corresponding corner. Timelines above each set of plots represent the indicated inducer conditions.

FIG. 15A-FIG. 15B depict non-limiting exemplary data related to raw flow cytometry analysis of the MultiFate-2.2 line. Each plot represents one of three biological replicates at the indicated time point (cf. FIG. 3C, MultiFate-2.2 columns). Initial A-only, B-only and A+B cells (rows) were sorted under the media conditions indicated on the top, and initial OFF cells came directly from cells in regular CHO media without any inducers. Each 2-dimensional flow cytometry plot was divided at mCherry=2×104 and mCitrine=3×104 into four quadrants, representing four states. For each plot, the percentage of cells in each of the four states is labeled on the corresponding corner. Timelines above each set of plots represent the indicated inducer conditions.

FIG. 16A-FIG. 16B depict non-limiting exemplary data related to raw flow cytometry analysis of the MultiFate-2.3 line. Each plot represents one of three biological replicates at the indicated time point (cf. FIG. 3C, MultiFate-2.3 columns). Initial A-only, B-only and A+B cells (rows) were sorted under the media conditions indicated on the top, and initial OFF cells came directly from cells in regular CHO media without any inducers. Each 2-dimensional flow cytometry plot was divided at mCherry=104 and mCitrine=104 into four quadrants, representing four states. For each plot, the percentage of cells in each of the four states is labeled on the corresponding corner. Timelines above each set of plots represent the indicated inducer conditions.

FIG. 17A-FIG. 17D depict non-limiting exemplary data showing raw time-lapse images separated by channels. Representative time-lapse images are separated by channels. The brightness and contrast for images in the same movie were adjusted to be the same. For MultiFate-2.3 time-lapse images in FIG. 17A, the intensity range of mCherry channel and mCitrine channel are [550, 800] and [300, 800], respectively. For MultiFate-3 time-lapse images, the intensity range of mCherry channel (FIG. 17B), mCitrine channel (FIG. 17C) and mTurquoise2 channel (FIG. 17D) are [600, 1400], [350, 800] and [1000, 1500], respectively.

FIG. 18A-FIG. 18B depict exemplary time-lapse movies that allow direct visualization of rare spontaneous state-switching events. FIG. 18A depicts non-limiting exemplary data showing two colonies (white boxes) in a MultiFate-2.3 time-lapse movie exhibited spontaneous state-switching events. Each example movie is shown as a filmstrip (See, FIG. 18A (continued)). Arrowheads indicate the cell that switched states. In the first event (top), a pair of A+B cells (yellow) appear from a colony started in the A-only (red) state (arrowheads). A similar transition was also identified in the second event (bottom, white arrowhead). FIG. 18B shows non-limiting exemplary data related to identification of four state-switching events (highlighted in white rectangles) in a MultiFate-3 time-lapse movie. Filmstrips (See, FIG. 18B (continued)) show the events. White arrowheads indicate cells that have switched states. The first event (top row) shows a transition from A+B+C (white) to A+B (yellow) and another transition to A-only state (red). Events 2 and 3 (second and third rows) show a transition from C-only (blue) to B+C (cyan). Event 4 (fourth row) shows a transition from A-only (red) to A+B (yellow). Time points of spontaneous state-switching are labeled in red type. Scale bar is 500 μm for the wide field image, and 100 μm for zoomed in images.

FIG. 19A-FIG. 19D depict non-limiting exemplary MultiFate state transition matrices showing low transition rates out of stable states, and distinct transition preferences out of unstable states for different cell lines. FIG. 19A depicts transition matrices for MultiFate-2.1. FIG. 19B depicts transition matrices for MultiFate-2.2. FIG. 19C depicts transition matrices for MultiFate-2.3. FIG. 19D depicts transition matrices for MultiFate-3. These transition matrices show that each predicted stable state (black bold text) has a very low level of cell transition out of the state (<1% every 3 days) as shown by weak off-diagonal blocks. The preferences of cells in each predicted unstable state (red bold text) to transition to different stable states are shown by the intensity of off diagonal blocks, and are consistent with results in FIG. 3C and FIG. 5B. The transition matrix among MultiFate states for different MultiFate lines from multi-day flow cytometry data (cf. FIG. 14A-FIG. 16B and other two biological replicates) was calculated using a method developed in T. Buder, A. Deutsch, M. Seifert, A. Voss-Böhme, CellTrans: An R Package to Quantify Stochastic Cell State Transitions. Bioinformatics and Biology Insights. 11 (2017), p. 117793221771224.

FIG. 20A-FIG. 20B depict non-limiting exemplary embodiments showing simulated cell fractions from best-fitted asymmetry parameter sets recapitulate experimental cell fractions of different MultiFate-2 lines in various conditions. Using stochastic asymmetry MultiFate models, the best-fitted parameter set was obtained to recapitulate the experimental data of each MultiFate cell line (See, Example 1 below). Each pair of start-end plots in ‘Model fitting’ columns were generated by stochastic simulations of 400 cells starting from each initial state in each condition. The asymmetry parameters for each MultiFate line were shown on the left, and the symmetry parameters were shown on the phase diagrams. K_{d}=1 and n=1.5. Note that the 3D phase diagrams in FIG. 20B were tilted slightly differently compared with those in FIG. 1D and FIG. 7A, for additional visualization of asymmetry.

FIG. 21A-FIG. 21C depict non-limiting exemplary data related to raw flow cytometry analysis of MultiFate-2.3 state-switching. Each plot represents one of three biological replicates at the indicated time point (cf. FIG. 4B). Initial A-only, B-only and A+B cells (rows) were sorted under the media conditions indicated on the top. Each 2-dimensional flow cytometry plot was divided at mCherry=104 and mCitrine=104 into four quadrants, representing four states. For each plot, the percentage of cells in each of the four states is labeled on the corresponding corner. Timelines above each set of plots represent the time and indicated inducer conditions. Note that since the response elements for 4-OHT or Dox are adjacent to the transcription factor homodimer binding sites (Table 2), the addition of 4-OHT or Dox increases A or B expression up to, but not substantially beyond, the level produced by transcription factor self-activation. For example, in FIG. 21C (top row), transcription factor A self-activation resulted in a 125-fold increase in expression (fold increase is calculated by dividing mCherry median in Day 18 by mCherry median in Day 0, mean of three replicates). Compared with transcription factor self-activation (Day 18), additional 4-OHT activation resulted in only another 2.8-fold increase in A expression (Day 6 versus Day 18). Similarly, in FIG. 21B (top row), transcription factor B self-activation resulted in a 48-fold increase in expression (Day 18 versus Day 0). Additional Dox activation further increased the B expression only by another 1.4 fold (Day 6 versus Day 18).

FIG. 22A-FIG. 22H depict non-limiting exemplary data related to raw flow cytometry data of MultiFate-3 line under the High TMP condition. Each plot represents one of three biological replicates at the indicated time point (cf. FIG. 5B, High TMP). Initial A-only, B-only and A+B cells were sorted under the media conditions indicated on the top, and initial OFF cells came directly from cells in regular CHO media without any inducers. Each 3-dimensional flow cytometry plot was divided at mCherry=2×104, mCitrine=4×104 and mTurquoise2=9×103 into eight octants, representing eight states. For each plot, the percentage of cells in each of the 7 states (excluding the OFF state) is labeled on the corresponding octant, as shown in legend at top-right. The timeline (top) represents the indicated inducer conditions. OFF state percentages are usually very low (<1%) across all conditions, and are separately labeled if the percentage is greater than 1%. One of three replicates of cells from each of the 7 initial states (excluding the OFF state) were continuously cultured beyond 18 days. In all 7 states, >90% of cells remained in their original state at day 37 (see indicated percentages).

FIG. 23A-FIG. 23B depict non-limiting exemplary data showing inducer withdrawal and reintroduction experiments showed MultiFate dependency on positive autoregulation and ruled out the possibility of mixed clones. Sorted cells in seven different states were transferred from AP1903+TMP media into regular media without any inducers. Most cells returned to the OFF state within 3 days (second column). After 6 days, AP1903+TMP was added back to the media, and cells were measured by flow cytometry after another 3 days. The resulting state distributions (fourth column) were similar to each other, suggesting that sorted cells in seven different states come from the same monoclonal MultiFate-3 line. Each plot represents the mean fractions of three biological replicates.

FIG. 24A-FIG. 24H depict non-limiting exemplary data related to raw flow cytometry data of MultiFate-3 line under the Intermediate TMP condition. Each plot represents one of three biological replicates at the indicated time point (cf. FIG. 5B, Intermediate TMP). Initial A-only, B-only and A+B cells were sorted under the media conditions indicated on the top, and initial OFF cells came directly from cells in regular CHO media without any inducers. For each plot, the percentage of cells in each of the 7 states (excluding the OFF state) is labeled on the corresponding octant. The timeline (top) represents the indicated inducer conditions. OFF state percentages are usually very low (<1%) across all conditions, and are separately labeled if the percentage is greater than 1%. Cells from A+B+C initial state were continuously cultured beyond 18 days and measured at day 31. This extended analysis revealed that cells continuously escaped from A+B+C state, as predicted, under the Intermediate TMP condition.

FIG. 25A-FIG. 25H depict non-limiting exemplary data related to raw flow cytometry data of MultiFate-3 line under the Low TMP condition. Each plot represents one of three biological replicates at the indicated time point (cf. FIG. 5B, Low TMP). Initial A-only, B-only and A+B cells were sorted under the media conditions indicated on the top, and initial OFF cells came directly from cells in regular CHO media without any inducers. For each plot, the percentage of cells in each of the 7 states (excluding the OFF state) is labeled on the corresponding octant. The timeline (top) represents the indicated inducer conditions. OFF state percentages are usually very low (<1%) across all conditions, and are separately labeled if the percentage is greater than 1%. Cells from A+B+C, A+B, A+C and B+C initial states were continuously cultured beyond 18 days. This extended analysis revealed that cells continuously escaped from these unstable states, as predicted, under the Low TMP condition.

FIG. 26A-FIG. 26E depict non-limiting exemplary embodiments showing MultiFate-3 exhibits predicted hysteresis. When transferred from High to Intermediate or Low TMP conditions, cells transition out of destabilized states, as expected. These transitions were irreversible, as shown by both modeling (FIG. 26A-FIG. 26E top) and experiments (FIG. 26A-FIG. 26E bottom). The model used here is symmetric and non-dimensionalized, with K_{d}=1, and n=1.5. The x, y and z axes are total dimensionless concentrations of TF A, B and C, respectively. Simulated cells on phase diagrams were calculated using the Gillespie algorithm. The left model in FIG. 26A-FIG. 26E shows initial conditions in simulations, with all cells in a single state at High TMP. The middle model in FIG. 26A-FIG. 26E shows steady-state density of cells in different states under Low or Intermediate TMP conditions. The right model in FIG. 26A-FIG. 26E shows that cells remain in states in the middle column after switching back to the High TMP condition. FIG. 26A-FIG. 26E (bottom) depict exemplary experiments showing similar hysteretic behaviors, largely consistent with modeling. In each row, initial cells for indicated states were sorted from the High TMP condition, where they were cultured for at least 3 days, and immediately transferred to Intermediate or Low TMP on day 0. They were then maintained in that condition for 18 or 31 days, as indicated, and then transferred back to the High TMP condition. The color indicates density of cells in each of the indicated states, as in FIG. 5A-FIG. 5D. Note that, in some embodiments, a difference between the simulations and experimental results is that actual cells escaping from destabilized states preferentially occupied the A-only state or states containing high A expression, instead of evenly distributing themselves across all states. This reflects some asymmetry of the experimental MultiFate-3 circuit. High TMP condition=100 nM AP1903+100 nM TMP; Intermediate TMP condition=100 nM AP1903+40 nM TMP; Low TMP condition=100 nM AP1903+10 nM TMP. Each plot represents the mean fractions of three biological replicates.

FIG. 27A-FIG. 27C depict non-limiting exemplary modeling of the robustness of MultiFate against intrinsic biological noise. FIG. 27A depicts non-limiting exemplary data showing that the OFF state is less robust against intrinsic noise compared with B-only state. (Left) The model used here is symmetric and non-dimensionalized, with K_{d}=1, and n=1.5. The x, y and z axes are total dimensionless concentrations of TF A, B and C, respectively. (Middle and Right) Cells starting from OFF state spontaneously switch out of OFF state, while cells from B-only state stay in their original state at the end of simulation. The traces are generated by Gillespie algorithm. The concentration is dimensionless. For MultiFate-3 type I quadrastable regime (See, FIG. 27A (continued)), larger relative basin size corresponds to higher robustness. Robustness score is defined as the fraction of cells not changing state at the end of simulation. The filled dots are the mean robustness scores of 50 simulated cells, and the error bar is the 95% confidence interval generated by bootstrapping. FIG. 27B-FIG. 27C depict non-limiting exemplary data showing that for both MultiFate-2 and MultiFate-3, robustness score is positively correlated with attractor basin size, as shown by the positive Spearman's p values. Spearman's p was used since it could assess non-linear monotonicity. 100 sets of parameters each were simulated with different combinations of α and β for MultiFate-2 and MultiFate-3. For each parameter set, the basin size of each stable fixed point is calculated, and the robustness of each stable fixed point is quantified by simulating 50 cells starting from that fixed point. For each fixed point, the relative basin size (FIG. 27B) is calculated by dividing its basin size by the average basin size of all fixed points from one parameter set. The characteristic length (FIG. 27C) is the Nth root (N is the dimension, either 2 or 3) of basin size, enabling comparison of basin sizes between MultiFate-2 and MultiFate-3.

FIG. 28A-FIG. 28C depict non-limiting exemplary embodiments showing the number of robust stable fixed points increased as MultiFate was expanded to include more transcription factors. (FIG. 28A left, FIG. 28B-FIG. 28C, top) The number of stable fixed points (blue dots) mostly increased monotonically with the number of transcription factors (except for MultiFate-10 and 11 in FIG. 28B), at a rate slower than the theoretical limit of 2N (N is the total number of transcription factors). This increase rate (the slope of blue dots) can be modulated up or down by α and β values. Since the model is non-dimensionalized, α and β can be tuned by transcriptional activation strength, protein stability and zinc finger DNA-binding affinity. In some embodiments, the theoretical limit is 2N because each transcription factor can be in either highly expressed or basally expressed state for any fixed points, which resulted in 2N points considering all combinations of binary states from N transcription factors. Among all stable fixed points, most (orange square) were robust to intrinsic biological noise, thus the number of robust stable fixed points followed the monotonic increasing trend of total stable fixed points. The robustness of a fixed point was quantified by a robustness score, which was the fraction of simulated cells not escaping from that fixed point at the end of stochastic simulation (See, Example 1 below). (FIG. 28A right, FIG. 28B-FIG. 28C, bottom) The number of fixed points grew more slowly than the theoretical limit because each parameter set only supports fixed points with up to a certain number of transcription factors simultaneously expressed at high level (denoted as number of TF ON). For each parameter set, all stable fixed points were plotted in the same plot, with number of TF ON on x axis and robustness score on y axis. Fixed points from different MultiFate systems were labeled with different colors. Since symmetric models were used, stable fixed points that have the same number of TF ON should have the same robustness score, thus each dot is an overlap of many fixed points. Small deviations resulted from stochasticity in the simulations. Low α and β values (FIG. 28A) only supported fixed points up to one transcription factor highly expressed. High α and β values (FIG. 28B) supported fixed points with up to four transcription factors highly expressed. Among them, fixed points with up to three transcription factors simultaneously ON were robust. A parameter set with even higher α and β values (FIG. 28C) supported fixed points with more transcription factors simultaneously ON. In these regimes, OFF fixed points sometimes were not stable or not robust. Here symmetric, non-dimensionalized and expanded MultiFate models with K_{d}=1 and n=1.5 were used.

FIG. 29 depicts non-limiting exemplary data showing that basal promoter expression can be modulated by modifying promoter sequences. Basal promoter expression and spontaneous self-activation in CHO cells can be increased by inserting GACGCTGCT (Table 5) repeats in the promoter. Note that GACGCTGCT is also the sequence motif bound by 42ZF, but its effect in increasing basal promoter expression does not require 42ZF. (Top) Schematics of three self-activation constructs different only in the number of GACGCTGCT repeats in the promoter. ZF=BCRZFR39A. Detailed construct maps are in Table 2. (Bottom) Increasing the number of GACGCTGCT repeats in the promoter increased the basal promoter expression, which can be observed by increased right shifts of mCherry− cell populations in regular media (red>orange>black). Higher basal promoter expression resulted in an increase in the fractions of cells (mCherry+ cells) that can spontaneously self-activate upon the addition of 100 nM AP1903 and 10 μM TMP (pink*>∀blue>‡magenta). Each of the three polyclonal cell populations was generated by stably integrating each construct in the CHO-K1 cells (Table 3) (See, Example 1 below). Different from FIG. 2C, polyclonal cell population was transferred directly from regular media to media containing AP1903 and TMP (instead of adding transient Dox treatment in between) to test spontaneous self-activation. Cells were harvested and measured by flow cytometry after 48 hours in regular media or AP1903+TMP media.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include nucleic acid compositions. In some embodiments, the nucleic acid composition comprises: a first promoter operably linked to a first polynucleotide encoding a first transcription factor (TF) and to a second polynucleotide encoding one or more first payloads. In some embodiments, the first promoter comprises one or more pairs of first TF binding sites. In some embodiments, the first TF comprises a first DNA-binding domain capable of binding a first TF binding site. In some embodiments, the first TF comprises a dimerization domain. In some embodiments, the dimerization domain of two first TF are capable of associating to generate a first TF homodimer. In some embodiments, a first TF homodimer is capable of binding the pair of first TF binding sites. In some embodiments, the dimerization domain of each of two first TF are capable of associating to generate the first TF homodimer in the presence of a dimerization ligand. In some embodiments, the dimerization ligand is a dimeric ligand. In some embodiments, the dimerization domain of each of two first TF are incapable of associating to generate the first TF homodimer in the absence of the dimerization ligand.

Disclosed herein include nucleic acid compositions. In some embodiments, the nucleic acid composition comprises: a second promoter operably linked to a third polynucleotide encoding a second transcription factor (TF) and to a fourth polynucleotide encoding one or more second payloads. In some embodiments, the second promoter comprises one or more pairs of second TF binding sites. In some embodiments, the second TF comprises a second DNA-binding domain capable of binding a second TF binding site. In some embodiments, the second TF comprises a dimerization domain. In some embodiments, the dimerization domain of two second TF are capable of associating to generate a second TF homodimer. In some embodiments, a second TF homodimer is capable of binding the pair of second TF binding sites. In some embodiments, the dimerization domain of each of two second TF are capable of associating to generate the second TF homodimer in the presence of a dimerization ligand. In some embodiments, the dimerization ligand is a dimeric ligand. In some embodiments, the dimerization domain of each of two second TF are incapable of associating to generate the second TF homodimer in the absence of the dimerization ligand.

Disclosed herein include nucleic acid compositions. In some embodiments, the nucleic acid composition comprises: a third promoter operably linked to a fifth polynucleotide encoding a third transcription factor (TF) and to a sixth polynucleotide encoding one or more third payloads. In some embodiments, the third promoter comprises one or more pairs of third TF binding sites. In some embodiments, the third TF comprises a third DNA-binding domain capable of binding a third TF binding site. In some embodiments, the third TF comprises a dimerization domain. In some embodiments, the dimerization domain of two third TF are capable of associating to generate a third TF homodimer. In some embodiments, a third TF homodimer is capable of binding the pair of third TF binding sites. In some embodiments, the dimerization domain of each of two third TF are capable of associating to generate the third TF homodimer in the presence of a dimerization ligand. In some embodiments, the dimerization ligand is a dimeric ligand. In some embodiments, the dimerization domain of each of two third TF are incapable of associating to generate the third TF homodimer in the absence of the dimerization ligand.

Disclosed herein include nucleic acid compositions. In some embodiments, the nucleic acid composition comprises: n supplemental promoters each operably linked to a nth supplemental polynucleotide encoding an nth supplemental transcription factor (sTF) and to a (n+1)th supplemental polynucleotide encoding one or more nth supplemental payloads. In some embodiments, n is 1, 2, 3, 4, 5, or 6. In some embodiments, the nth supplemental promoter comprises one or more pairs of nth supplemental TF binding sites. In some embodiments, the nth supplemental TF comprises a nth supplemental DNA-binding domain capable of binding a nth supplemental TF binding site. In some embodiments, the nth supplemental TF comprises a dimerization domain. In some embodiments, the dimerization domain of two nth supplemental TF are capable of associating to generate a nth supplemental TF homodimer. In some embodiments, a nth supplemental TF homodimer is capable of binding the pair of nth supplemental TF binding sites. In some embodiments, the dimerization domain of each of two nth supplemental TF are capable of associating to generate the nth supplemental TF homodimer in the presence of a dimerization ligand. In some embodiments, the dimerization ligand is a dimeric ligand. In some embodiments, the dimerization domain of each of two nth supplemental TF are incapable of associating to generate the nth supplemental TF homodimer in the absence of the dimerization ligand.

Disclosed herein include nucleic acid compositions. In some embodiments, the nucleic acid composition comprises: two or more of the nucleic acid compositions (e.g., circuits) disclosed herein. Disclosed herein include compositions. In some embodiments, the composition comprises: one or more nucleic acid compositions provided herein.

Disclosed herein include cells. In some embodiments, the cell comprises: one or more of the nucleic acid compositions provided herein.

Disclosed herein include cell populations. In some embodiments, the cell population comprises a plurality of cells. In some embodiments, each cell comprises one or more of the nucleic acid compositions provided herein.

Disclosed herein include method of treating a disease or disorder in a subject. In some embodiments, the method comprises: introducing into one or more cells one or more of the nucleic acid compositions provided herein or one or more of the compositions provided herein; and administering to the subject an effective amount of the one or more cells, or a cell population derived therefrom.

Disclosed herein include method of treating a disease or disorder in a subject. In some embodiments, the method comprises: administering to the subject an effective amount of cell(s) or cell population(s) provided herein.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, refers to a recombinant nucleic acid that has been generated for the purpose of the expression of a specific nucleotide sequence(s), or that is to be used in the construction of other recombinant nucleotide sequences.

As used herein, the term “plasmid” refers to a nucleic acid that can be used to replicate recombinant DNA sequences within a host organism. The sequence can be a double stranded DNA.

The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, refers to a recombinant nucleic acid that has been generated for the purpose of the expression of a specific nucleotide sequence(s), or that is to be used in the construction of other recombinant nucleotide sequences.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The term “antibody fragment” shall be given its ordinary meaning, and shall also refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

The term “autologous” shall be given its ordinary meaning, and shall also refer to any material derived from the same individual to whom it is later to be re-introduced into the individual.

The term “allogeneic” shall be given its ordinary meaning, and shall also refer to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “stimulation,” shall be given its ordinary meaning, and shall also refer to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

As used herein, 2A sequences or elements refer to small peptides introduced as a linker between two proteins, allowing autonomous intraribosomal self-processing of polyproteins (See e.g., de Felipe. Genetic Vaccines and Ther. 2: 13 (2004); de Felipe et al. Traffic 5:616-626 (2004)). These short peptides allow co-expression of multiple proteins from a single vector. Many 2A elements are known in the art. Examples of 2A sequences that can be used in the methods and system disclosed herein, without limitation, include 2A sequences from the foot-and-mouth disease virus (F2A), equine rhinitis A virus (E2A), Thosea asigna virus (T2A), and porcine teschovirus-1 (P2A).

### Multistable Synthetic Circuits

There are provided, in some embodiments, methods and compositions wherein single circuits can generate multiple molecularly and functionally distinct states that are each stable across multiple cell division cycles. Synthetic circuits provided herein can demonstrate multistability, defined as the ability of the circuit to stably exist in multiple distinct states characterized by differences in the concentrations and expression levels of its components. In the absence of changes to the external environment, each of these states can be stable.

There is provided herein a new circuit architecture termed MultiFate that advantageously allows the engineering of multistable circuits. The system is based, in some embodiments, on a few key properties. First, in some embodiments, it uses transcription factors that activate when dimerized, with much weaker activity as monomers. Second, in some embodiments, it incorporates positive autoregulation, in which each transcription factor homodimer activates expression of its own gene. Third, in some embodiments, the transcription factors can also form mixed heterodimers with one another that do not strongly activate any genes in the system, and therefore represent approximately ‘inert’ chemical species.

Different embodiments of the MultiFate synthetic circuits provided herein can use different numbers of transcription factors to produce a growing number of stable states. For example, two transcription factors can generate as many as 4 stable states; three transcription factors can generate 8 stable fixed points; and so on. In some embodiments, the MultiFate architecture allows external control of the states of the system, either using specific chemical inducers engineered to modify the state of the circuit, or by introducing transiently transfected transcription factor copies at the DNA, mRNA, or protein level. In some embodiments, the architecture has useful properties in that the number and locations of fixed points can be varied by externally controlling key properties such as dimerization strength or the protein stability of the engineered transcription factors. This allows a single circuit to support multiple fixed point configurations. Finally, in some embodiments, the circuit is extendable, such that addition of a new engineered transcription factor to an existing version of the multistable circuit can add additional stable states. With this feature, this architecture can scale to produce a rapidly increasing number of stable fixed points as one extends it with additional transcription factors. The MultiFate synthetic circuits provided herein can be useful for its power, controllability, extensibility, and for its compact design. The MultiFate synthetic circuits provided herein can have numerous applications, such as, for example, in, synthetic biology, engineered cell therapies, and regenerative medicine.

Synthetic tissue engineering. There is a need for methods of engineering synthetic tissues that can replace functions of damaged or lost tissues in injury or disease. These engineered tissues, like their natural counterparts, require interactions among multiple cell types. In some embodiments, MultiFate synthetic circuits provided herein enable the engineering of a single therapeutic cell type that can grow and “differentiate” into distinct MultiFate states that each provide different overall functions or different components of a single function. This capability exceeds what is possible with ordinary engineered cells that typically remain in a single state, or differentiate only through their natural fate control programs. MultiFate states can differentially regulate any number of endogenous or synthetic programs by linking the expression of MultiFate transcription factors to regulation of endogenous or synthetic genes.

Engineered cell therapies. Engineered cell therapies are an emerging field in biomedicine. In these approaches, cells are engineered to express synthetic proteins or, more complex regulatory circuits, and introduced into patients. One of the most successful examples to date are chimeric antigen receptor T (CAR-T) cells, which use an engineered receptor protein to selectively target cell populations, including tumor cells, senescent cells, and other therapeutically useful targets. Existing engineered cell therapies do not have the capability of operating in multiple distinct states. MultiFate can, in some embodiments, provide that capability. With the MultiFate synthetic circuits provided herein, these therapies can be engineered to control the number or percentage of cells in distinct states, allowing a single engineered cell population to diversify into distinct subpopulations that interact to perform a more powerful function. For example, different subpopulations of an engineered T cell can express distinct receptors specific for different antigens, and be engineered to signal to one another if all antigens are present in the same environment. In microencapsulation therapeutic strategies, the MultiFate synthetic circuits provided herein can also allow a single engineered cell type to produce a mixture of cell states at defined ratios that would together operate more efficiently to provide a therapeutically needed function. For example, in some embodiments, a first payload protein comprises a CAR targeting a first antigen, a second payload protein comprises a CAR targeting a second antigen, and a third payload protein comprises a cytokine. In some embodiments, such payloads would be expressed by the same or different subpopulations, and the state-specific program could be activated and/or tuned by the cellular environment (input signals between cells if all antigens are present in the same environment) and/or exogenous factors (e.g., a degron stabilizing molecule, a transactivator-binding compound, a dimerization ligand, or any combination thereof).

Classification of input signals in engineered biosensors. In many applications, cells need to be engineered to classify information encoded in multiple input signals. For example, it is useful to execute one genetic program when one input exceeds the level of a second input, and execute a distinct genetic program in the opposite signal regime. The MultiFate synthetic circuits disclosed herein provide, in some embodiments, a tunable system that can classify transient input signals into permanent (mitotically heritable) output states. In some embodiments, these input signals can directly or indirectly control the expression of the MultiFate transcription factors. In some embodiments, a cell can be engineered to respond to different natural or synthetic input signals by expressing transcription factors of MultiFate, or activators or repressors of MultiFate transcription factors. The multistable property of the MultiFate synthetic circuits provided herein can then cause the circuit to choose one of the stable states depending on the levels of all inputs. In this way, it effectively classifies the inputs into a specific number of discrete and exclusive output states. This can be useful for, e.g., classifying the immune environment around a cell population depending on the levels of cytokines and other signals, or in classifying a target cell based on its expression of multiple antigens.

MultiFate systems in other organisms. The MultiFate synthetic circuits provided herein can also be adapted to microbial systems, such as, for example, yeast, probiotic bacterial species, other prokaryotic microbes, plants and other eukaryotic species. While some embodiments herein relate to mammalian cells, the MultiFate architecture described herein can be extended to non-mammalian eukaryotic cell models (e.g., plants) as well as prokaryotic cell models (e.g., bacteria) wherein functions require the differentiation of cell populations into metabolically or functionally distinct subpopulations. The MultiFate synthetic circuits disclosed herein provides an efficient way to generate and control these distinct states. For example, the compositions and methods provided herein enable one to engineer a single microbial strain whose cells could occupy any of its distinct states, each activating a state-specific program. For example, in some embodiments, the first payload protein comprises an enzyme catalyzing a first anabolic reaction, the second payload protein comprises an enzyme catalyzing a second anabolic reaction (that starts with the product of the first anabolic reaction), and the third payload protein comprises an enzyme catalyzing a third anabolic reaction (that starts with the product of the second anabolic reaction). Said first payload protein, second payload protein, and third payload protein can be expressed in the same or different subpopulations of cells.

MultiFate can use diverse DNA binding proteins. In some embodiments, the MultiFate architecture can also work with a broad variety of transcription factors, using non-zinc finger DNA binding domains. Non-limiting examples include TALE DNA binding domain, catalytically dead CRISPR/Cas9 (dCas9), and others provided herein.

MultiFate can also use more complex multimerization schemes. In some embodiments, MultiFate systems can use other multimerization domains to mediate interactions among individual components. These include, e.g., homo- or hetero-dimerizing or multimerizing leucine zippers, PDZ domains, SH3 domains, GBD domains, and others. In some embodiments, these variant circuits can allow more complex distributions of states. In some embodiments, these multimerization schemes can also utilize condensate-based mechanisms in which sets of factors condense through multivalent interactions. The promoters can be configured based on the degree of multimerization of TFs. For example, in some embodiments wherein TFs are configured to form homotrimers, the promoter can be configured to bind said homotrimers (e.g., comprise three tandem repeats of a TF binding site).

Non-transcriptional MultiFate systems. In some embodiments, the MultiFate architecture can be implemented at multiple levels of biological regulation. In some embodiments, a protein-level MultiFate system can be engineered that has the same multistable properties as the transcriptional MultiFate system. For example, such a system may use, e.g., engineered auto-activating proteases in place of the transcription factors. If implemented with split protease variants reconstituted through modular dimerization domains, auto-activation can be made dependent on dimerization of complementary protease halves, and be inhibited by formation of mixed protease species halves. In some embodiments, the same circuit architecture can also be constructed using DNA- or RNA-based components by taking advantage of, e.g., branch migration and other programmable DNA and RNA molecular computing interactions.

Multiplexed binary switches. In some embodiments, a version of MultiFate can be engineered in which transcription factors homodimerize to self-activate, but do not heterodimerize. Without being bound by any particular theory, because each transcription factor species forms a bistable switch, this system can produce a set of independent binary switches in the same cell. This capability allows one to engineer cells that can occupy 2N different states, where N is the number of distinct transcription factor species, and allow the engineering of a set of independently switchable programs.

Differentially coupled MultiFate systems. In some embodiments, MultiFate can be engineered in which transcription factors homodimerize and/or heterodimerize with a subset of other MultiFate transcription factors. Each homodimer or heterodimer species can either be inert or activate one or several of MultiFate transcription factors. In some embodiments, this flexibility allows one to engineer cells that have cellular states and follow bifurcation trajectories that are different from the MultiFate systems that each transcription factor homodimerizes to activate itself and heterodimerize with all other transcription factors.

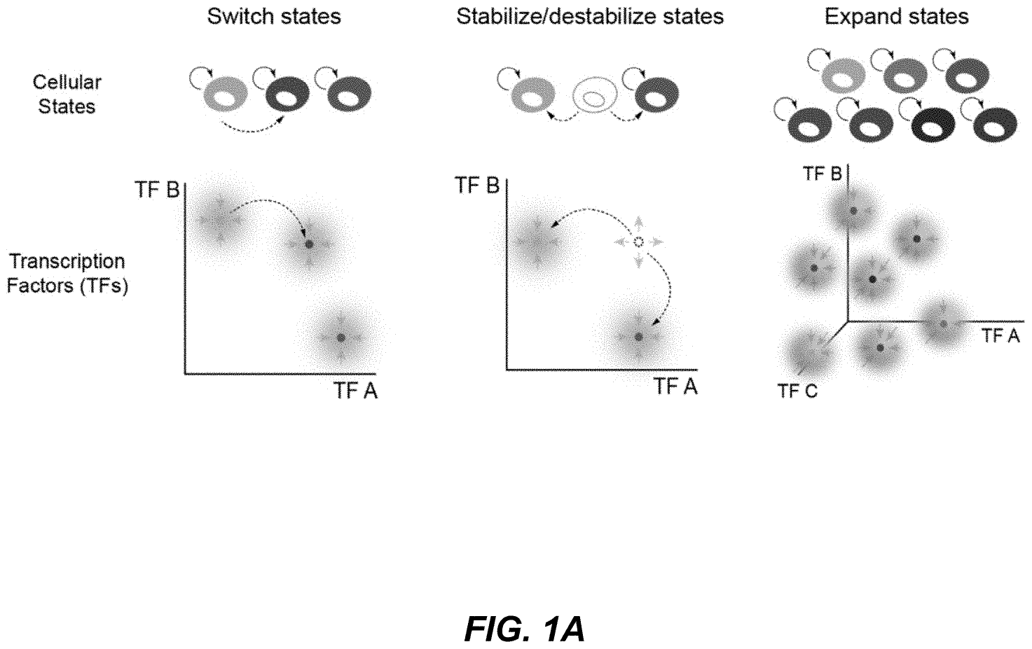

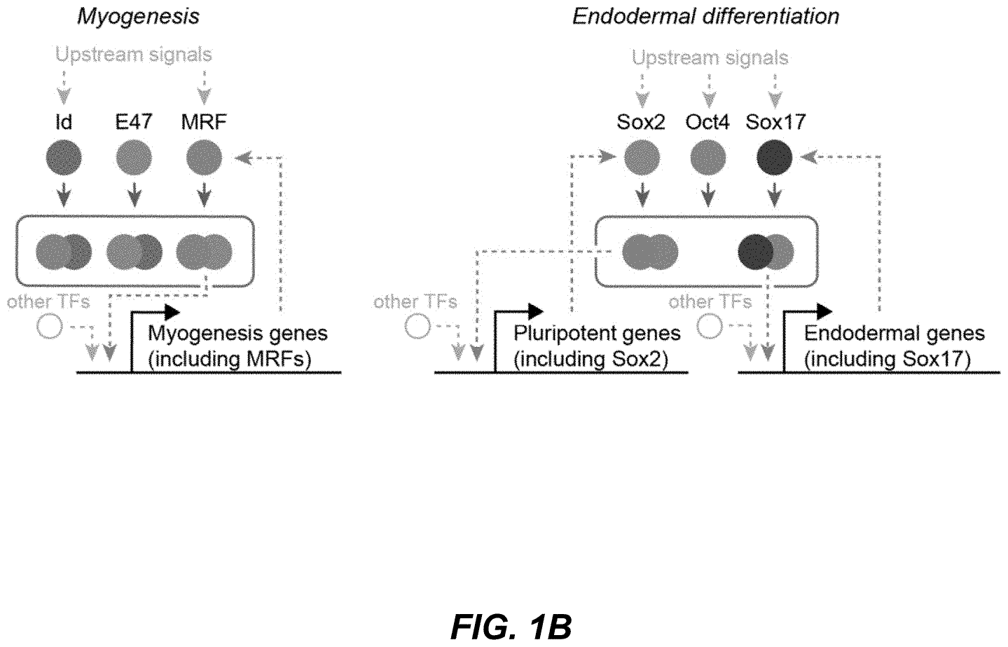

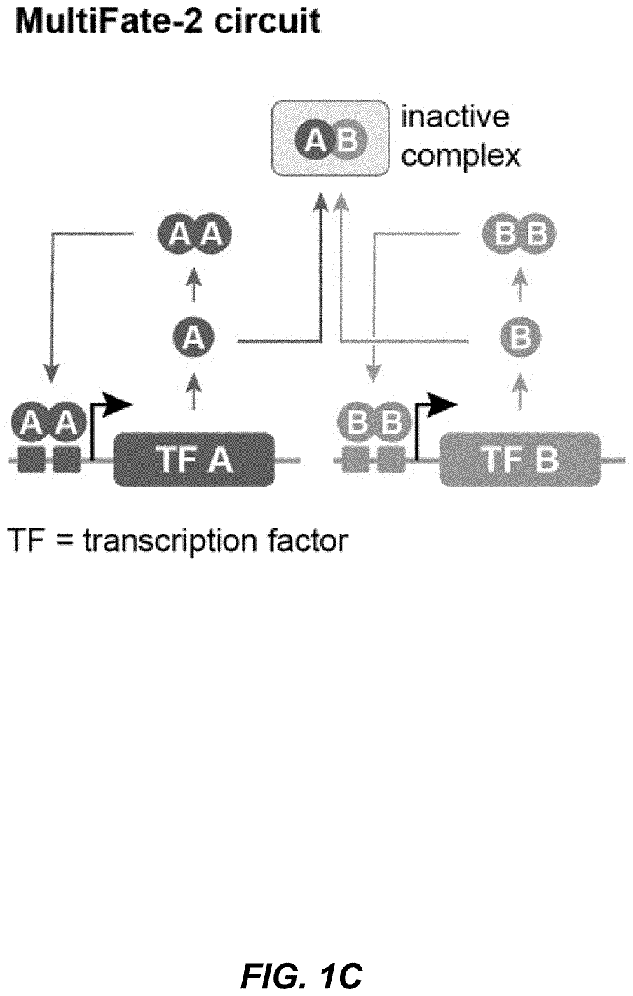

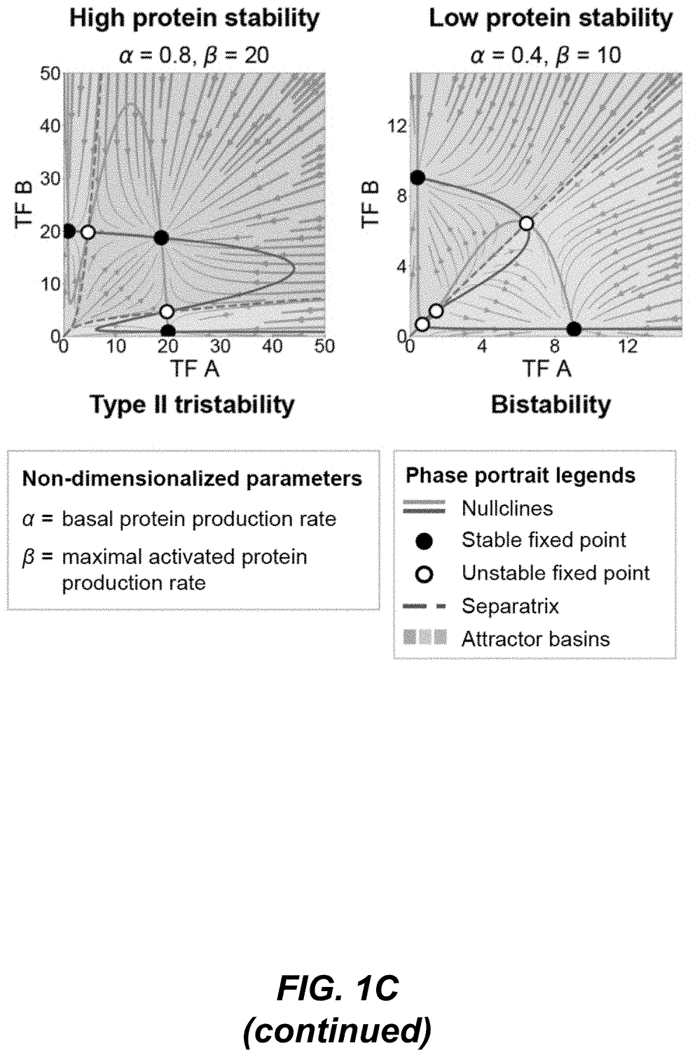

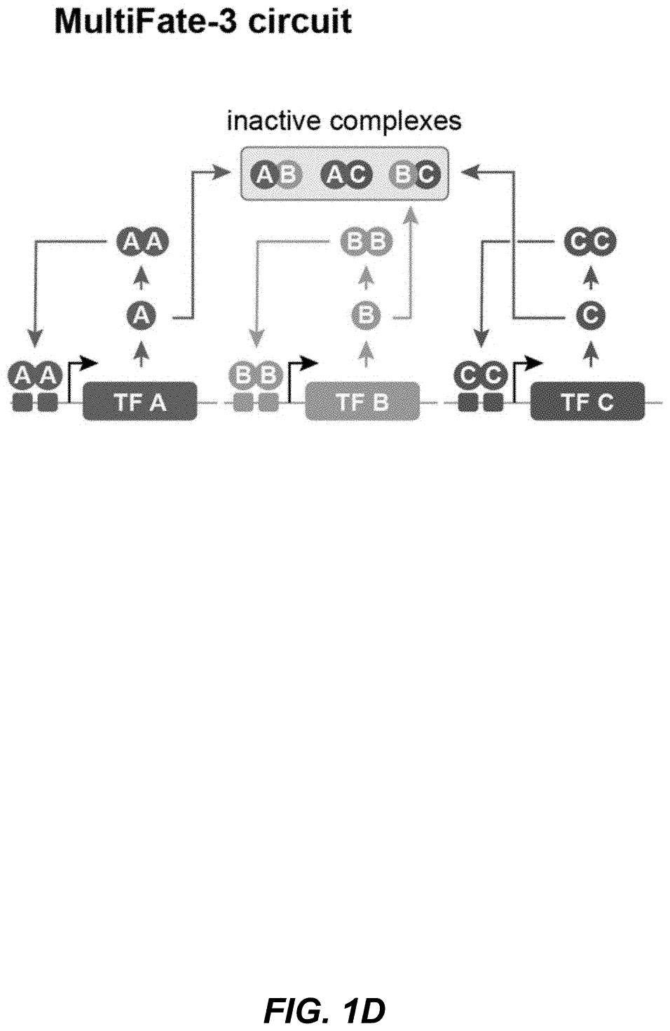

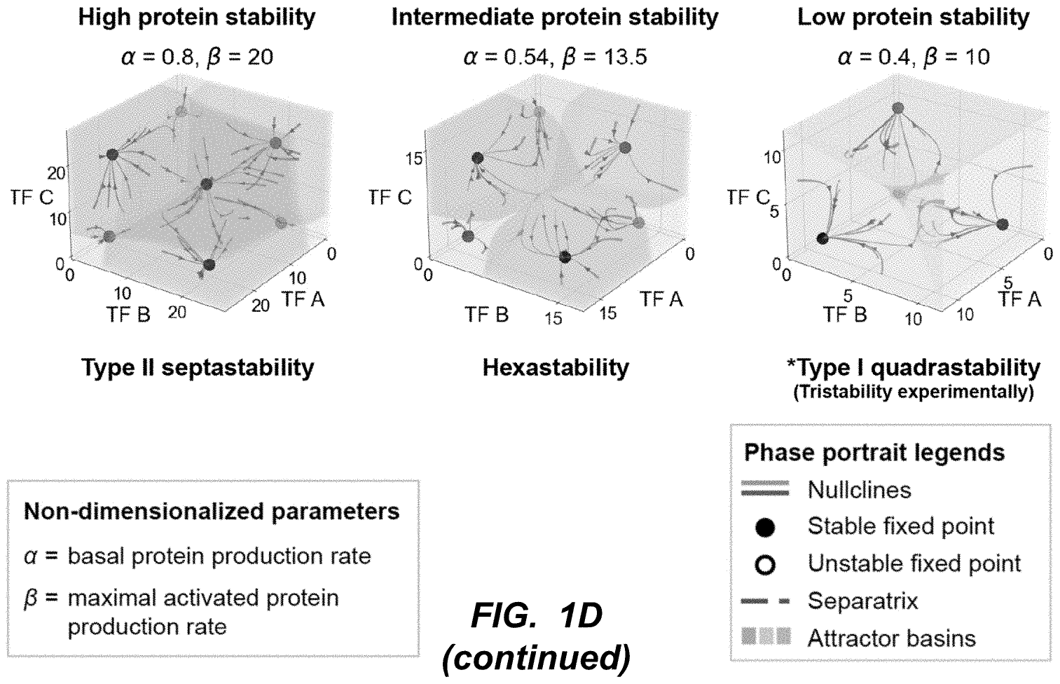

Multistability allows genetically identical cells to exist in thousands of molecularly distinct and mitotically stable cell types or states. Understanding natural multistable circuits and engineering synthetic ones have been long-standing challenges in developmental and synthetic biology. Building synthetic multistable circuits would provide insight into the minimal circuitry sufficient for multistability, and establish a foundation for exploiting multicellularity in engineered cell therapies. However, efforts in mammalian cells have been limited to two-state systems or used architectures that cannot be easily expanded to larger numbers of states. In some embodiments, an ideal synthetic multistable system would allow cells to remain in any of a set of distinct expression states over many cell cycles, despite biological noise. In addition, it would provide three key capabilities exhibited by its natural counterparts (FIG. 1A): First, it would permit transient external inputs to switch cells between states, similar to the way signaling pathways direct fate decisions. Second, it would support control over the stability of different states, and enable irreversible transitions, similar to those that occur during natural differentiation. Third, it would be expandable by introducing additional components without re-engineering an existing functional circuit, analogous to expansion of cell types during evolution.

Natural mammalian multistable circuits can provide inspiration for such a synthetic architecture. In many natural fate control systems, transcription factors positively autoregulate their own expression, and competitively interact with one another to form a variety of homodimers, heterodimers, and higher order multimeric forms (FIG. 1). For example, during myogenesis, muscle regulatory factors (MRF) such as MyoD heterodimerize with E proteins to activate their own expression and the broader myogenesis program, while Id family proteins disrupt this process through competitive dimerization. Similarly, during embryogenesis, Sox2 and Sox17 competitively interact with Oct4 to control fate decisions between pluripotency and endodermal differentiation. Without being bound by any particular theory, related combinations of positive autoregulation and cross-inhibition may extend multistability behaviors beyond bistability and generate bifurcation dynamics that explain the partial irreversibility of cell differentiation. Provided herein are nucleic acid compositions, cells, cell populations, and methods for a synthetic multistable system which can generate robust, controllable, expandable multistability in cells (e.g., mammalian cells).

### Circuit Components

Disclosed herein include nucleic acid compositions. In some embodiments, the nucleic acid composition comprises: a first promoter operably linked to a first polynucleotide encoding a first transcription factor (TF) and to a second polynucleotide encoding one or more first payloads.

In some embodiments, the first promoter comprises one or more pairs of first TF binding sites. The first TF can comprise a first DNA-binding domain capable of binding a first TF binding site. The first TF can comprise a dimerization domain. The dimerization domain of two first TF can be capable of associating to generate a first TF homodimer. A first TF homodimer can be capable of binding the pair of first TF binding sites. The dimerization domain of each of two first TF can be capable of associating to generate the first TF homodimer in the presence of a dimerization ligand. The dimerization ligand can be a dimeric ligand. The dimerization domain of each of two first TF can be incapable of associating to generate the first TF homodimer in the absence of the dimerization ligand.

The first TF can comprise a transactivation domain. In some embodiments, the first TF further comprises a degron capable of binding a degron stabilizing molecule. In some embodiments, the first TF changes from a destabilized state to a stabilized state when the degron binds to the degron stabilizing molecule. The one or more first payloads can comprise one or more first payload proteins and/or one or more first payload RNA agents. Upon the first TF homodimer binding a pair of first TF binding sites, the first promoter can be capable of inducing transcription of the first polynucleotide and the second polynucleotide to generate a first polycistronic transcript. The first polynucleotide and the second polynucleotide can be operably linked to a tandem gene expression element. The tandem gene expression element can be an internal ribosomal entry site (RES). The first polycistronic transcript can be capable of being translated to generate the first TF and the one or more first payloads. In some embodiments, the transcription factors and/or the payload protein(s) (e.g., a first payload, a second payload, a third payload, a supplemental payload) provided herein are co-expressed and comprise “self-cleaving” peptides (e.g., P2A, T2A, E2A and F2A).

In some embodiments, the first promoter further comprises one or more copies of a transactivator recognition sequence that a transactivator is capable of binding. In the presence of the transactivator and a transactivator-binding compound, the first promoter can be capable of inducing transcription of the first polynucleotide and the second polynucleotide to generate the first polycistronic transcript. In some embodiments, the first promoter further comprises one or more copies of a basal expression motif capable of inducing transcription of the first polynucleotide and the second polynucleotide to generate the first polycistronic transcript. The basal expression motif can comprise (GACGCTGCT). In some embodiments, the first promoter further comprises one or more first input elements capable of inducing or repressing transcription of the first polynucleotide and the second polynucleotide upon a first input reaching a threshold first input level.

Disclosed herein include nucleic acid compositions. In some embodiments, the nucleic acid composition comprises: a second promoter operably linked to a third polynucleotide encoding a second transcription factor (TF) and to a fourth polynucleotide encoding one or more second payloads.

The second promoter can comprise one or more pairs of second TF binding sites. The second TF can comprise a second DNA-binding domain capable of binding a second TF binding site. The second TF can comprise a dimerization domain. The dimerization domain of two second TF can be capable of associating to generate a second TF homodimer. A second TF homodimer can be capable of binding the pair of second TF binding sites. The dimerization domain of each of two second TF can be capable of associating to generate the second TF homodimer in the presence of a dimerization ligand. The dimerization ligand can be a dimeric ligand. The dimerization domain of each of two second TF can be incapable of associating to generate the second TF homodimer in the absence of the dimerization ligand.

The second TF can comprise a transactivation domain. In some embodiments, the second TF further comprises a degron capable of binding a degron stabilizing molecule. In some embodiments, the second TF changes from a destabilized state to a stabilized state when the degron binds to the degron stabilizing molecule. The one or more second payloads can comprise one or more second payload proteins and/or one or more second payload RNA agents. Upon the second TF homodimer binding a pair of second TF binding sites, the second promoter can be capable of inducing transcription of the third polynucleotide and the fourth polynucleotide to generate a second polycistronic transcript. The third polynucleotide and the fourth polynucleotide can be operably linked to a tandem gene expression element. The tandem gene expression element can be an internal ribosomal entry site (IRES). The second polycistronic transcript can be capable of being translated to generate the second TF and the one or more second payloads.

In some embodiments, the second promoter further comprises one or more copies of a transactivator recognition sequence that a transactivator is capable of binding. In the presence of the transactivator and a transactivator-binding compound, the second promoter can be capable of inducing transcription of the third polynucleotide and the fourth polynucleotide to generate the second polycistronic transcript. In some embodiments, the second promoter further comprises one or more copies of a basal expression motif capable of inducing transcription of the third polynucleotide and the fourth polynucleotide to generate the second polycistronic transcript. The basal expression motif can comprise (GACGCTGCT). In some embodiments, the second promoter further comprises one or more second input elements capable of inducing or repressing transcription of the third polynucleotide and the fourth polynucleotide upon a second input reaching a threshold second input level.

Disclosed herein include nucleic acid compositions. In some embodiments, the nucleic acid composition comprises: a third promoter operably linked to a fifth polynucleotide encoding a third transcription factor (TF) and to a sixth polynucleotide encoding one or more third payloads.

The third promoter can comprise one or more pairs of third TF binding sites. The third TF can comprise a third DNA-binding domain capable of binding a third TF binding site. The third TF can comprise a dimerization domain. The dimerization domain of two third TF can be capable of associating to generate a third TF homodimer. A third TF homodimer can be capable of binding the pair of third TF binding sites. The dimerization domain of each of two third TF can be capable of associating to generate the third TF homodimer in the presence of a dimerization ligand. The dimerization ligand can be a dimeric ligand. The dimerization domain of each of two third TF can be incapable of associating to generate the third TF homodimer in the absence of the dimerization ligand.

The third TF can comprise a transactivation domain. In some embodiments, the third TF further comprises a degron capable of binding a degron stabilizing molecule. In some embodiments, the third TF changes from a destabilized state to a stabilized state when the degron binds to the degron stabilizing molecule. The one or more third payloads can comprise one or more third payload proteins and/or one or more third payload RNA agents. Upon the third TF homodimer binding a pair of third TF binding sites, the third promoter can be capable of inducing transcription of the fifth polynucleotide and the sixth polynucleotide to generate a third polycistronic transcript. The fifth polynucleotide and the sixth polynucleotide can be operably linked to a tandem gene expression element. The tandem gene expression element can be an internal ribosomal entry site (IRES). The third polycistronic transcript can be capable of being translated to generate the third TF and the one or more third payloads.

In some embodiments, the third promoter further comprises one or more copies of a transactivator recognition sequence that a transactivator is capable of binding. In the presence of the transactivator and a transactivator-binding compound, the third promoter can be capable of inducing transcription of the fifth polynucleotide and the sixth polynucleotide to generate the third polycistronic transcript. In some embodiments, the third promoter further comprises one or more copies of a basal expression motif capable of inducing transcription of the fifth polynucleotide and the sixth polynucleotide to generate the third polycistronic transcript. The basal expression motif can comprise (GACGCTGCT). In some embodiments, the third promoter further comprises one or more third input elements capable of inducing or repressing transcription of the fifth polynucleotide and the sixth polynucleotide upon a third input reaching a threshold third input level.

Disclosed herein include nucleic acid compositions. In some embodiments, the nucleic acid composition comprises: n supplemental promoters each operably linked to a nth supplemental polynucleotide encoding an nth supplemental transcription factor (sTF) and to a (n+1)th supplemental polynucleotide encoding one or more nth supplemental payloads.

In some embodiments, n can be 1, 2, 3, 4, 5, 6, 7, 8, or 9. The nth supplemental promoter can comprise one or more pairs of nth supplemental TF binding sites. The nth supplemental TF can comprise a nth supplemental DNA-binding domain capable of binding a nth supplemental TF binding site. The nth supplemental TF can comprise a dimerization domain. The dimerization domain of two nth supplemental TF can be capable of associating to generate a nth supplemental TF homodimer. A nth supplemental TF homodimer can be capable of binding the pair of nth supplemental TF binding sites. The dimerization domain of each of two nth supplemental TF can be capable of associating to generate the nth supplemental TF homodimer in the presence of a dimerization ligand. The dimerization ligand can be a dimeric ligand. The dimerization domain of each of two nth supplemental TF can be incapable of associating to generate the nth supplemental TF homodimer in the absence of the dimerization ligand.

The nth supplemental TF can comprise a transactivation domain. In some embodiments, the nth supplemental TF further comprises a degron capable of binding a degron stabilizing molecule. In some embodiments, the nth supplemental TF changes from a destabilized state to a stabilized state when the degron binds to the degron stabilizing molecule. The one or more nth supplemental payloads can comprise one or more nth supplemental payload proteins and/or one or more nth supplemental payload RNA agents. Upon the nth supplemental TF homodimer binding a pair of nth supplemental TF binding sites, the nth supplemental promoter can be capable of inducing transcription of the nth supplemental polynucleotide and the (n+1)th supplemental polynucleotide to generate a nth supplemental polycistronic transcript. The nth supplemental polynucleotide and the (n+1)th supplemental polynucleotide can be operably linked to a tandem gene expression element. The tandem gene expression element can be an internal ribosomal entry site (TRES). The nth supplemental polycistronic transcript can be capable of being translated to generate the nth supplemental TF and the one or more nth supplemental payloads.

In some embodiments, the nth supplemental promoter further comprises one or more copies of a transactivator recognition sequence that a transactivator is capable of binding. In the presence of the transactivator and a transactivator-binding compound, the nth supplemental promoter can be capable of inducing transcription of the nth supplemental polynucleotide and the (n+1)th supplemental polynucleotide to generate the nth supplemental polycistronic transcript. In some embodiments, the nth supplemental promoter further comprises one or more copies of a basal expression motif capable of inducing transcription of the nth supplemental polynucleotide and the (n+1)th supplemental polynucleotide to generate the nth supplemental polycistronic transcript. The basal expression motif can comprise (GACGCTGCT). In some embodiments, the nth supplemental promoter further comprises one or more nth supplemental input elements capable of inducing or repressing transcription of the nth supplemental polynucleotide and the (n+1)th supplemental polynucleotide upon a nth supplemental input reaching a threshold nth supplemental input level.

The first TF, the second TF, the third TF, and/or nth sTF can be capable of self-activating and sustaining its own expression. The first TF, the second TF, the third TF, and/or nth sTF can comprise an amino acid sequence at least 50%, 51%, 52%, 53%, 54%, 55%, 56%, 57%, 58%, 59%, 60%, 61%, 62%, 63%, 64%, 65%, 66%, 670%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to NLS-FKBP12F36V-37ZFR2AR11AR39AR67A-VP16-NLS-DHFR (SEQ ID NO: 32), NLS-FKBP12F36V-BCRZFR39A-VP16-NLS-DHFR (SEQ ID NO: 33), or NLS-FKBP12F36V-ErbB2ZFR2AR39A-VP16-NLS-DHFR (SEQ ID NO: 34). One or more of the first TF, the second TF, the third TF, and/or nth sTF can be configured to homodimerize and to not heterodimerize with another TF. One or more of the first TF, the second TF, the third TF, and/or nth sTF can be configured to homodimerize and to heterodimerize with a subset of TFs.

An input element can comprise a heterologous promoter element and/or an endogenous promoter element. The heterologous promoter element can be capable of being bound by a component of a synthetic protein circuit. The endogenous promoter element can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. The tissue specific promoter can be a neuron-specific promoter. The neuron-specific promoter can comprise a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Naᵥ1.7 promoter, a Naᵥ1.8 promoter, a Naᵥ1.9 promoter, or an Advillin promoter. The tissue specific promoter can be a muscle-specific promoter. The muscle-specific promoter can comprise a creatine kinase (MCK) promoter. In some embodiments, a synthetic protein circuit component modulates the expression and/or activity of one or more TFs and/or one or more payloads. A Synthetic Notch (SynNotch) receptor, a Modular Extracellular Sensor Architecture (MESA) receptor, Tango, dCas9-synR, or any combination thereof, can be capable of modulating the expression and/or activity of one or more TFs and/or one or more payloads.

A first promoter, second promoter, third promoter, and/or nth supplemental promoter can comprise a minimal promoter (e.g., TATA, miniCMV, and/or miniPromo). A TF, a payload, and/or a transactivator can comprise a constitutive signal peptide for protein degradation (e.g., PEST). A TF, a payload, and/or a transactivator can comprise a nuclear localization signal (NLS) or a nuclear export signal (NES). The first polynucleotide, the second polynucleotide, the third polynucleotide, the fourth polynucleotide, the fifth polynucleotide, the sixth polynucleotide, the nth supplemental polynucleotide, and/or (n+1)th supplemental polynucleotide can be operably linked to a tandem gene expression element. The tandem gene expression element can be an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof. In some embodiments, the first polynucleotide, the second polynucleotide, the third polynucleotide, the fourth polynucleotide, the fifth polynucleotide, the sixth polynucleotide, the nth supplemental polynucleotide, and/or (n+1)th supplemental polynucleotide further comprises a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof. The first polynucleotide, the second polynucleotide, the third polynucleotide, the fourth polynucleotide, the fifth polynucleotide, the sixth polynucleotide, the nth supplemental polynucleotide, and/or (n+1)th supplemental polynucleotide can be evolutionarily stable for at least about 10 days, about 20 days, about 40 days, about 80 days, about 80 days, or about 100 days, of serial passaging. In some embodiments, a TF is not linked to a payload. In some such embodiments, a promoter is not linked to a polynucleotide encoding a payload.

Disclosed herein include nucleic acid compositions. In some embodiments, the nucleic acid composition comprises: two or more of the nucleic acid compositions disclosed herein. In some embodiments, the nucleic acid composition comprises: one more polynucleotides encoding at least one synthetic protein circuit component. The nucleic acid composition can comprise one or more vectors. At least one of the one or more vectors can be a viral vector, a plasmid, a transposable element, a naked DNA vector, a lipid nanoparticle, or any combination thereof. The viral vector can be an AAV vector, a lentivirus vector, a retrovirus vector, an integration-deficient lentivirus (IDLV) vector. The transposable element can be piggybac transposon or sleeping beauty transposon.

Disclosed herein include compositions. In some embodiments, the composition comprises: one or more nucleic acid compositions provided herein. In some embodiments, the composition comprises one or more vectors, a ribonucleoprotein (RNP) complex, a liposome, a nanoparticle, an exosome, a microvesicle, or any combination thereof. The vector can be a viral vector, a plasmid, a transposable element, a naked DNA vector, a lipid nanoparticle, or any combination thereof. The transposable element can be piggybac transposon or sleeping beauty transposon. The viral vector can be an AAV vector, a lentivirus vector, a retrovirus vector, an integration-deficient lentivirus (IDLV) vector. The AAV vector can comprise single-stranded AAV (ssAAV) vector or a self-complementary AAV (scAAV) vector.

Dimerization Domains

The dimerization domain can comprise or can be derived from GCN4, FKBP, cyclophilin, steroid binding protein, estrogen binding protein, glucocorticoid binding protein, vitamin D binding protein, tetracycline binding protein, extracellular domain of a cytokine receptor, a receptor tyrosine kinase, a TNFR-family receptor, an immune co-receptor, or any combination thereof. The dimerization domain can comprise an amino acid sequence at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to FKBP12F36V (SEQ ID NO: 5).

The dimerization domain can comprise or can be derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof.

The dimerization domain can be a homodimerization domain or a multimerization domain (e.g., a homo- or hetero-dimerizing or multimerizing leucine zipper, a PDZ domains, a SH3 domain, aGBD domain, or any combination thereof). The dimerization ligand can comprise or can be derived from AP1903, AP20187, dimeric FK506, a dimeric FK506-like analog, derivatives thereof, or any combination thereof. In some embodiments, the dimerization domain enables dose-dependent control of TF activation by the dimerization ligand. The dimerization domain of the first TF, the second TF, the third TF, and/or nth sTF can be the same. The dimerization domain of the first TF, the second TF, the third TF, and/or nth sTF can be different.

In some embodiments, the dimerization domains of (i) a first TF and a second TF, (ii) a first TF and a third TF, (iii) a first TF and an nth sTF; (iv) a second TF and a third TF, (v) a second TF and a nth sTF, and/or (vi) a third TF and a nth sTF, are capable of associating to generate a TF heterodimer. In some embodiments, the dimerization domains of (i) a first TF and a second TF, (ii) a first TF and a third TF, (iii) a first TF and an nth sTF; (iv) a second TF and a third TF, (v) a second TF and a nth sTF, and/or (vi) a third TF and a nth sTF, are capable of associating to generate a TF heterodimer in the presence of a dimerization ligand. The dimerization ligand can be a dimeric ligand.

A TF heterodimer can have at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 400-fold, 600-fold, 800-fold, or a number or a range between any of these values, less binding affinity for a pair of TF binding sites as compared to a TF homodimer. In some embodiments, a TF heterodimer is not capable of binding a pair of TF binding sites. In some embodiments, a first promoter, second promoter, third promoter, and/or nth supplemental promoter induces transcription at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 400-fold, 600-fold, 800-fold, or a number or a range between any of these values, less in the presence of a TF heterodimer as compared to a TF homodimer. A TF heterodimer can be incapable of causing a first promoter, second promoter, third promoter, and/or nth supplemental promoter to induce transcription. A TF monomer can have at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 400-fold, 600-fold, 800-fold, or a number or a range between any of these values, less binding affinity for a pair of TF binding sites as compared to a TF homodimer. In some embodiments, a first promoter, second promoter, third promoter, and/or nth supplemental promoter induces transcription at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 400-fold, 600-fold, 800-fold, or a number or a range between any of these values, less in the presence of a TF monomer as compared to a TF homodimer. In some embodiments, TF homodimerization and heterodimerization occur with a substantially equal dissociation constant (K_{d}).

A dimerization domain provided herein may contain amino acid sequences of or derived from, for example, FKBPs, cyclophilins, steroid binding proteins, estrogen binding proteins, glucocorticoid binding proteins, vitamin D binding proteins, or tetracycline binding proteins. In some embodiments, a dimerization domain may contain amino acid sequences of, or derived from, the extracellular domains of a receptor (e.g., cytokine receptor). In some embodiments, a dimerization domain may contain an amino acid sequence of an FKBP comprising a modification selected from the group consisting of: (i) a FKBP polypeptide containing F38V substitution, (ii) a FKBP polypeptide containing F36V and L106P substitutions, (iii) a FKBP polypeptide containing E31G, F38V, R71G, and K105E substitutions, and (iv) two or three tandem repeats of any of these FKBP polypeptides. In some embodiments, a dimerization domain may be cyclophilin polypeptide amino acid sequence. Cyclophilins are proteins that bind to ciclosporin (cyclosporin A). Cyclophilins include, for example, cyclophilin A and cyclophilin D. As used herein, a “dimeric” ligand may optionally contain more than two copies of a suitable binding molecule (i.e. the ligand may be multimeric); however, such ligands may still be considered “dimeric” as used herein, based on the ability of such ligands to dimerize corresponding binding molecules. Similarly, in some embodiments, a “dimerization domain” as provided herein may be capable of supporting multimerization (e.g. in the event that multiple copies of the dimerization domain are provided in the same molecule); however, such domains may also still be considered “dimerization domains” as used herein, based on the ability of such domains to dimerize.

Dimerization domains can comprise or be derived from one or more of the following: 14-3-3 domains, ADF domains, ANK repeats, ARM repeats, the BAR domain of amphiphysin, the BEACH domain, Bcl-2 homology (BH) domains (e.g., BH1, BH2, BH3, BH4), BIR domains, BRCT domains, bromodomains, BTB/POZ domains, C1 domains, C2 domains, caspase recruitment domains (CARDs), clathrin assembly lymphoid myeloid (CALM) domains, calponin homology (CH) domains, chromatin organization modifier (CHROMO/Chr) domains, CUE domains, death (DD) domains, death-effector (DED) domains, DEP domains, Dbl homology (DH) domains, EF-hand (EFh) domains, Eps15 homology (EH) domains, epsin NH2-terminal homology (ENTH) domains, Ena/Vasp Homology domain 1 (EVH1 domains), F-box domains, FERM domains, FF domains, formin homology-2 (FH2) domains, Forkhead-Associated (FH) domains, FYVE (Fab-1, YGL023, Vps27, and EEA1) domains, GAT (GGA and Tom1) domains, gelsolin/severin/villin homology (GEL) domains, GLUE (from GRAM-like ubiquitin-binding in EAP45) domains, GRAM (from glucosyltransferases, Rab-like GTPase activators and myotubularins) domains, GRIP domains, glycine-tyrosine-phenylalanine (GYF) domains, HEAT (from Huntington, Elongation Factor 3, PR65/A, TOR) domains, HECT (from Homologous to the E6-AP Carboxyl Terminus) domains, IQ domains, LIM domains, leucine-rich repeat (LRR) domains, malignant brain tumor (MBT) domains, Mad homology 1 (MH1) domains, MH2 domains, MIU (from Motif Interacting with Ubiquitin) domains, NZF (Np14 zinc finger) domains, PAS (Per-ARNT-Sim) domains, Phox and Bem1 (PB1) domains, PDZ (from postsynaptic density 95, PSD-85; discs large, D1g; zonula occludens-1, ZO-1) domains, Pleckstrin-homology (PH) domains, Polo-Box domains, phosphotyrosine binding (PTB) domains, pumilio (Puf) domains, PWWP domains, Phox homology (PX) domains, RGS (Regulator of G protein Signaling) domains, RING finger domains, SAM (Sterile Alpha Motif) domains, shadow chromo (CSD or SC) domains, Src-homology 2 (SH2) domains, Src-homology 3 (SH3) domains, SOCS (from suppressors of cytokine signaling) box domains, SPRY domains, START (from steroidogenic acute regulatory protein (StAR) related lipid transfer) domains, SWIRM domains, Toll/Il-1 Receptor (TIR) domains, tetratricopeptide repeat (TPR) motif domains, TRAF domains, SNARE (from soluble NSF attachment protein (SNAP) receptors) domains (e.g., T-SNARE), Tubby domains, tudor domains, ubiquitin-associated (UBA) domains, UEV (Ubiquitin E2 variant) domains, ubiquitin-interacting motif (UIM) domains, beta-domains of the von Hippel-Lindau tumor suppressor protein (VHLO), VHS (from Vps27p, Hrs and STAM) domains, WD40 repeat domains, and WW domains.

DNA-Binding Domains

A DNA-binding domain (e.g., a first DNA-binding domain, a second DNA-binding domain, a third DNA-binding domain, a supplemental DNA-binding domain) can comprise or can be derived from: a TALE DNA binding domain 2, catalytically dead CRISPR/Cas9 (dCas9) 3-5, Gal4, hypoxia inducible factor (HIF), HIF1a, cyclic AMP response element binding (CREB) protein, LexA, rtTA, an endonuclease, a zinc finger (ZF) binding domain, a transcription factor, portions thereof, or any combination thereof. The DNA-binding domain can be a synthetic DNA-binding domain configured to decrease monomeric TF activity without reducing TF homodimer activity. A DNA-binding domain can comprise or can be derived from a zinc finger DNA-binding domain. The zinc finger (ZF) DNA-binding domain can comprise or can be derived from ErbB2 ZF, BCRZF, HIV1ZF, HIV2ZF, 37ZF (37-12 array), 42ZF (42-10 array), 43ZF (43-8 array), 92ZF (92-1 array), and/or 97ZF (97-4 array). The ZF DNA-binding domain can comprise one or more arginine-to-alanine mutations. The ZF DNA-binding domain can comprise three fingers that bind weakly as monomers to 9 bp target sites and bind at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 400-fold, 600-fold, 800-fold, or a number or a range between any of these values, more strongly as homodimers to 18 bp tandem binding site pairs.

A DNA-binding domain (e.g., a first DNA-binding domain, a second DNA-binding domain, a third DNA-binding domain, a supplemental DNA-binding domain) can comprise an amino acid sequence at least 50%, 51%, 52%, 53% 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to ErbB2ZFWT (SEQ ID NO: 6), ErbB2ZFR39A (SEQ ID NO: 7), ErbB2ZFR2AR39A (SEQ ID NO: 8), ErbB2ZFR2AR39AR67A (SEQ ID NO: 9), 37ZFWT (SEQ ID NO: 10), 37ZFR39A (SEQ ID NO: 11), 37ZFR2AR39A (SEQ ID NO: 12), 37ZFR2AR39AR67A (SEQ ID NO: 13), 42ZFR2AR39AR67A (SEQ ID NO: 14), 92ZFWT (SEQ ID NO: 15), 92ZFR39A (SEQ ID NO: 16), 92ZFR2AR39A (SEQ ID NO: 17), 92ZFR2AR39AR67A (SEQ ID NO: 18), 97ZFWT (SEQ ID NO: 19), 97ZFR39A (SEQ ID NO: 20), 97ZFR2AR39A (SEQ ID NO: 21), BCRZF (SEQ ID NO: 22), BCRZFR39A (SEQ ID NO: 23), HIV1ZFWT (SEQ ID NO: 24), HIV1ZFR39A (SEQ ID NO: 25), HIV1ZFR2AR39A (SEQ ID NO: 26), HIV1ZFR2AR39AR67A (SEQ ID NO: 27), HIV2ZFWT (SEQ ID NO: 28), HIV2ZFR39A (SEQ ID NO: 29), HIV2ZFR2AR39A (SEQ ID NO: 30), and/or HIV2ZFR2AR39AR67A (SEQ ID NO: 31).

In some embodiments, the first TF, the second TF, the third TF, and/or nth sTF share substantially identical biochemical parameters and differ only in their DNA binding site specificity. In some embodiments, the first TF, the second TF, the third TF, and/or nth sTF have orthogonal DNA-binding specificities. In some embodiments, the pair of first TF binding sites, the pair of second TF binding sites, the pair of third TF binding sites, and/or the pair of nth supplemental TF binding sites is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to 42bs_42bs (SEQ ID NO: 1), 37bs_37bs (SEQ ID NO: 2), BCRbs_BCRbs (SEQ ID NO: 3), ErbB2bs_ErbB2bs (SEQ ID NO: 4), portions thereof, or any combination thereof.

Transactivation Domains

A transactivation domain can comprise or can be derived from VP16, TA2, VP64 (a tetrameric repeat of the minimal activation domain of VP16), VP48 (a trimeric repeat of the minimal activation domain of VP16), signal transducer and activator of transcription 6 (STAT6), reticuloendotheliosis virus A oncogene (relA), TATA binding protein associated factor-1 (TAF-1), TATA binding protein associated factor-2 (TAF-2), glucocorticoid receptor TAU-1, or glucocorticoid receptor TAU-2, a steroid/thyroid hormone nuclear receptor transactivation domain, a polyglutamine transactivation domain, a basic or acidic amino acid transactivation domain, a GAL4 transactivation domain, an NF-κB transactivation domain, a p65 transactivation domain, a BP42 transactivation domain, HSF1, VP16, VP64, p65, MyoD1, RTA, SET7/9, VPR, histone acetyltransferase p300, an hydroxylase catalytic domain of a TET family protein (e.g., TETl hydroxylase catalytic domain), LSD1, CIB1, AD2, CR3, EKLF1, GATA4, PRVIE, p53, SP1, MEF2C, TAX, and PPARγ, Gal4, Gcn4, MLL, Rtg3, Gln3, Oaf1, Pip2, Pdr1, Pdr3, Pho4, Leu3, portions thereof having transcription activating activity, or any combination thereof. The transactivation domain of the first TF, the second TF, the third TF, and/or nth sTF can be the same. The transactivation domain of the first TF, the second TF, the third TF, and/or nth sTF can be different.

Transactivators

In some embodiments, the nucleic acid composition comprises: one or more polynucleotides encoding a transactivator. The one or more polynucleotides encoding a transactivator can be under the control of a ubiquitous promoter. The ubiquitous promoter can be selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

A transactivator recognition sequence can comprise a Tet3G binding site (TRE3G) or a ERT2-Gal4 binding site (UAS). The transactivator-binding compound can comprise 4-hydroxy-tamoxifen (4-OHT), Dox, derivatives thereof, or any combination thereof. In the presence of the transactivator and a transactivator-binding compound, the first promoter can be capable of inducing transcription up to, but not substantially beyond, the level produced by a TF homodimer binding a pair of TF binding sites. A transactivator recognition sequence can comprise an element of an inducible promoter. The inducible promoter can be a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, or an RU-486 responsive promoter.

Degrons

The degron can comprise a dihydrofolate reductase (DHFR) degron, a FKB protein (FKBP) degron (See, e.g., Banaszynski et al, 2006), derivatives thereof, or any combination thereof. The degron stabilizing molecule can comprise trimethoprim (TMP), Shield-1, derivatives thereof, or any combination thereof. The degron can comprise or be derived from one or more of the following: a furin degron (FurON) domain; a degron derived from an FKB protein (FKBP); a degron derived from dihydrofolate reductase (DHFR); a degron derived from an estrogen receptor (ER); a degron derived from an Ikaros family of transcription factors (e.g., IKZF1, or IKZF3); or a degron derived from a protein listed in Table 21 of International Application WO 2017/181119.

Additional Elements

The nucleic acid can comprise at least one regulatory element for expression of the synthetic protein circuit. The nucleic acid can comprise a vector, such as any of the viral vectors described in US2020/0071723, the content of which is incorporated herein by reference in its entirety. In some embodiments, the vector can comprise an adenovirus vector, an adeno-associated virus vector, an Epstein-Barr virus vector, a Herpes virus vector, an attenuated HIV vector, a retroviral vector, a vaccinia virus vector, or any combination thereof. In some embodiments, the vector can comprise an RNA viral vector. In some embodiments, the vector can be derived from one or more negative-strand RNA viruses of the order Mononegavirales. In some embodiments, the vector can be a rabies viral vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Retroviral vectors can be “defective”, i.e. unable to produce viral proteins required for productive infection. Replication of the vector can require growth in the packaging cell line. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., a synthetic protein circuit component) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

In some embodiment, the vectors can include a regulatory sequence that allows, for example, the translation of multiple proteins from a single mRNA. Non-limiting examples of such regulatory sequences include internal ribosome entry site (IRES) and 2A self-processing sequence. In some embodiments, the 2A sequence is a 2A peptide site from foot-and-mouth disease virus (F2A sequence). In some embodiments, the F2A sequence has a standard furin cleavage site. In some embodiments, the vector can also comprise regulatory control elements known to one of skill in the art to influence the expression of the RNA and/or protein products encoded by the polynucleotide within desired cells of the subject. In some embodiments, functionally, expression of the polynucleotide is at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an ration and the splice donor and splice acceptor sequences that regulate the splicing of said intron. Another example of a regulatory element is a transcription termination signal and/or a polyadenylation sequence.

Expression control elements and promoters include those active in a particular tissue or cell type, referred to herein as a “tissue-specific expression control elements/promoters.” Tissue-specific expression control elements are typically active in specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited, to the cytomegalovirus (CMV) immediate early promoter/enhancer sequences, the Rous sarcoma virus (RSV) promoter/enhancer sequences and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide m response to a signal or stimuli is also referred to as an “inducible element” (that is, it is induced by a signal). Particular examples include, but are not limited to, a hormone (for example, steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression

The nucleic acid composition can comprise one or more vectors. At least one of the one or more vectors can be a viral vector, a plasmid, a naked DNA vector, a lipid nanoparticle, or any combination thereof. The vector can be a transposable element (e.g., piggybac). The viral vector can be an AAV vector, a lentivirus vector, a retrovirus vector, an integration-deficient lentivirus (IDLV) vector. Disclosed herein include compositions. In some embodiments, the composition comprises: one or more of the nucleic acid compositions (e.g., circuits) provided herein. The composition can comprise one or more vectors, a ribonucleoprotein (RNP) complex, a liposome, a nanoparticle, an exosome, a microvesicle, or any combination thereof. The vector can be a viral vector, a plasmid, a naked DNA vector, a lipid nanoparticle, or any combination thereof. The viral vector can be an AAV vector, a lentivirus vector, a retrovirus vector, an integration-deficient lentivirus (IDLV) vector. The AAV vector can comprise single-stranded AAV (ssAAV) vector or a self-complementary AAV (scAAV) vector.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

The term “lentivirus” refers to a genus of the Retroviridae family Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector. Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

Vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

A number of viral based systems have been developed for gene transfer into mammalian cells (e.g., immune cells). For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

The nucleic acid composition can be single-stranded or double-stranded. The nucleic acid composition can contain two or more nucleic acids. The two or more nucleic acids can be in the same form (e.g., a first plasmid and a second plasmid) or different in forms (e.g., a first plasmid and a first viral vector).

Additional Synthetic Protein Circuits

In some embodiments of the circuits, compositions, nucleic acids, populations, systems, and methods provided herein, one or more components of the disclosed circuits interfaces with (e.g., modulates and/or is modulated by) another synthetic protein circuit component. The payload protein(s), transcription factor(s), promoter(s), transactivator (s), and/or input(s), described herein can comprise, be under the control of, or modulate (directly or indirectly) a synthetic protein circuit component. Synthetic biology allows for rational design of circuits that confer new functions in living cells. Many natural cellular functions are implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and PCT Application Publication No. WO 2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a payload at an efficacious level, such as those provided in US2021/0171582, the content of which is incorporated herein by reference in its entirety. The compositions, methods, systems and kits provided herein can be employed in concert with those described in International Patent Application No. PCT/US2021/048100, entitled “SYNTHETIC MAMMALIAN SIGNALING CIRCUITS FOR ROBUST CELL POPULATION CONTROL” filed on Aug. 27, 2021, the content of which is incorporated herein by reference in its entirety. Said reference discloses circuits, compositions, nucleic acids, populations, systems, and methods enabling cells to sense, control, and/or respond to their own population size and can be employed with the circuits provided herein. In some embodiments, an orthogonal communication channel allows specific communication between engineered cells. Also described therein, in some embodiments, is an evolutionarily robust ‘paradoxical’ regulatory circuit architecture in which orthogonal signals both stimulate and inhibit net cell growth at different signal concentrations. In some embodiments, engineered cells autonomously reach designed densities and/or activate therapeutic or safety programs at specific density thresholds.

### Payloads

The payload protein(s) (e.g., a first payload, a second payload, a third payload, a supplemental payload) can comprise a synthetic protein circuit component. In some embodiments, the payload comprises a bispecific T cell engager (BiTE). In some embodiments, the orthogonal signal triggers cellular differentiation. The payload protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The payload protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The payload protein can comprise a CRE recombinase, GCaMP, a cell therapy component, a knock-down gene therapy component, a cell-surface exposed epitope, or any combination thereof. The payload protein can comprise a diagnostic agent (e.g., green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3 rsCherry, rsCherryRev, derivatives thereof, or any combination thereof).

A payload protein can be capable of modulating the concentration, localization, stability, and/or activity of the one or more targets. A payload protein can be capable of repressing the transcription of the one or more targets. A target transcript can be capable of being translated to generate a target protein. A payload protein can be capable of reducing the concentration, localization, stability, and/or activity of the target protein. The concentration, localization, stability, and/or activity of the target protein can be inversely related to the concentration, localization, stability, and/or activity of a payload protein. A payload protein can comprise a protease. The target protein can comprise a degron and a cut site the protease can be capable of cutting to expose the degron. In some embodiments, the degron of the target protein being exposed changes the target protein to a target protein destabilized state. The protease can comprise tobacco etch virus (TEV) protease, tobacco vein mottling virus (TVMV) protease, hepatitis C virus protease (HCVP), derivatives thereof, or any combination thereof. In some embodiments, the target protein comprises a cage polypeptide, wherein the cage polypeptide comprises: (a) a helical bundle, comprising between 2 and 7 alpha-helices, wherein the helical bundle comprises: (i) a structural region; and (ii) a latch region, wherein the latch region comprises a degron located within the latch region, wherein the structural region interacts with the latch region to prevent activity of the degron; and (b) amino acid linkers connecting each alpha helix. A payload protein can comprise a key polypeptide capable of binding to the cage polypeptide structural region, thereby displacing the latch region and activating the degron.

The payload can comprise a pro-death protein capable of halting cell growth and/or inducing cell death. The pro-death protein can comprise cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. The pro-death protein can be capable of halting cell growth and/or inducing cell death in the presence of a pro-death agent. In some embodiments, the pro-death protein comprises Caspase-9 and the pro-death agent comprises AP1903; the pro-death protein comprises HSV thymidine kinase (TK) and the pro-death agent Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT); the pro-death protein comprises Cytosine Deaminase (CD) and the pro-death agent comprises 5-fluorocytosine (5-FC); the pro-death protein comprises Purine nucleoside phosphorylase (PNP) and the pro-death agent comprises 6-methylpurine deoxyriboside (MEP) and/or fludarabine (FAMP); the pro-death protein comprises a Cytochrome p450 enzyme (CYP) and the pro-death agent comprises Cyclophosphamide (CPA), Ifosfamide (IFO), and/or 4-ipomeanol (4-IM); the pro-death protein comprises a Carboxypeptidase (CP) and the pro-death agent comprises 4-[(2-chloroethyl)(2-mesyloxyethyl)amino]benzoyl-L-glutamic acid (CMDA), Hydroxy- and amino-aniline mustards, Anthracycline glutamates, and/or Methotrexate α-peptides (MTX-Phe); the pro-death protein comprises Carboxylesterase (CE) and the pro-death agent comprises Irinotecan (IRT), and/or Anthracycline acetals; the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones; the pro-death protein comprises Horse radish peroxidase (HRP) and the pro-death agent comprises Indole-3-acetic acid (IAA) and/or 5-Fluoroindole-3-acetic acid (FIAA); the pro-death protein comprises Guanine Ribosyltransferase (XGRTP) and the pro-death agent comprises 6-Thioxanthine (6-TX); the pro-death protein comprises a glycosidase enzyme and the pro-death agent comprises HM1826 and/or Anthracycline acetals; the pro-death protein comprises Methionine-α,γ-lyase (MET) and the pro-death agent comprises Selenomethionine (SeMET); and/or the pro-death protein comprises thymidine phosphorylase (TP) and the pro-death agent comprises 5′-Deoxy-5-fluorouridine (5′-DFU). Methods disclosed herein can comprise administering a pro-death agent (e.g., administering to a subject).

A payload protein can be associated with an agricultural trait of interest selected from the group consisting of increased yield, increased abiotic stress tolerance, increased drought tolerance, increased flood tolerance, increased heat tolerance, increased cold and frost tolerance, increased salt tolerance, increased heavy metal tolerance, increased low-nitrogen tolerance, increased disease resistance, increased pest resistance, increased herbicide resistance, increased biomass production, male sterility, or any combination thereof. A payload protein can be associated with a biological manufacturing process selected from the group comprising fermentation, distillation, biofuel production, production of a compound, production of a polypeptide, or any combination thereof.

In some embodiments, the payload protein(s) (e.g., a first payload, a second payload, a third payload, a supplemental payload) can diminish immune cell function. The payload protein(s) can be an activity regulator. The activity regulator can be capable of reducing T cell activity. The activity regulator can comprise a ubiquitin ligase involved in TCR/CAR signal transduction selected from the group comprising c-CBL, CBL-B, ITCH, R F125, R F128, WWP2, or any combination thereof. The activity regulator can comprise a negative regulatory enzyme selected from the group comprising SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof. The activity regulator can be a negative regulatory scaffold/adapter protein selected from the group comprising PAG, LIME, NTAL, LAX31, SIT, GAB2, GRAP, ALX, SLAP, SLAP2, DOK1, DOK2, or any combination thereof. The activity regulator can be a dominant negative version of an activating TCR signaling component selected from the group comprising ZAP70, LCK, FYN, NCK, VAV1, SLP76, ITK, ADAP, GADS, PLCgamma1, LAT, p85, SOS, GRB2, NFAT, p50, p65, API, RAPI, CRKII, C3G, WAVE2, ARP2/3, ABL, ADAP, RIAM, SKAP55, or any combination thereof. The activity regulator can comprise the cytoplasmic tail of a negative co-regulatory receptor selected from the group comprising CD5, PD1, CTLA4, BTLA, LAG3, B7-H1, B7-1, CD160, TFM3, 2B4, TIGIT, or any combination thereof. The activity regulator can be targeted to the plasma membrane with a targeting sequence derived from LAT, PAG, LCK, FYN, LAX, CD2, CD3, CD4, CD5, CD7, CD8a, PD1, SRC, LYN, or any combination thereof. In some embodiments, the activity regulator reduces or abrogates a pathway and/or a function selected from the group comprising Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, or any combination thereof.

The payload protein(s) can comprise a factor locally down-regulating the activity of endogenous immune cells. In some embodiments, the payload protein(s) comprises a prodrug-converting enzyme (e.g., HSV thymidine kinase (TK), Cytosine Deaminase (CD), Purine nucleoside phosphorylase (PNP), Cytochrome p450 enzymes (CYP), Carboxypeptidases (CP), Caspase-9, Carboxylesterase (CE), Nitroreductase (NTR), Horse radish peroxidase (HRP), Guanine Ribosyltransferase (XGRTP), Glycosidase enzymes, Methionine-α,γ-lyase (MET), Thymidine phosphorylase (TP)).

In some embodiments, the payload gene encodes a payload RNA agent. A payload RNA agent can comprise one or more of dsRNA, siRNA, shRNA, pre-miRNA, pri-miRNA, miRNA, stRNA, IncRNA, piRNA, and snoRNA. In some embodiments, the payload gene encodes a siRNA, a shRNA, an antisense RNA oligonucleotide, an antisense miRNA, a trans-splicing RNA, a guide RNA, single-guide RNA, crRNA, a tracrRNA, a trans-splicing RNA, a pre-mRNA, a mRNA, or any combination thereof.

The payload protein(s) (e.g., a first payload, a second payload, a third payload, a supplemental payload) can comprise a cytokine. The cytokine can be selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof.

The payload protein(s) (e.g., a first payload, a second payload, a third payload, a supplemental payload) can comprise a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. The payload protein(s) can comprise a member of the TNF family of cytokines selected from the group consisting of TNF-alpha, TNF-beta, LT-beta, CD40 ligand, Fas ligand, CD 27 ligand, CD 30 ligand, and 4-1 BBL. The payload protein(s) can comprise a member of the immunoglobulin superfamily of cytokines selected from the group consisting of B7.1 (CD80) and B7.2 (B70). The payload protein(s) can comprise an interferon. The interferon can be selected from interferon alpha, interferon beta, or interferon gamma. The payload protein(s) can comprise a chemokine. The chemokine can be selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. The payload protein(s) can comprise a interleukin. The interleukin can be selected from IL-10 IL-12, IL-1, IL-6, IL-7, IL-15, IL-2, IL-18 or IL-21. The payload protein(s) can comprise a tumor necrosis factor (TNF). The TNF can be selected from TNF-alpha, TNF-beta, TNF-gamma, CD252, CD154, CD178, CD70, CD153, or 4-1BBL.

The payload protein(s) (e.g., a first payload, a second payload, a third payload, a supplemental payload) can comprise a CRE recombinase, GCaMP, a cell therapy component, a knock-down gene therapy component, a cell-surface exposed epitope, or any combination thereof. The payload protein(s) can comprise a chimeric antigen receptor.

The payload protein(s) (e.g., a first payload, a second payload, a third payload, a supplemental payload) can comprise a programmable nuclease. In some embodiments, the programmable nuclease is selected from the group comprising: SpCas9 or a derivative thereof; VRER, VQR, EQR SpCas9; xCas9-3.7; eSpCas9; Cas9-HIF1; HypaCas9; evoCas9; HiFi Cas9; ScCas9; StCas9; NmCas9; SaCas9; CjCas9; CasX; Cas9 H940A nickase; Cas12 and derivatives thereof, dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. The nucleic acid composition can comprise a polynucleotide encoding (i) a targeting molecule and/or (ii) a donor nucleic acid. The targeting molecule can be capable of associating with the programmable nuclease. The targeting molecule can comprise single strand DNA or single strand RNA. The targeting molecule can comprise a single guide RNA (sgRNA). A payload can comprise (i) a targeting molecule and/or (ii) a donor nucleic acid.

In some embodiments, the payload protein(s) (e.g., a first payload, a second payload, a third payload, a supplemental payload) is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as -glucosidase, imiglucarase, β-glucocerebrosidase, and alglucerase; enzyme activators, such as tissue plasminogen activator; chemokines, such as IP-10, monokine induced by interferon-gamma (Mig), Gro/IL-8, RANTES, MIP-1, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other non-limiting examples of payload protein(s) include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In some embodiments, the payload protein(s) (e.g., a first payload, a second payload, a third payload, a supplemental payload) is an active fragment of a protein, such as any of the aforementioned proteins. In some embodiments, the payload protein(s) is a fusion protein comprising some or all of two or more proteins. In some embodiments a fusion protein can comprise all or a portion of any of the aforementioned proteins.

In some embodiments, the payload protein(s) (e.g., a first payload, a second payload, a third payload, a supplemental payload) is a multi-subunit protein. For examples, the payload protein(s) can comprise two or more subunits, or two or more independent polypeptide chains. In some embodiments, the payload protein(s) can be an antibody. Examples of antibodies include, but are not limited to, antibodies of various isotypes (for example, IgG1, IgG2, IgG3, IgG4, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody.

In some embodiments, the payload protein(s) is a pro-survival protein (e.g., Bel-2, Bcl-XL, Mcl-1 and A1). In some embodiments, the payload gene encodes a apoptotic factor or apoptosis-related protein such as, for example, AIF, Apaf (e.g., Apaf-1, Apaf-2, and Apaf-3), oder APO-2 (L), APO-3 (L), Apopain, Bad, Bak, Bax, Bcl-2, Bcl-x_{L}, Bcl-x_{S}, bik, CAD, Calpain, Caspase (e.g., Caspase-1, Caspase-2, Caspase-3, Caspase-4, Caspase-5, Caspase-6, Caspase-7, Caspase-8, Caspase-9, Caspase-10, and Caspase-11), ced-3, ced-9, c-Jun, c-Myc, crm A, cytochrom C, CdR1, DcR1, DD, DED, DISC, DNA-PKcs, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, INK, Lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, and/or transglutaminase.

In some embodiments, the payload protein(s) (e.g., a first payload, a second payload, a third payload, a supplemental payload) is a cellular reprogramming factor capable of converting an at least partially differentiated cell to a less differentiated cell, such as, for example, Oct-3, Oct-4, Sox2, c-Myc, Klf4, Nanog, Lin28, ASCL1, MYT1 L, TBX3b, SV40 large T, hTERT, miR-291, miR-294, miR-295, or any combinations thereof. In some embodiments, the payload protein(s) is a programming factor that is capable of differentiating a given cell into a desired differentiated state, such as, for example, nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof.

In some embodiments, the payload protein(s) (e.g., a first payload, a second payload, a third payload, a supplemental payload) is a human adjuvant protein capable of eliciting an innate immune response, such as, for example, cytokines which induce or enhance an innate immune response, including IL-2, IL-12, IL-15, IL-18, IL-21CCL21, GM-CSF and TNF-alpha; cytokines which are released from macrophages, including IL-1, IL-6, IL-8, IL-12 and TNF-alpha; from components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, C1qR, C1INH, C4 bp, MCP, DAF, H, I, P and CD59; from proteins which are components of the signaling networks of the pattern recognition receptors including TLR and IL-1 R1, whereas the components are ligands of the pattern recognition receptors including IL-1 alpha, IL-1 beta, Beta-defensin, heat shock proteins, such as HSP10, HSP60, HSP65, HSP70, HSP75 and HSP90, gp96, Fibrinogen, TypIII repeat extra domain A of fibronectin; the receptors, including IL-1 RI, TLR1, TLR2, TLR3, TLR4, TLR5, TLR6, TLR7, TLR8, TLR9, TLR10, TLR11; the signal transducers including components of the Small-GTPases signaling (RhoA, Ras, Rac1, Cdc42 etc.), components of the PIP signaling (PI3K, Src-Kinases, etc.), components of the MyD88-dependent signaling (MyD88, IRAK1, IRAK2, etc.), components of the MyD88-independent signaling (TICAM1, TICAM2 etc.); activated transcription factors including e.g. NF-κB, c-Fos, c-Jun, c-Myc; and induced target genes including e.g. IL-1 alpha, IL-1 beta, Beta-Defensin, IL-6, IFN gamma, IFN alpha and IFN beta; from costimulatory molecules, including CD28 or CD40-ligand or PD1; protein domains, including LAMP; cell surface proteins; or human adjuvant proteins including CD80, CD81, CD86, trif, flt-3 ligand, thymopentin, Gp96 or fibronectin, etc., or any species homolog of any of the above human adjuvant proteins.

As described herein, the nucleotide sequence encoding the payload protein(s) (e.g., a first payload, a second payload, a third payload, a supplemental payload) can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of payload protein(s) (e.g., a first payload, a second payload, a third payload, a supplemental payload) expression in the cell can vary. The amount of the payload protein(s) expressed in the subject (e.g., the serum of the subject) can vary. For example, in some embodiments the protein can be expressed in the serum of the subject in the amount of at least about 9 μg/ml, at least about 10 μg/ml, at least about 50 μg/ml, at least about 100 μg/ml, at least about 200 g/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload protein(s) is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a payload protein(s) is needed for the method to be effective can vary depending on non-limiting factors such as the particular payload protein(s) and the subject receiving the treatment, and an effective amount of the protein can be readily determined by a skilled artisan using conventional methods known in the art without undue experimentation.

A payload protein(s) (e.g., a first payload, a second payload, a third payload, a supplemental payload) encoded by a payload gene can be of various lengths. For example, the payload protein(s) can be at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, or longer in length. In some embodiments, the payload protein(s) is at least about 480 amino acids in length. In some embodiments, the payload protein(s) is at least about 500 amino acids in length. In some embodiments, the payload protein(s) is about 750 amino acids in length.

The payload genes can have different lengths in different implementations. The number of payload genes can be different in different embodiments. In some embodiments, the number of payload genes in a nucleic acid composition can be, or can be about, 1, 2, 3, 4, 5, 6, 7, 8, 9, 10, 11, 12, 13, 14, 15, 16, 17, 18, 19, 20, 21, 22, 23, 24, 25, or a number or a range between any two of these values. In some embodiments, the number of payload genes in a nucleic acid composition can be at least, or can be at most, 1, 2, 3, 4, 5, 6, 7, 8, 9, 10, 11, 12, 13, 14, 15, 16, 17, 18, 19, 20, 21, 22, 23, 24, 25. In some embodiments, a payload genes is, or is about, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, nucleotides in length. In some embodiments, a payload gene is at least, or is at most, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, or 10000 nucleotides in length.

The payload can be an inducer of cell death. The payload can be induce cell death by a non-endogenous cell death pathway (e.g., a bacterial pore-forming toxin). In some embodiments, the payload can be a pro-survival protein. In some embodiments, the payload is a modulator of the immune system. The payload protein can comprise a CRE recombinase, GCaMP, a cell therapy component, a knock-down gene therapy component, a cell-surface exposed epitope, or any combination thereof.

Chimeric Antigen Receptors and Engineered T Cell Receptors

The payload protein(s) (e.g., a first payload, a second payload, a third payload, a supplemental payload) can comprise a chimeric antigen receptor (CAR) or T-cell receptor (TCR). In some embodiments, the CAR comprises a T-cell receptor (TCR) antigen binding domain. The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. The terms “CAR” and “CAR molecule” are used interchangeably. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some aspects, the set of polypeptides are contiguous with each other. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

The CAR and/or TCR can comprise one or more of an antigen binding domain, a transmembrane domain, and an intracellular signaling domain. The CAR or TCR further can comprise a leader peptide. The TCR further can comprise a constant region and/or CDR4. The term “signaling domain” refers to the functional portion of a protein which acts by transmitting information within the cell to regulate cellular activity via defined signaling pathways by generating second messengers or functioning as effectors by responding to such messengers. An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12.

The intracellular signaling domain can comprise a primary signaling domain, a costimulatory domain, or both of a primary signaling domain and a costimulatory domain. The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CD5, ICAM-1, LFA-1 (CD11a/CD18), ICOS (CD278), and 4-1BB (CD137). Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. The intracellular signaling domain can comprise the entire intracellular portion, or the entire native intracellular signaling domain, of the molecule from which it is derived, or a functional fragment or derivative thereof.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability. It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain). A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. The primary signaling domain can comprise a functional signaling domain of one or more proteins selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12, or a functional variant thereof.

In some embodiments, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue. The costimulatory domain can comprise a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

The portion of the CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In a further aspect, the CAR comprises an antibody fragment that comprises a scFv.

In some embodiments, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR of the invention include those associated with viral, bacterial and parasitic infections, autoimmune disease and cancer cells.

In some embodiments, the CAR-mediated T-cell response can be directed to an antigen of interest by way of engineering an antigen binding domain that specifically binds a desired antigen into the CAR. In some embodiments, the portion of the CAR comprising the antigen binding domain comprises an antigen binding domain that targets a tumor antigen, e.g., a tumor antigen described herein. The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In some embodiments, the antigen binding domain is humanized.

The antigen binding domain can comprise an antibody, an antibody fragment, an scFv, a Fv, a Fab, a (Fab′)2, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Va and V3 genes from a T cell clone linked by a linker (e.g., a flexible peptide). This approach is very useful to cancer associated target that itself is intracellar, however, a fragment of such antigen (peptide) is presented on the surface of the cancer cells by MHC.

In some embodiments, the antigen binding domain is a multispecific antibody molecule. In some embodiments, the multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

The antigen binding domain can be configured to bind to a tumor antigen. The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The tumor antigen can be a solid tumor antigen. The tumor antigen can be selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

The tumor antigen can be selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

The antigen binding domain can be connected to the transmembrane domain by a hinge region. In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge.

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane domain is one that is associated with one of the other domains of the CAR e.g., in one embodiment, the transmembrane domain may be from the same protein that the signaling domain, costimulatory domain or the hinge domain is derived from. In some embodiments, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some embodiments, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain can comprise a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Rα, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11 b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target.

### Cells, Cell Populations, and Subpopulations

Disclosed herein include cells. In some embodiments, the cell comprises: one or more of the nucleic acid compositions (e.g., circuits) provided herein. Disclosed herein include cell populations. In some embodiments, the cell population comprises a plurality of cells. In some embodiments, each cell comprises one or more of the nucleic acid compositions (e.g., circuits) provided herein.

The cell population can comprise a plurality of monoclonal cells. The cell population can comprise one or more subpopulations of cells. Subpopulations can be metabolically or functionally distinct subpopulations. Each subpopulation of cells can be characterized by differences in the concentration and/or expression level of one or more TFs and one or more payloads. Each subpopulation of cells can be characterized by a distinct expression state. The expression state can be mitotically heritable. An expression state can be stable across multiple cell division cycles. The expression state can be robust to biological gene expression noise. In some embodiments, less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, of cells within a subpopulation transition to another expression state due to intrinsic noise.

One or more subpopulations can comprise: a first subpopulation of cells characterized by a first expression state. The first expression state can comprise: tuned expression levels of the first TF and first payload(s), and depleted expression levels of the second TF, second payload(s), third TF, and/or third payload(s). One or more subpopulations can comprise: a second subpopulation of cells characterized by a second expression state. The second expression state can comprise: tuned expression levels of the second TF and second payload(s), and depleted expression levels of the first TF, first payload(s), third TF, and/or third payload(s). One or more subpopulations can comprise: a third subpopulation of cells characterized by a third expression state. The third expression state can comprise: tuned expression levels of the third TF and third payload(s), and depleted expression levels of the first TF, first payload(s), second TF, and/or second payload(s). One or more subpopulations can comprise: a fourth subpopulation of cells characterized by a fourth expression state. The fourth expression state can comprise: tuned expression levels of the first TF, first payload(s), second TF, and second payload(s), and. The fourth expression state can comprise: depleted expression levels of the third TF, and/or third payload(s). One or more subpopulations can comprise: a fifth subpopulation of cells characterized by a fifth expression state. The fifth expression state can comprise: tuned expression levels of the first TF, first payload(s), third TF, and third payload(s), and depleted expression levels of the second TF, and/or second payload(s). One or more subpopulations can comprise: a sixth subpopulation of cells characterized by a sixth expression state. The sixth expression state can comprise: tuned expression levels of the second TF, second payload(s), third TF, and third payload(s), and depleted expression levels of the first TF, and/or first payload(s). One or more subpopulations can comprise: a seventh subpopulation of cells characterized by a seventh expression state. The seventh expression state can comprise: tuned expression levels of the first TF, first payload(s), second TF, second payload(s), third TF, and third payload(s). In some embodiments, one or more subpopulations are configured to express one or more targeting moieties configured to bind a component of a target site of a subject. Additional subpopulations of cells, each comprising a distinct additional expression state, are also contemplated herein, and can grow in number as the number of TFs expands. For example, cells can comprise nucleic acid composition(s) comprising n supplemental promoters each operably linked to a nth supplemental polynucleotide encoding an nth supplemental transcription factor (sTF) and to a (n+1)th supplemental polynucleotide encoding one or more nth supplemental payloads, thereby generating still further multistable and distinct subpopulations and expression states in addition to those described above in cells expressing a first TF, a second TF, and/or a third TF.

In some embodiments, tuned expression levels range between a lower tuned threshold and an upper tuned threshold of a tuned expression range. The tuned expression range can be capable of being tuned by modulating one or more of dimerization domain affinity, TF protein stability, transactivation domain strength, DNA-binding domain, or any combination of thereof. The difference between the lower untuned threshold and the upper untuned threshold of the tuned expression range can be greater than about one order of magnitude. The difference between the lower untuned threshold and the upper untuned threshold of the tuned expression range can be less than about one order of magnitude. Depleted expression levels can comprise basal expression levels. Depleted expression levels can comprise absent expression. Tuned expression levels can be at least about 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 400-fold, 600-fold, 800-fold, or a number or a range between any of these values, greater than depleted expression levels. Expression levels can comprise transcript levels and/or protein levels.

Transient induction of expression of one or more TFs can be capable of transitioning cells from one expression state to another expression state. Transient induction of one or more TFs can be capable of irreversibly transitioning cells from one expression state to another expression state. In some embodiments, a transactivator-binding compound causes transient induction of expression of the one or more TFs. One or more subpopulations can comprise: an eighth subpopulation of cells characterized by an off expression state. The off expression state can comprise: depleted expression levels of the first TF, first payload(s), second TF, second payload(s), third TF, and/or third payload(s). Some or all cells of the cell population can be capable of transitioning to the off state in the absence of the degron stabilizing molecule and the dimerization ligand. Some or all cells of the cell population can be capable of transitioning from the off state to the first expression state, second expression state, third expression state, fourth expression state, fifth expression state, sixth expression state, and/or seventh expression state, in the presence of a first threshold level of the degron stabilizing molecule and the dimerization ligand.

In some embodiments, the number of expression states increases monotonically with the number of distinct TF species in the cell population. In some embodiments, the number of robust expression states decreases monotonically with TF protein stability. In some embodiments, the number of robust expression states decreases monotonically with the concentration of the degron stabilizing molecule. Reducing TF stability can be capable of transitioning cells from one expression state to another expression state. Reducing TF stability can be capable of irreversibly transitioning cells from one expression state to another expression state. In some embodiments, restoring TF stability is not capable of causing cells to return to previously destabilized states. Restoring TF stability can comprise increasing the concentration of the degron stabilizing molecule. In some embodiments, below a second threshold level of the degron stabilizing molecule, the seventh expression state is destabilized. In some embodiments, below a second threshold level of the degron stabilizing molecule, the seventh expression state is destabilized irreversibly. In some embodiments, below a third threshold level of the degron stabilizing molecule, the fourth expression state, the fifth expression state, and/or the sixth expression state, is destabilized. In some embodiments, below a third threshold level of the degron stabilizing molecule, the fourth expression state, the fifth expression state, and/or the sixth expression state, is destabilized irreversibly.

In some embodiments, tuned expression levels, the number of subpopulations, the types of subpopulations, the relative number of cells within each subpopulation, and/or the expression state of one or more cells can be configured to be responsive to changes in: the local concentration of a degron stabilizing molecule, a transactivator-binding compound, a dimerization ligand, or any combination thereof, cell environment (e.g., location relative to a target site of a subject and/or changes in the presence and/or absence of target cell(s) comprising target-specific antigen(s)); one or more signal transduction pathways regulating cell survival, cell growth, cell proliferation, cell adhesion, cell migration, cell metabolism, cell morphology, cell differentiation, apoptosis, or any combination thereof, input(s) of a synthetic cell-cell communication system (e.g., Synthetic Notch (SynNotch) receptor, a Modular Extracellular Sensor Architecture (MESA) receptor, a synthekine, engineered GFP, and/or auxin); and/or T cell activity (e.g., T cell simulation, T cell activation, cytokine secretion, T cell survival, T cell proliferation, CTL activity, T cell degranulation, and T cell differentiation).

A synthetic protein circuit component can be capable of modulating the expression and/or activity of a TF. The expression and/or activity of a TF can be configured to be responsive to immune cell stimulation. In some embodiments, immune cell stimulation can comprise signal transduction induced by binding of a stimulatory molecule with its cognate ligand on the surface of an immune cell. The cognate ligand can be a CAR or a TCR. One or more of the expression states can be configured to activate a state-specific program. The state-specific program can be a therapeutic program. The population of cells can be configured to generate mixture of subpopulations at defined ratios. The defined ratio can be selected to generate synergy between the state-specific programs of said subpopulations. The one or more subpopulations can comprise and/or are capable of differentiating into two or more cell types. In some embodiments, the two or more cell types are capable of providing different overall functions and/or different components of a single function. In some embodiments, the two or more cell types are found within the same tissue. The population of cells can be configured to respond to the inputs of a synthetic cell-cell communication system. The tuned expression levels and/or the expression state of one or more cells can be configured to be responsive to changes in one or more inputs (e.g., a threshold input level). The input level can be sensed by an engineered biosensor. The tuned expression levels and/or the expression state of one or subpopulations can be capable of being modulated by one or more of a Synthetic Notch (SynNotch) receptor, a Modular Extracellular Sensor Architecture (MESA) receptor, Tango, dCas9-synR, or any combination thereof.

One or more cells of the population of cells can be configured to activate a therapeutic program in the presence of an input threshold (e.g., a local input threshold at a target site. The therapeutic program can comprise expression of one or more payloads. One or more cells of the population of cells are immune cells can be configured to switch from an immune cell inactivated state to an immune cell activated state in the presence of an input threshold (e.g., a local input threshold at a target site). One or more cells of the population of cells can be configured to differentiate into one or more cell types in the presence of an input threshold (e.g., a local input threshold at a target site). In some embodiments, the population of cells are capable of being employed in synthetic organogenesis and/or tissue repair.

The cell(s) can comprise a eukaryotic cell. The eukaryotic cell can comprise an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof. The cell(s) can be a bacterial cell, a yeast cell, a fungal cell, a mammalian cell, a human cell, a stem cell, a progenitor cell, an induced pluripotent stem cell, a human induced pluripotent stem cell, a plant cell or an animal cell.

Targeting

A payload can comprise one or more receptors and/or a targeting moiety configured to bind a component of a target site of a subject. One or more subpopulations can comprise one or more receptors and/or one or more targeting moieties configured to bind a component of a target site of a subject. The one or more receptors and/or one or more targeting moieties can be selected from the group comprising mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. The one or more receptors and/or one or more targeting moieties can comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof.

The antibody or antigen-binding fragment thereof can comprise a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

A payload can comprise one or more receptors and/or a targeting moiety configured to bind a component of a target site of a subject. The one or more receptors and/or one or more targeting moieties can be configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11(C—C motif chemokine 11), CCR4 (C—C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B 1, CYP1B 1, Cytomegalovirus, Cytomegalovirus glycoprotein B, Dabigatran, DLL4 (delta-like—ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor a-chain, Growth differentiation factor 8, GP100, GPNMB (Transmembrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-7, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αᵤβ₃, ανβ3, α₄β₇, α5β1, α6β4, α7β7, α11β3, α5β5, ανβ5), Interferon gamma-induced protein, ITGA2, ITGB2, KIR2D, LCK, Le, Legumain, Lewis-Y antigen, LFA-1(Lymphocyte function-associated antigen 1, CD11a), LHRH, LINGO-1, Lipoteichoic acid, LIVIA, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRATLR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

### Methods of Treating a Disease or Disorder

Disclosed herein include methods of treating a disease or disorder in a subject. In some embodiments, the method comprises: introducing into one or more cells one or more of the nucleic acid compositions (e.g., circuits) provided herein or one or more of the compositions provided herein; and administering to the subject an effective amount of the one or more cells, or a cell population derived therefrom.

In some embodiments, the method comprises: isolating the one or more cells from the subject prior to the introducing step. The introducing step can be performed in vivo, in vitro, and/or ex vivo. The introducing step can comprise calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof.

Disclosed herein include method of treating a disease or disorder in a subject. In some embodiments, the method comprises: administering to the subject an effective amount of cell(s), cell population(s), and/or subpopulation(s) provided herein.

The subject can be a mammal. In some embodiments, the disease is associated with expression of a tumor antigen, wherein the disease associated with expression of a tumor antigen is selected from the group consisting of a proliferative disease, a precancerous condition, a cancer, and a non-cancer related indication associated with expression of the tumor antigen. The disease or disorder can be a cancer (e.g., a solid tumor). The cancer can be selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

The cancer can be a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

The disease or disorder can be an autoimmune disorder. An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barre syndrome, Hashimoto's thyroiditis, and cardiomyopathy.

In some embodiments, the method comprises: administering to the subject an effective amount of a degron stabilizing molecule, a pro-death agent, a transactivator-binding compound, a dimerization ligand, or any combination thereof, prior to, during, and/or after administration of the disclosed engineered cells (e.g., cell(s), cell population(s), and/or subpopulation(s) disclosed herein). The administration of said agents can modulate the tuned expression levels, the number of subpopulations, the types of subpopulations, the relative number of cells within each subpopulation, and/or the expression state of one or more cells in the subject as described herein, and can be adjusted as needed throughout treatment. In some embodiments, the engineered cells (e.g., cell(s), cell population(s), and/or subpopulation(s) disclosed herein) activate a state-specific program(s) in the subject, such as, for example, killing of target cells at a target site (e.g., a target site-specific therapeutic program).

Administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The disclosed engineered cells (e.g., cell(s), cell population(s), and/or subpopulation(s) disclosed herein) can be administered at a therapeutically effective amount. For example, a therapeutically effective amount of the disclosed engineered cells (e.g., cell(s), cell population(s), and/or subpopulation(s) disclosed herein) can be at least about 10² cells, at least about 10³ cells, at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the disclosed engineered cells (e.g., cell(s), cell population(s), and/or subpopulation(s) disclosed herein) is about 10⁴ cells, about 10⁵ cells, about 10⁶ cells, about 10⁷ cells, or about 10⁸ cells. In one particular embodiment, the therapeutically effective amount of the disclosed engineered cells (e.g., cell(s), cell population(s), and/or subpopulation(s) disclosed herein) is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

The disclosed engineered cells (e.g., cell(s), cell population(s), and/or subpopulation(s) disclosed herein) herein may be included in a composition for therapy. In some embodiments, the composition comprises a population of disclosed engineered cells (e.g., cell(s), cell population(s), and/or subpopulation(s) disclosed herein). The composition may include a pharmaceutical composition and further include a pharmaceutically acceptable carrier. A therapeutically effective amount of the pharmaceutical composition comprising the disclosed engineered cells (e.g., cell(s), cell population(s), and/or subpopulation(s) disclosed herein) may be administered. The cells provided herein may be administered either alone, or as a pharmaceutical composition in combination with diluents and/or with other components such as IL-2 or other cytokines or cell populations. Ex vivo procedures are well known in the art. Briefly, cells are isolated from a mammal (e.g., a human) and genetically modified (i.e., transduced or transfected in vitro) with a nucleic acid composition (e.g., a vector) disclosed herein or a composition disclosed herein, thereby generating an engineered population of cells. The disclosed engineered cells (e.g., cell(s), cell population(s), and/or subpopulation(s) disclosed herein) can be administered to a mammalian recipient to provide a therapeutic benefit. The mammalian recipient may be a human and the disclosed engineered cells can be autologous with respect to the recipient. Alternatively, the disclosed engineered cells can be allogeneic, syngeneic or xenogeneic with respect to the recipient.

Target Sites

In some embodiments, a target site of a subject comprises a site of disease or disorder or is proximate to a site of a disease or disorder. In some embodiments, the target site comprises a tissue. The target site can comprise a solid tumor. The target site can comprise a site of disease or disorder or can be proximate to a site of a disease or disorder. The location of the one or more sites of a disease or disorder can be predetermined, can be determined during the method, or both. The target site can be an immunosuppressive environment. The target site can comprise a tissue. The tissue can be inflamed tissue and/or infected tissue. The tissue can comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. The tissue can comprise: (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue. In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, of the disclosed engineered cells (e.g., cell(s), cell population(s), and/or subpopulation(s) disclosed herein) at the target site activate the target site-specific therapeutic program (e.g., CAR activation). In some embodiments, less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or a number or a range between any two of these values, of the disclosed engineered cells (e.g., cell(s), cell population(s), and/or subpopulation(s) disclosed herein) at a site other than the target site activate the target site-specific therapeutic program (e.g., CAR activation).

The ratio of the concentration of payload protein at the subject's target site to the concentration of payload protein in subject's blood, serum, or plasma can be vary. In some embodiments, the ratio of the concentration of payload protein at the subject's target site to the concentration of payload protein in subject's blood, serum, or plasma can be, or be about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values. In some embodiments, the ratio of the concentration of payload protein at the subject's target site to the concentration of payload protein in subject's blood, serum, or plasma can be at least, or be at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, or 10000:1.

The target site can comprise target cells. The target cells can be tumor cells (e.g., solid tumor cells). In some embodiments, the administration of engineered cells (e.g., cell(s), cell population(s), and/or subpopulation(s) disclosed herein) provided herein results in the death of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, of the target cells. Non-target cells can comprise cells of the subject other than target cells. The ratio of target cell death to non-target cell death after administration of engineered cells (e.g., cell(s), cell population(s), and/or subpopulation(s) disclosed herein) provided herein can be at least about 2:1. In some embodiments, the ratio of target cell death to non-target cell death after administration of engineered cells (e.g., cell(s), cell population(s), and/or subpopulation(s) disclosed herein) provided herein can be, or be about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values. In some embodiments, the ratio of target cell death to non-target cell death after administration of engineered cells (e.g., cell(s), cell population(s), and/or subpopulation(s) disclosed herein) provided herein can be at least, or be at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, or 10000:1.

Additional Agents

In some embodiments, the method comprises administering one or more additional agents to the subject. In some embodiments, the one or more additional agents increases the efficacy of the population of cells. The one or more additional agents can comprise a protein phosphatase inhibitor, a kinase inhibitor, a cytokine, an inhibitor of an immune inhibitory molecule, and/or or an agent that decreases the level or activity of a T_{REG} cell. The one or more additional agents can comprise an immune modulator, an anti-metastatic, a chemotherapeutic, a hormone or a growth factor antagonist, an alkylating agent, a TLR agonist, a cytokine antagonist, a cytokine antagonist, or any combination thereof. The one or more additional agents can comprise an agonistic or antagonistic antibody specific to a checkpoint inhibitor or checkpoint stimulator molecule such as PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, TIM-3.

The one or more additional agents can be selected from the group consisting of alkylating agents (nitrogen mustards, ethylenimine derivatives, alkyl sulfonates, nitrosoureas and triazenes); uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®); bendamustine (Treakisym®, Ribomustin®, Treanda®); chlormethine (Mustargen®); cyclophosphamide (Cytoxan®, Neosar®, Clafen®, Endoxan®, Procytox®, Revimmune™); ifosfamide (Mitoxana®); melphalan (Alkeran®); Chlorambucil (Leukeran®); pipobroman (Amedel®, Vercyte®); triethylenemelamine (Hemel®, Hexylen®, Hexastat®); triethylenethiophosphoramine; Temozolomide (Temodar®); thiotepa (Thioplex®); busulfan (Busilvex®, Myleran®); carmustine (BiCNU®); lomustine (CeeNU®); streptozocin (Zanosar®); estramustine (Emcyt®, Estracit®); fotemustine; irofulven; mannosulfan; mitobronitol; nimustine; procarbazine; ranimustine; semustine; triaziquone; treosulfan; and Dacarbazine (DTIC-Dome®); anti-EGFR antibodies (e.g., cetuximab (Erbitux®), panitumumab (Vectibix®), and gefitinib (Iressa®)); anti-Her-2 antibodies (e.g., trastuzumab (Herceptin®) and other antibodies from Genentech); antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), carmofur, cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®), decitabine (Dacogen®), enocitabine (Sunrabin®), sapacitabine, tegafur-uracil, tiazofurine, tioguanine, trofosfamide, and gemcitabine (Gemzar®); vinca alkaloids: vinblastine (Velban®, Velsar®), vincristine (Vincasar®, Oncovin®), vindesine (Eldisine®), vinorelbine (Navelbine®), vinflunine (Javlor®); platinum-based agents: carboplatin (Paraplat®, Paraplatin®), cisplatin (Platinol®), oxaliplatin (Eloxatin®), nedaplatin, satraplatin, and triplatin; anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®), aclarubicin, amrubicin, liposomal doxorubicin, liposomal daunorubicin, pirarubicin, pixantrone, and zorubicin; topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101), belotecan, and rubitecan; taxanes: paclitaxel (Taxol®), docetaxel (Taxotere®), larotaxel, cabazitaxel, ortataxel, and tesetaxel; antibiotics: actinomycin (Cosmegen®), bleomycin (Blenoxane®), hydroxyurea (Droxia®, Hydrea®), mitomycin (Mitozytrex®, Mutamycin®); immunomodulators: lenalidomide (Revlimid®), thalidomide (Thalomid®); immune cell antibodies: alemtuzamab (Campath®), gemtuzumab (Myelotarg®), rituximab (Rituxan®), tositumomab (Bexxar®); interferons (e.g., IFN-alpha (Alferon®, Roferon-A®, Intron®-A) or IFN-gamma (Actimmune®)); interleukins: IL-1, IL-2 (Proleukin®), IL-24, IL-6 (Sigosix®), IL-12; HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”); anti-androgens which include, without limitation nilutamide (Nilandron®) and bicalutamide (Caxodex®); antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride; anti-hypercalcaemia agents which include without limitation gallium (III) nitrate hydrate (Ganite®) and pamidronate disodium (Aredia®); apoptosis inducers which include without limitation ethanol, 2-[[3-(2,3-dichlorophenoxy)propyl]amino]-(9Cl), gambogic acid, elesclomol, embelin and arsenic trioxide (Trisenox®); Aurora kinase inhibitors which include without limitation binucleine 2; Bruton's tyrosine kinase inhibitors which include without limitation terreic acid; calcineurin inhibitors which include without limitation cypermethrin, deltamethrin, fenvalerate and tyrphostin 8; CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide; CD45 tyrosine phosphatase inhibitors which include without limitation phosphonic acid; CDC25 phosphatase inhibitors which include without limitation 1,4-naphthalene dione, 2,3-bis[(2-hydroxyethyl)thio]-(9Cl); CHK kinase inhibitors which include without limitation debromohymenialdisine; cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid); cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl); cyclin dependent kinase inhibitors which include without limitation olomoucine and its derivatives, purvalanol B, roascovitine (Seliciclib®), indirubin, kenpaullone, purvalanol A and indirubin-3′-monooxime; cysteine protease inhibitors which include without limitation 4-morpholinecarboxamide, N-[(1S)-3-fluoro-2-oxo-1-(2-phenylethyl)propyl]amino]-2-oxo-1-(phenylmeth-yl)ethyl]-(9Cl); DNA intercalators which include without limitation plicamycin (Mithracin®) and daptomycin (Cubicin®); DNA strand breakers which include without limitation bleomycin (Blenoxane®); E3 ligase inhibitors which include without limitation N-((3,3,3-trifluoro-2-trifluoromethyl)propionyl)sulfanilamide; EGF Pathway Inhibitors which include, without limitation tyrphostin 46, EKB-569, erlotinib (Tarceva®), gefitinib (Iressa®), lapatinib (Tykerb®) and analogues; farnesyltransferase inhibitors which include without limitation ahydroxyfarnesylphosphonic acid, butanoic acid, 2-[(2S)-2-[[(2S,3S)-2-[[(2R)-2-amino-3-mercaptopropyl]amino]-3-methylpent-yl]oxy]-1-oxo-3-phenylpropyl]amino]-4-(methylsulfonyl)-1-methylethylester (2S)-(9Cl), tipifarnib (Zarnestra®), and manumycin A; Flk-1 kinase inhibitors which include without limitation 2-propenamide, 2-cyano-3-[4-hydroxy-3,5-bis(1-methylethyl)phenyl]-N-(3-phenylpropyl)-(2E-)-(9Cl); glycogen synthase kinase-3 (GSK3) inhibitors which include without limitation indirubin-3′-monooxime; histone deacetylase (HDAC) inhibitors which include without limitation suberoylanilide hydroxamic acid (SAHA), [4-(2-amino-phenylcarbamoyl)-benzyl]carbamic acid pyridine-3-ylmethylester and its derivatives, butyric acid, pyroxamide, trichostatin A, oxamflatin, apicidin, depsipeptide, depudecin, trapoxin, vorinostat (Zolinza®), and compounds disclosed in WO 02/22577; I-kappa B-alpha kinase inhibitors (IKK) which include without limitation 2-propenenitrile, 3-[(4-methylphenyl)sulfonyl]-(2E)-(9Cl); imidazotetrazinones which include without limitation temozolomide (Methazolastone®, Temodar® and its derivatives (e.g., as disclosed generically and specifically in U.S. Pat. No. 5,260,291) and Mitozolomide; insulin tyrosine kinase inhibitors which include without limitation hydroxyl-2-naphthalenylmethylphosphonic acid; c-Jun-N-terminal kinase (JNK) inhibitors which include without limitation pyrazoleanthrone and epigallocatechin gallate; mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hy-droxyethyl)-4-methoxy-(9Cl); MDM2 inhibitors which include without limitation trans-4-iodo, 4′-boranyl-chalcone; MEK inhibitors which include without limitation butanedinitrile, bis[amino[2-aminophenyl)thio]methylene]-(9Cl); MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996; mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD; NGFR tyrosine kinase inhibitors which include without limitation tyrphostin AG 879; p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl); p56 tyrosine kinase inhibitors which include without limitation damnacanthal and tyrphostin 46; PDGF pathway inhibitors which include without limitation tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854; phosphatidylinositol 3-kinase inhibitors which include without limitation wortmannin, and quercetin dihydrate; phosphatase inhibitors which include without limitation cantharidic acid, cantharidin, and L-leucinamide; protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid; PKC inhibitors which include without limitation 1-H-pyrollo-2,5-dione, 3-[1-3-(dimethylamino)propyl]-1H-indol-3-yl]-4-(1H-indol-3-yl)-(9Cl), Bisindolylmaleimide IX, Sphinogosine, staurosporine, and Hypericin; PKC delta kinase inhibitors which include without limitation rottlerin; polyamine synthesis inhibitors which include without limitation DMFO; PTP1B inhibitors which include without limitation L-leucinamide; protein tyrosine kinase inhibitors which include, without limitation tyrphostin Ag 216, tyrphostin Ag 1288, tyrphostin Ag 1295, geldanamycin, genistein and 7H-pyrrolo[2,3-d]pyrimidine derivatives as generically and specifically described in PCT Publication No.: WO 03/013541 and U.S. Publication No.: 2008/0139587; SRC family tyrosine kinase inhibitors which include without limitation PP1 and PP2; Syk tyrosine kinase inhibitors which include without limitation piceatannol; Janus (JAK-2 and/or JAK-3) tyrosine kinase inhibitors which include without limitation tyrphostin AG 490 and 2-naphthyl vinyl ketone; retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®); RNA polymerase H elongation inhibitors which include without limitation 5,6-dichloro-1-beta-D-ribofuranosylbenzimidazole; serine/Threonine kinase inhibitors which include without limitation 2-aminopurine; sterol biosynthesis inhibitors which include without limitation squalene epoxidase and CYP2D6; VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

### EXAMPLES

Some aspects of the embodiments discussed above are disclosed in further detail in the following examples, which are not in any way intended to limit the scope of the present disclosure.

### Example 1

### Synthetic Multistability in Mammalian Cells

### The MultiFate Circuit Architecture Generates Diverse Types of Multistability Through a Set of Promiscuously Interacting, Autoregulatory Dimer-Dependent Transcription Factors.

In this example, design of a new synthetic multistable system called MultiFate is described. In some embodiments of the MultiFate system described herein, transcription factors share a common dimerization domain, allowing them to competitively form both homodimers and heterodimers. The promoter of each transcription factor gene can contain binding sites that can be strongly bound only by its own homodimers, allowing homodimer-dependent self-activation. By contrast, in some embodiments, heterodimers do not efficiently bind to any promoter in this design. Heterodimerization can thus act to mutually inhibit the activity of both constituent transcription factors.

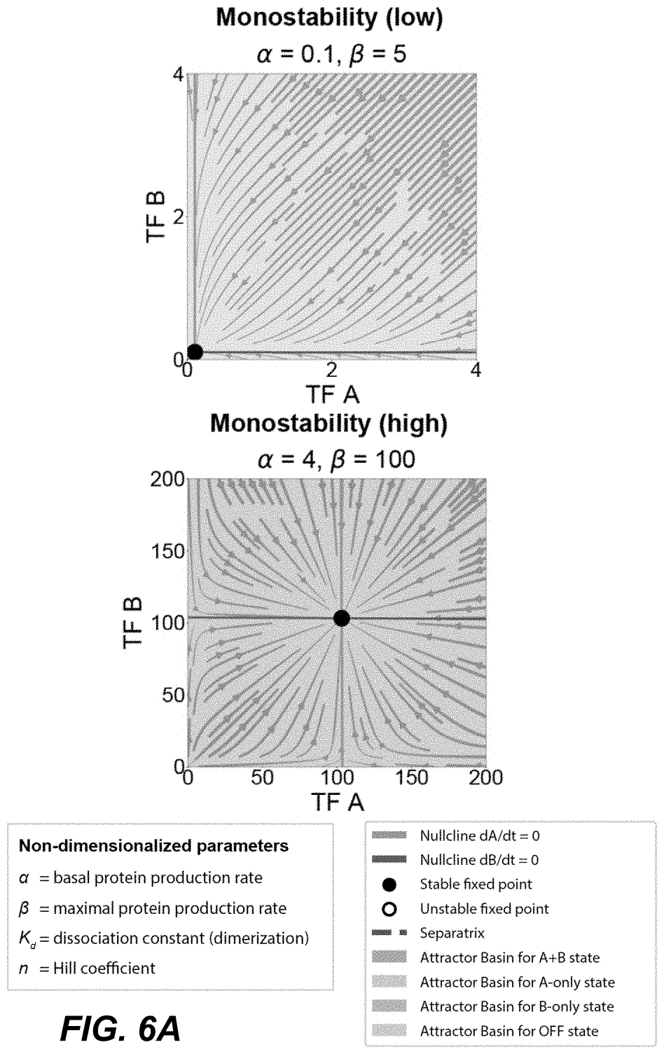

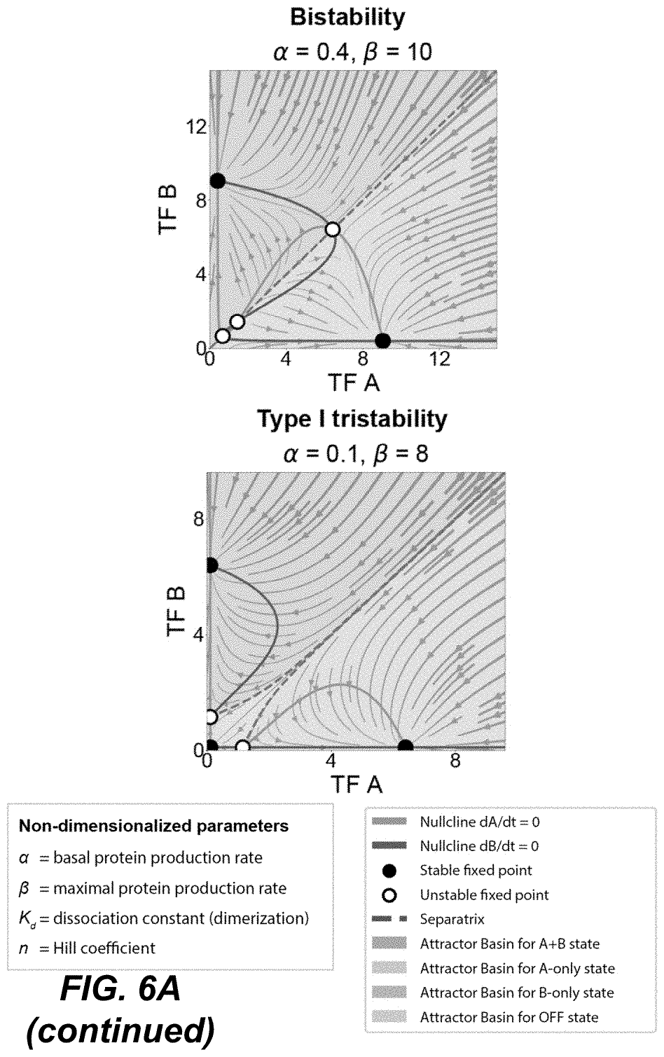

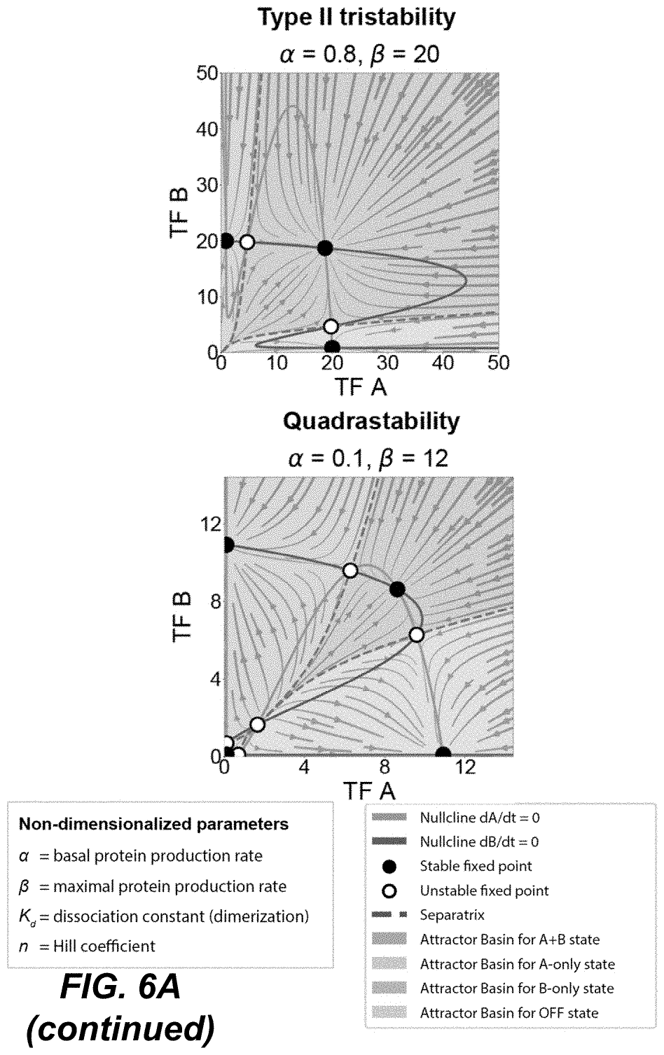

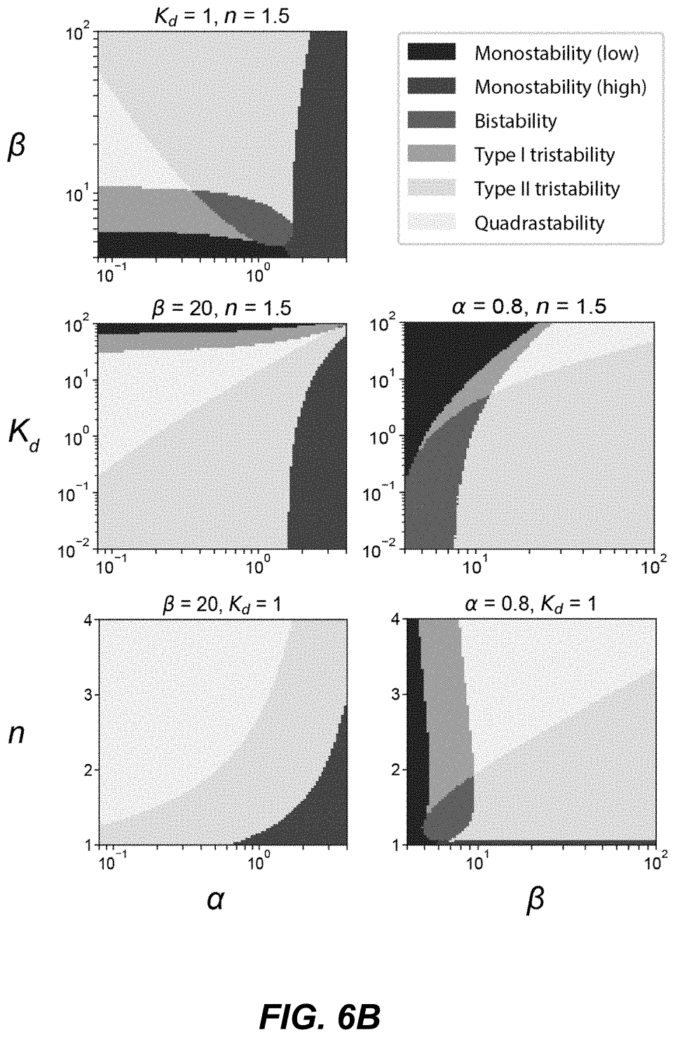

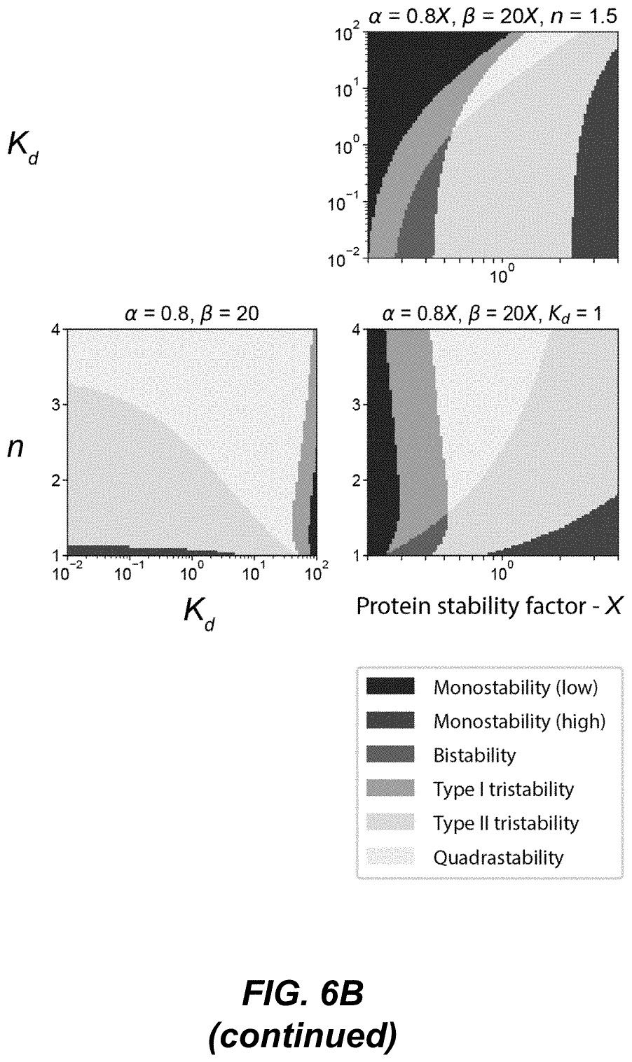

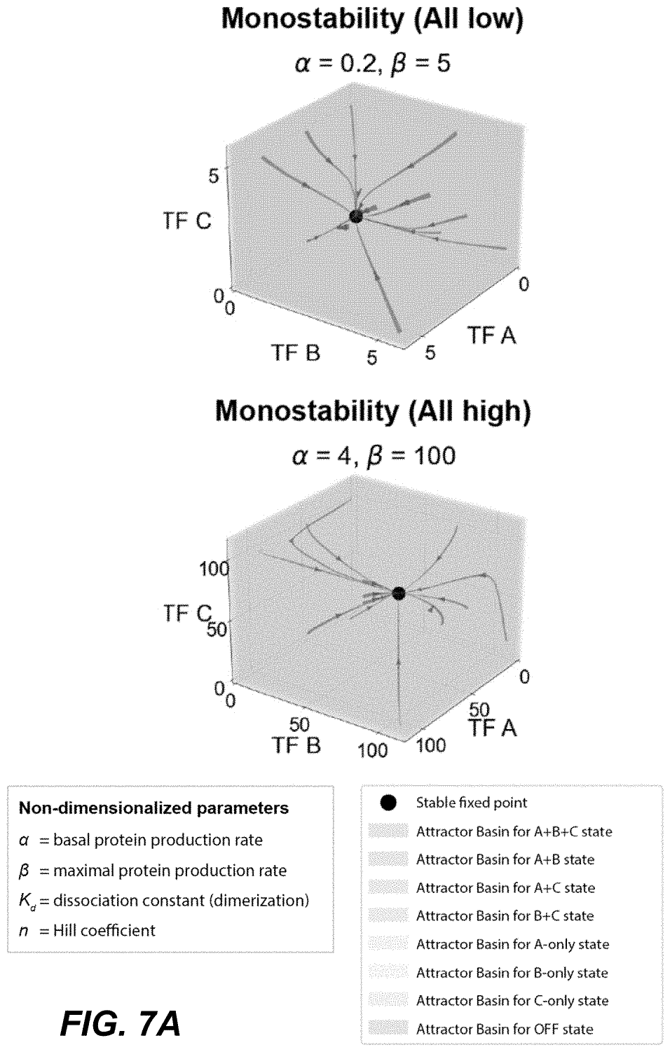

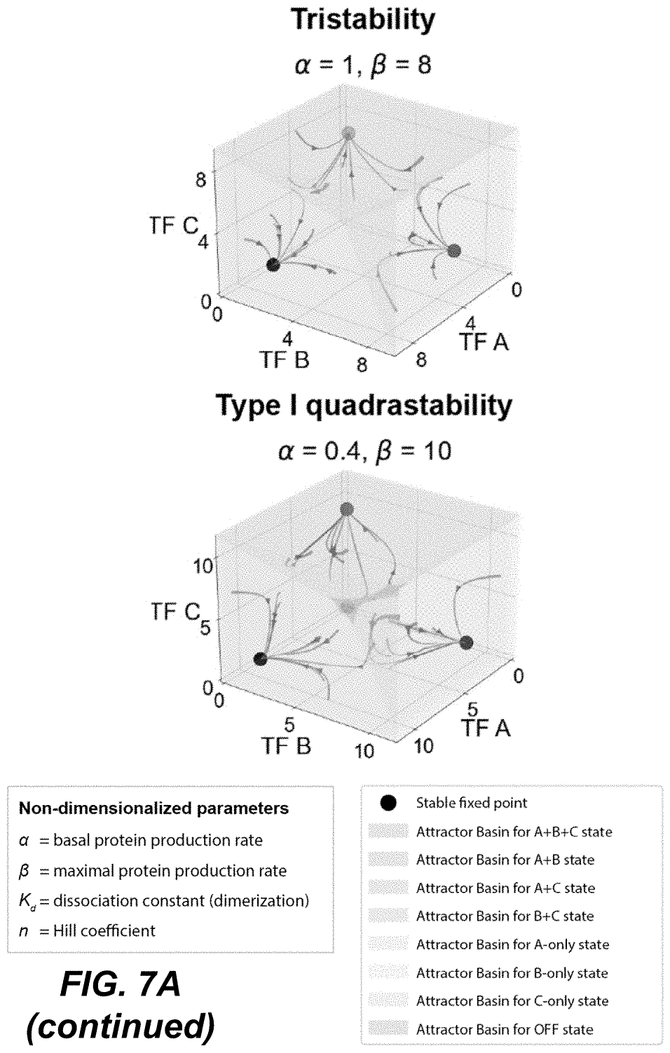

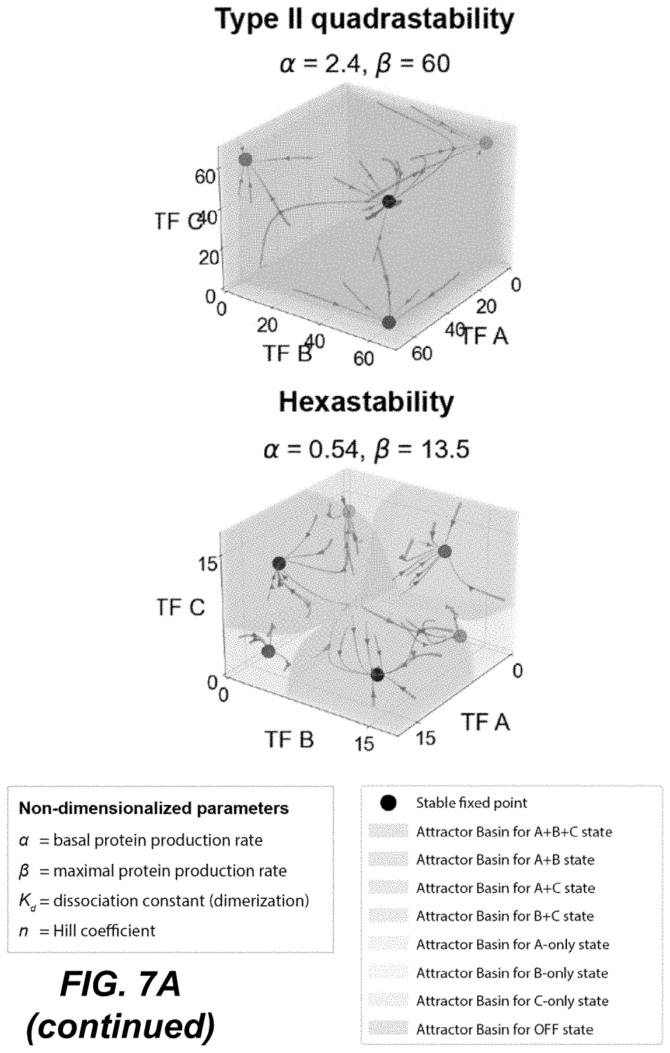

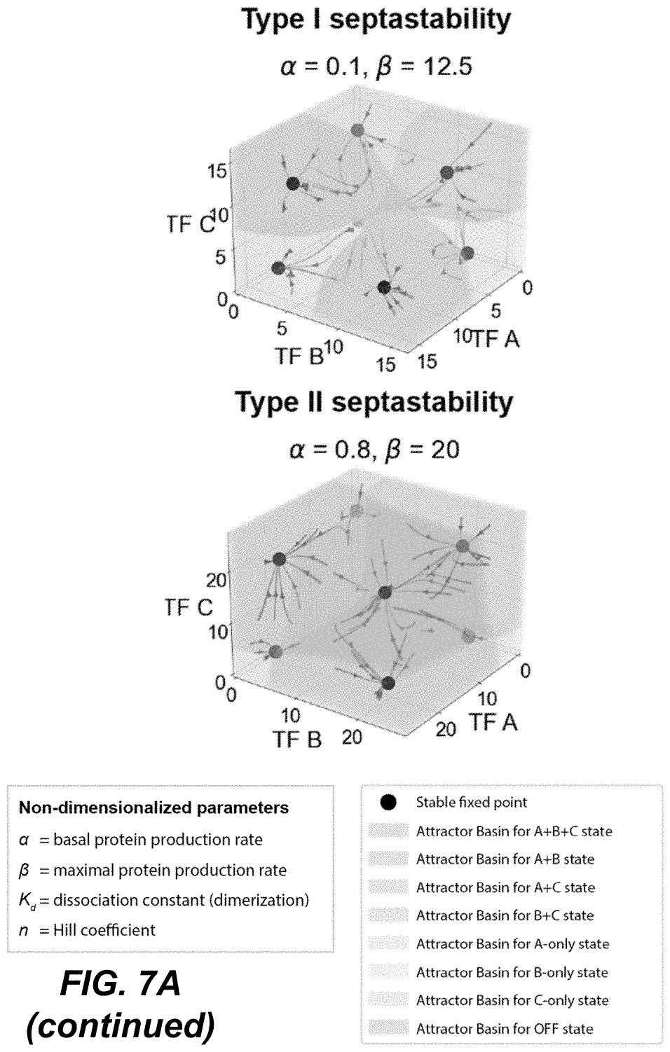

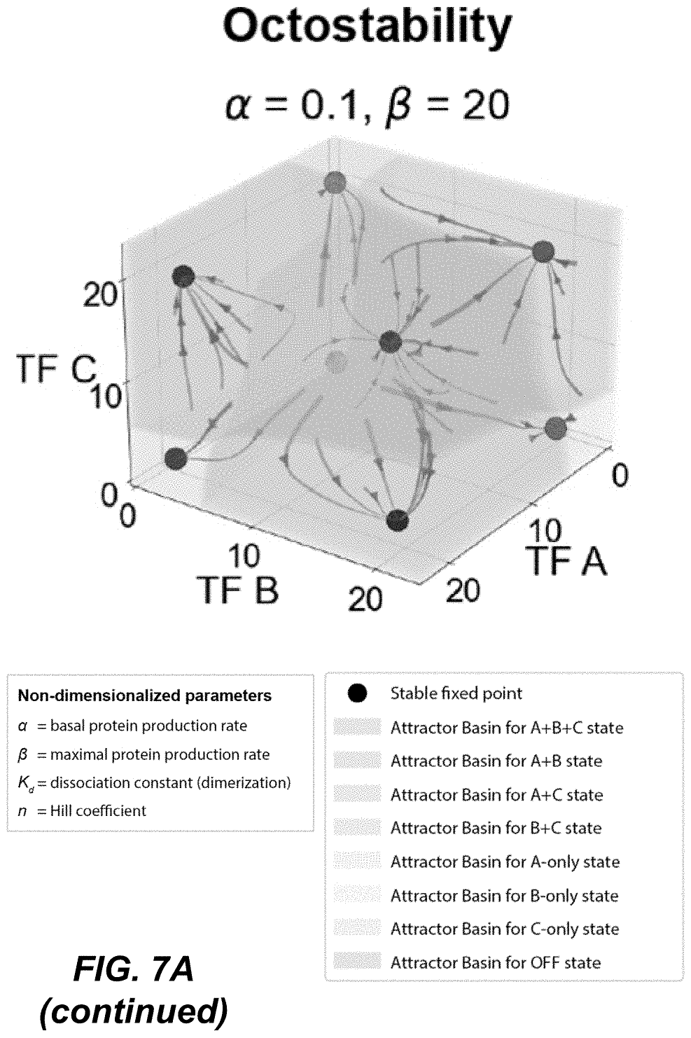

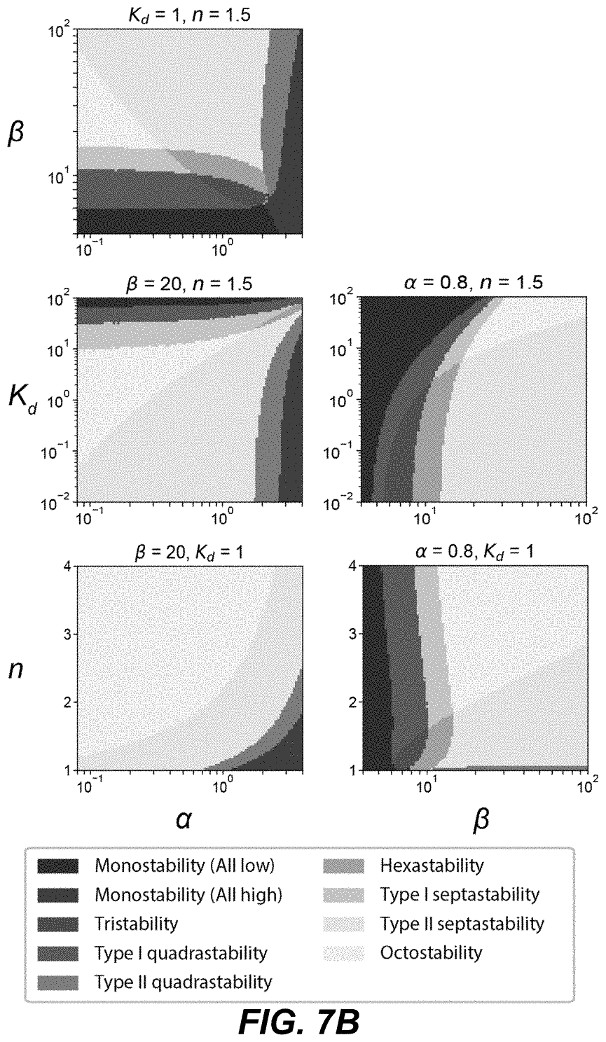

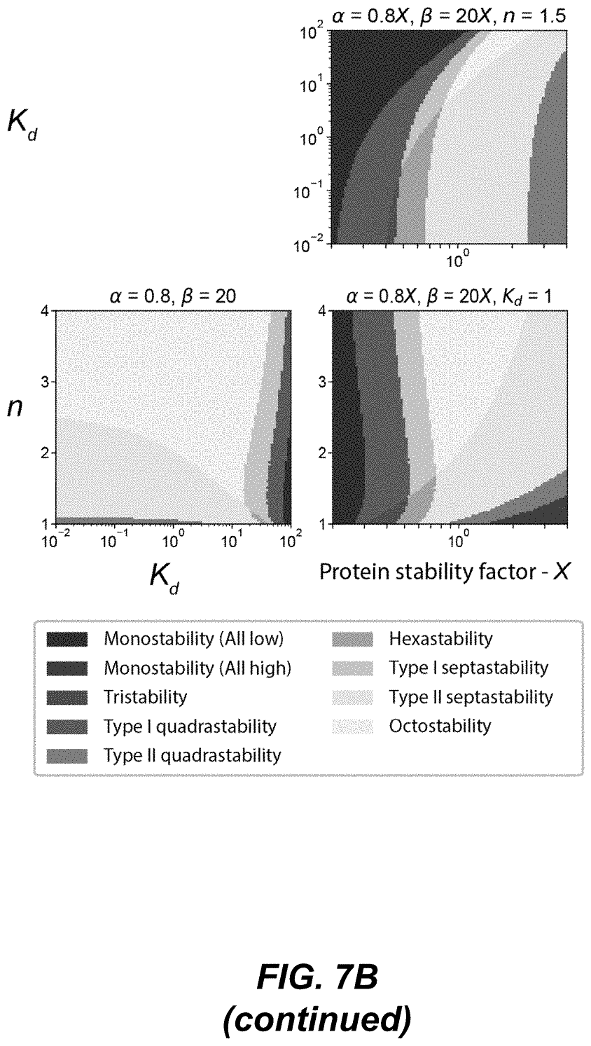

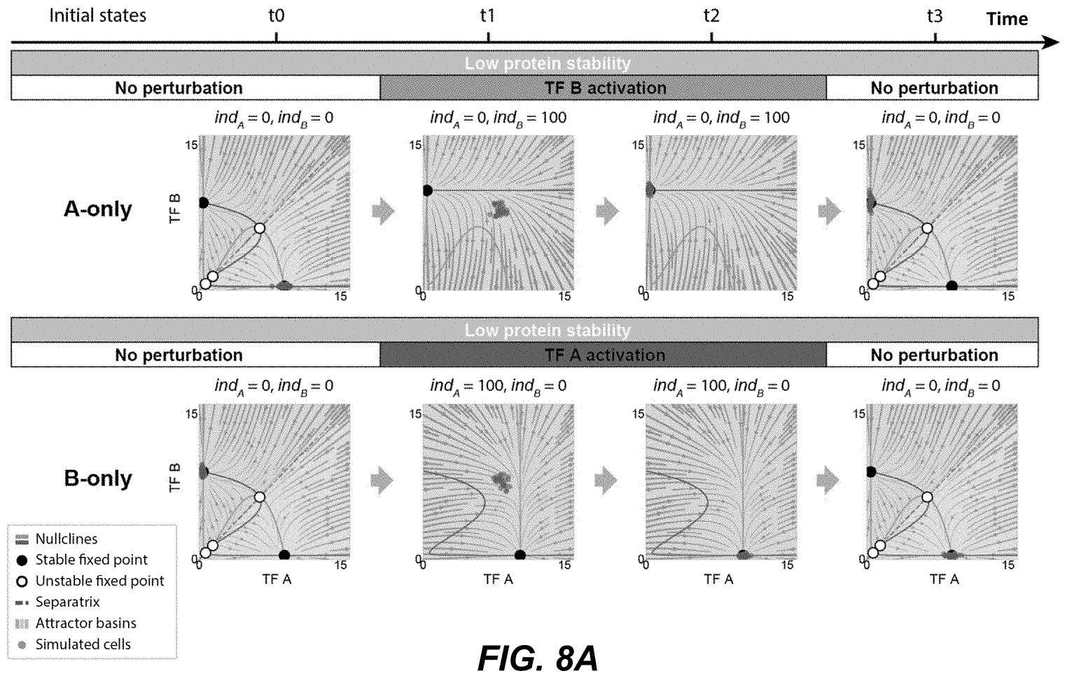

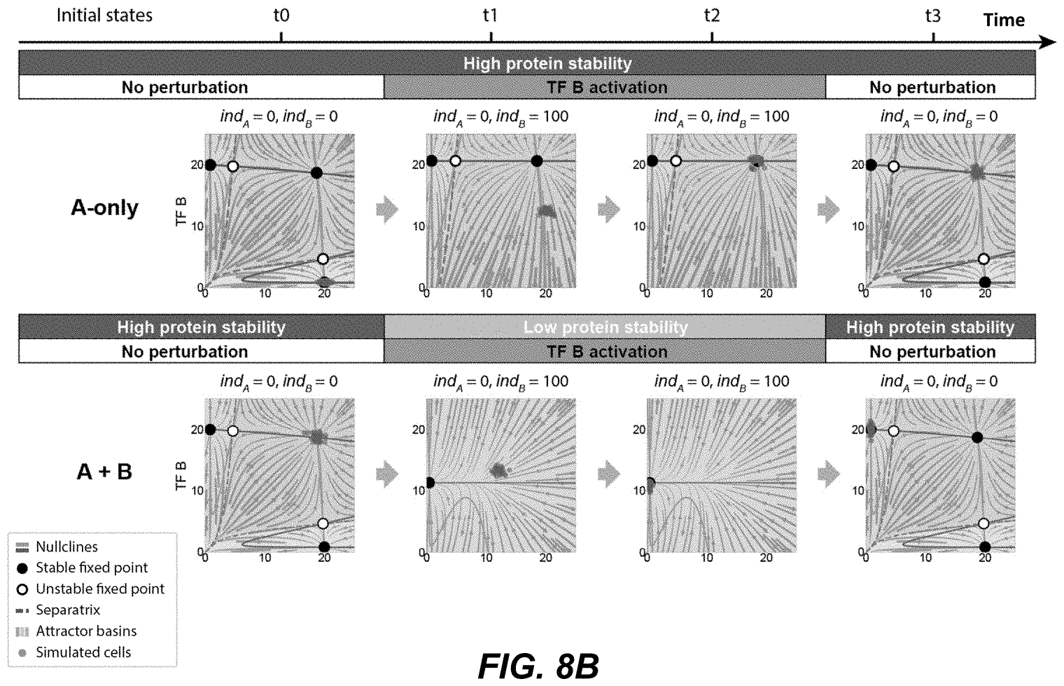

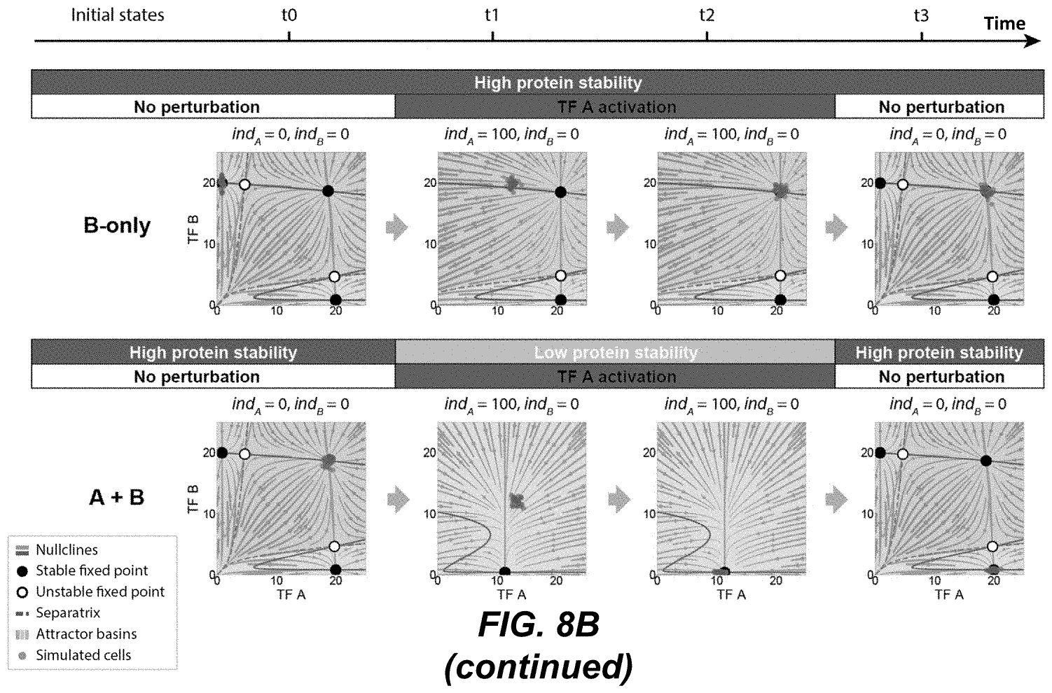

Mathematical modeling shows how the MultiFate architecture provides each of the desired capabilities (FIG. 1A) in physiologically reasonable parameter regimes (See, “Design of the MultiFate Circuit” below and Table 1). A MultiFate circuit with just two transcription factors, designated MultiFate-2, can produce diverse types of multistability containing 2, 3, or 4 stable fixed points depending on protein stability and other parameter values (FIG. 1C and FIG. 6A). A regime designated type II tristability is analogous to multilineage priming in uncommitted progenitor cells, with the double positive state playing the role of a multipotent progenitor. Transient expression of one transcription factor can switch cells between states (FIG. 8A-FIG. 8B). Reducing the protein stability of transcription factors can cause bifurcations that selectively destabilize certain states (FIG. 1C and FIG. 6A). Finally, the model is expandable: addition of a new transcription factor to the MultiFate-2 model can generate a MultiFate-3 circuit that supports additional stable states with the same parameter values (FIG. 1D and FIG. 7A). Together, these modeling results show that the MultiFate architecture can support a rich array of multistable behaviors.

### Engineered Zinc Finger Transcription Factors Enable Homodimer-Dependent Self-Activation and Heterodimer-Dependent Inhibition.

Synthetic zinc finger (ZF) transcription factors provide, in some embodiments, a platform to implement the MultiFate circuit. They can recognize and activate a promoter containing target DNA binding sites with high specificity. Further, engineered ZF DNA-binding domains containing three fingers bind weakly as monomers to 9 bp target sites, but can bind much more strongly as homodimers to 18 bp tandem binding site pairs. Without being bound by any particular theory, this property enables homodimer-dependent transcriptional activity and inhibition through heterodimerization.

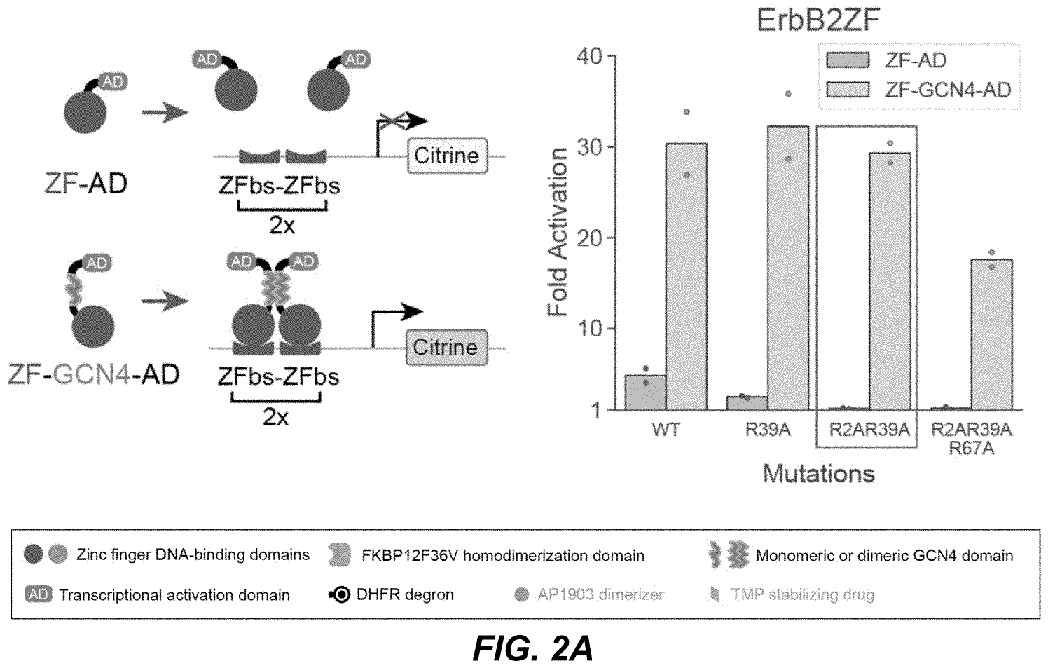

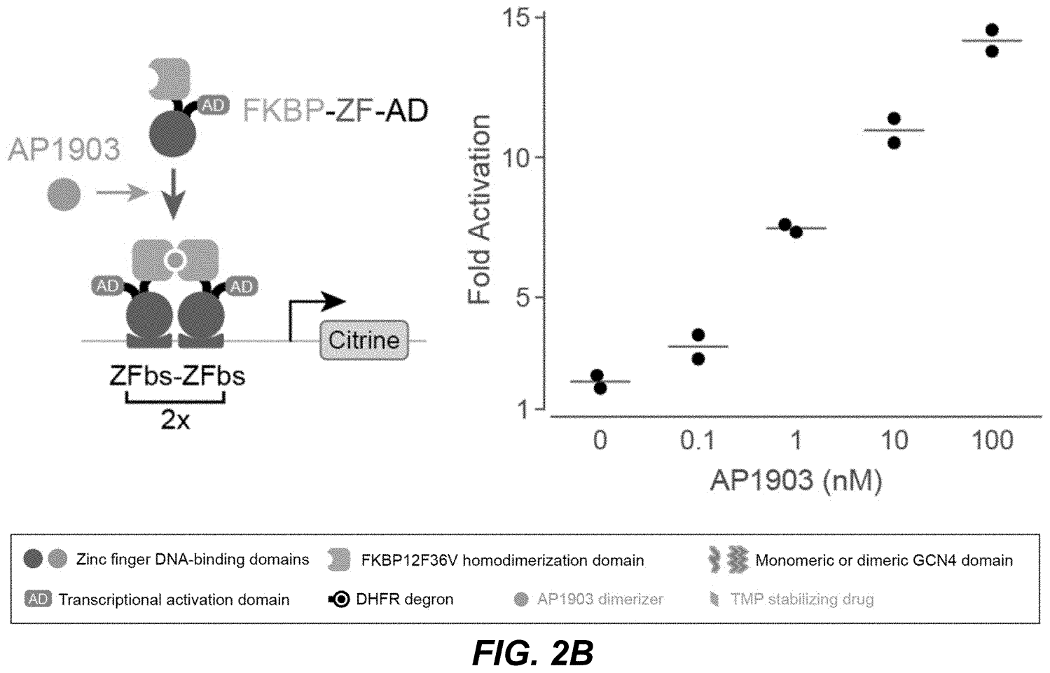

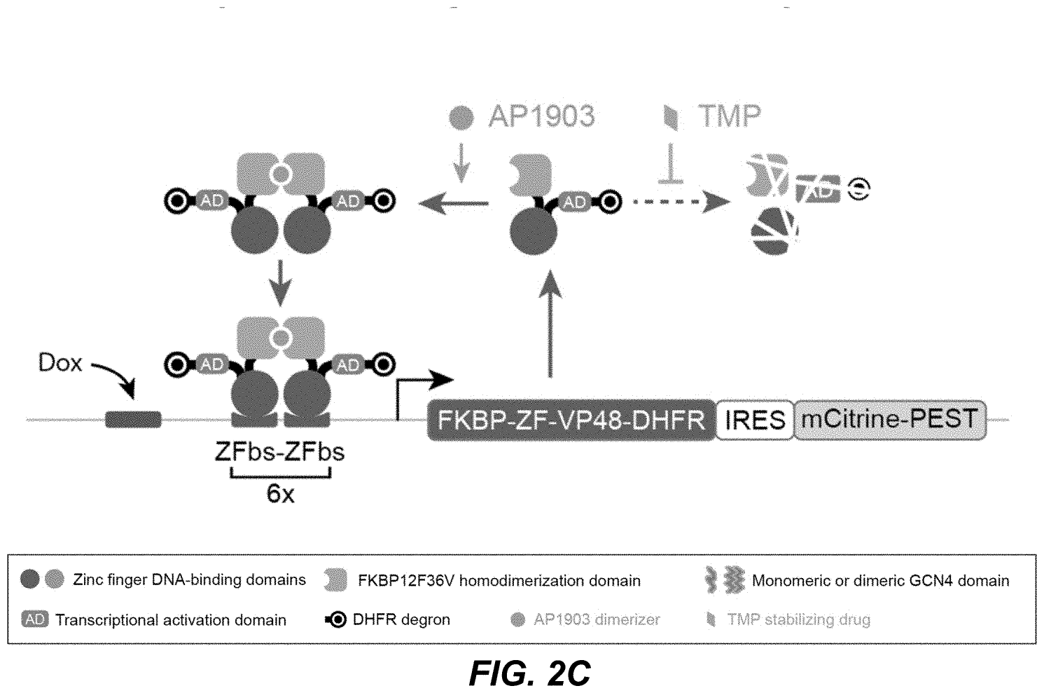

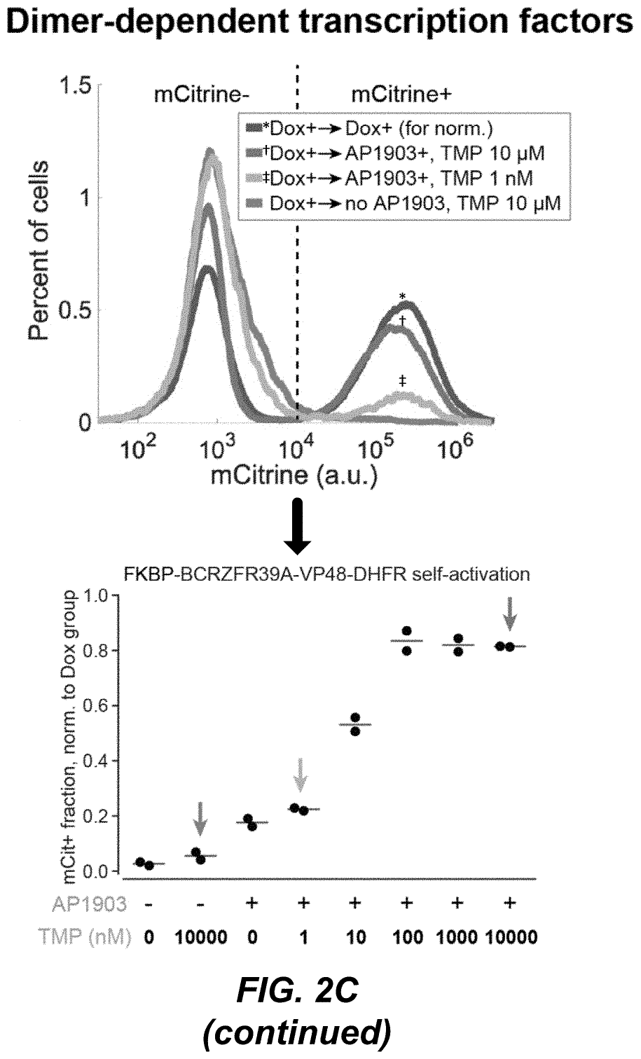

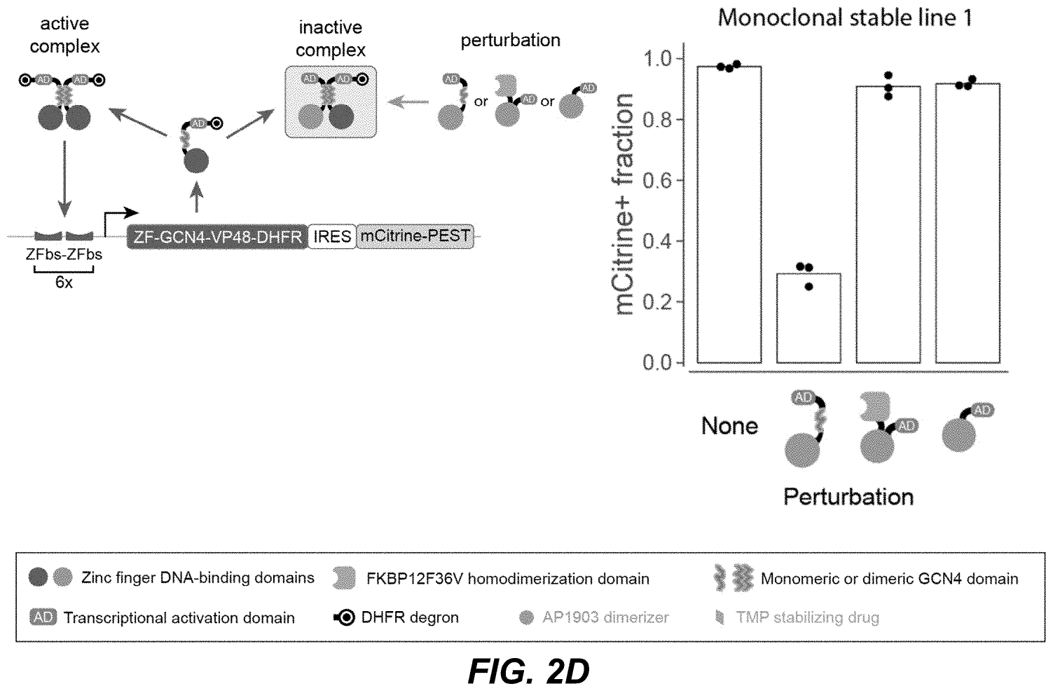

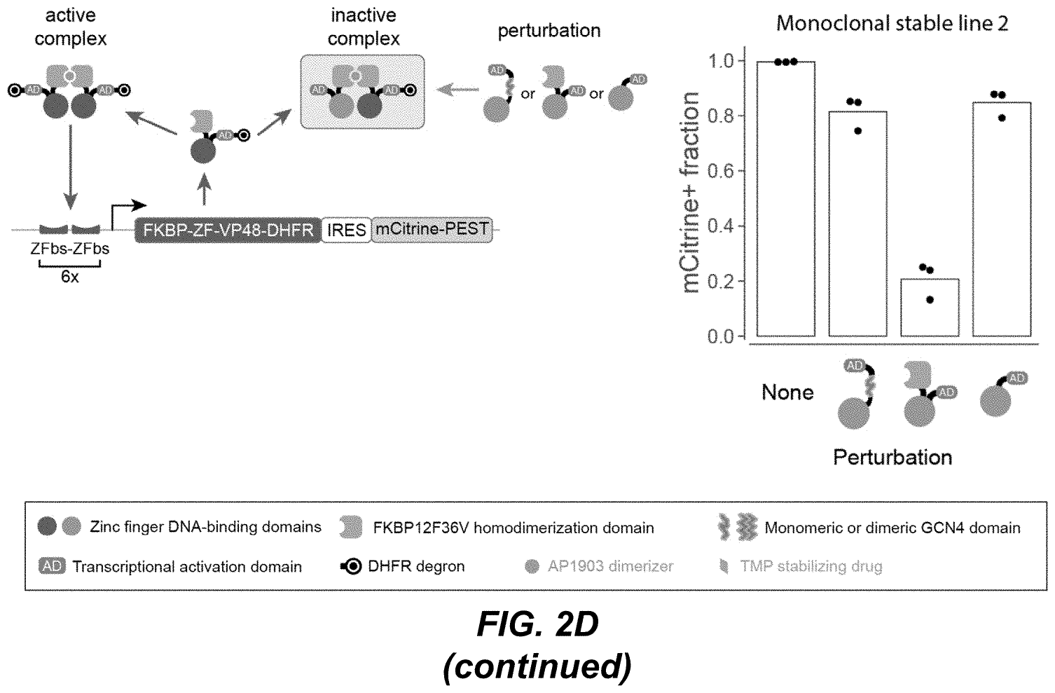

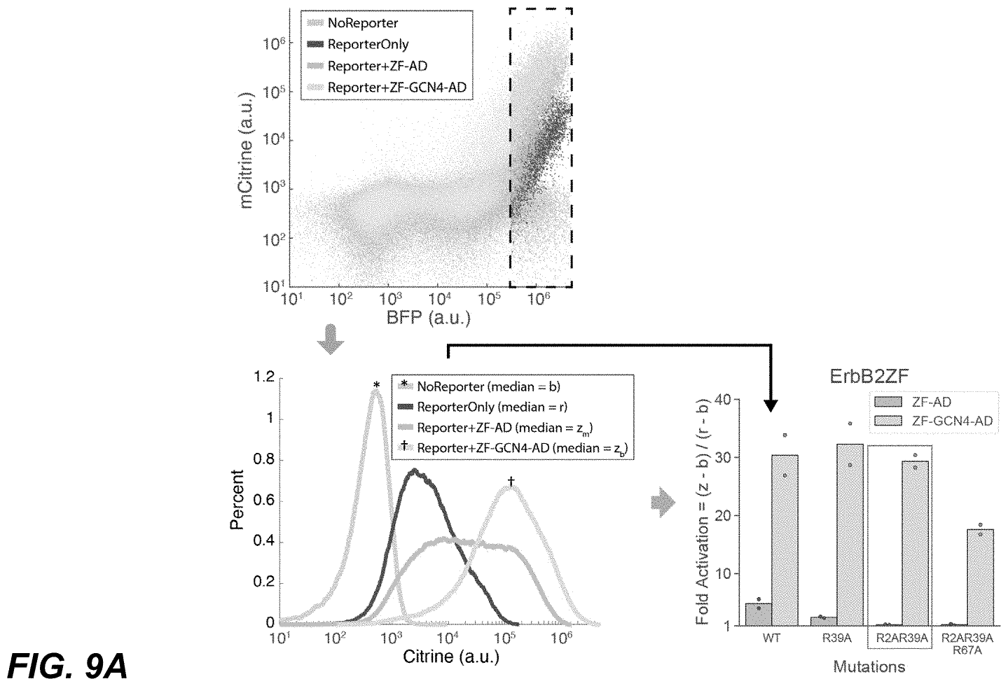

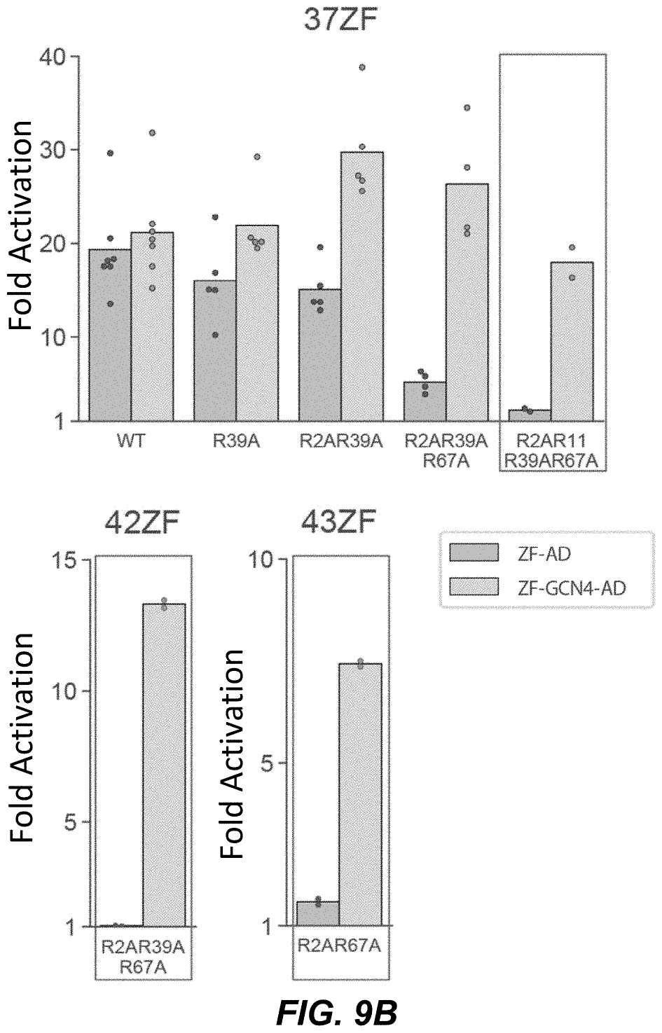

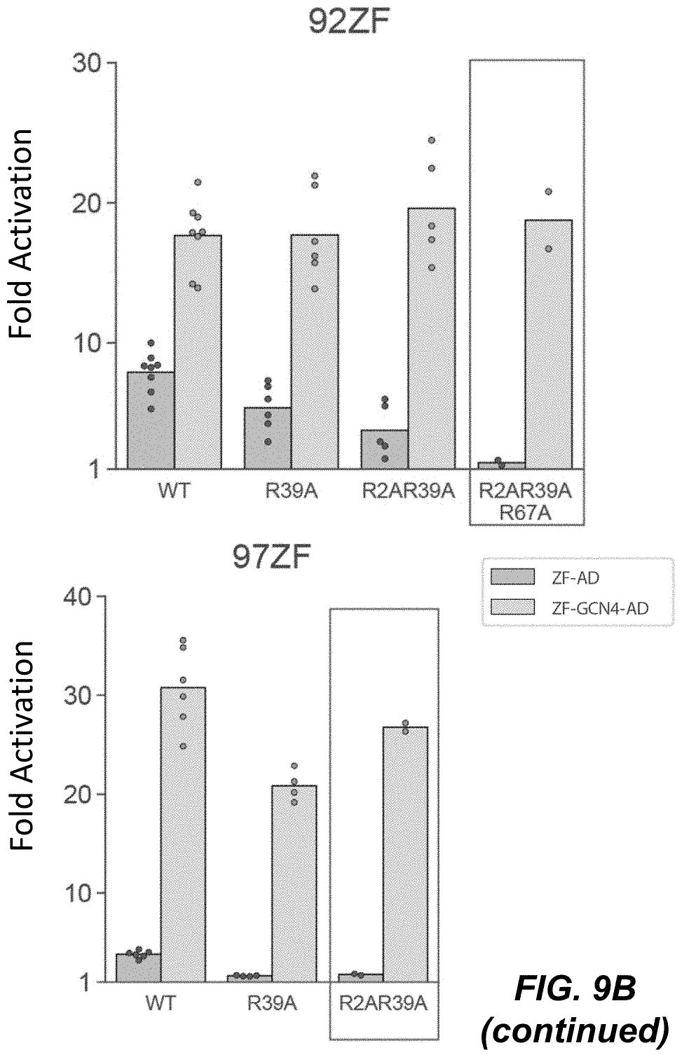

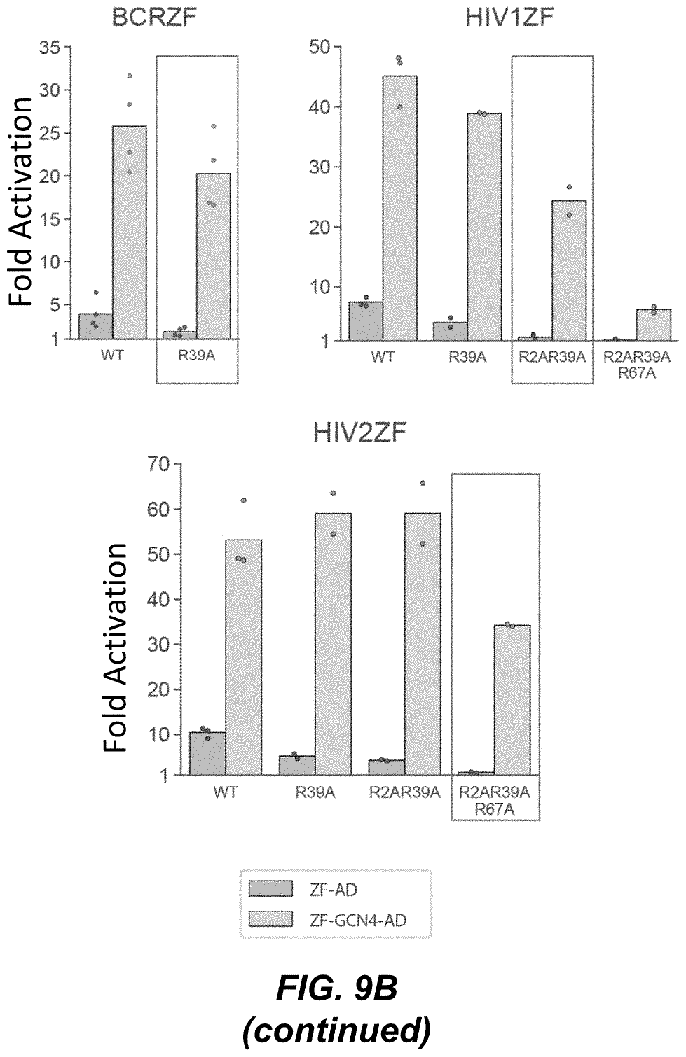

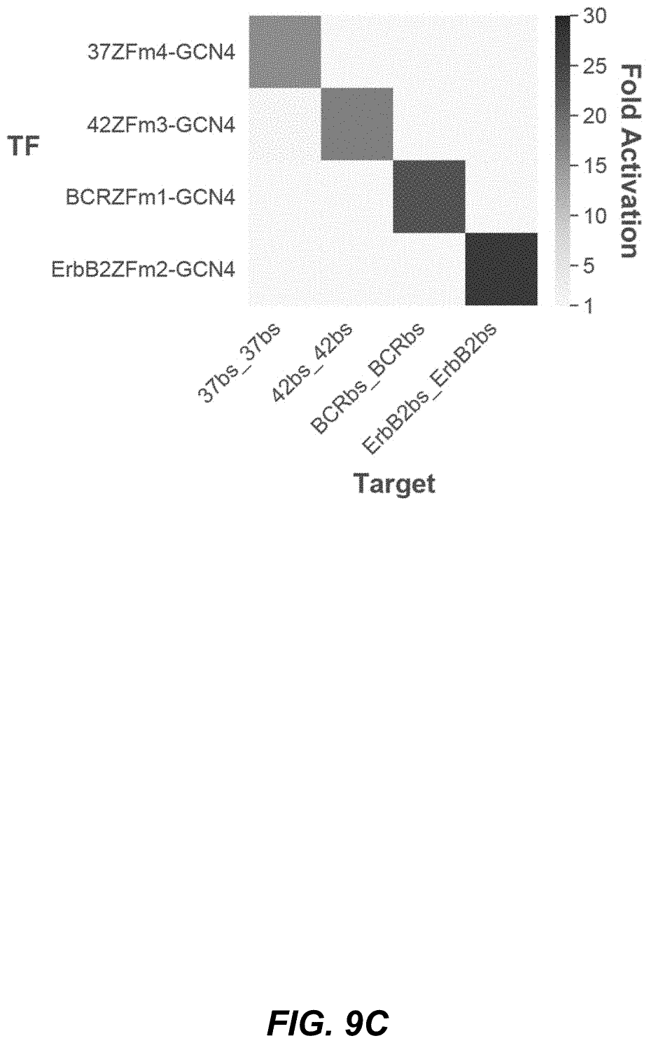

To engineer ZF transcription factors, the ErbB2 ZF DNA-binding domain was fused to a GCN4 homodimerization domain and a VP48 transcriptional activation domain to create the synthetic transcription factor, termed ZF-GCN4-AD (FIG. 2A). A transcription factor (ZF-AD) lacking GCN4 was used as a monomeric control. To assay their transcriptional activity, a reporter was constructed containing 18 bp homodimer binding sites driving the expression of Citrine. Each transcription factor was then co-transfected together with the reporter and an mTagBFP2 co-transfection marker into Chinese hamster ovary K1 (CHO-K1) cells, and analyzed for Citrine expression by flow cytometry 36 hours later (FIG. 2A and FIG. 9A) (See, “Additional Methods” below). The wild-type (WT) ZF-GCN4-AD factors strongly activated the reporter whereas ZF-AD exhibited weaker basal activity (FIG. 2A and FIG. 9B). Arginine-to-alanine mutations were introduced at key positions in the ZF known to weaken DNA binding, which decreased monomeric activity without reducing homodimer activity (FIG. 2A, red square). Replacing the GCN4 with the FKBP12F36V (FKBP) homodimerization domain allowed for dose-dependent control of dimerization with the small molecule AP1903 (FIG. 2B). Finally, this general design was repeated to engineer a set of additional homodimer-dependent ZF transcription factors with orthogonal DNA-binding specificities (FIG. 9B and FIG. 9C).

In some embodiments, the MultiFate circuit design requires that each transcription factor positively autoregulates its own expression in a homodimer-dependent manner. To validate this capability, a self-activation construct was designed (FIG. 2C), in which a transcription factor with a FKBP dimerization domain is expressed from a promoter containing its own 18 bp homodimer binding sites (Table 2). This construct allowed independent Dox-inducible activation through upstream Tet3G (Takara Bio) binding sites. It also incorporated a dihydrofolate reductase (DHFR) degron, which can be inhibited by trimethoprim (TMP), permitting control of protein stability. Finally, a destabilized mCitrine was incorporated for dynamic readout of construct expression. This construct was integrated into Tet3G-expressing CHO-K1 cells, generating a stable polyclonal population for further analysis (Table 3) (See, “Additional Methods” below).

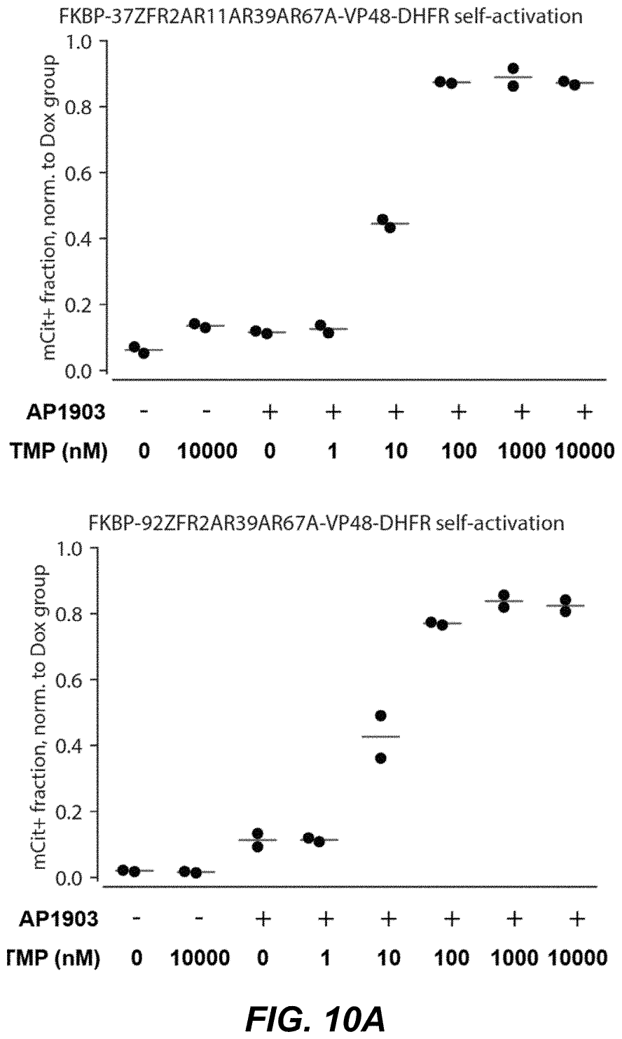

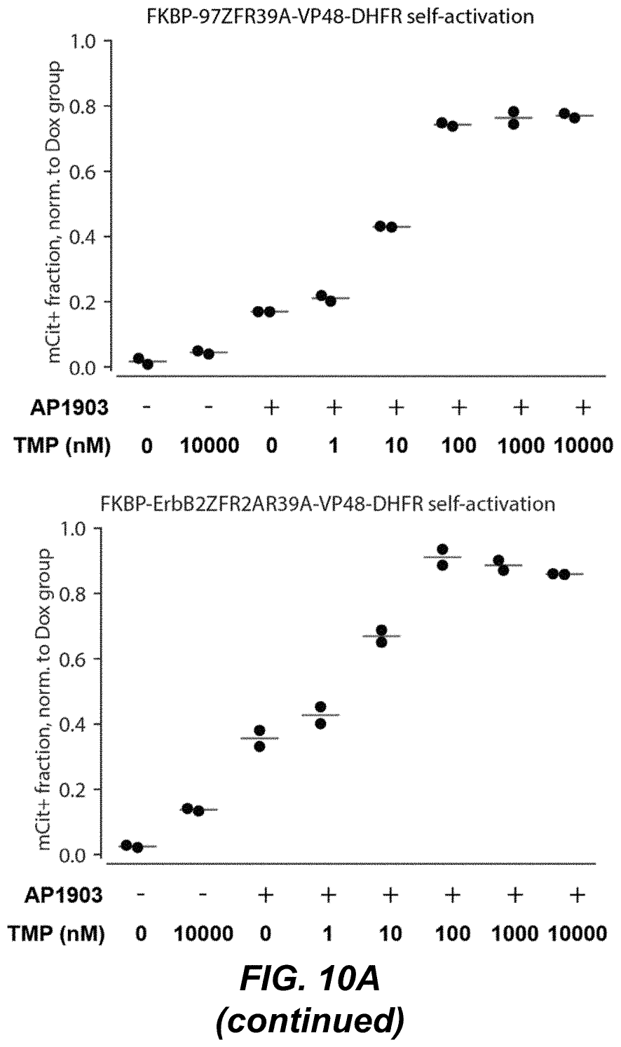

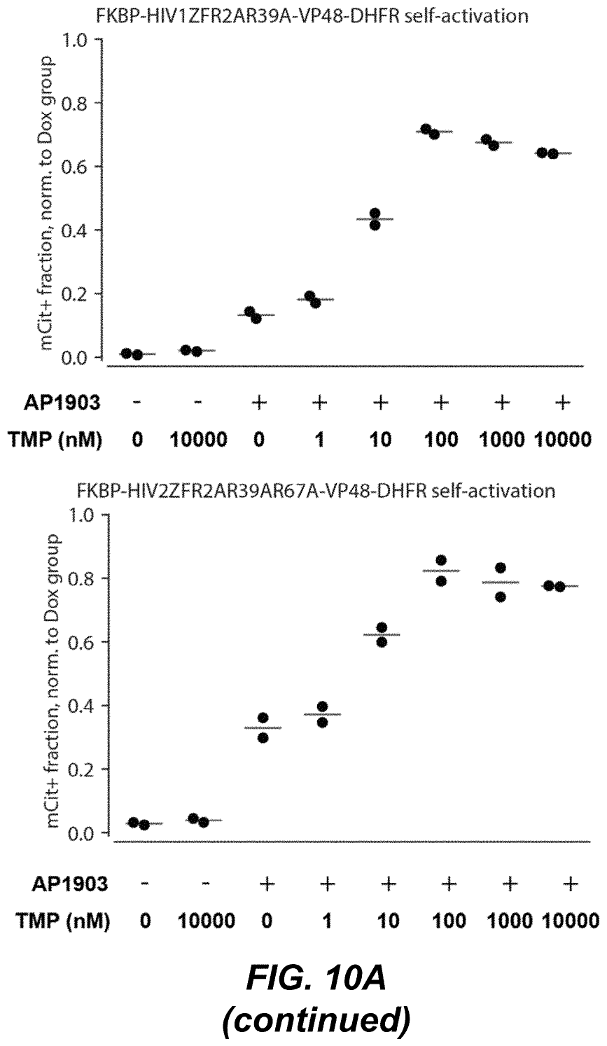

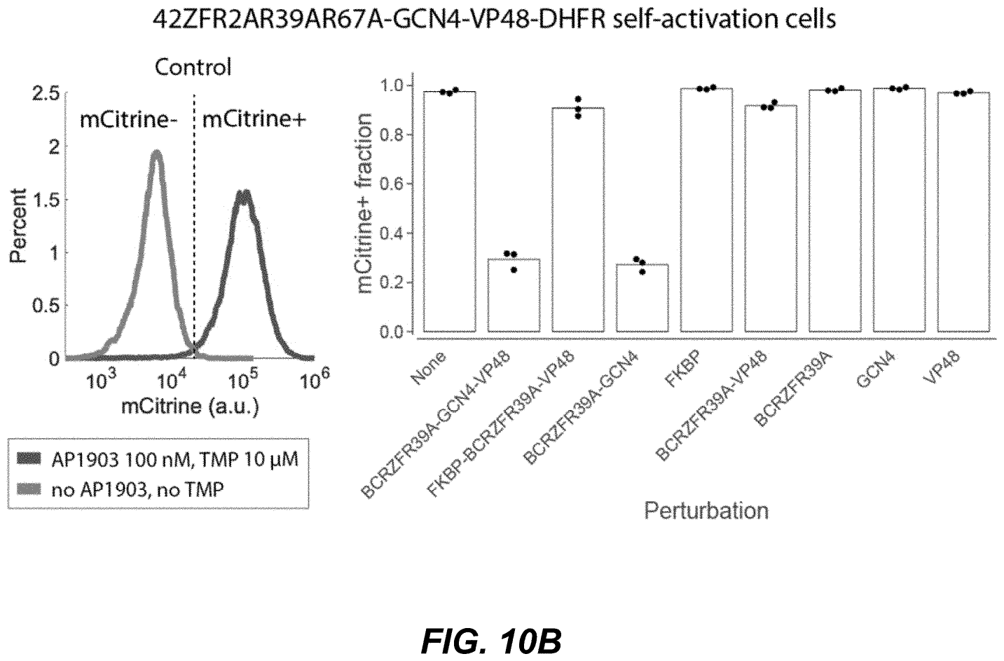

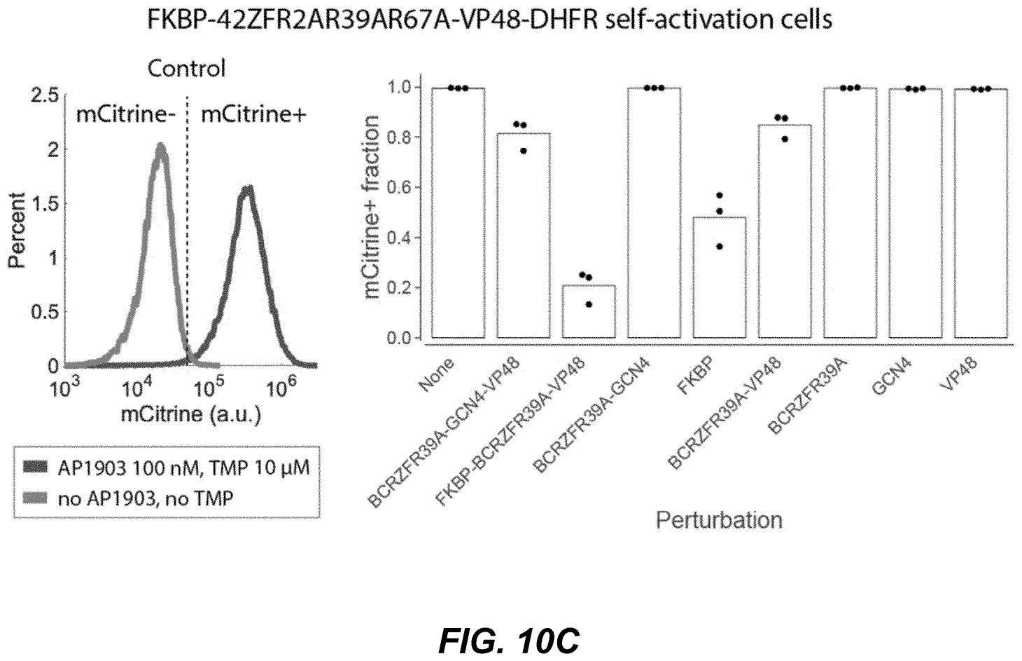

To test for self-activation, transcription factor expression was transiently induced for 24 hours with Dox, and then Dox was withdrawn and cells were checked as to whether they could sustain circuit activation when dimerization strength and protein stability were varied by AP1903 and TMP, respectively. In the presence, but not the absence, of AP1903, cells exhibited a bimodal distribution of mCitrine fluorescence, with well-separated peaks (FIG. 2C, top graph), consistent with homodimer-dependent self-activation in a subset of cells. TMP, by stabilizing transcription factors, also promoted self-activation in a dose-dependent manner (FIG. 2C and FIG. 10A). Thus a single dimer-dependent transcription factor can self-activate and sustain its own expression in a controllable manner.

In some embodiments, MultiFate's final requirement is the ability of one transcription factor to effectively inhibit another through heterodimerization. To test this, monoclonal cell lines with the self-activating circuits were selected, and then constructs expressing proteins were stably integrated with a different ZF DNA-binding domain and a matching or mismatching dimerization domain to generate a polyclonal cell population for each perturbation construct (Table 2 and Table 3) (See, “Additional Methods” below). Consistent with inhibition through heterodimerization, the proteins with matching dimerization domains strongly inhibited the self-activating transcription factor, while similar proteins with non-matching dimerization domains exhibit much weaker inhibition. Without being bound by any particular theory, this may be through non-specific mechanisms (FIG. 2D and FIG. 10B-FIG. 10C). Taken together, these results provided a set of engineered ZF transcription factors that exhibit controllable homodimer-dependent activation and heterodimer-dependent inhibition.

### The MultiFate-2 Circuit Generates Tristability.

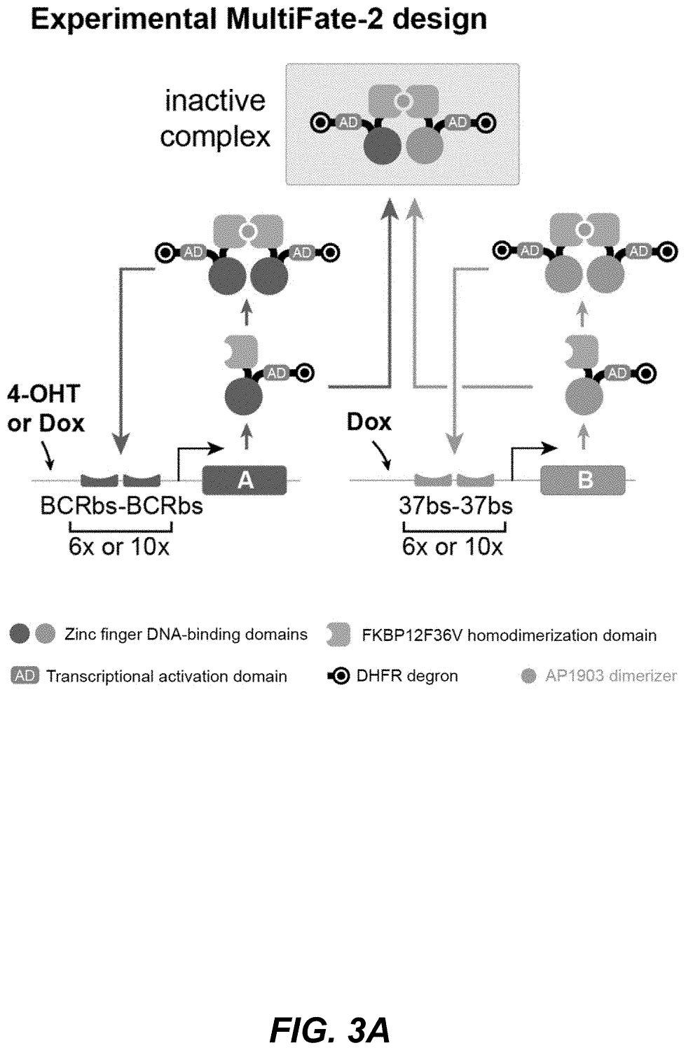

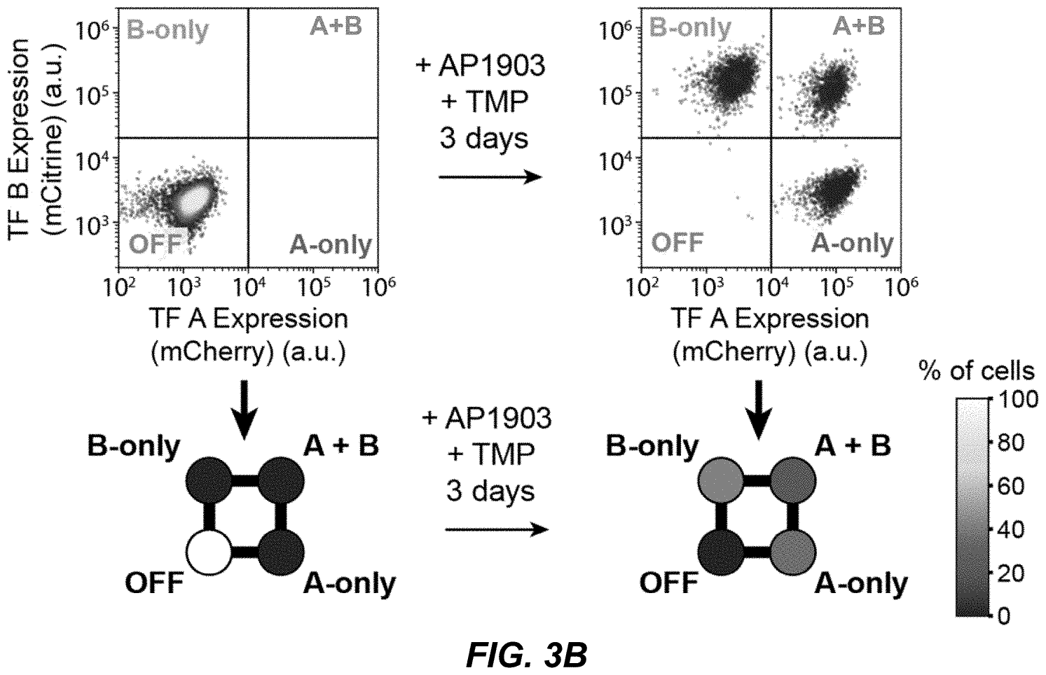

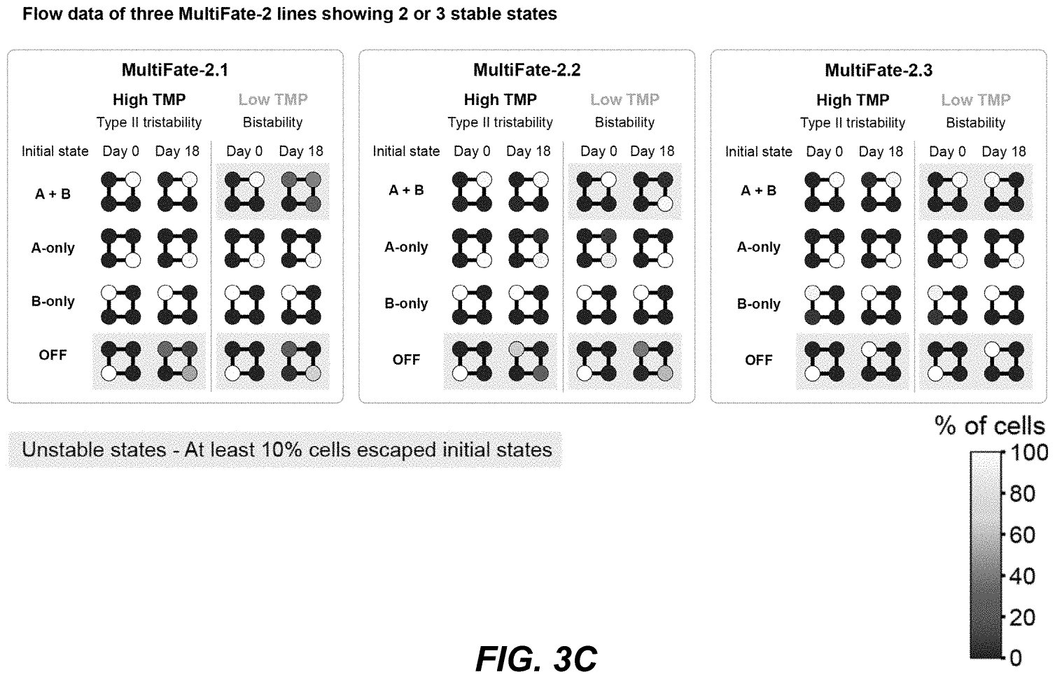

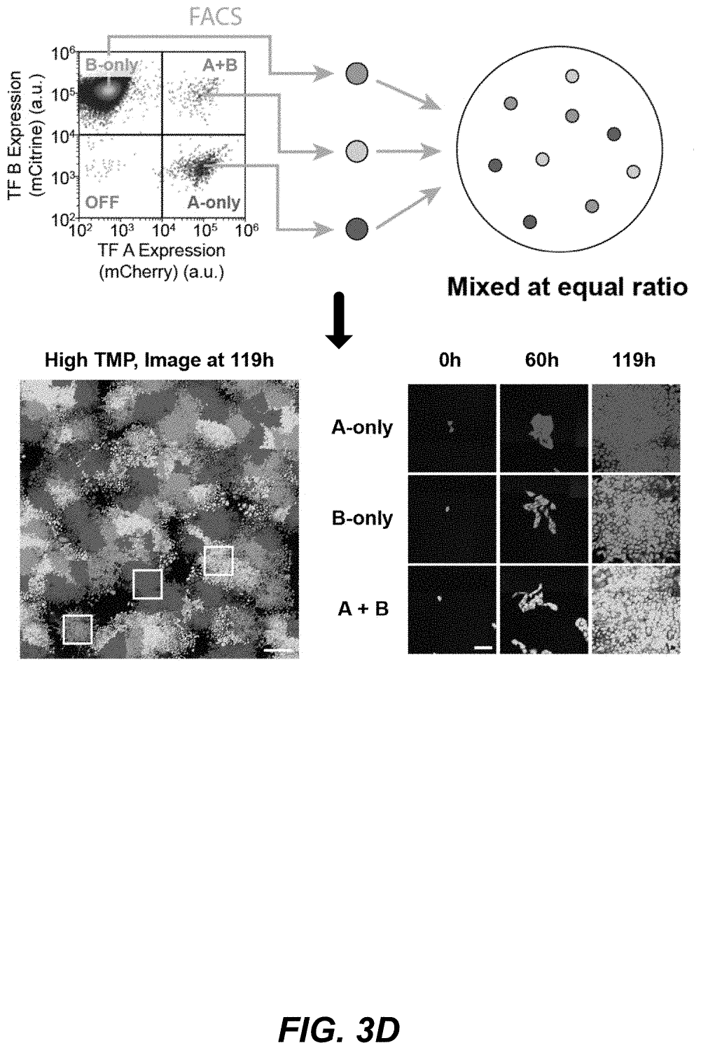

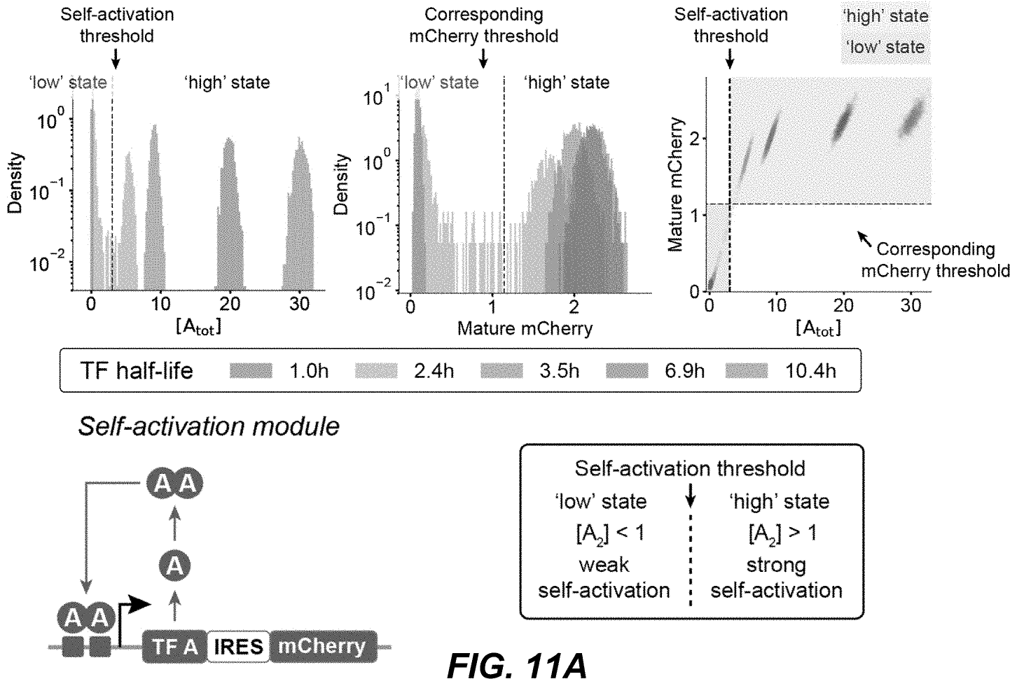

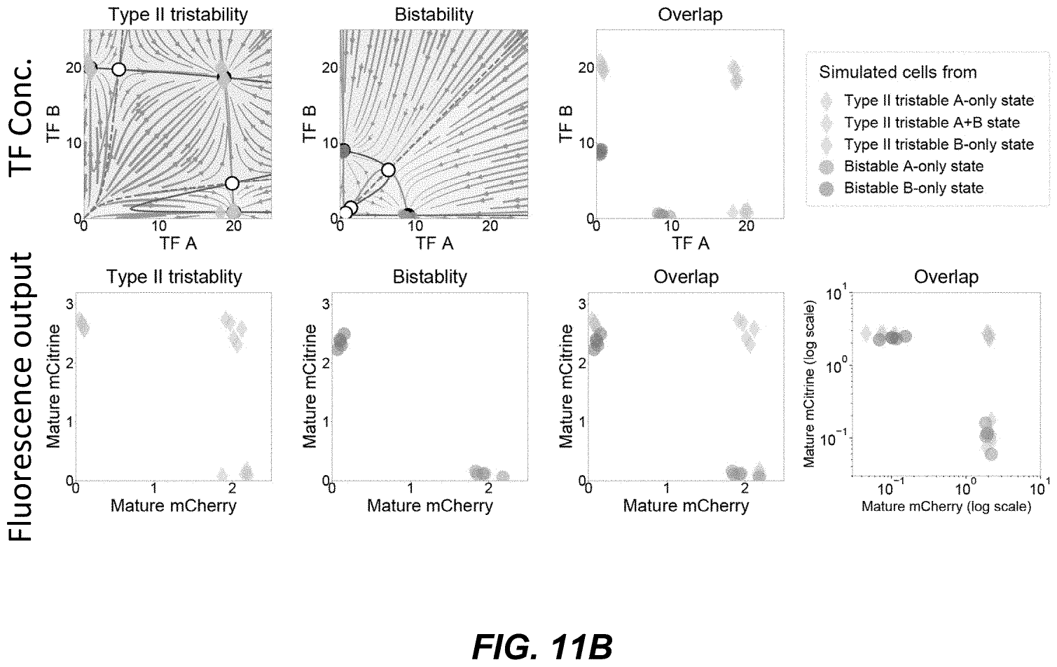

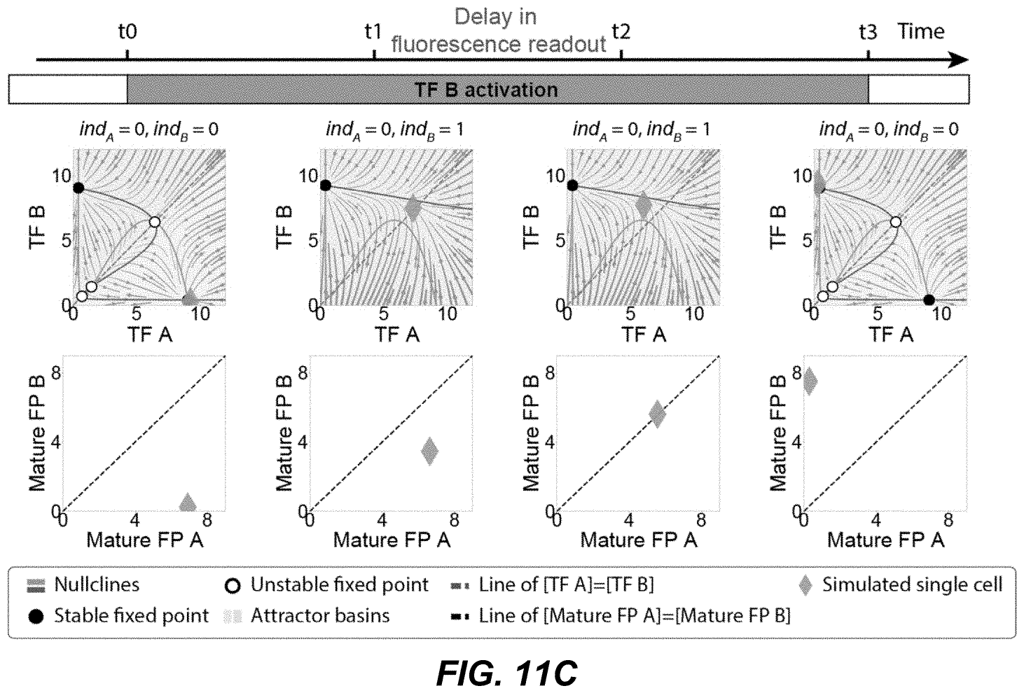

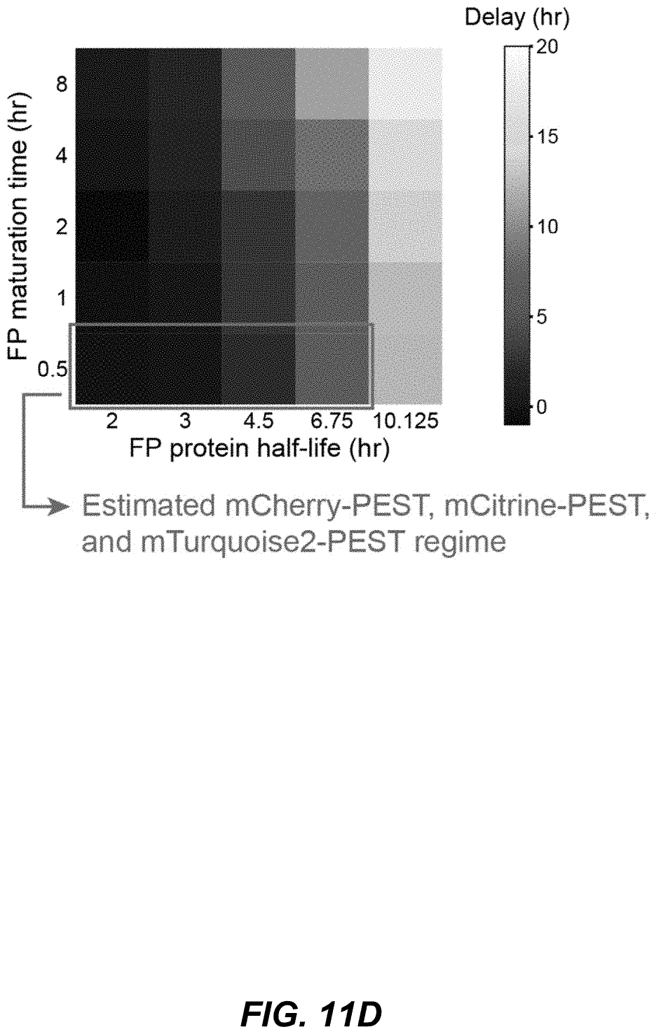

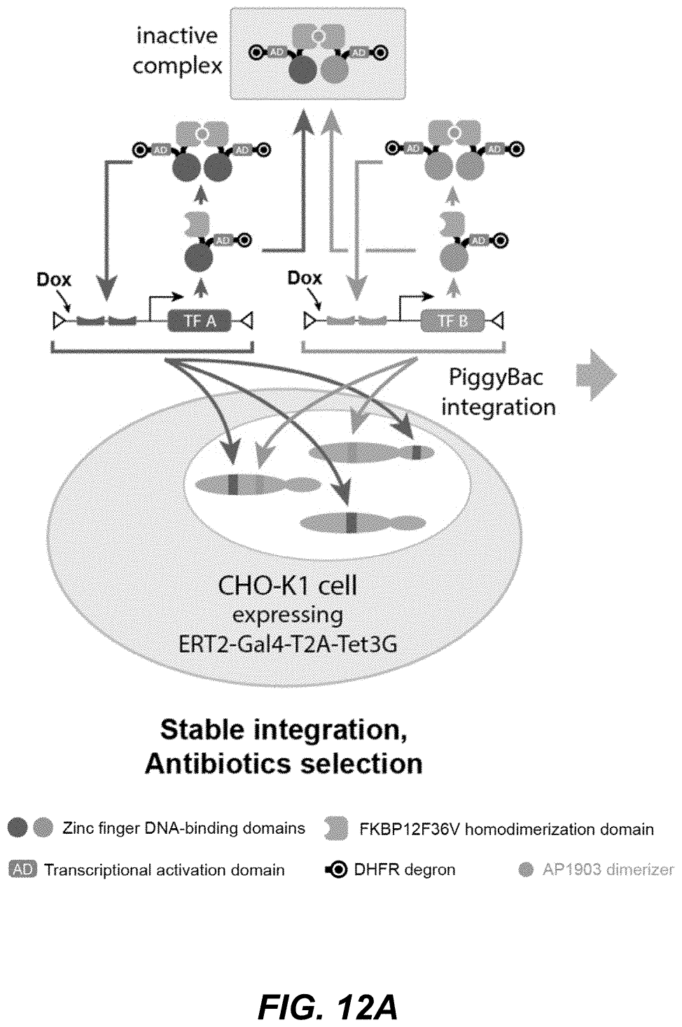

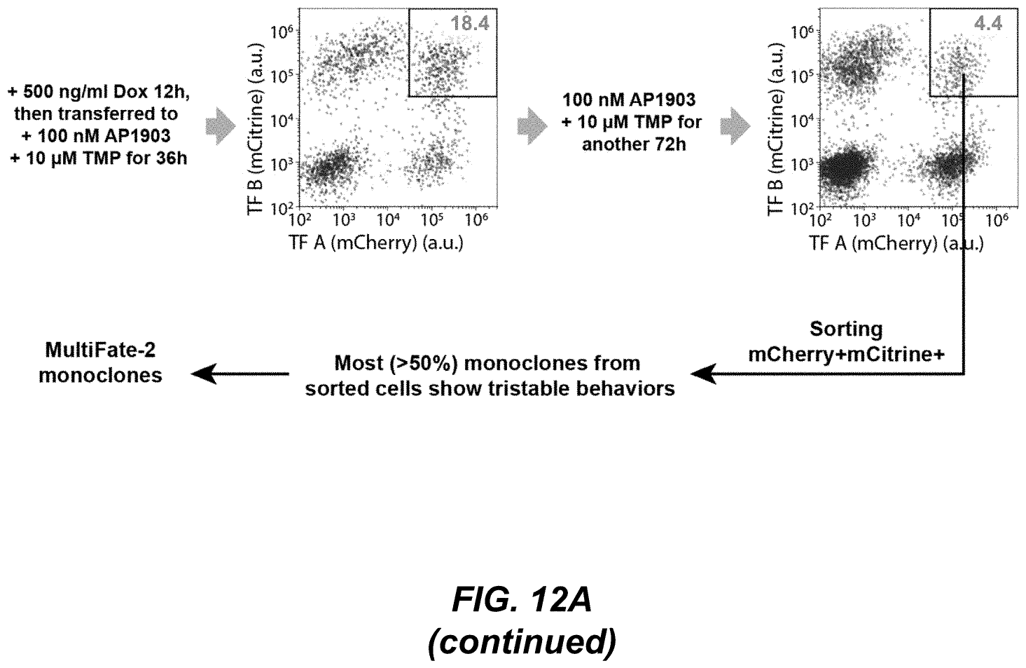

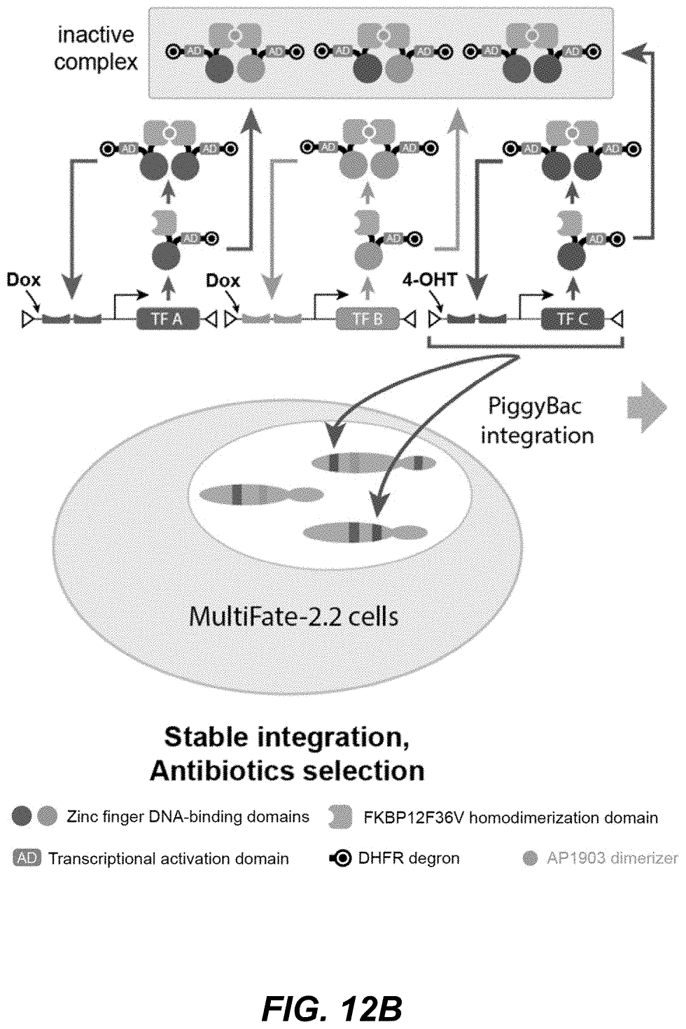

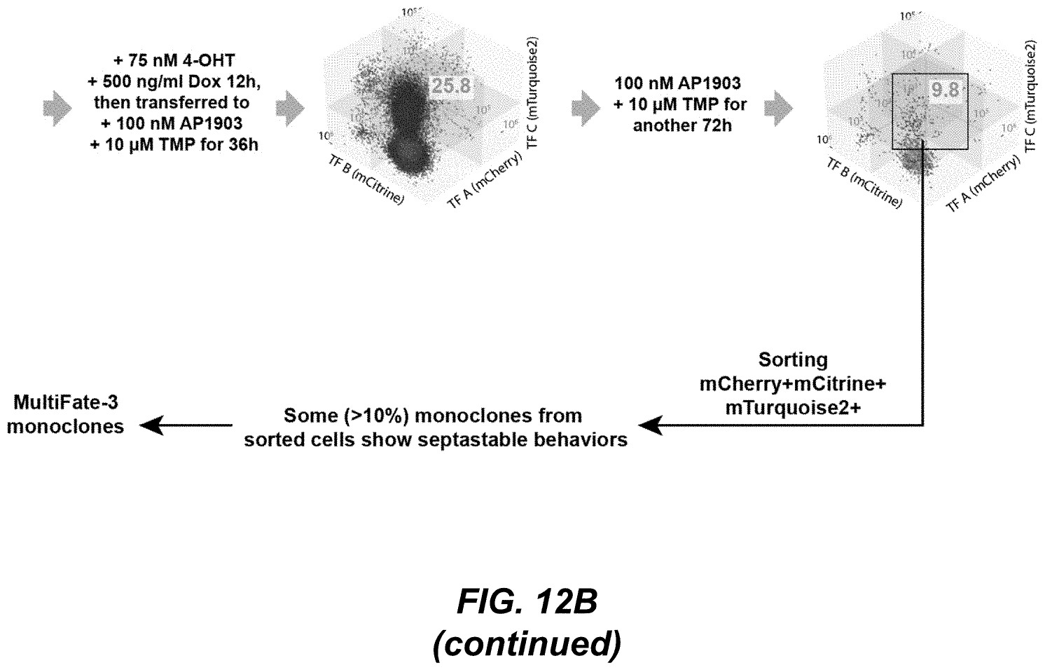

To construct a complete MultiFate circuit, two dimer-dependent transcription factors were selected, henceforth designated A and B, with distinct DNA binding specificities but the same FKBP homodimerization domain. Their expressions are driven by promoters containing multiple repeats of their corresponding 18 bp homodimer binding sites (FIG. 3A and Table 2). The promoters also incorporated Tet3G or ERT2-Gal4 response elements to allow independent external activation of transcription. A and B were transcriptionally co-expressed with destabilized mCherry or mCitrine fluorescent proteins, respectively, each placed after an internal ribosome entry site (IRES), allowing fluorescent readout of transcription rates in individual cells (FIG. 11A-FIG. 11D). Both genes were stably integrated simultaneously in CHO-K1 cells expressing Tet3G and ERT2-Gal4 proteins, and three stable monoclonal cell lines, designated MultiFate-2.1, MultiFate-2.2 and MultiFate-2.3 with different promoter configurations were selected and further characterized (FIG. 12A and Table 3) (See, “Additional Methods” below).

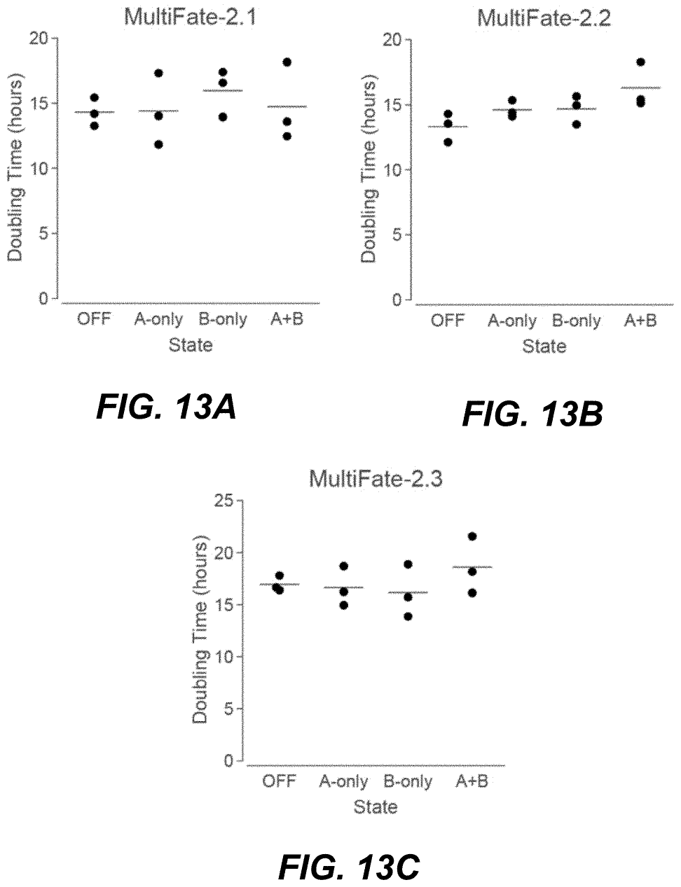

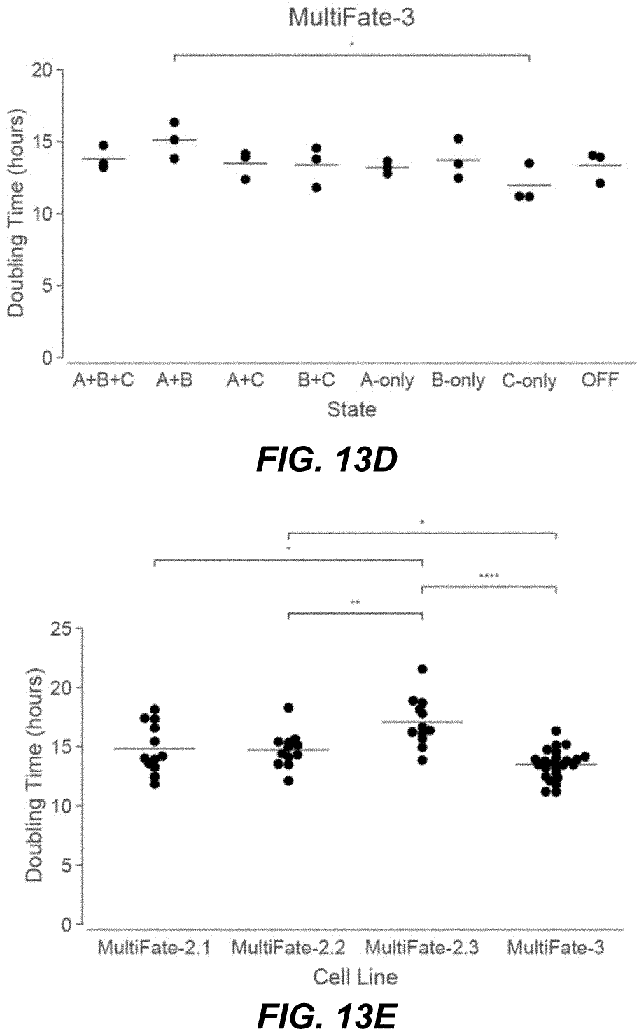

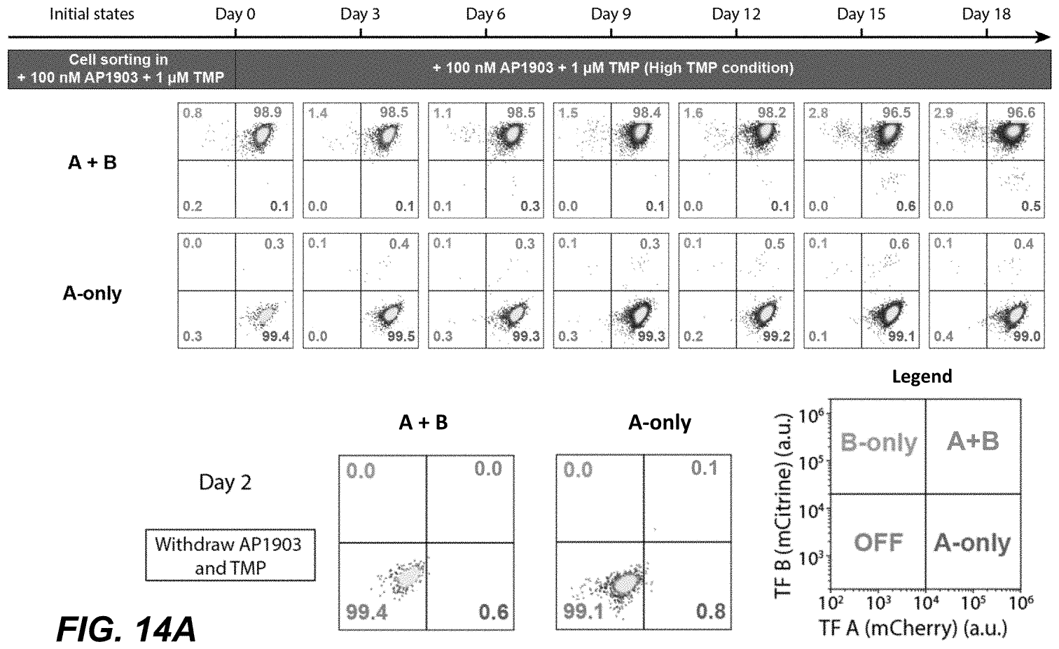

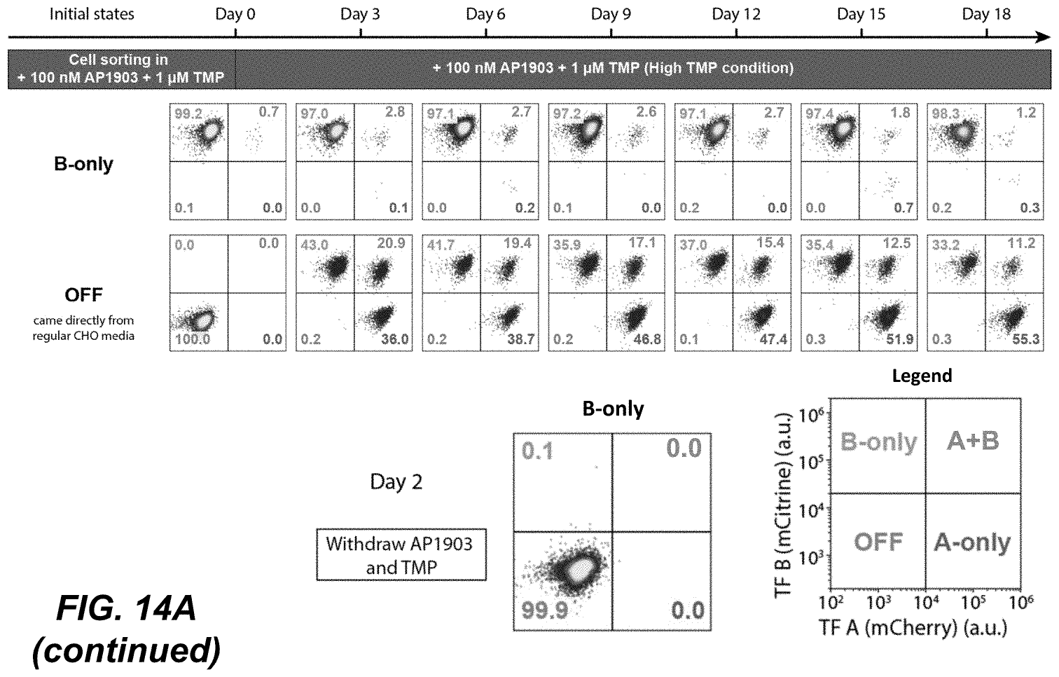

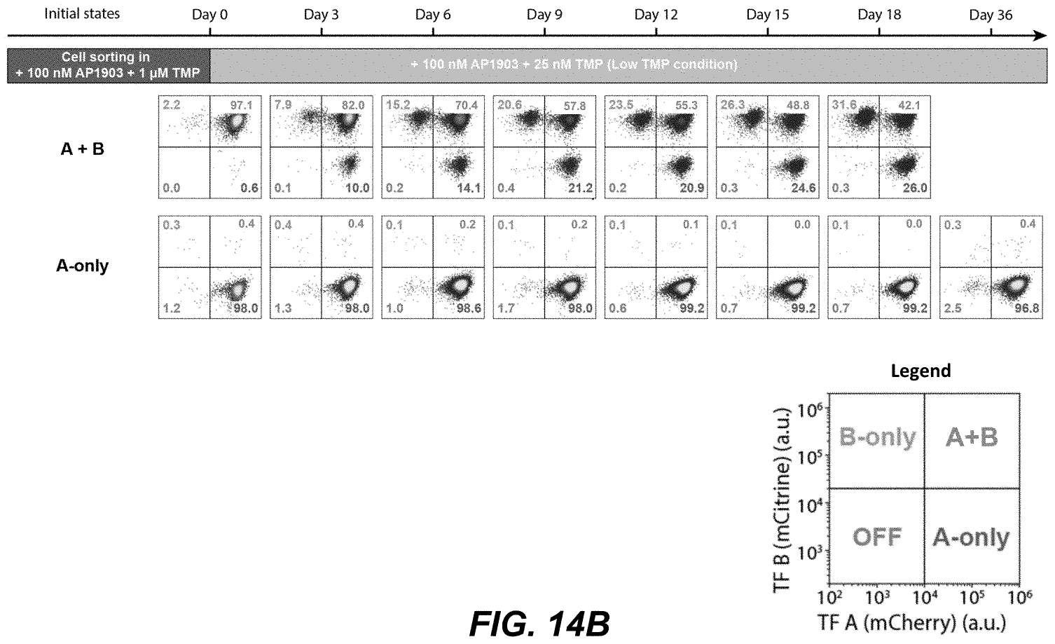

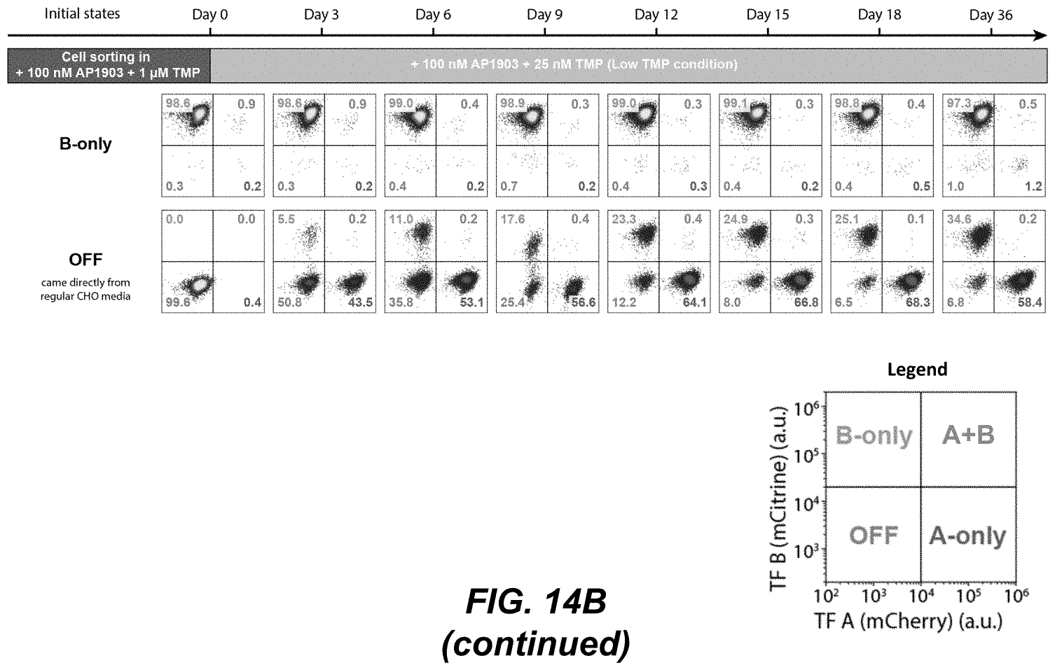

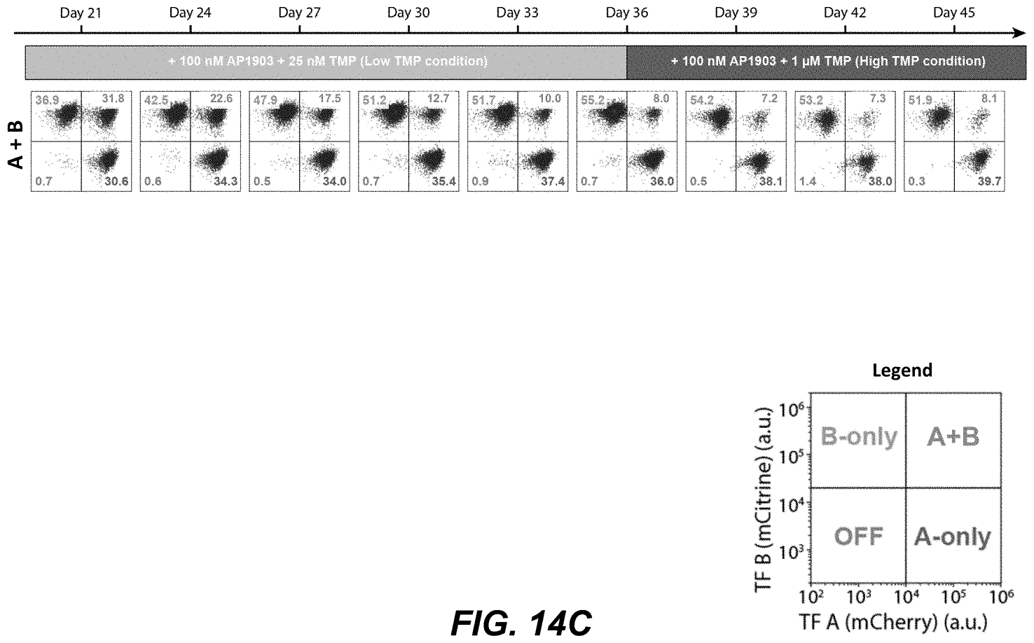

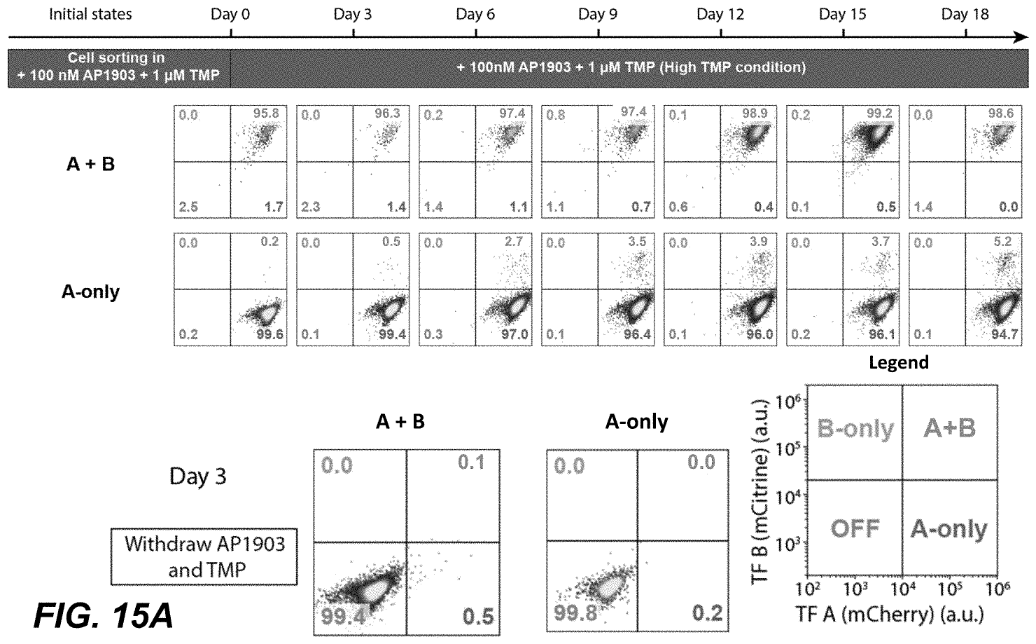

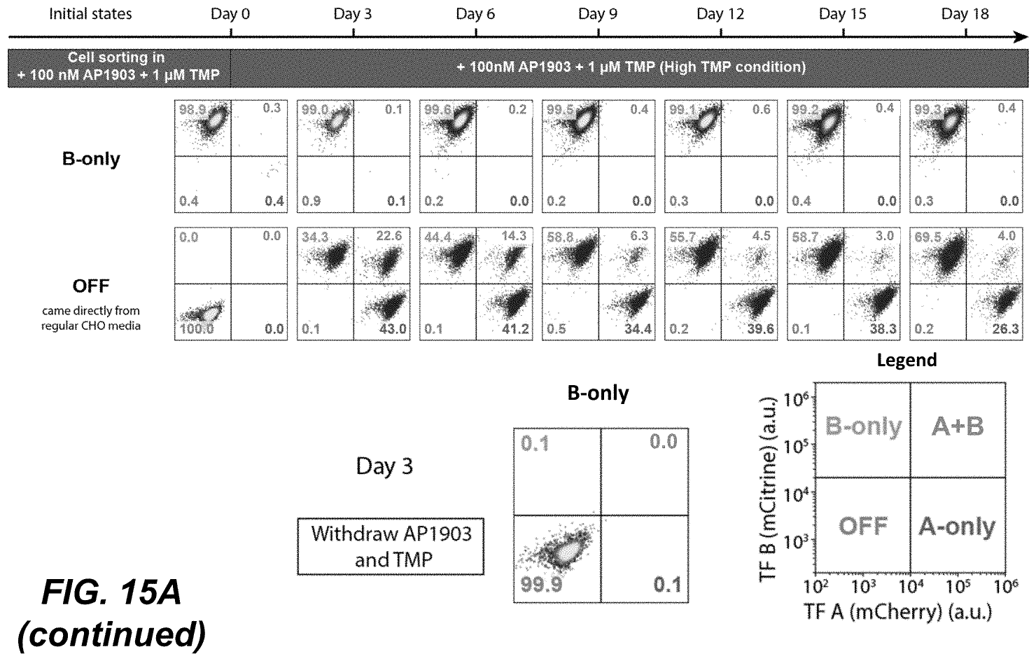

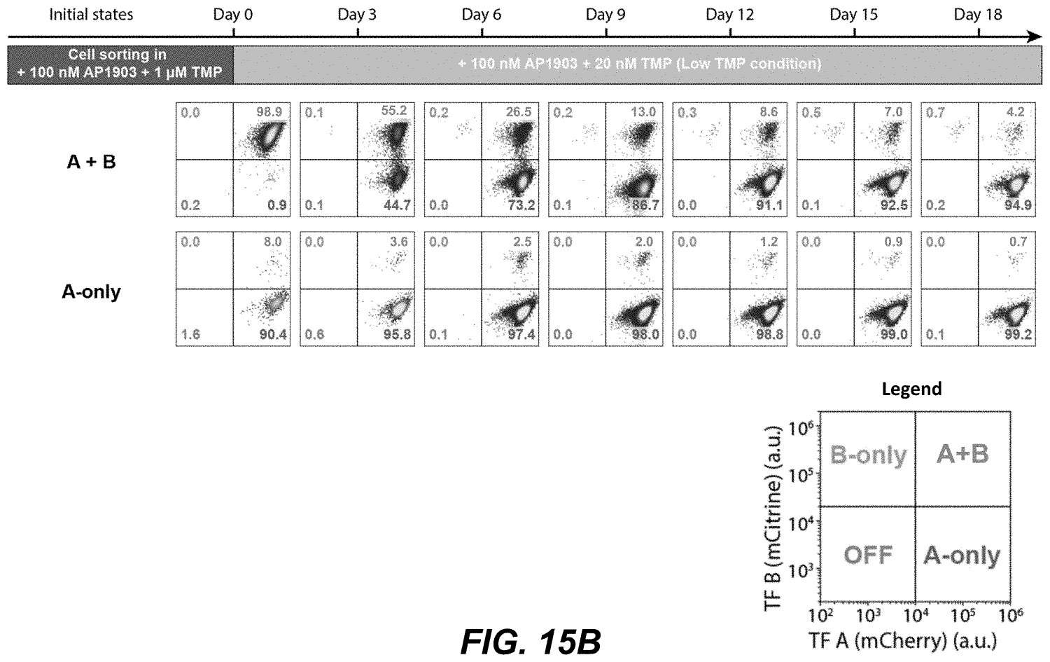

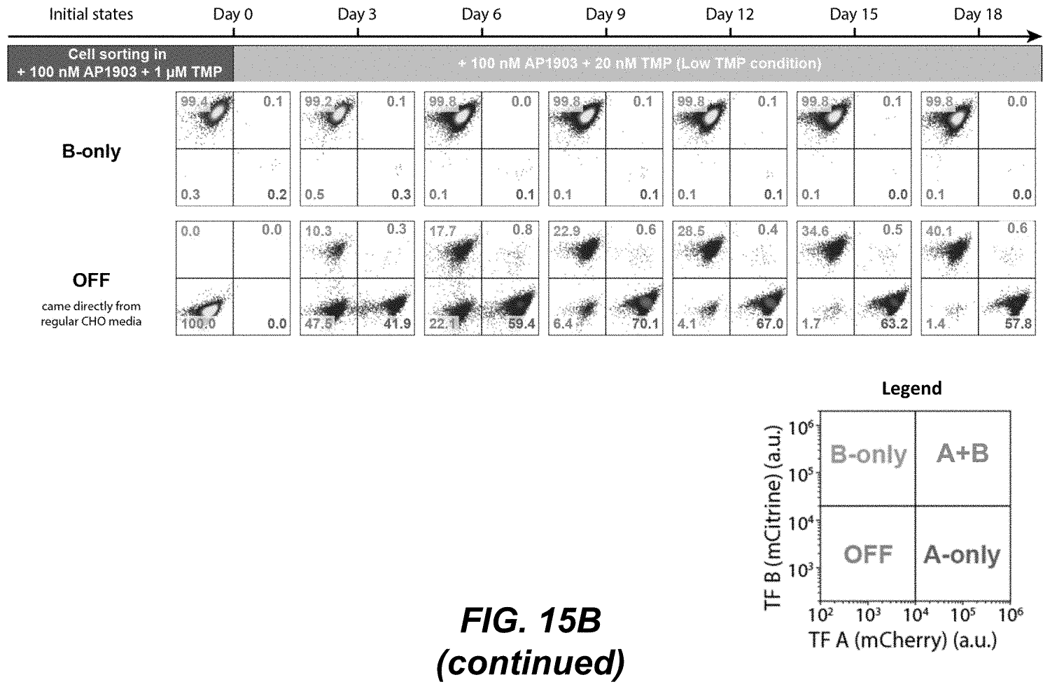

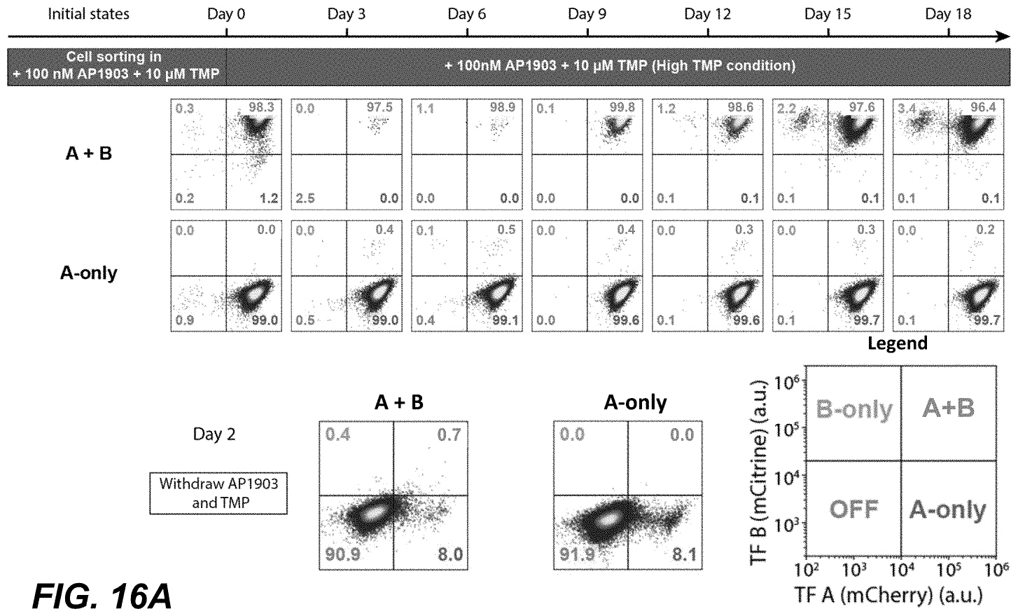

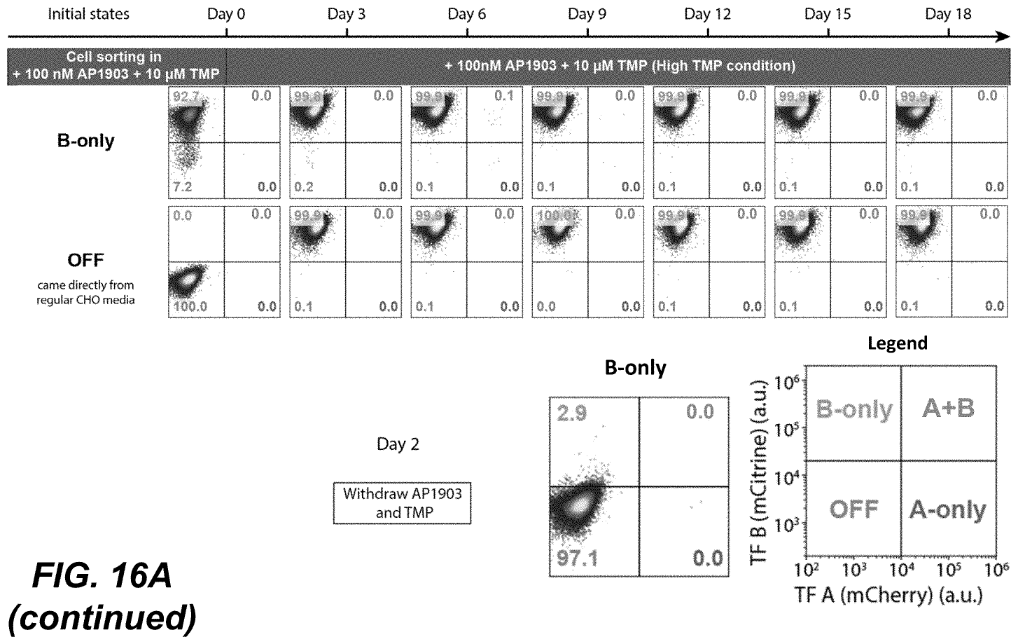

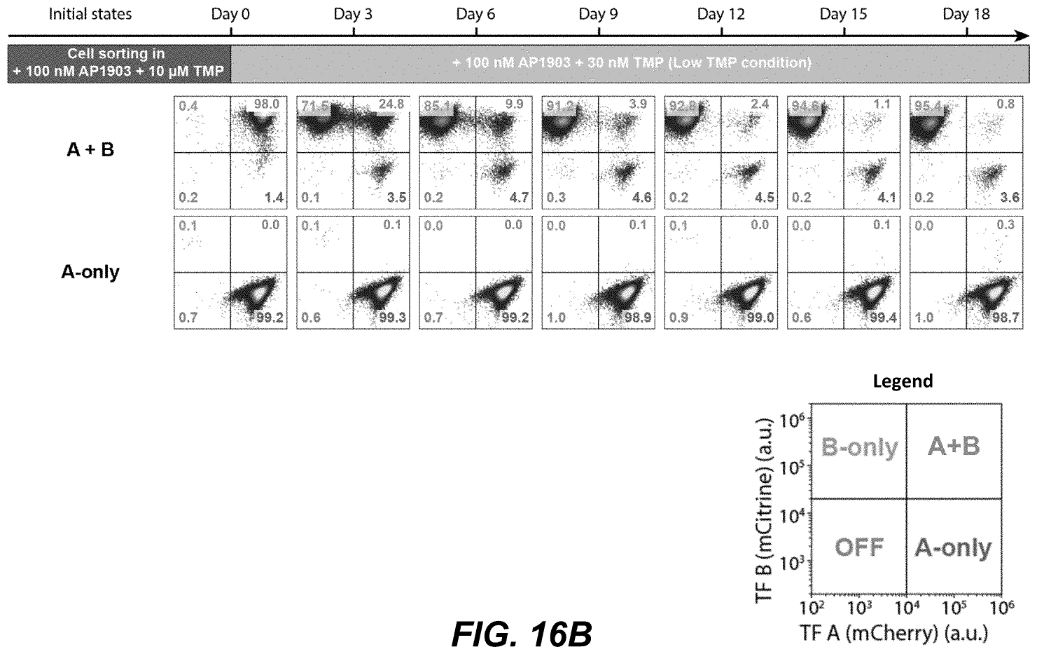

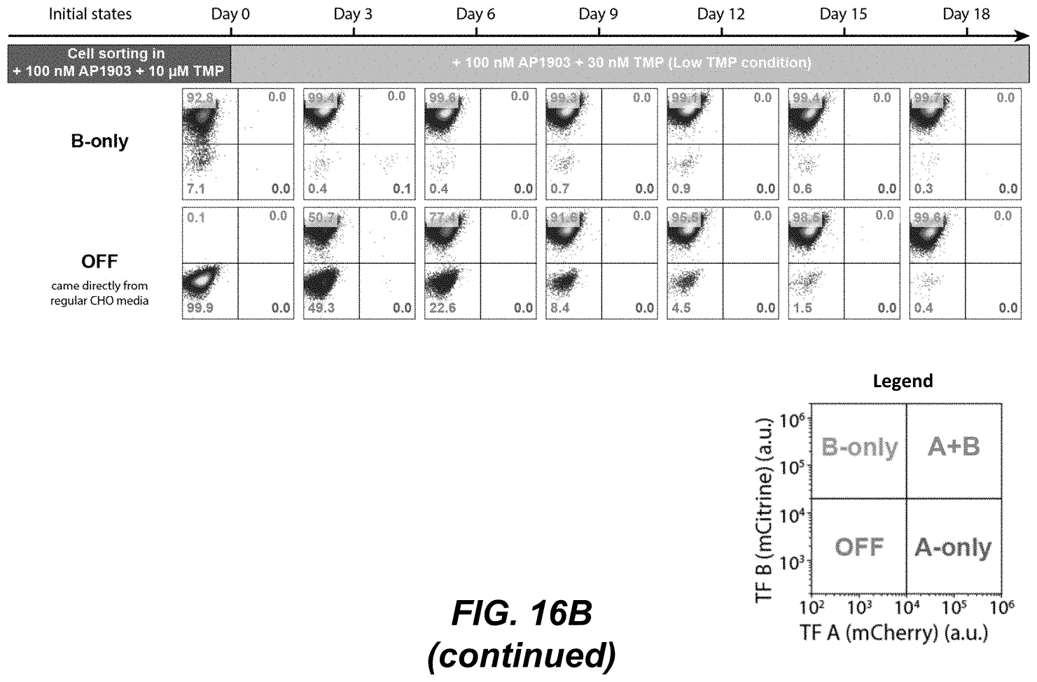

To test whether MultiFate circuits support multistability, the circuit was activated by transferring MultiFate-2.1 cells to media containing AP1903 and TMP to allow dimerization and stabilizing the transcription factors. As expected in the regime of type II tristability (FIG. 1C), cells went from low expression of both transcription factors (OFF state) to one of three distinct states, with either A, B, or both transcription factors highly expressed (FIG. 3B). These states were designated A-only, B-only and A+B, respectively. The three states were well-separated by ˜25-to-50-fold differences in either mCherry or mCitrine expression, and cells grew at similar rates among states (FIG. 13A-FIG. 13E). To assess their stability, cells from each of these states were sorted and cultured continuously for 18 days (See, “Additional Methods” below). Strikingly, nearly all cells remained in the sorted state for this extended period (FIG. 3C, MultiFate-2.1 High TMP columns, and FIG. 14A-FIG. 14C), despite gene expression noise (observable from the spread of cellular fluorescence on flow cytometry plots). This showed that cells were attracted to these states. In some embodiments, stability required positive autoregulation, as withdrawal of AP1903 and TMP collapsed expression of both factors within 2 days (FIG. FIG. 14A-FIG. 14C). Similar overall behavior was also observed in MultiFate-2.2 and MultiFate-2.3 (FIG. 3C, FIG. 15A-FIG. 15B and FIG. 16A-FIG. 16B). All three MultiFate-2 cell lines thus exhibited dynamics consistent with type II tristablity (FIG. 1C).

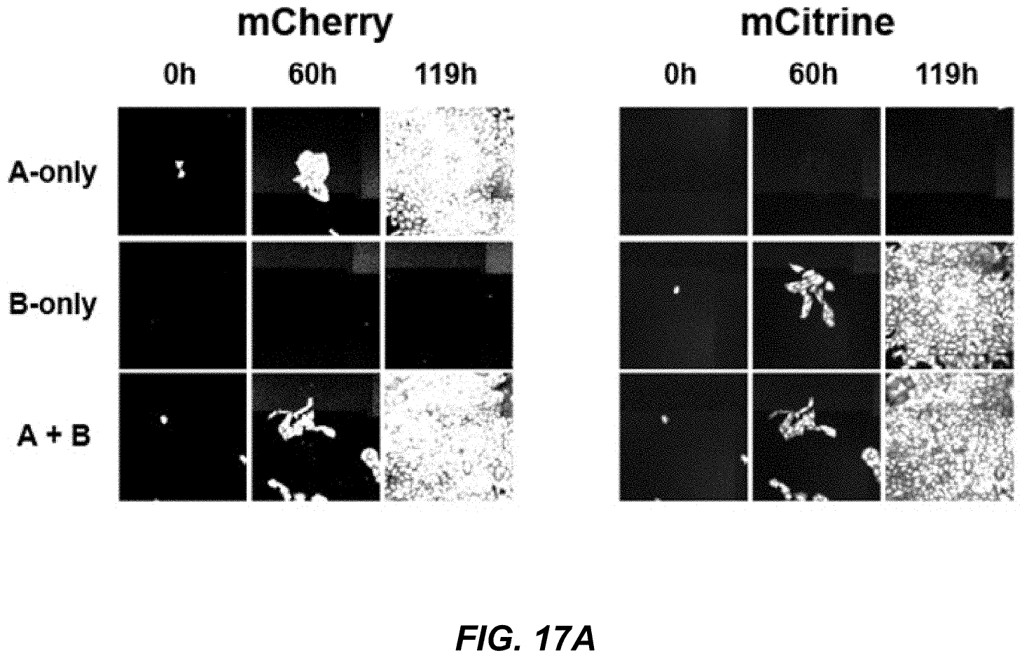

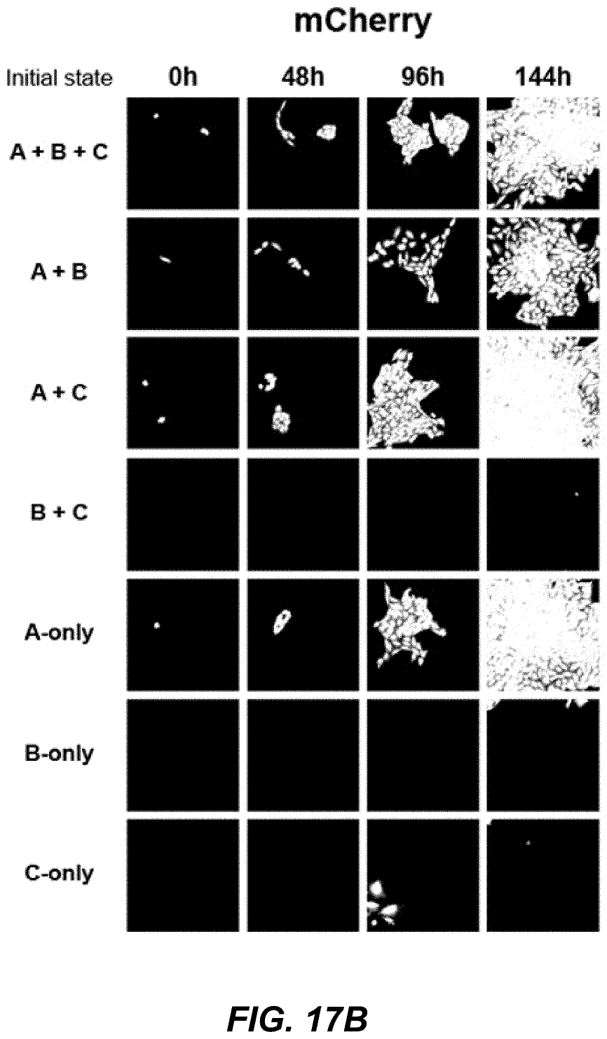

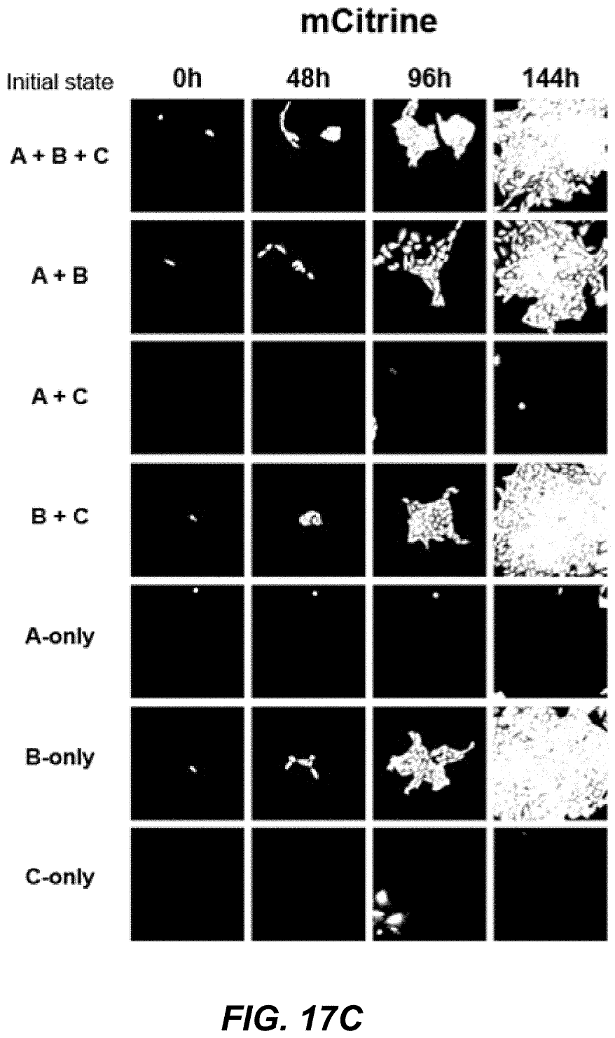

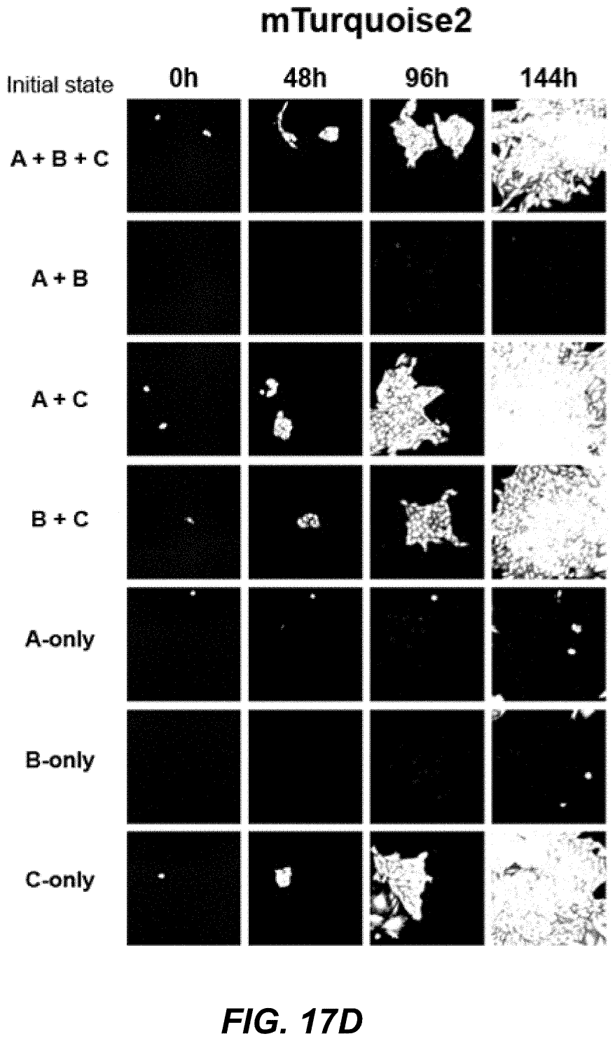

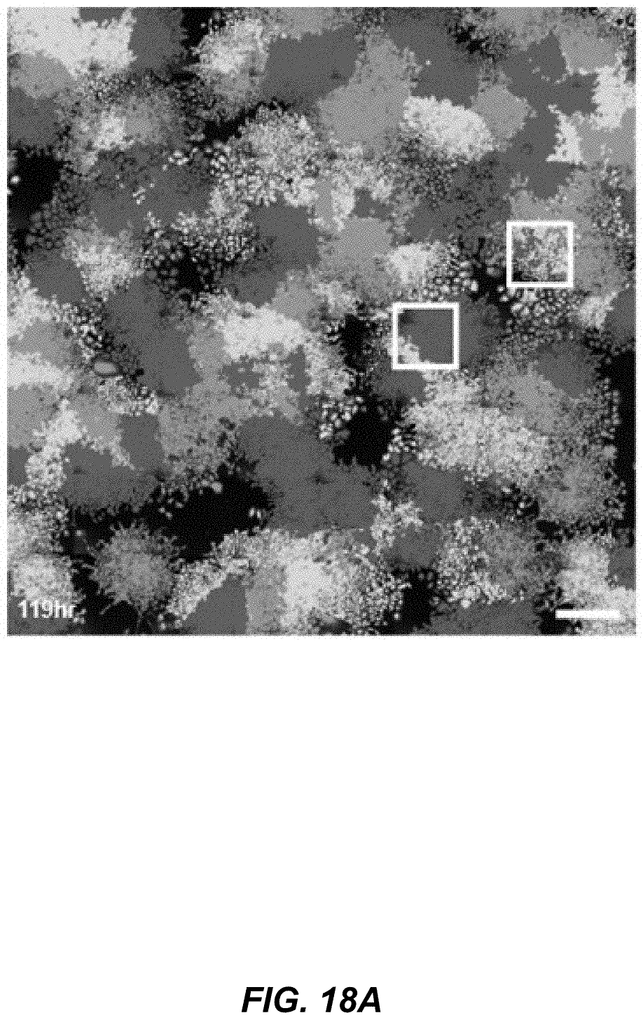

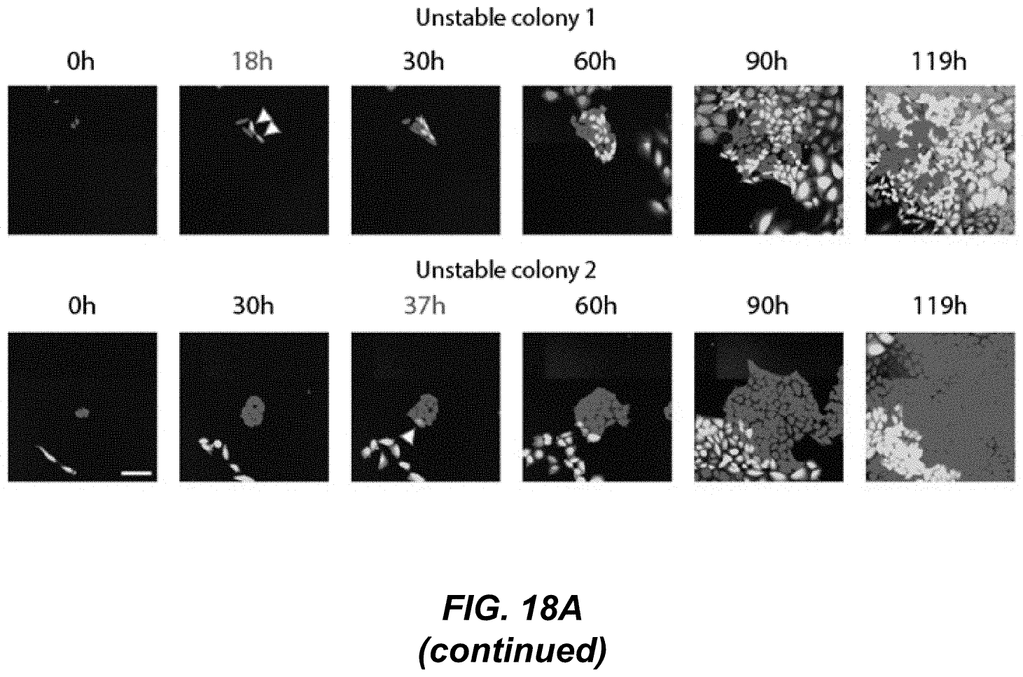

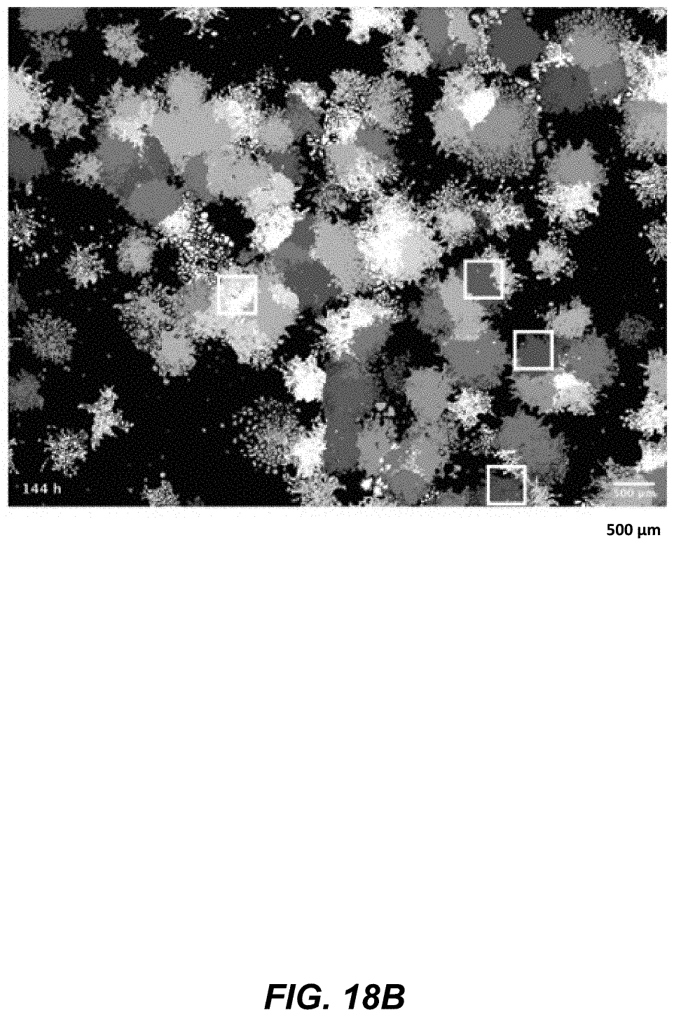

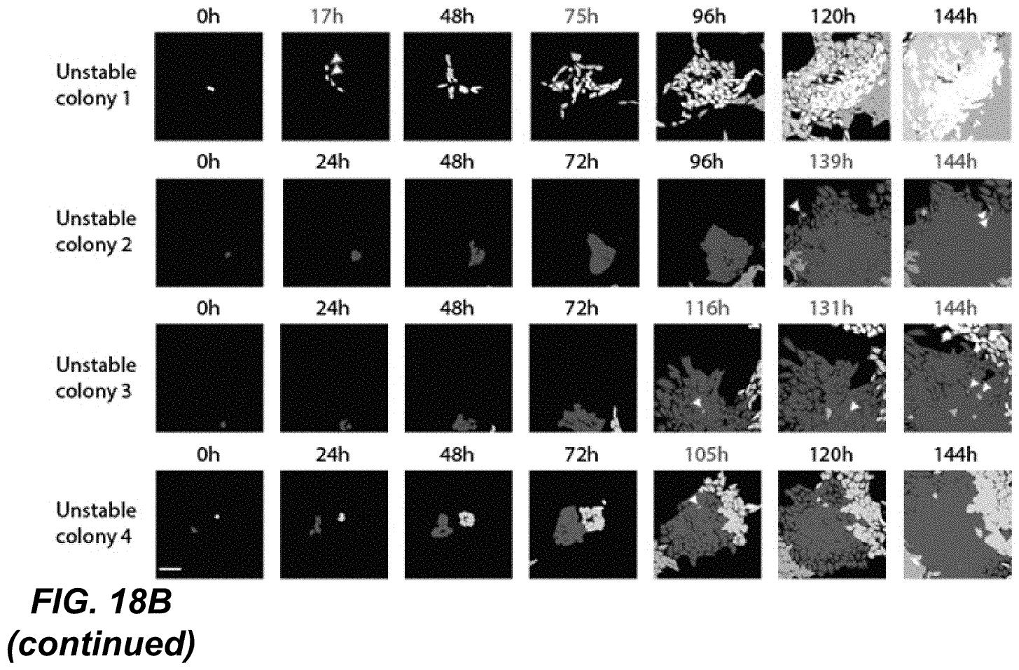

Time-lapse imaging provided a more direct view of multistability. An equal ratio of single cells sorted from three different initial states were cultured in the same well and cells were imaged as they developed into colonies (FIG. 3D) (See, “Additional Methods” below). In almost all colonies (132 of 134), all cells maintained their initial states for the full duration of the movie, at least 5 days or 7 to 8 cell cycles (FIG. 3D, FIG. 17A, FIG. 18A). Together with the flow cytometry analysis, these results demonstrate that all three MultiFate-2 lines can sustain long-term tristability.

### MultiFate-2 Supports Modulation of State Stability and Allows Controlled State-Switching.

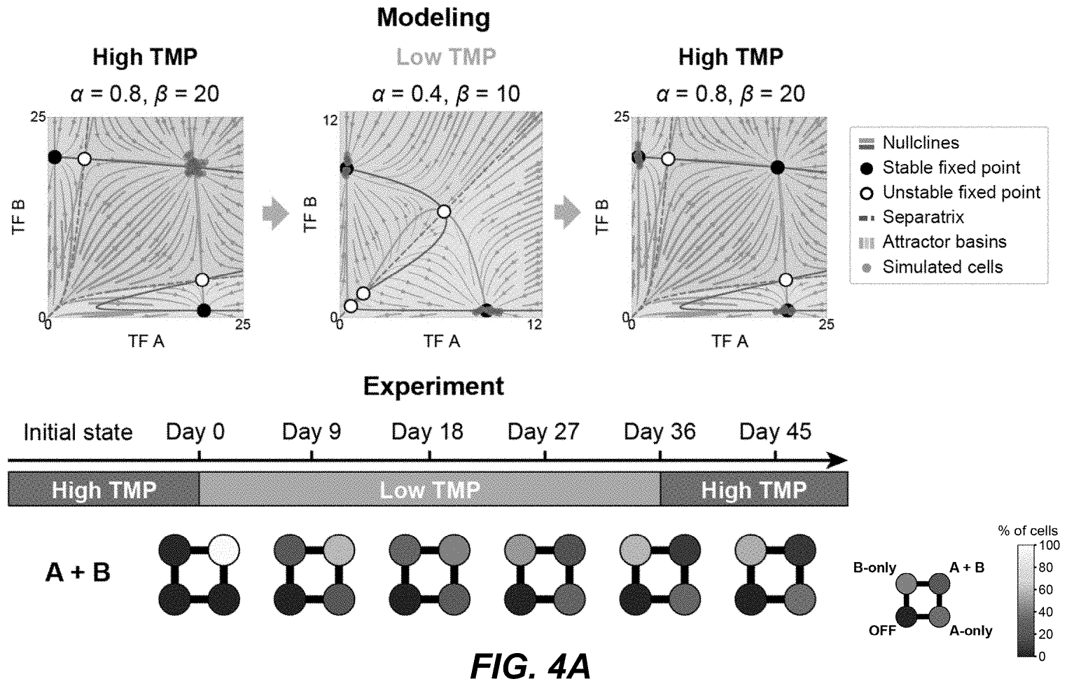

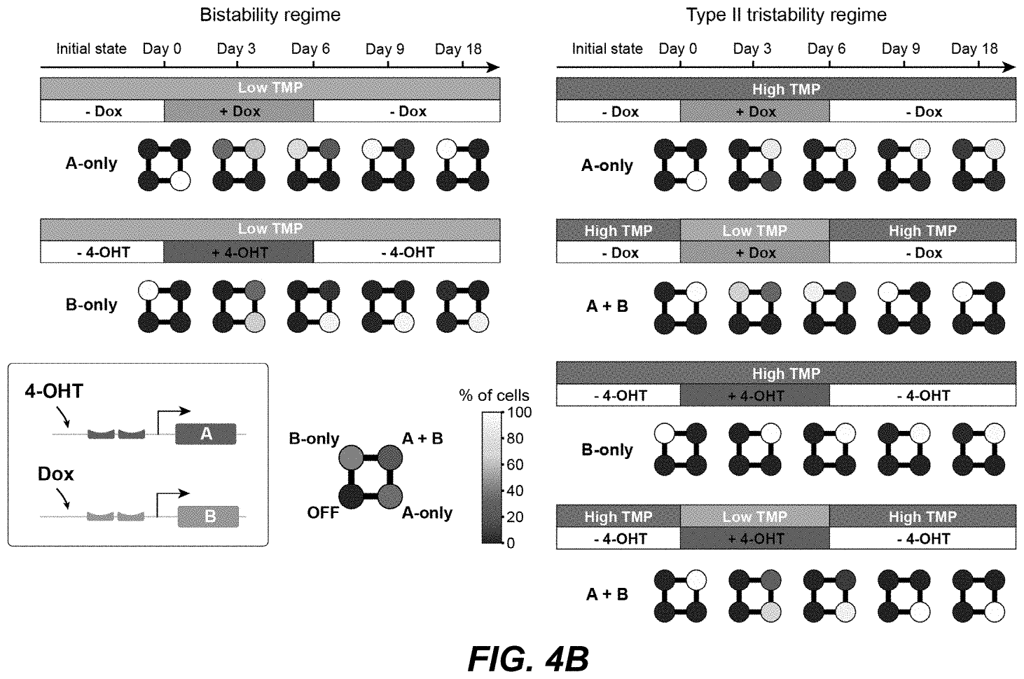

The ability of a transient stimulus to destabilize multipotent states and trigger an irreversible fate change is a hallmark of many cell fate control systems. In the model, reducing protein stability can eliminate the A+B state but preserve A-only and B-only states (FIG. 1C). As a result, cells initially occupying the A+B state transit to A-only or B-only states (FIG. 4A, top). When protein stability is restored to its initial value, the A+B attractor reappears. However, for the parameter sets analyzed here, cells remain within the attractor basins of A-only and B-only states, and therefore do not return to the A+B state (FIG. 4A, top). Stochastic simulations of single cell dynamics confirmed this irreversible (hysteretic) behavior (FIG. 4A, top).

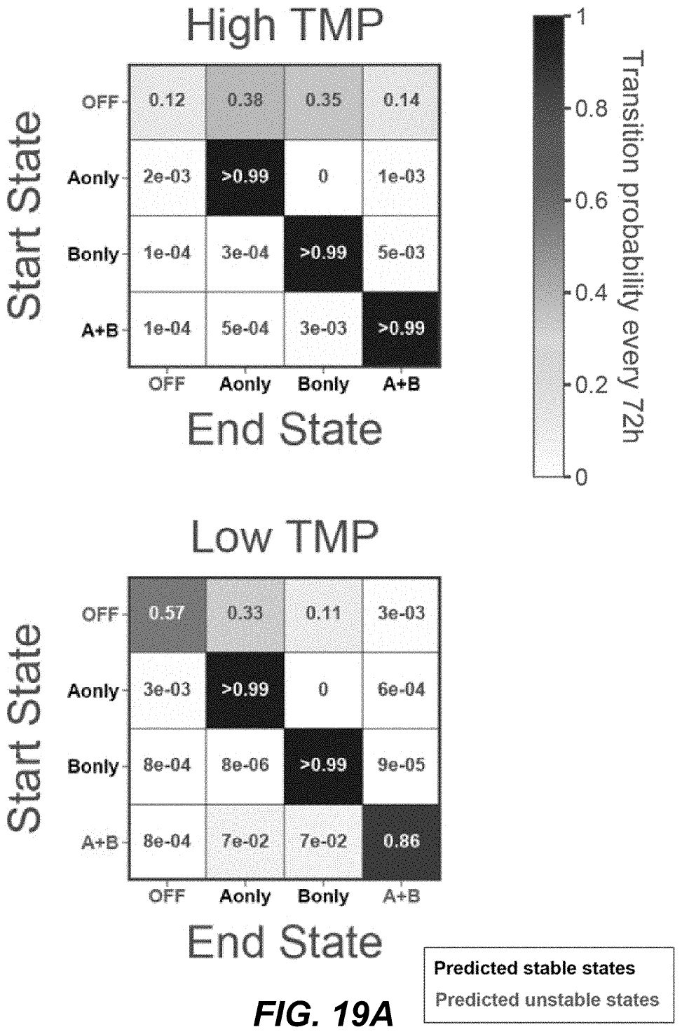

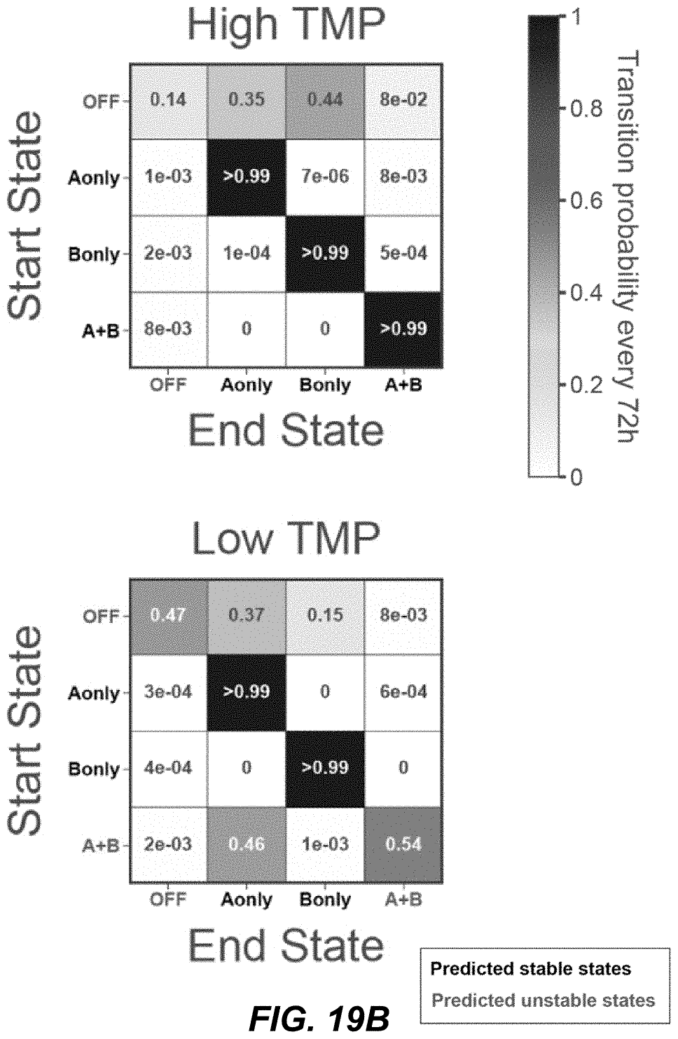

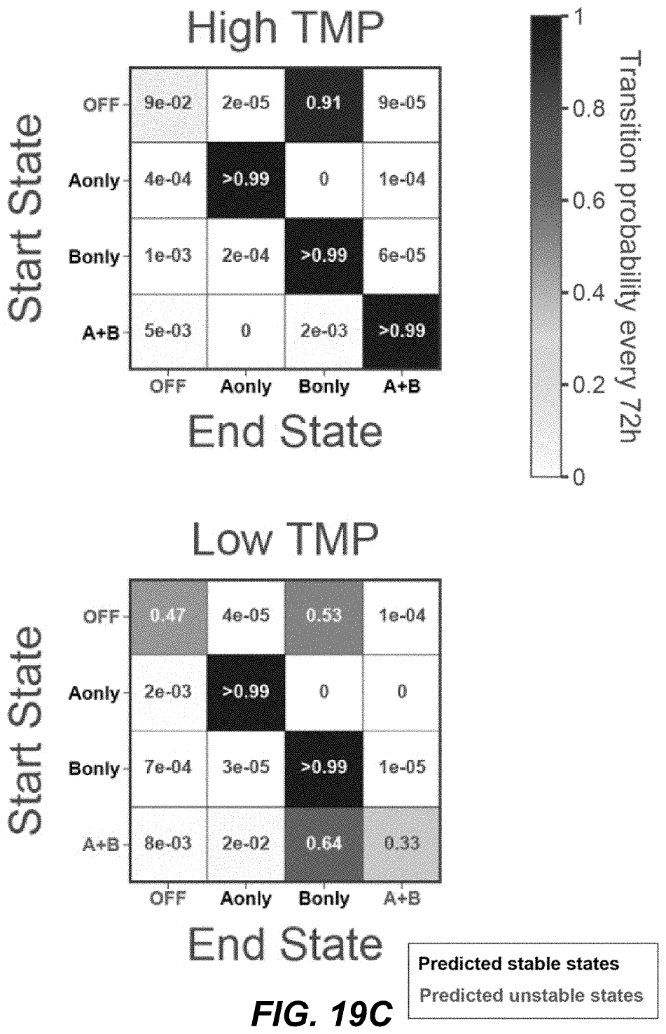

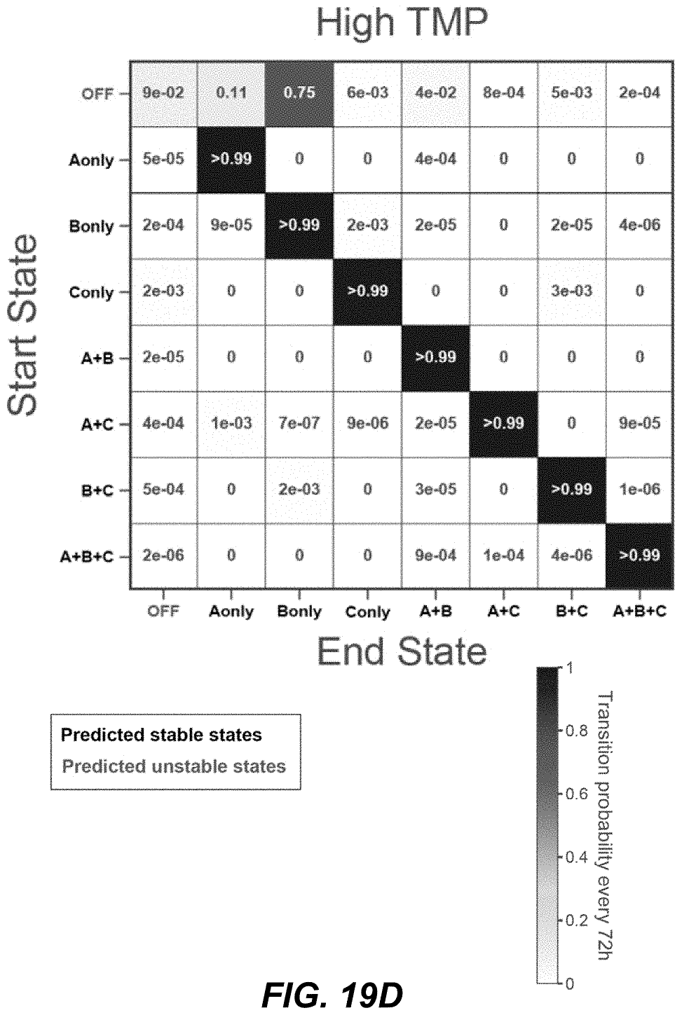

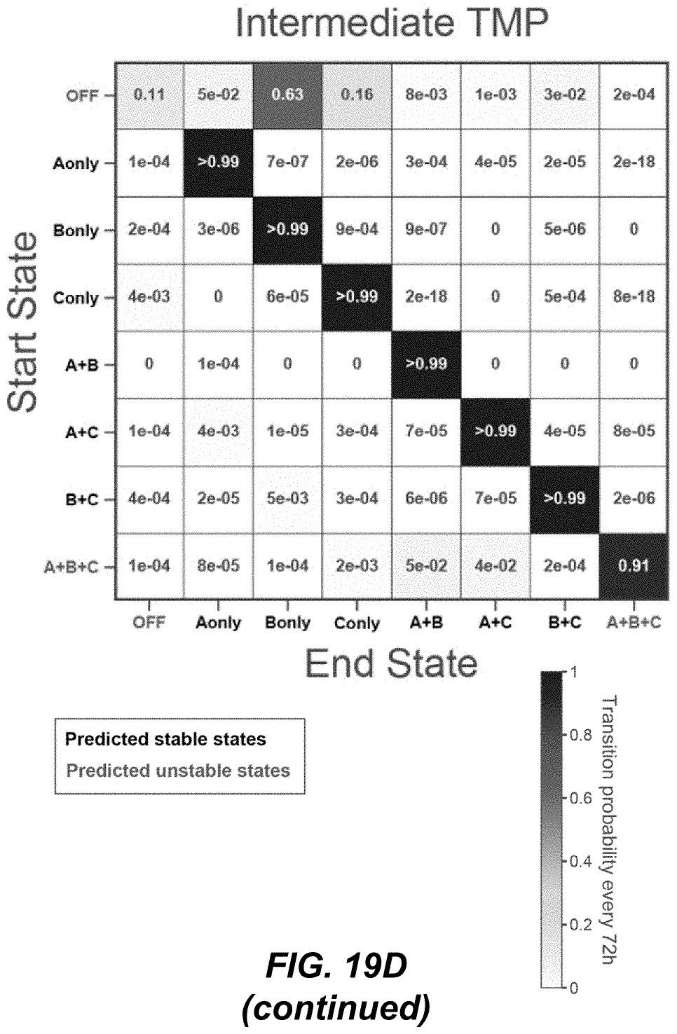

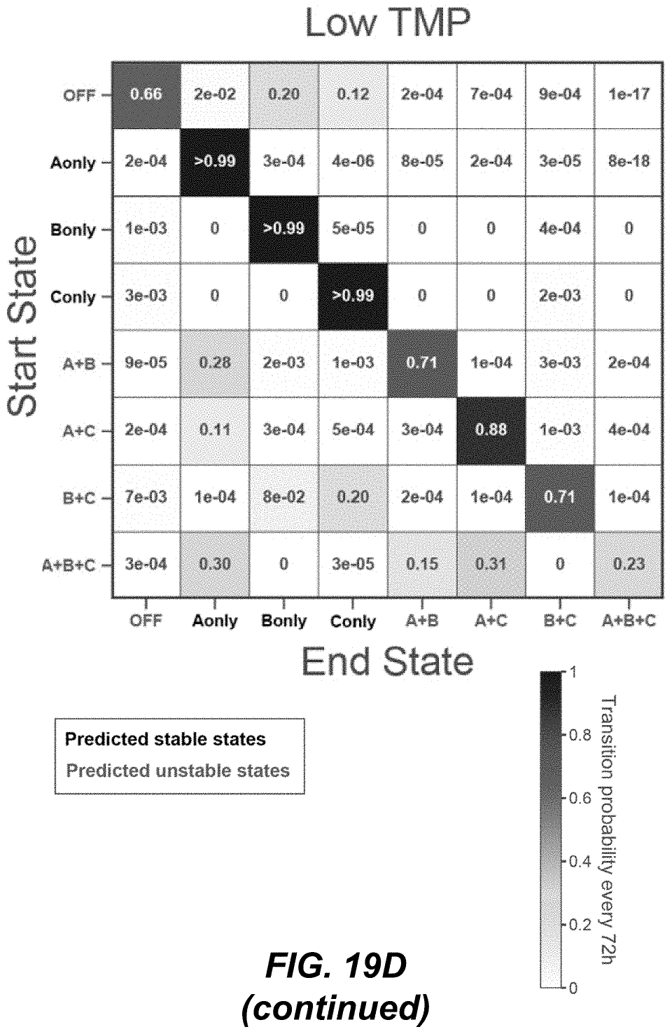

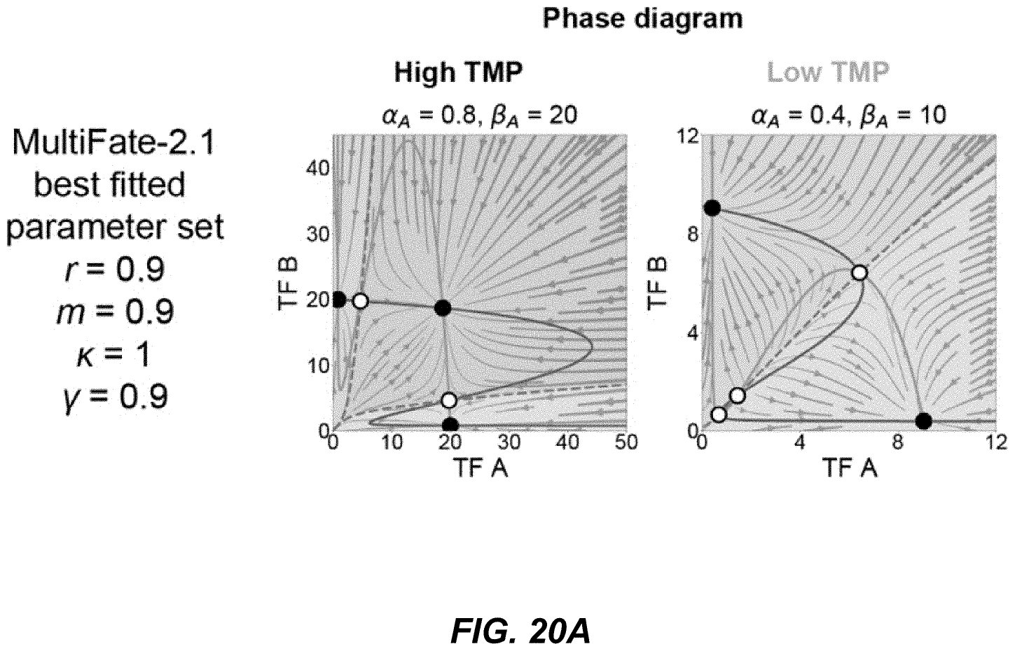

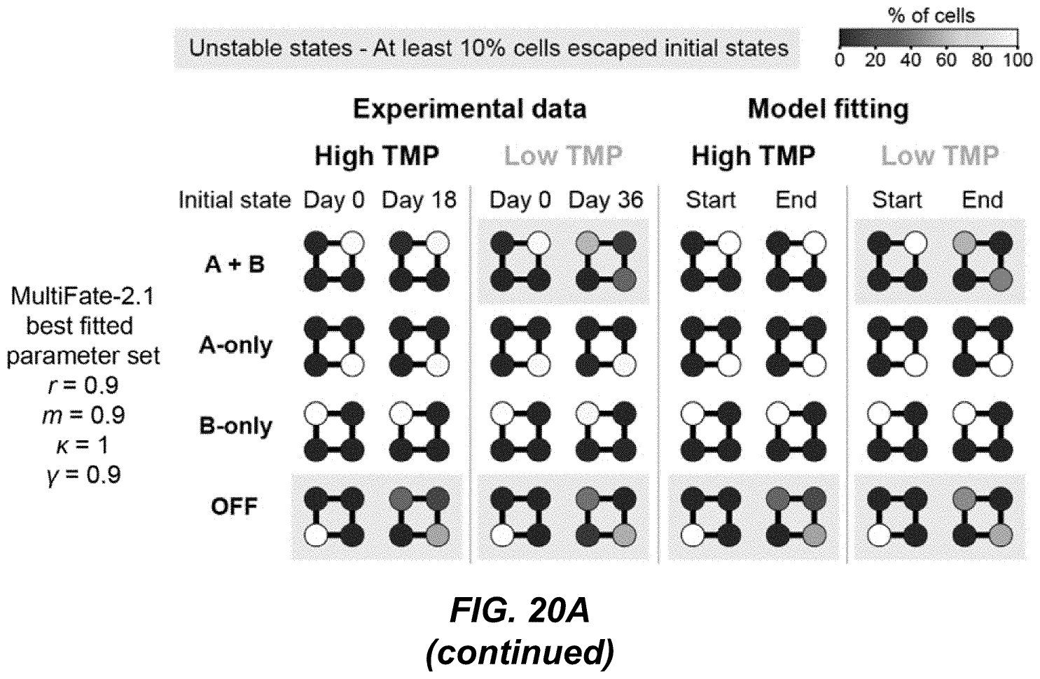

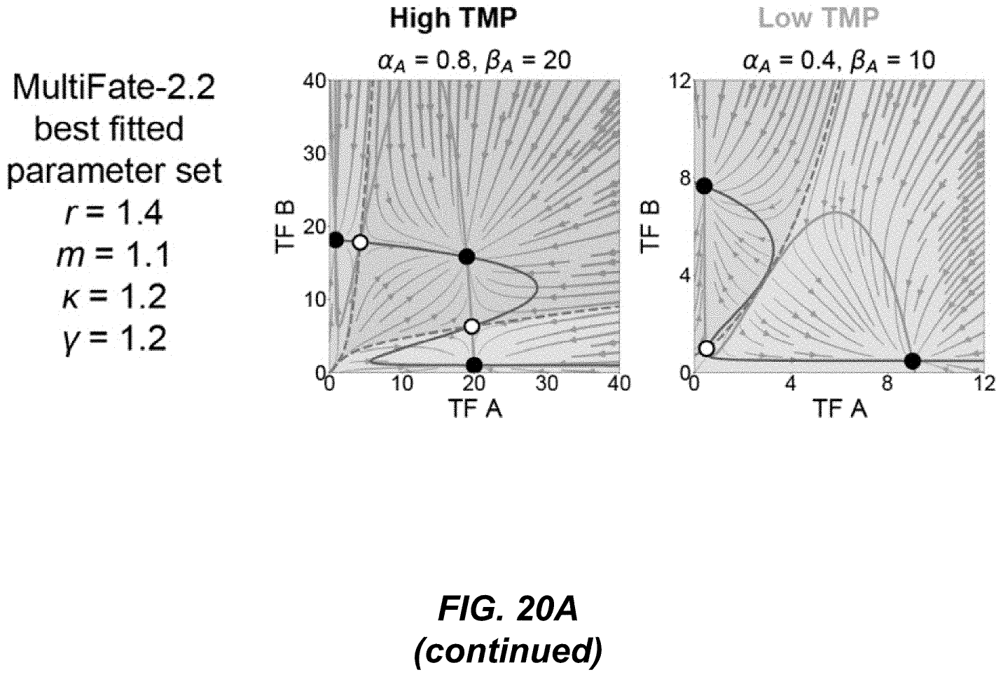

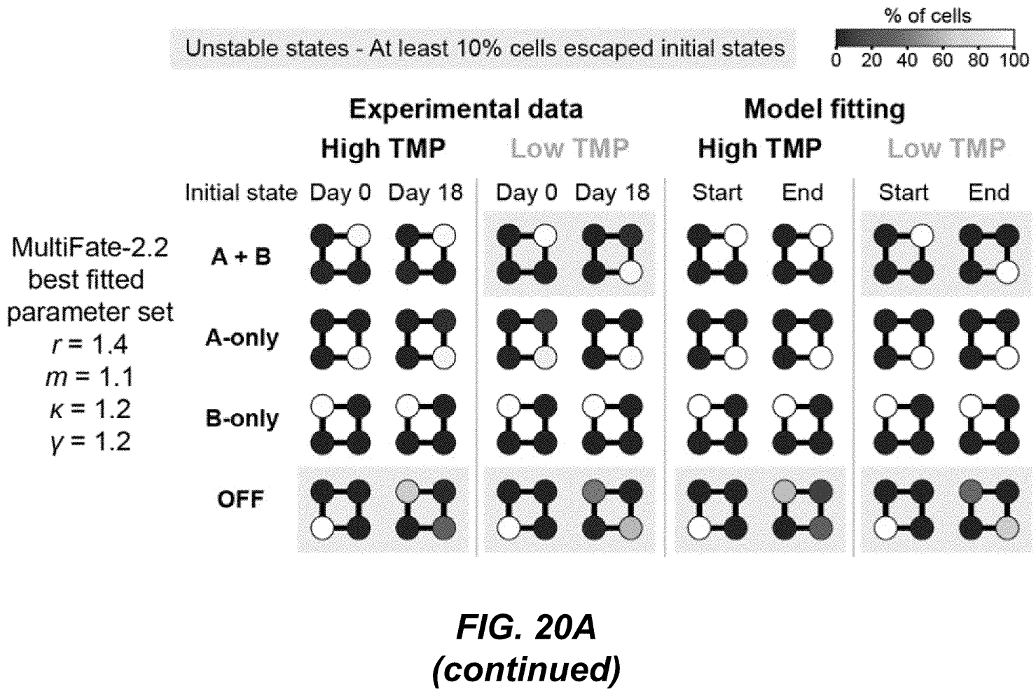

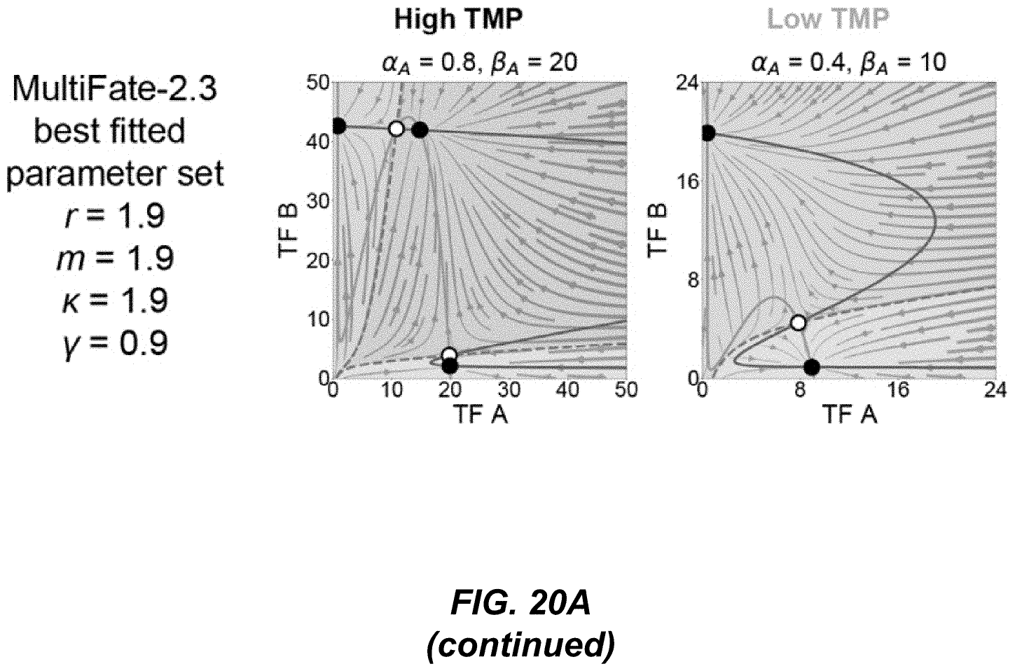

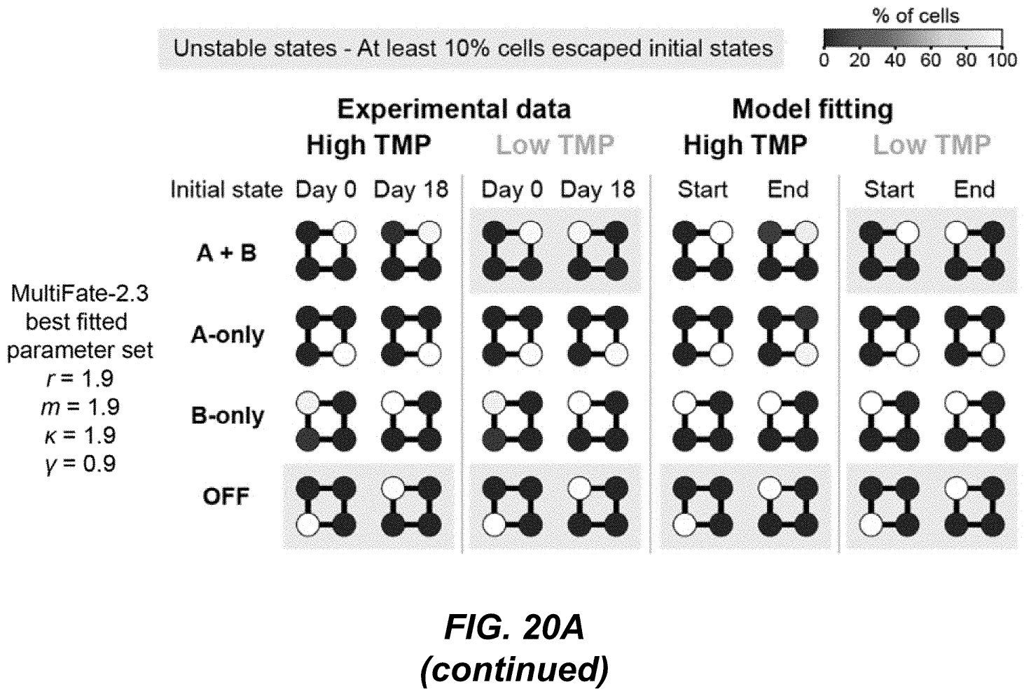

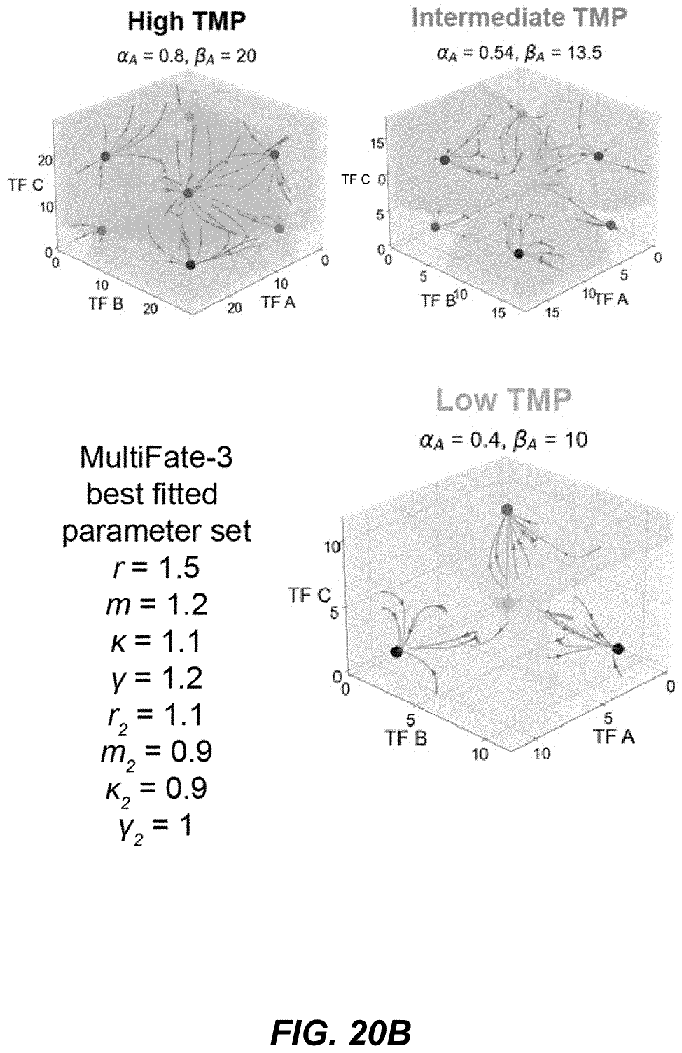

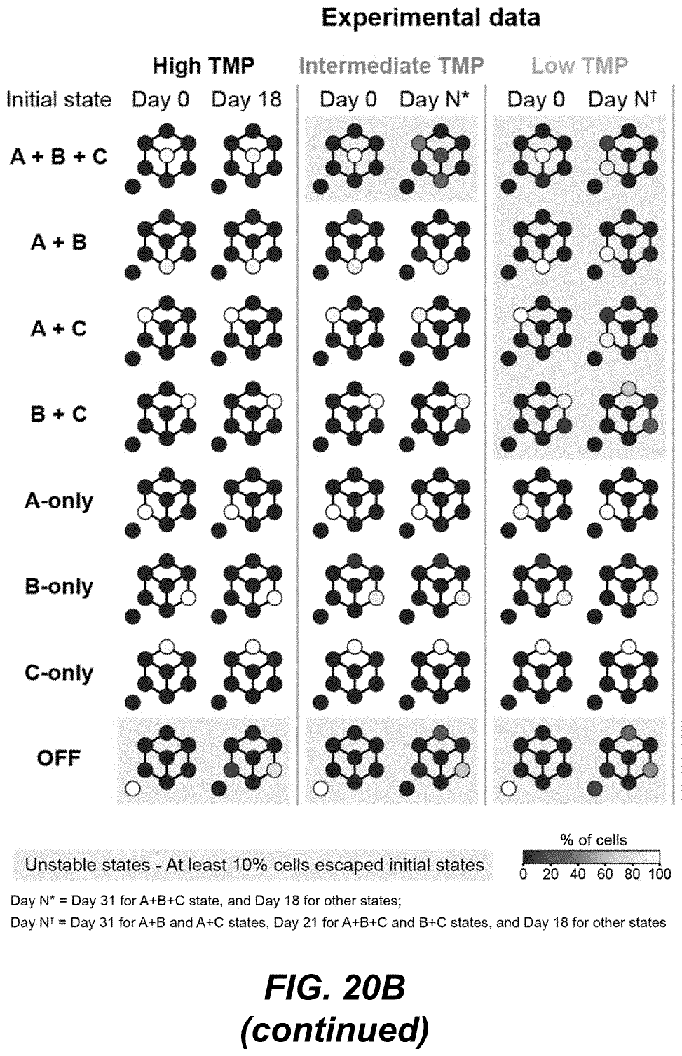

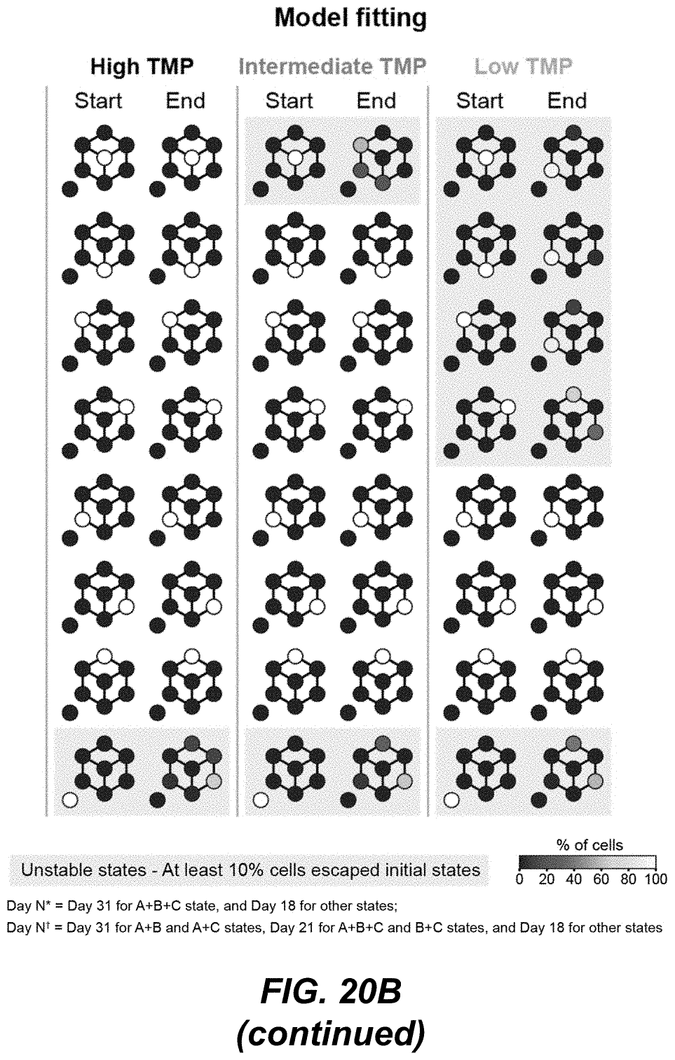

To test whether similar bifurcation and hysteretic dynamics occur in the experimental system, A-only, B-only and A+B cells were transferred from media containing high TMP concentrations (“High TMP”) to similar media with reduced TMP concentrations (“Low TMP”), which decreased protein stability by permitting degron function. As predicted, reducing protein stability selectively destabilized the A+B state, but not the A-only and B-only states, shifting cells from A+B state to the A-only or B-only states (FIG. 3C, Low TMP columns, FIG. 4A, bottom). Different MultiFate-2 cell lines exhibited different transition biases, reflecting clone-specific asymmetries in the experimental MultiFate-2 systems (FIG. 3C, FIG. 14A-FIG. 16B), in a manner consistent with an asymmetric MultiFate model (FIG. 19A-FIG. 19D and FIG. 20A-FIG. 20B) (See, “Additional Methods” below). Escape from the destabilized A+B state was irreversible, as cells remained in the A-only or B-only state even after they were transferred back to the High TMP media (FIG. 4A, bottom, and FIG. 14A-FIG. 14C). Thus, MultiFate's ability to support irreversible transitions allows it to produce behaviors resembling stem cell differentiation.

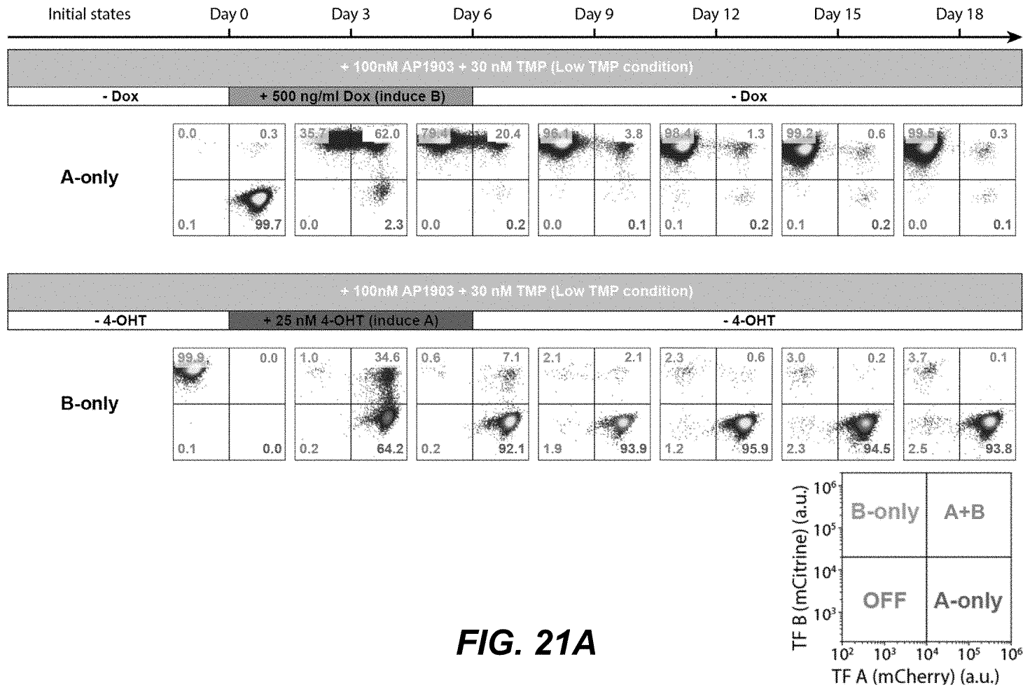

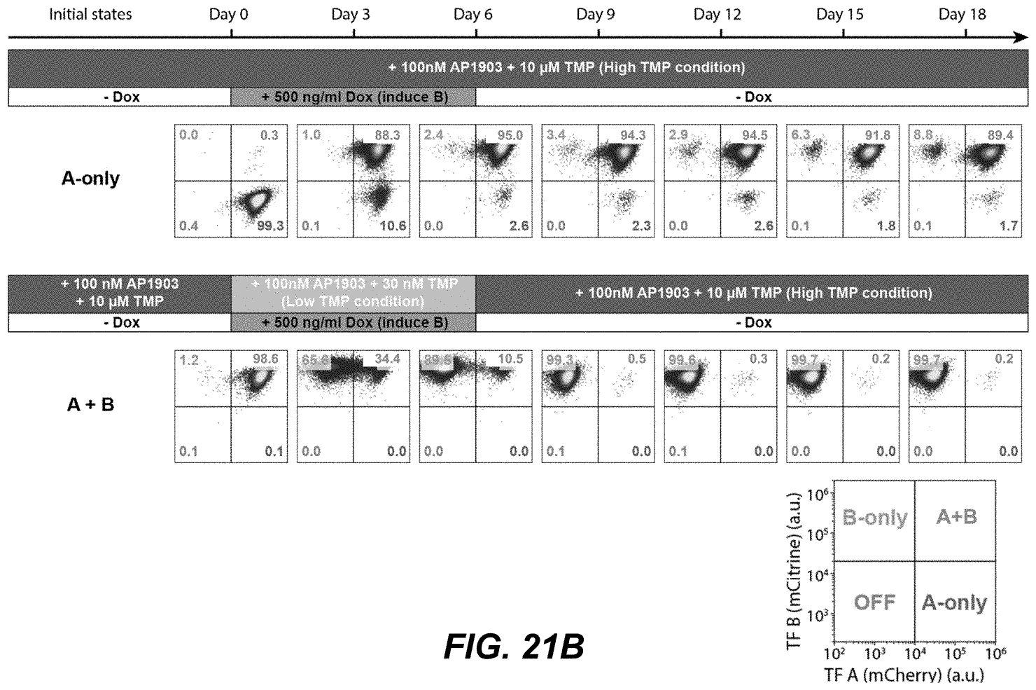

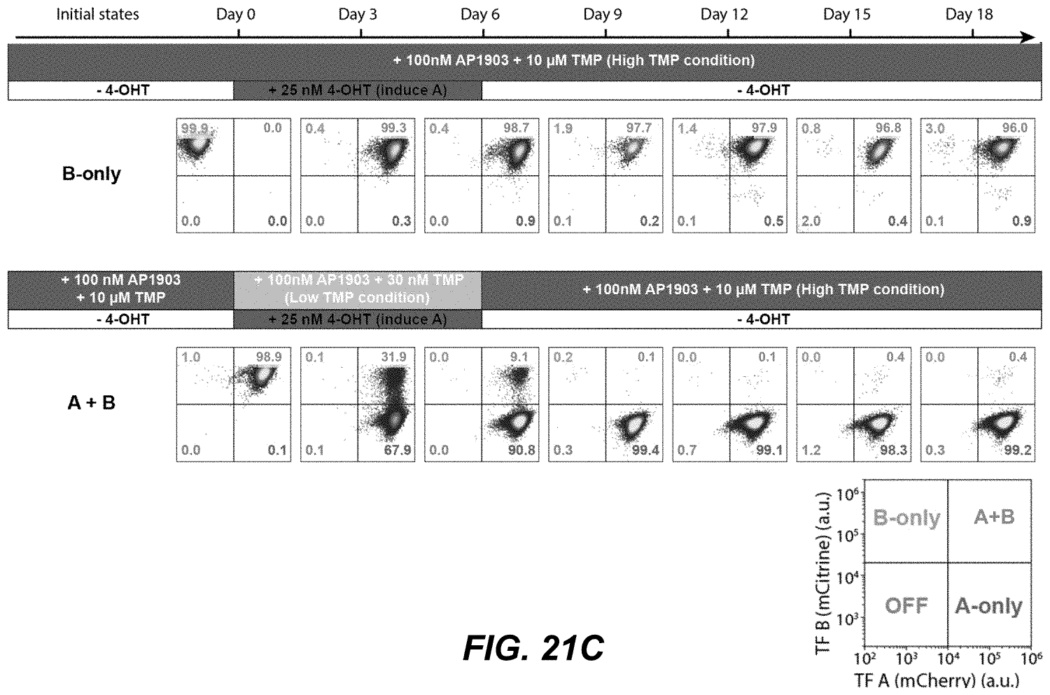

Finally, it was asked to what extent cells could be deliberately switched from one state to another through transient perturbations. MultiFate-2.3, in which the A and B genes can be independently activated by 4-hydroxy-tamoxifen (4-OHT) and Dox, respectively, was used to address this question. In this cell-line, the response elements for the inducers are adjacent to the homodimer binding sites. Therefore, the addition of inducers increases A or B expression up to, but not substantially beyond, the level produced by self-activation (FIG. 2C and FIG. 21A-FIG. 21C). In the bistable regime, transient induction of either transcription factor switched cells into the corresponding state, where they remained in the absence of further induction (FIG. 8A, FIG. 4B, left, and FIG. 21A). In the tristable regime, the model predicted, and experiments confirmed, that transient induction of B by Dox can switch A-only cells to the A+B state, but not beyond it to the B-only state (FIG. 8B first row, FIG. 4B, top right, and FIG. 21). Combining transient Dox addition to induce B expression with TMP reduction to destabilize the A+B state successfully transitioned cells from the A+B to the B-only state (FIG. 8B, second row, and FIG. 4B, right second row). The reciprocal experiments, in which A expression was induced with 4-OHT with or without reduced TMP, produced equivalent results (FIG. 4B, right column, lower two rows). Taken together, these results demonstrate that MultiFate-2 circuits allow modulation of state stability, irreversible cell state transitions, and direct control of state-switching with transient external inducers.

### MultiFate is Expandable.

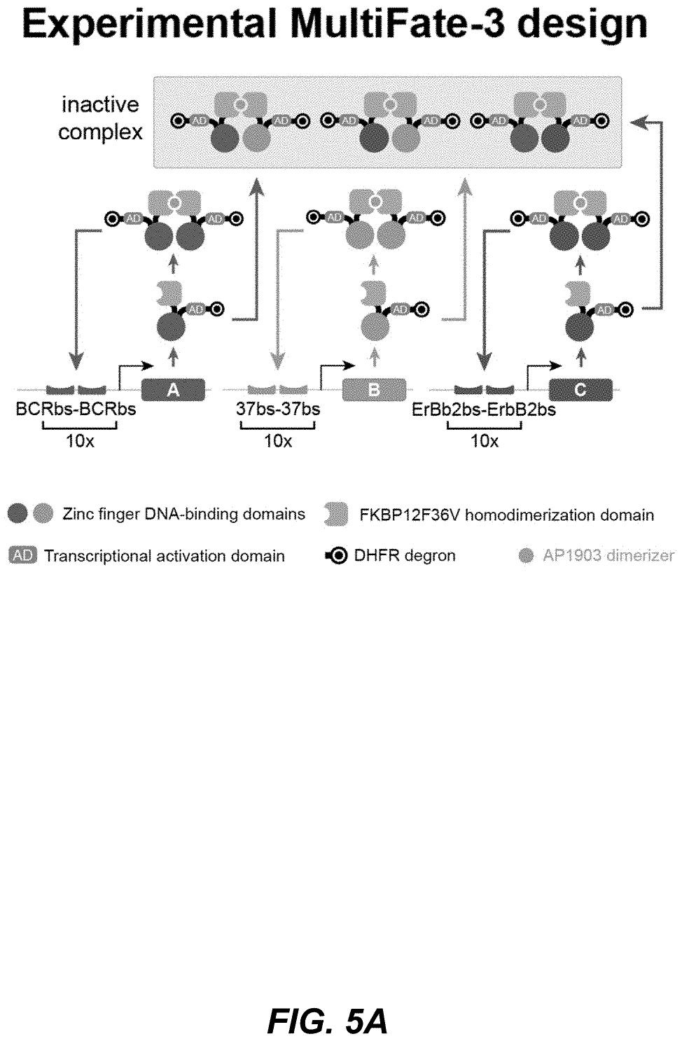

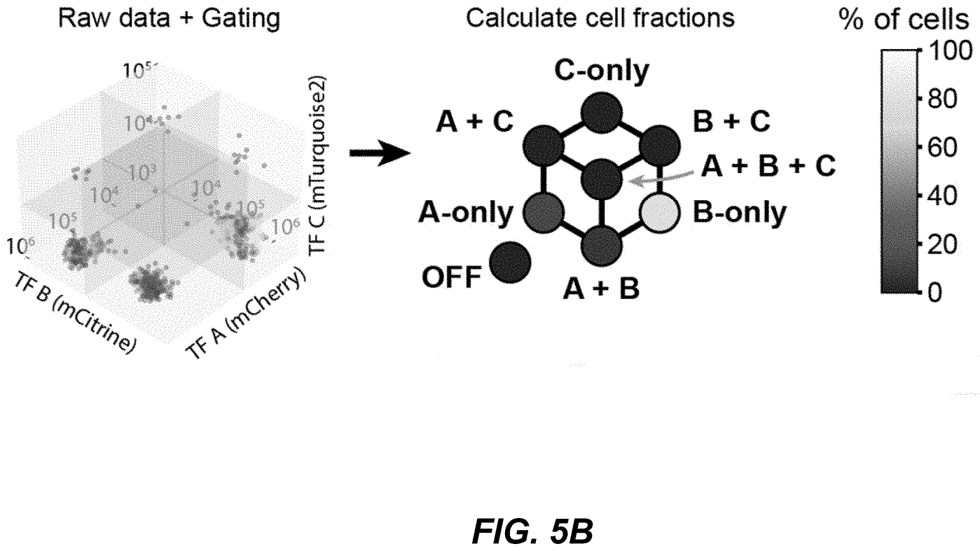

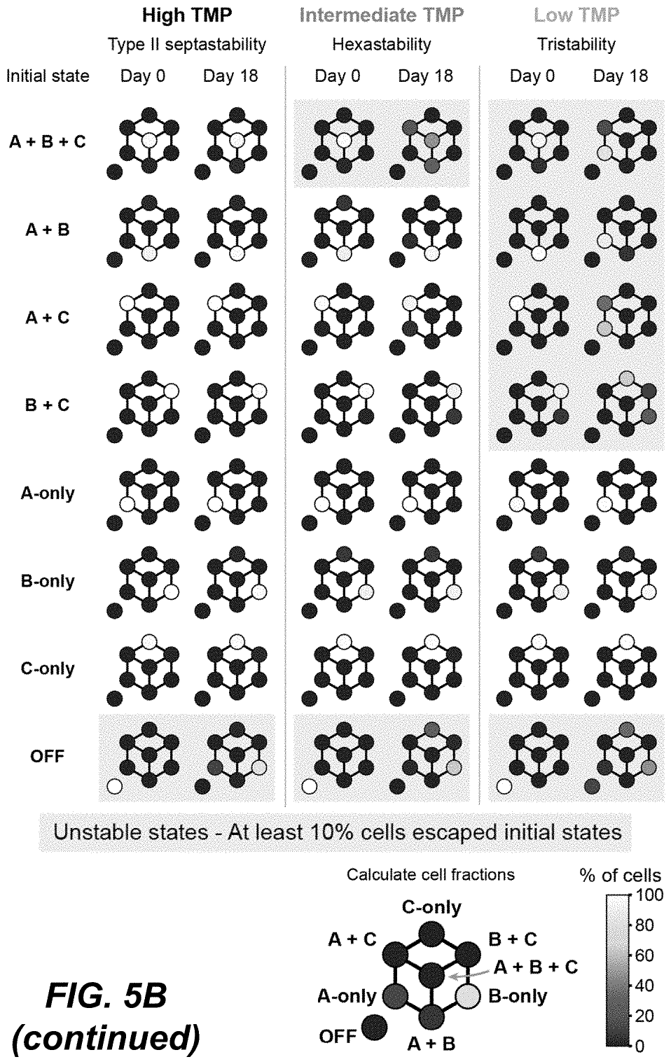

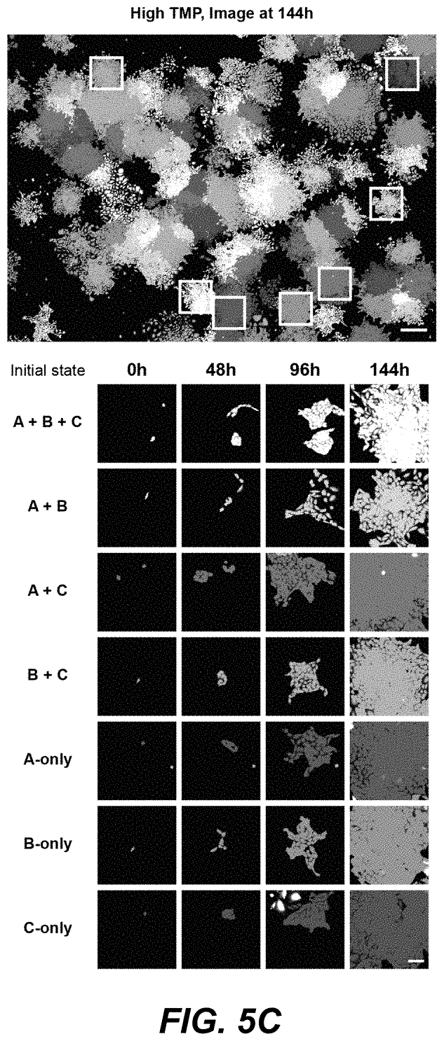

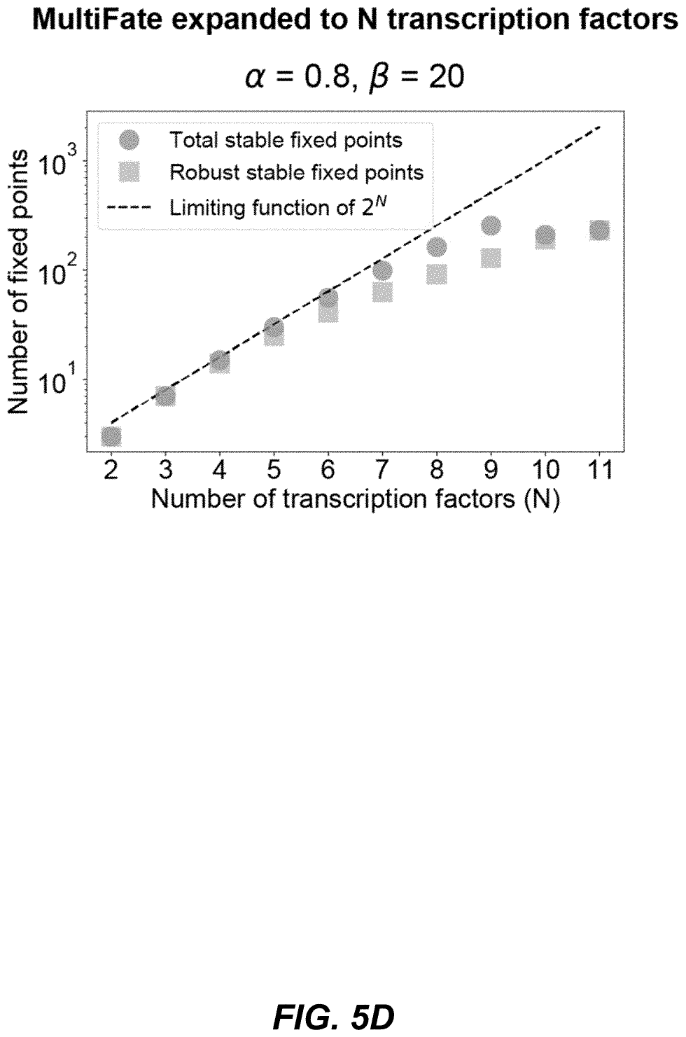

Without being bound by any particular theory, because the MultiFate system implements mutual inhibition among transcription factors through heterodimerization, it can be, in some embodiments, expanded by adding additional transcription factors, without re-engineering existing components. In the model, adding a third transcription factor to a MultiFate-2 circuit produces a range of new stability regimes containing 3, 4, 6, 7, or 8 stable fixed points, depending on parameter values (FIG. 1D and FIG. 7A-FIG. 7B) (See, “Additional Methods” below). To test whether experimental MultiFate-2 circuits can be similarly expanded, a third ZF transcription factor, denoted C, containing the same FKBP dimerization domain as A and B, co-expressed with a third fluorescent protein, mTurgoise2, was stably integrated into the MultiFate-2.2 cell line to obtain the MultiFate-3 cell line (FIG. 5A, FIG. 12B and Table 3) (See, “Additional Methods” below).

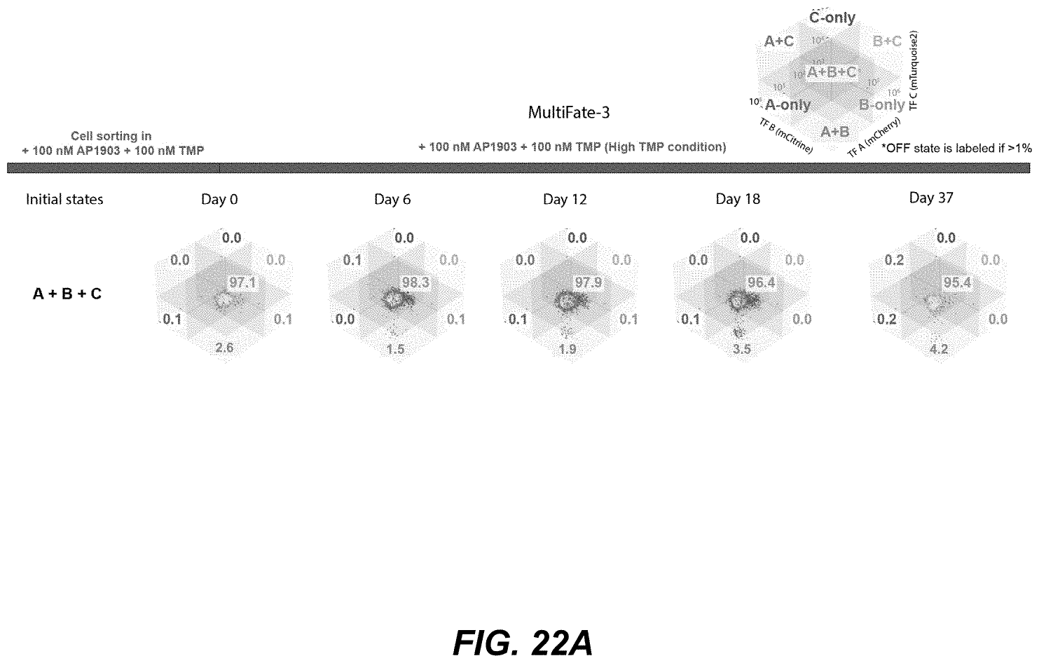

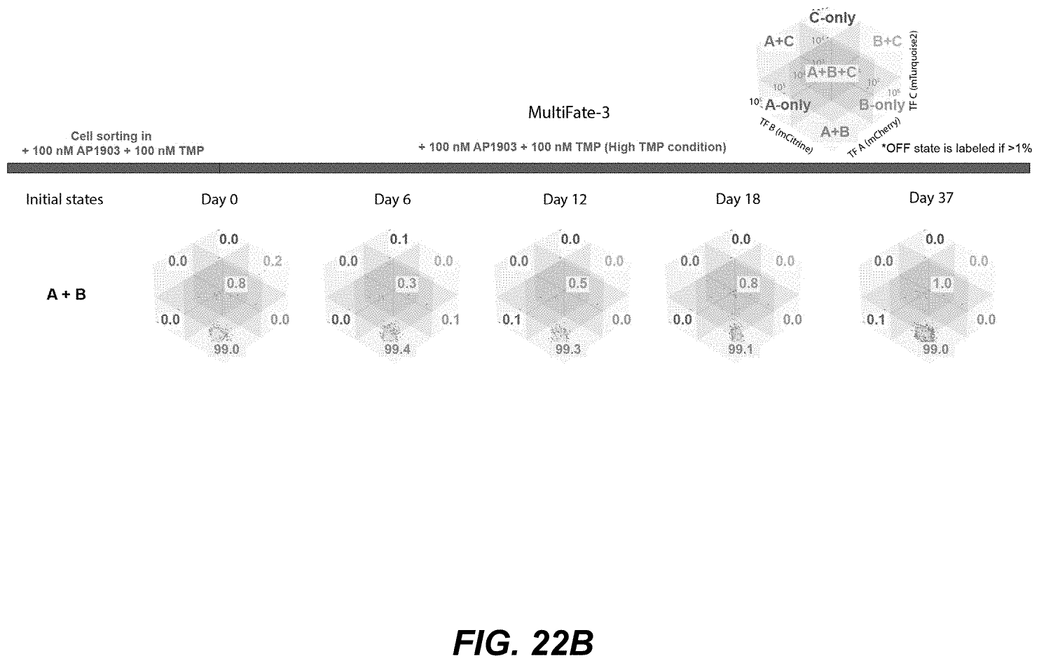

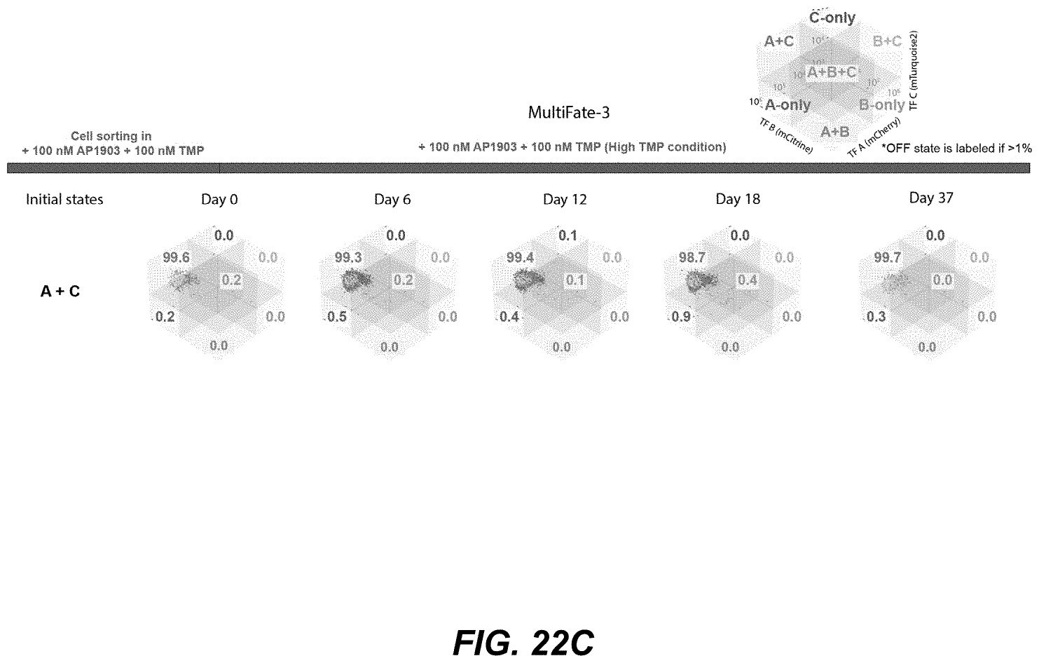

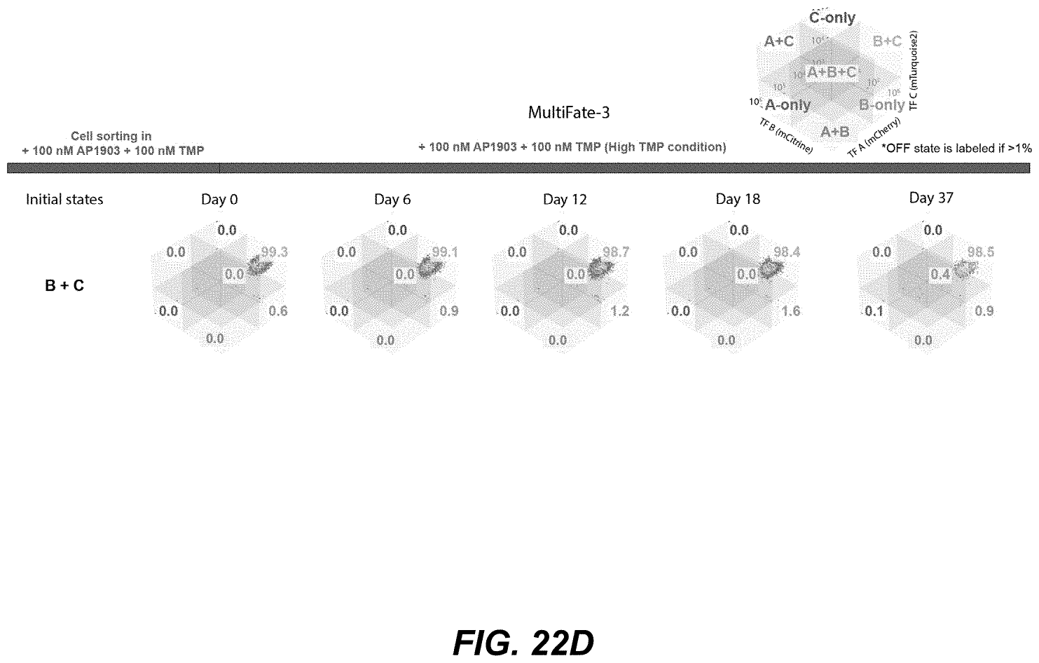

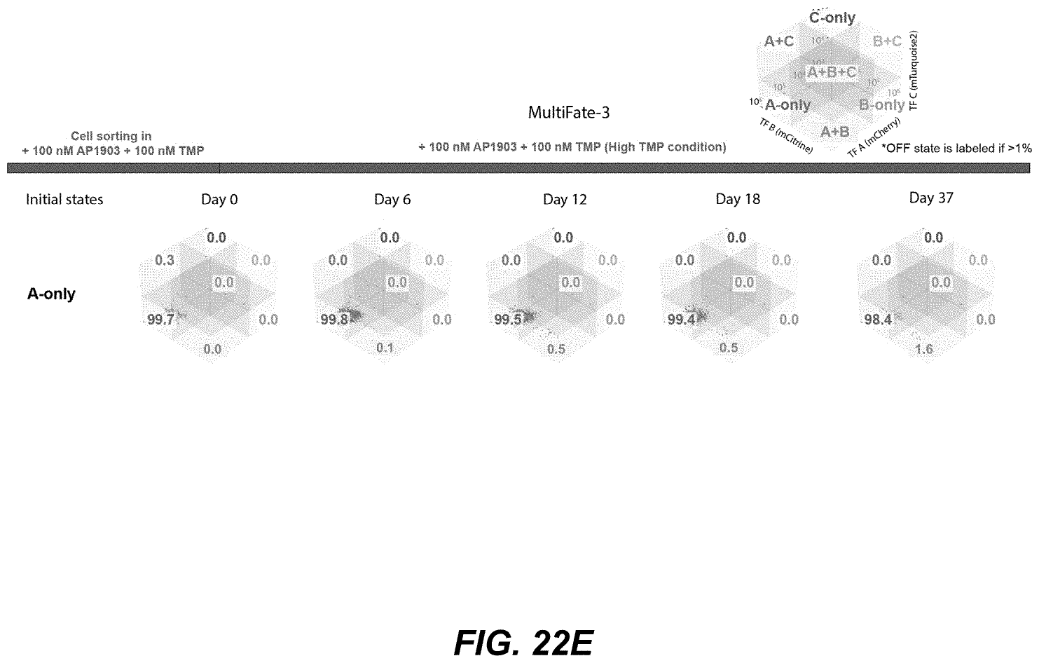

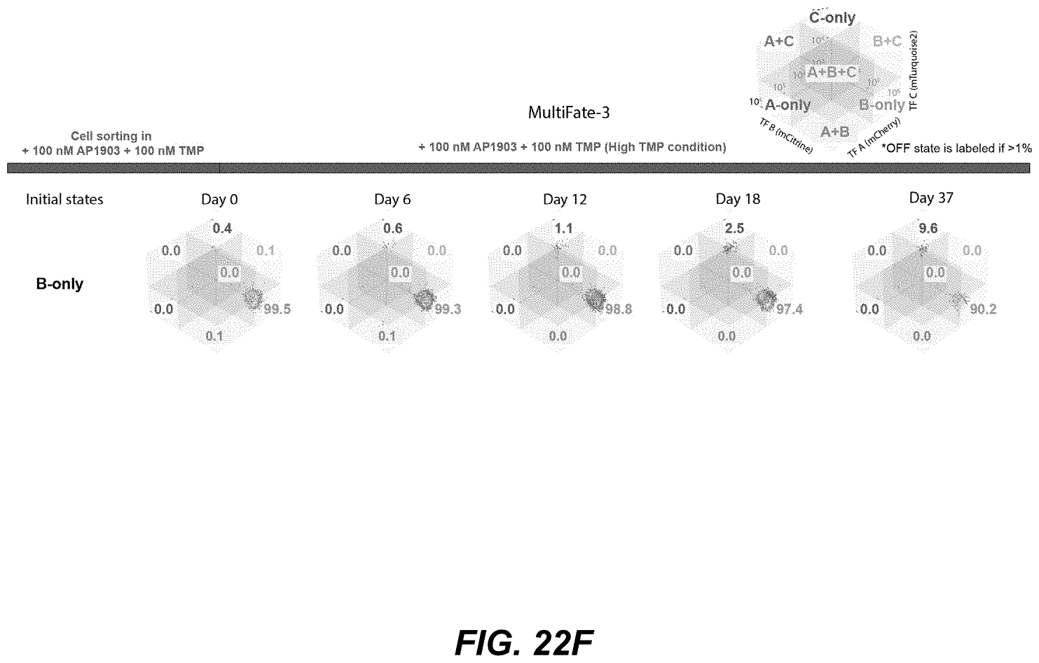

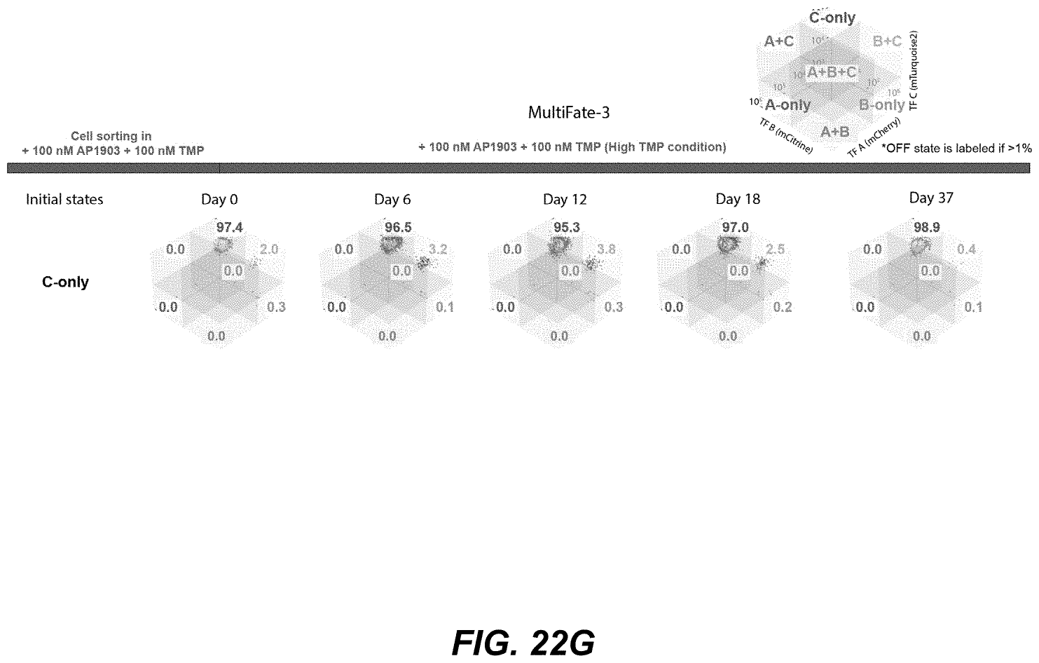

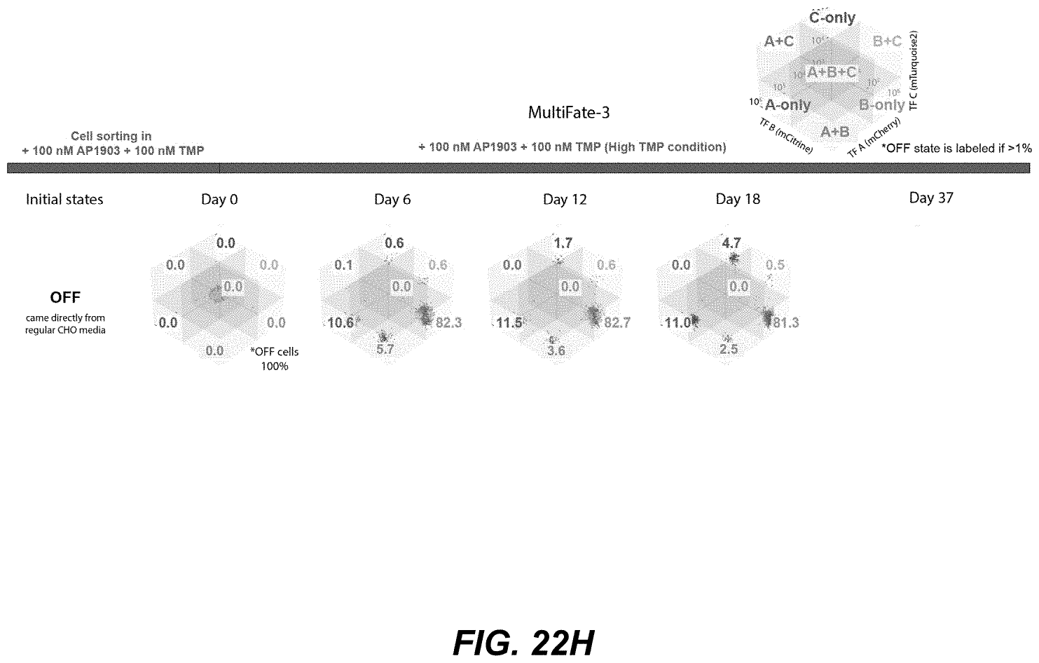

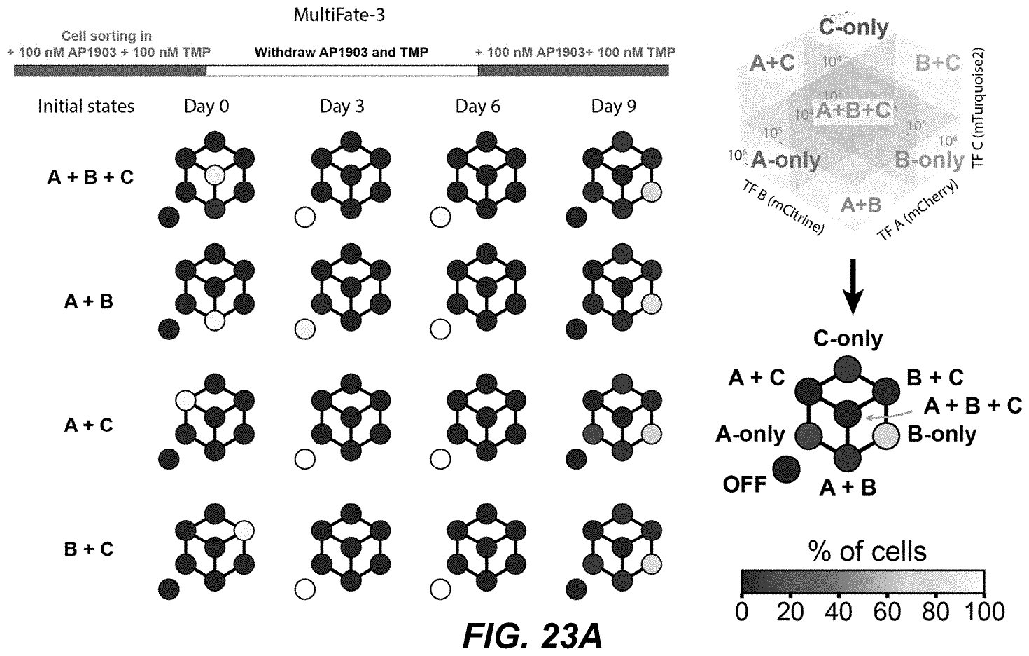

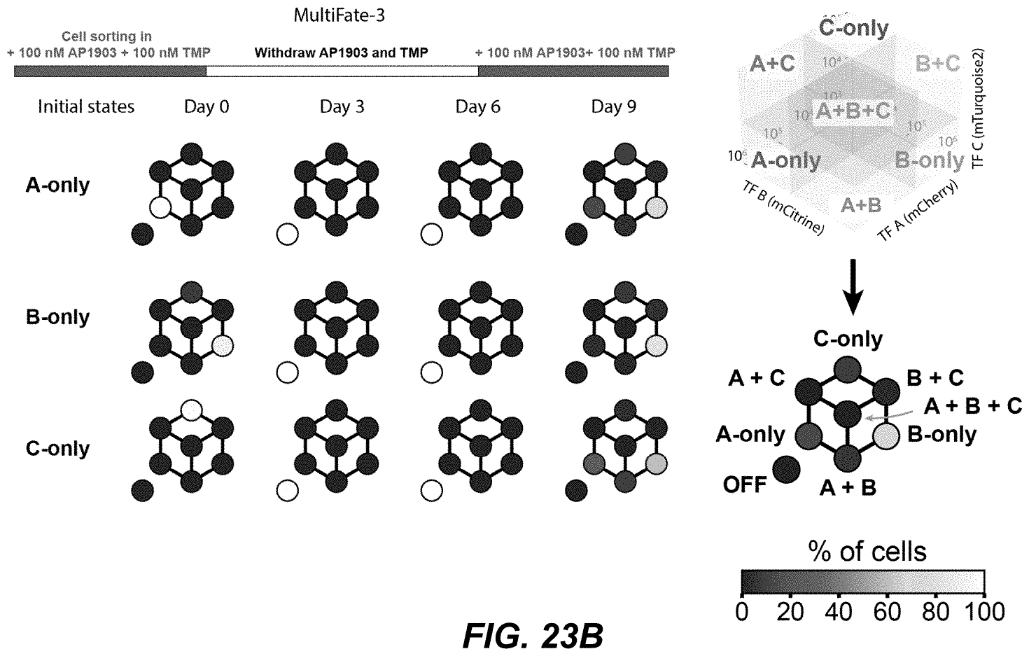

After the addition of AP1903 and TMP, MultiFate-3 cells went from low expression of all genes (OFF state) to one of seven distinct expression states, termed A-only, B-only, C-only, A+B, A+C, B+C, and A+B+C states (FIG. 5B), consistent with a type II septastability regime (FIG. 1D and FIG. 7A). Most cells occupied the B-only state (79.5%±0.3%), reflecting asymmetries within the circuit (FIG. 19A-FIG. 20B). To assess the stability of these states, cells from each of the seven states were sorted and continuously cultured in media containing AP1903 and TMP. The cultures were analyzed every 3 days by flow cytometry (See, “Additional Methods” below). Each of the seven states was stable for the full 18-day duration of the experiment (FIG. 5B, High TMP columns, and FIG. 22A-FIG. 22H). Long-term stability required AP1903 and TMP (FIG. 23A-FIG. 23B). Finally, cells from each state were able to be reset by withdrawal of AP1903 and TMP and then re-differentiated into all 7 states when AP1903 and TMP were added back (FIG. 23A-FIG. 23B). This shows that the observed stability is not the result of a mixture of clones permanently locked into distinct expression states.

To directly visualize the septastable dynamics of MultiFate-3, single cells sorted from each of the seven states were co-cultured and live imaging was performed as they grew into colonies (See, “Additional Methods” below). Consistent with the flow cytometry results, cells retained their initial states for the full 6-day duration of the experiment in almost every colony (153 of 157) (FIG. 5C, FIG. 17B-FIG. 17D, FIG. 18B).

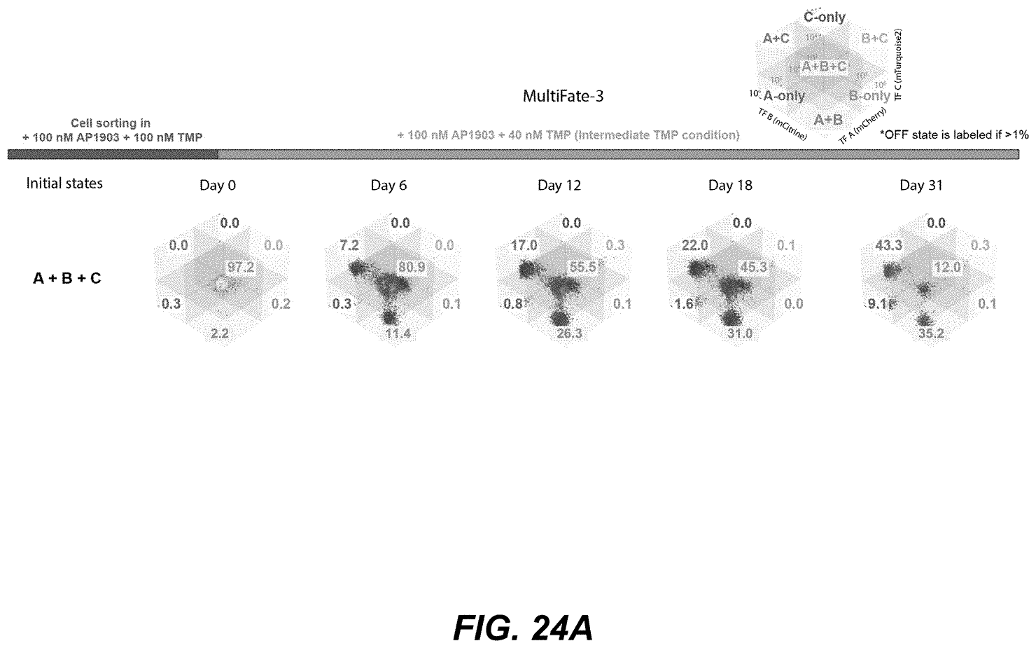

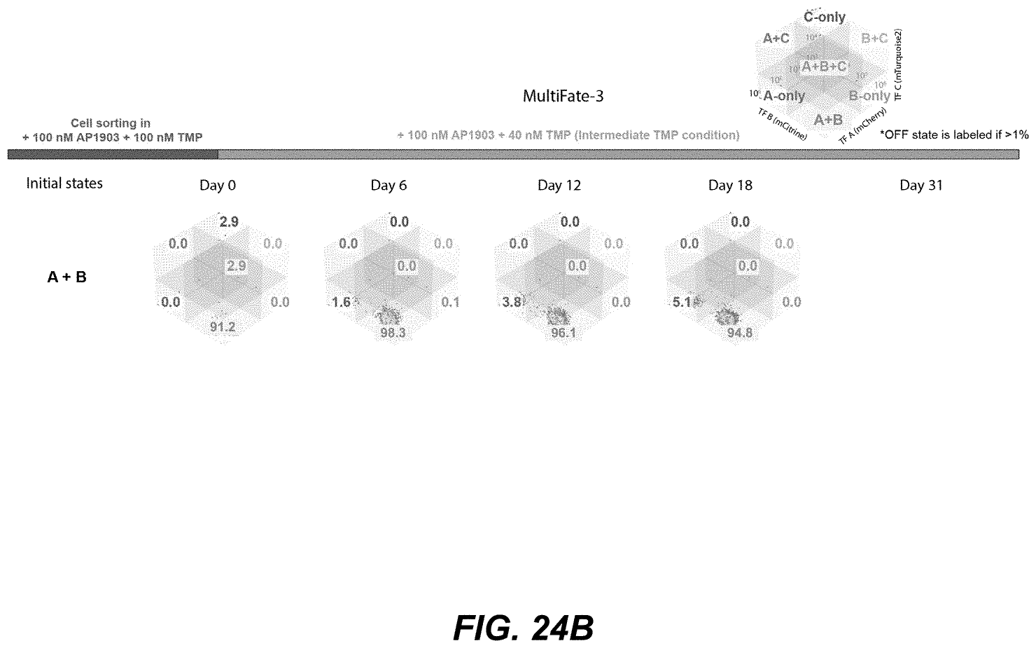

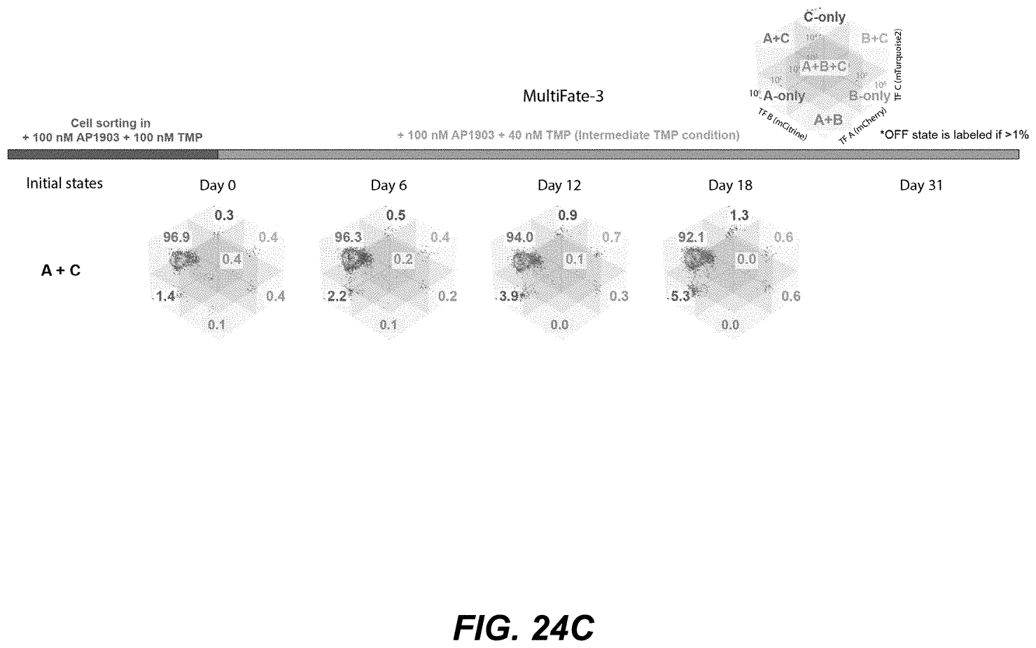

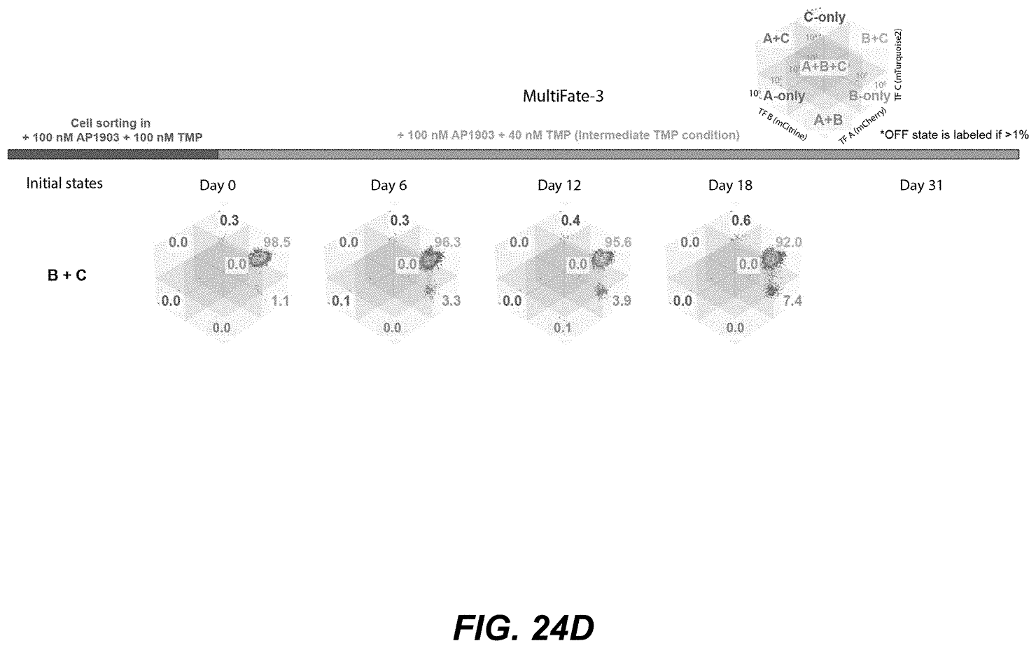

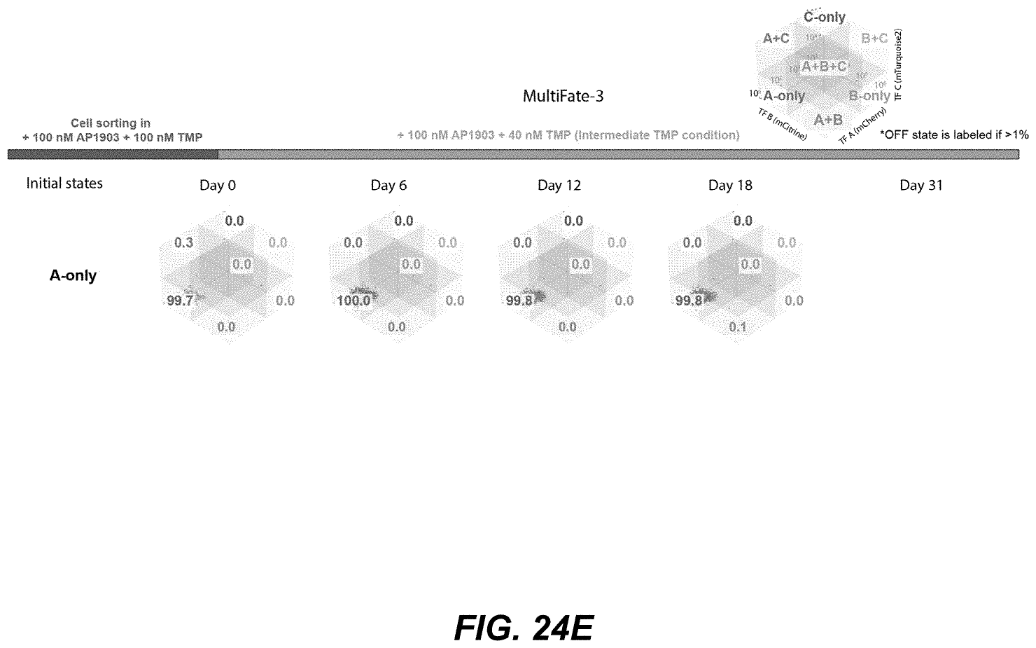

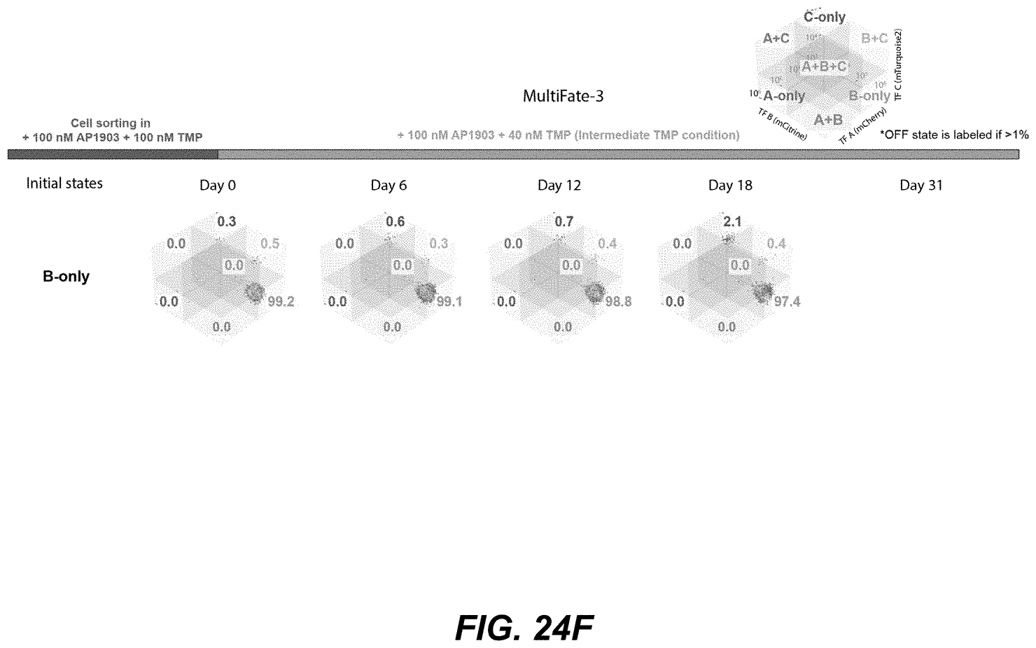

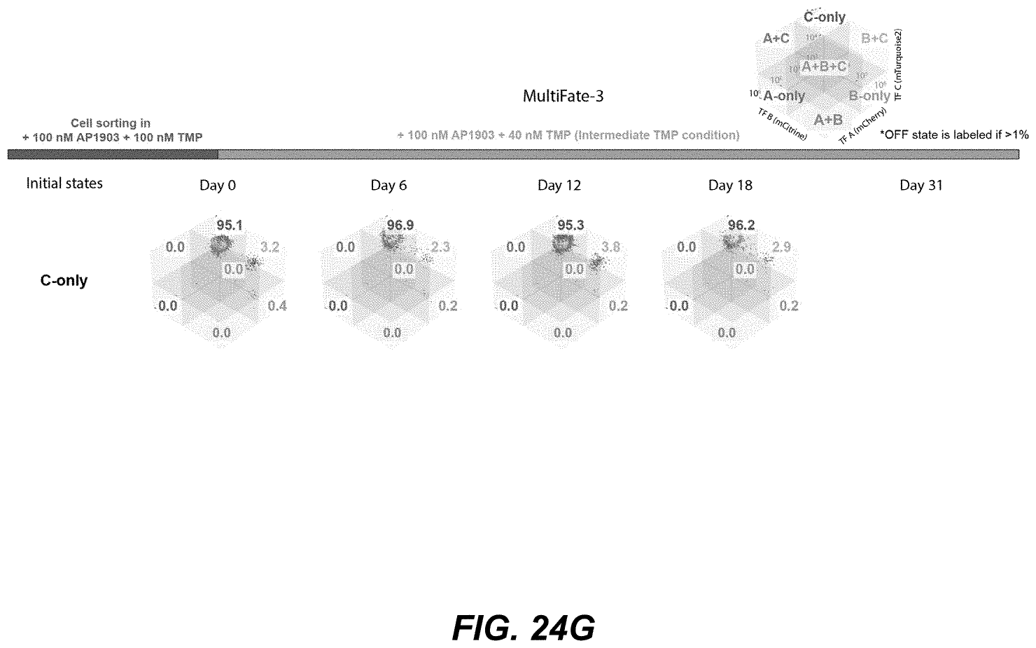

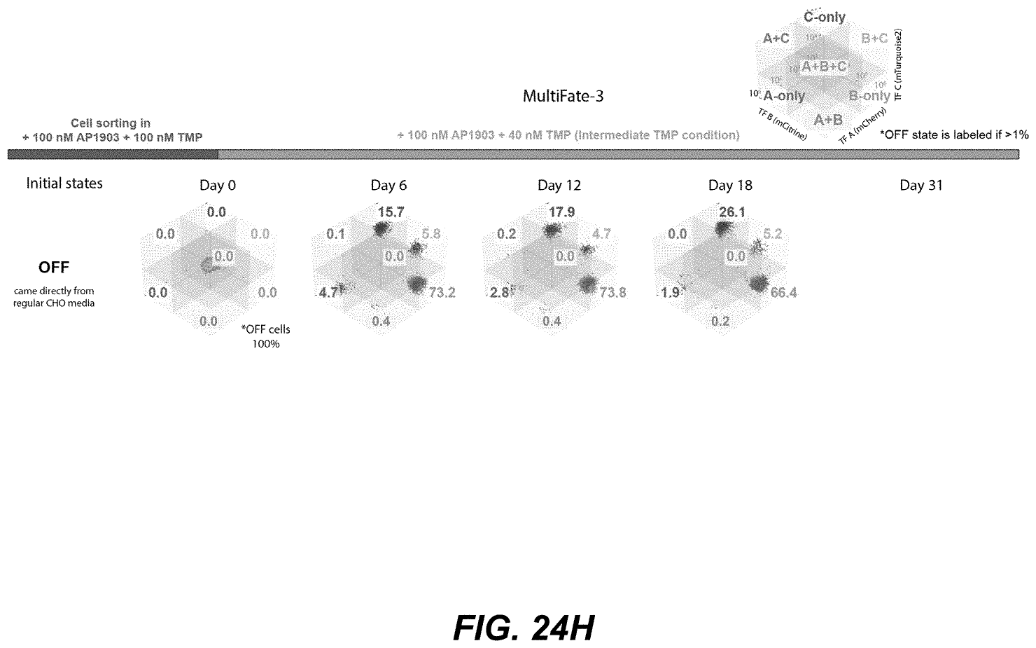

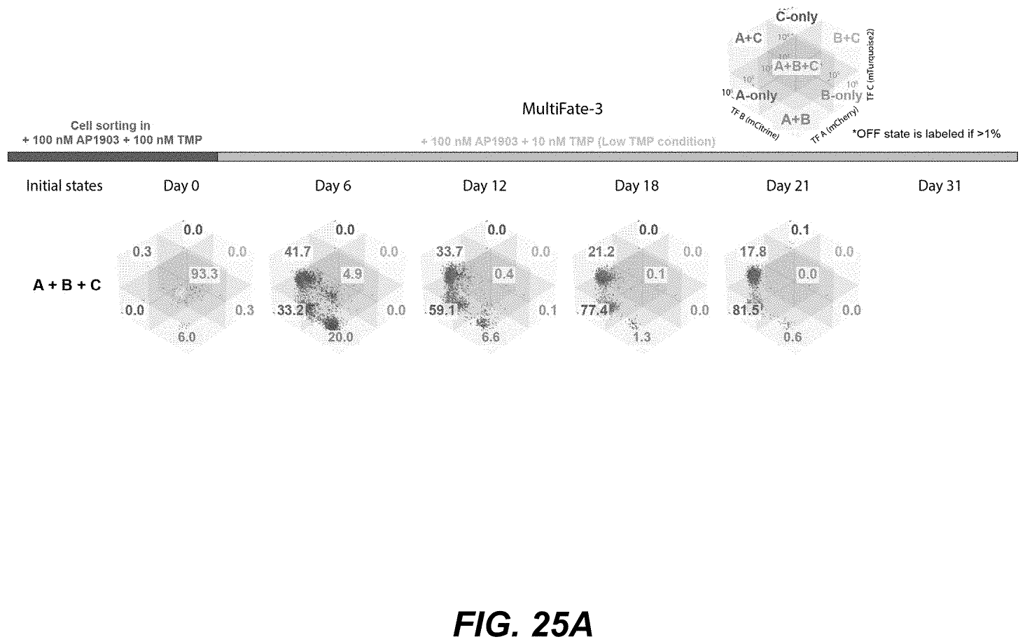

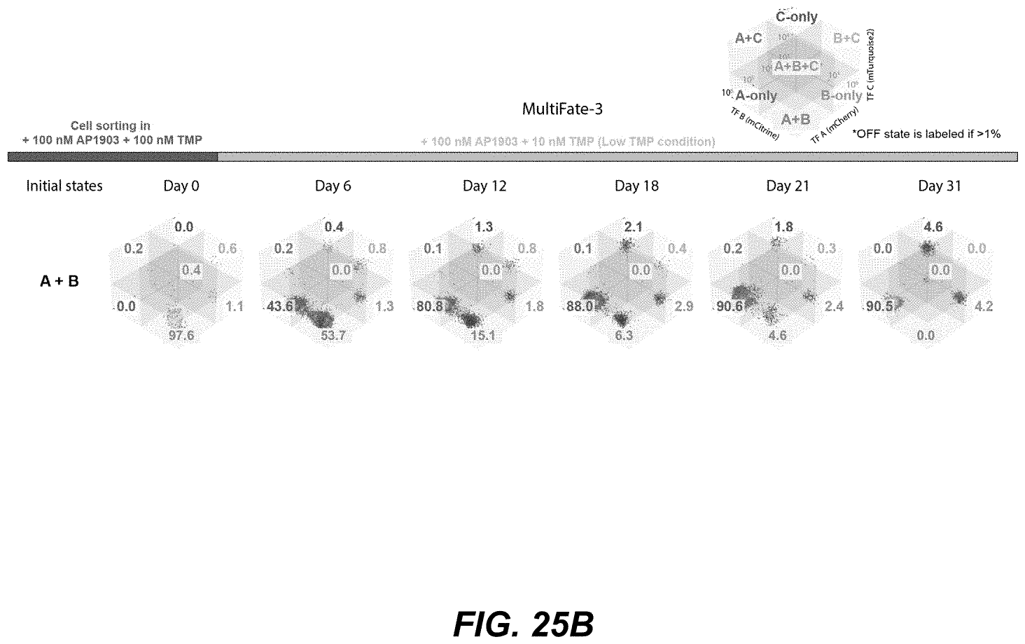

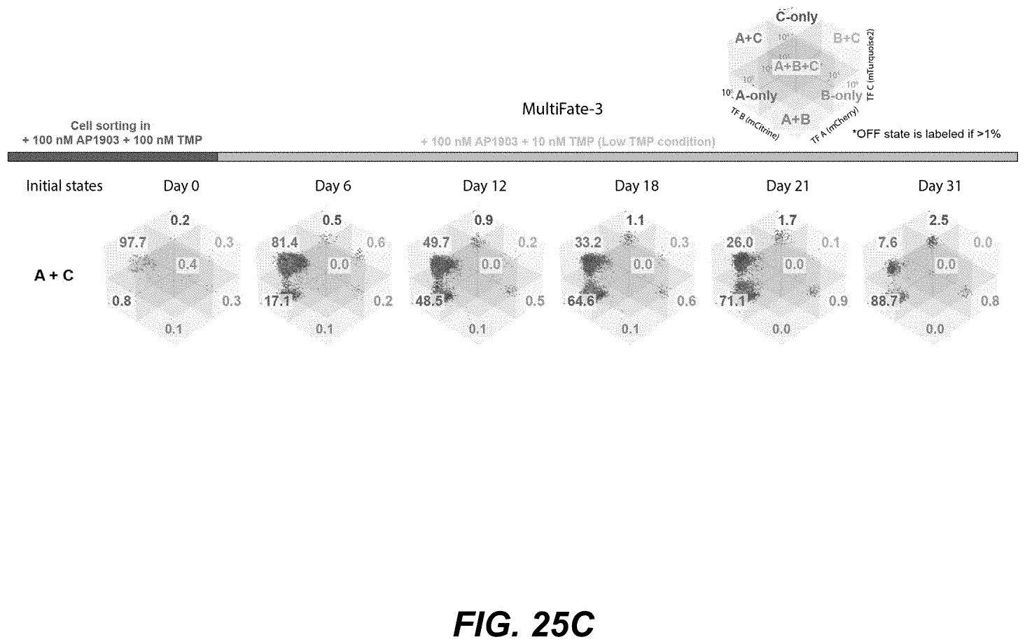

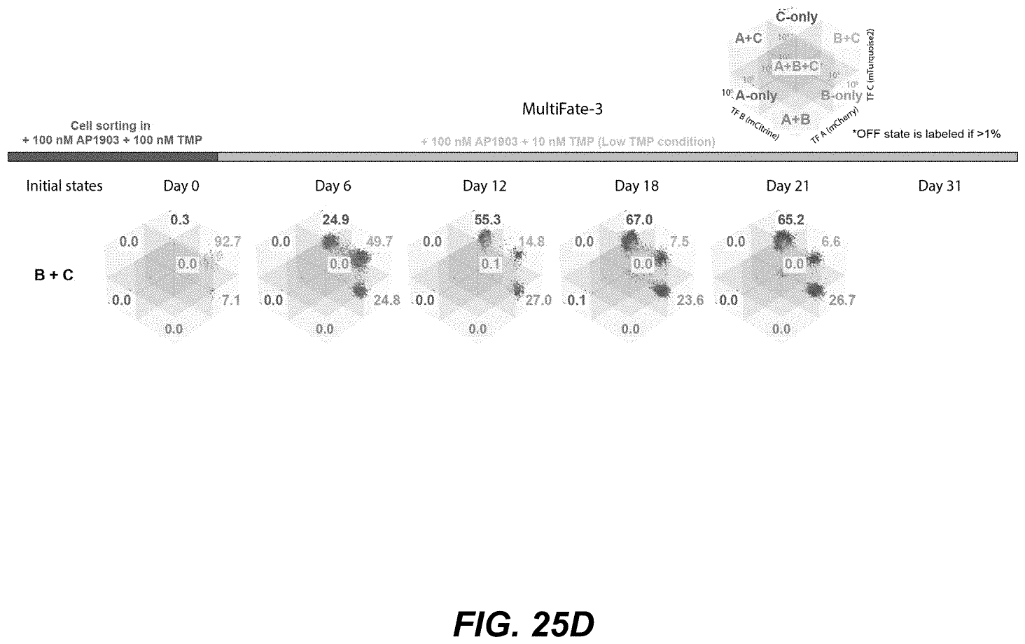

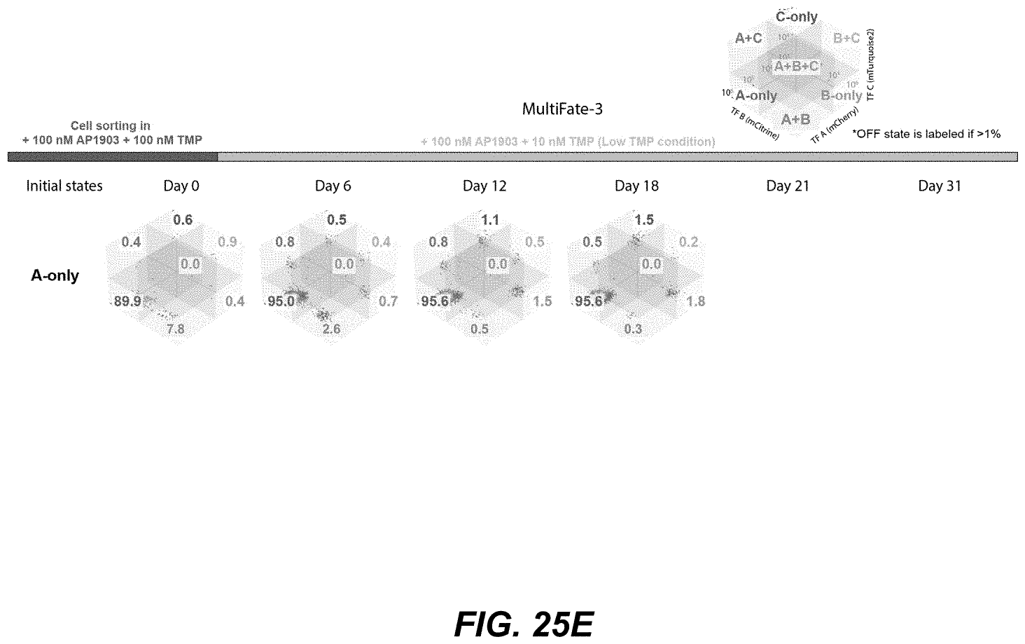

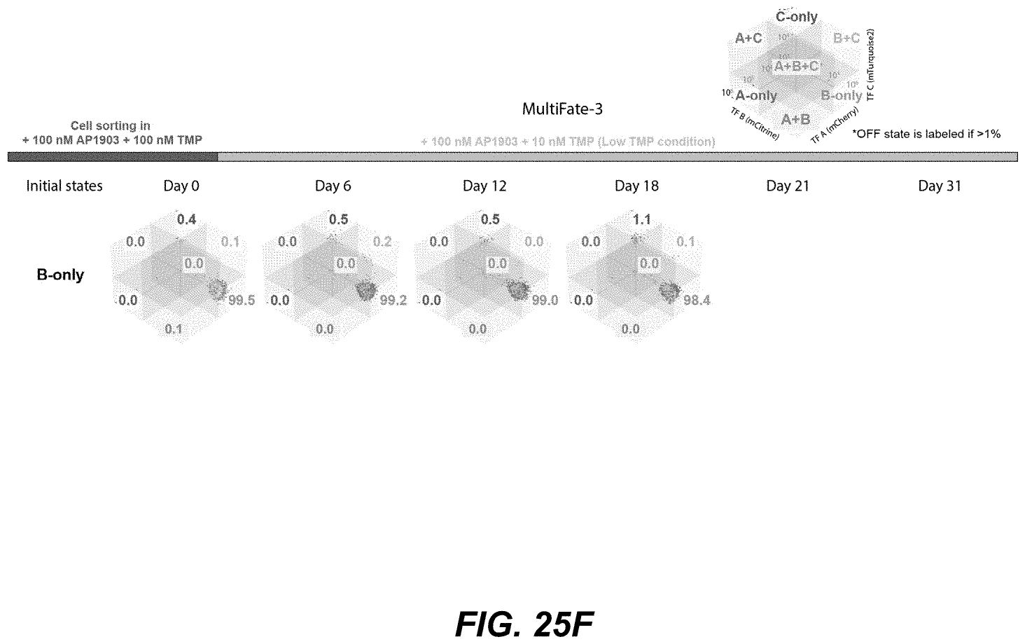

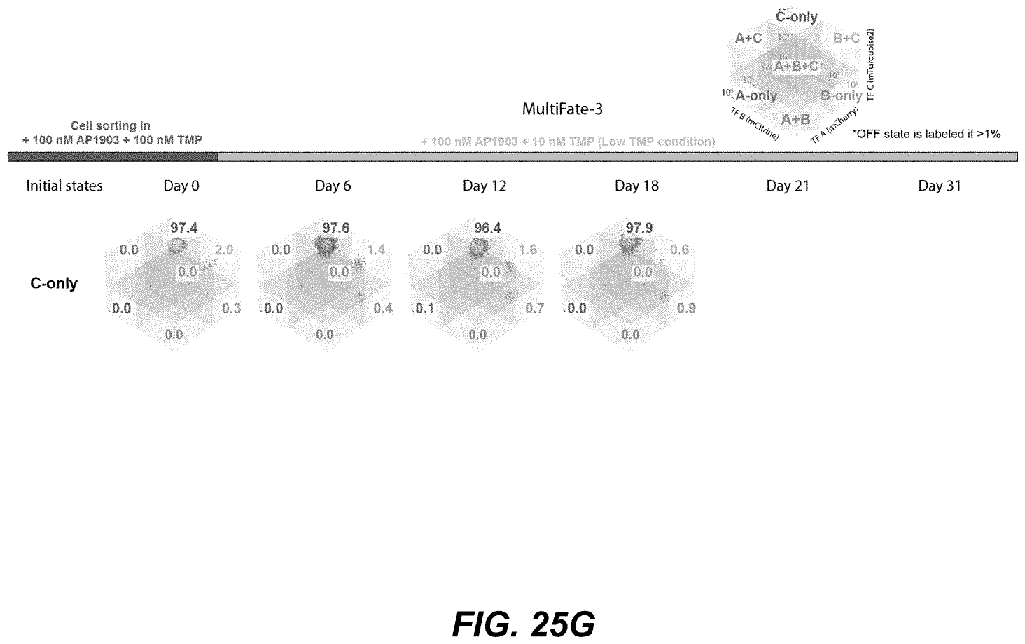

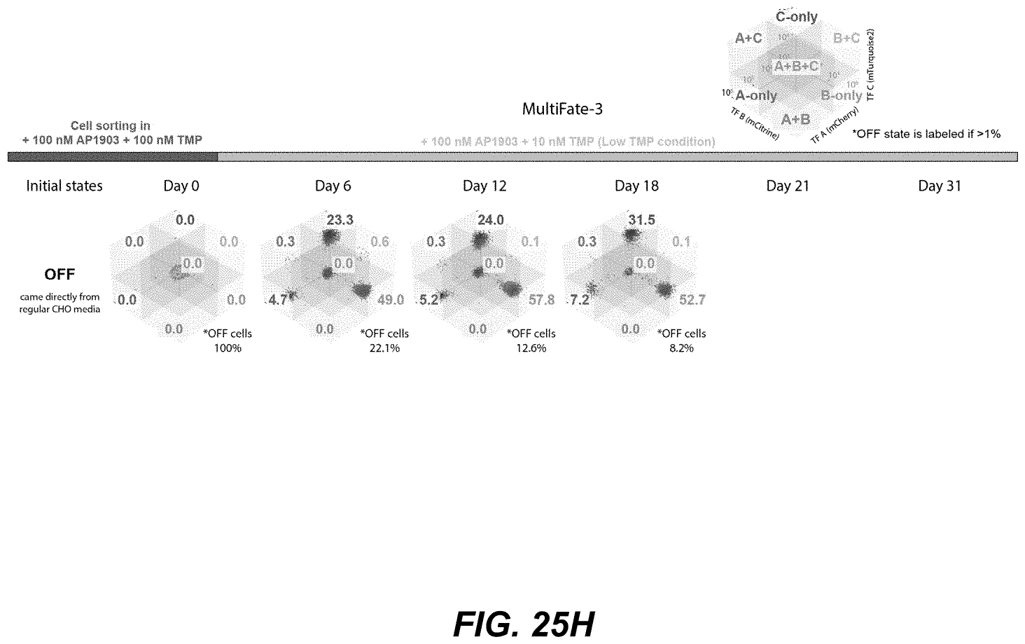

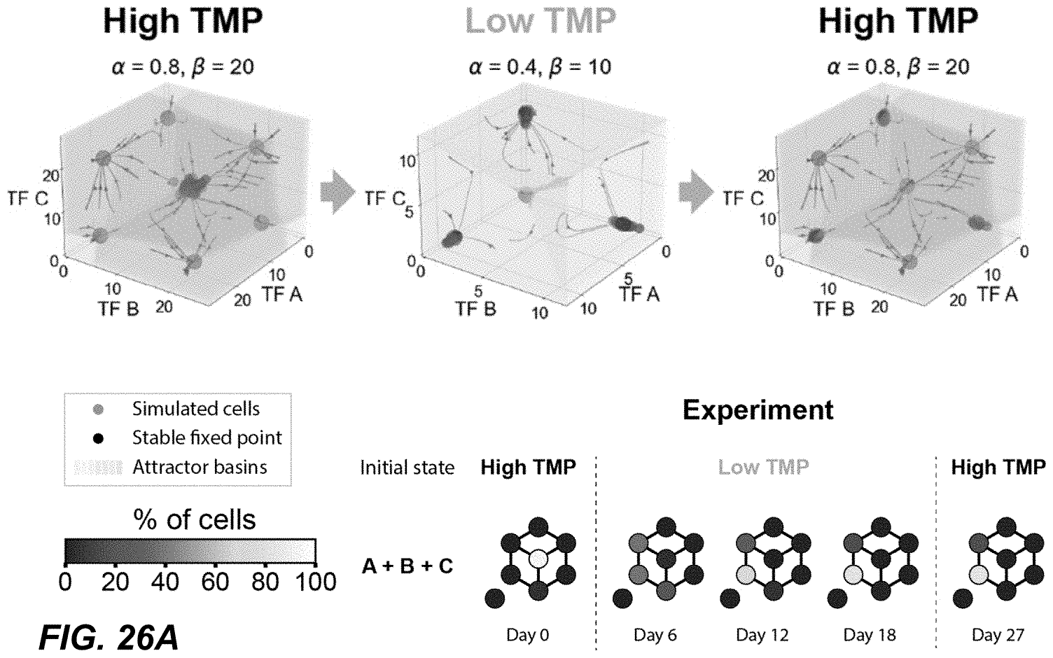

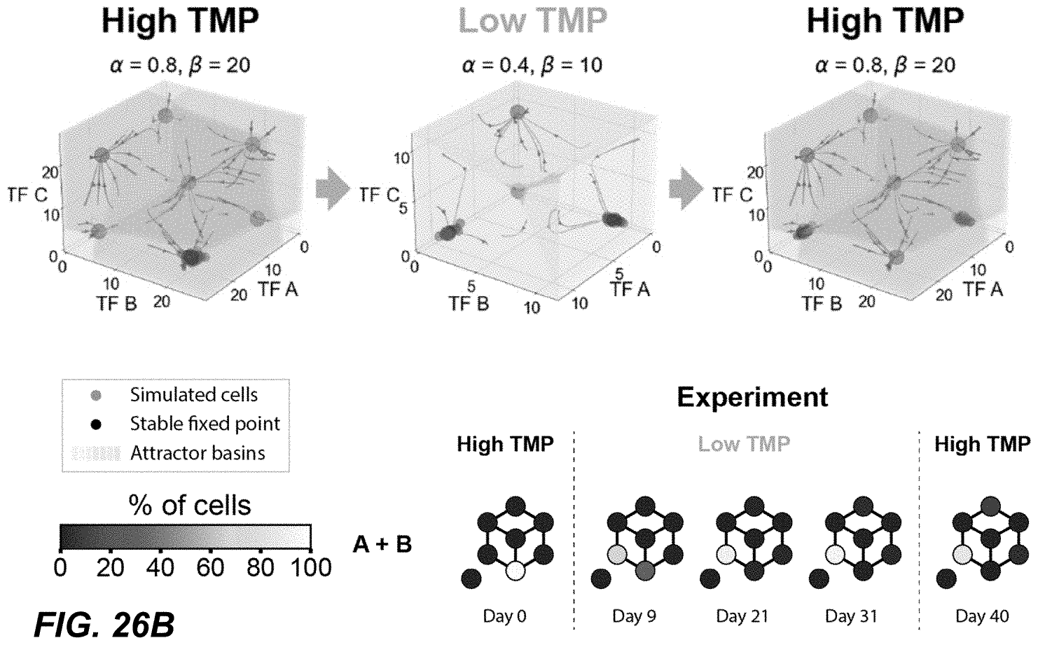

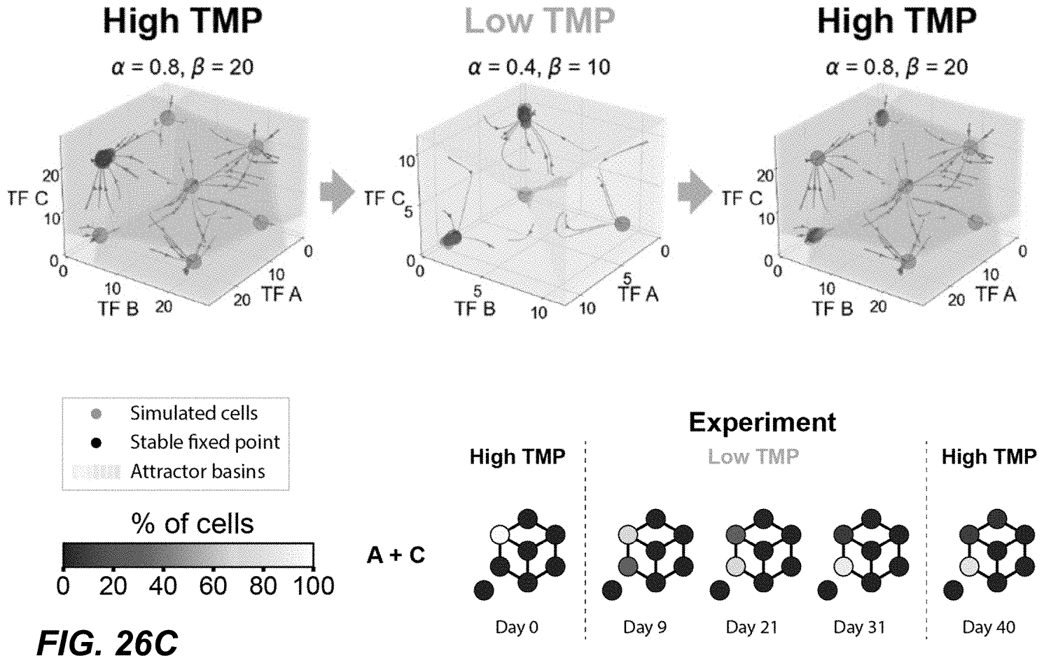

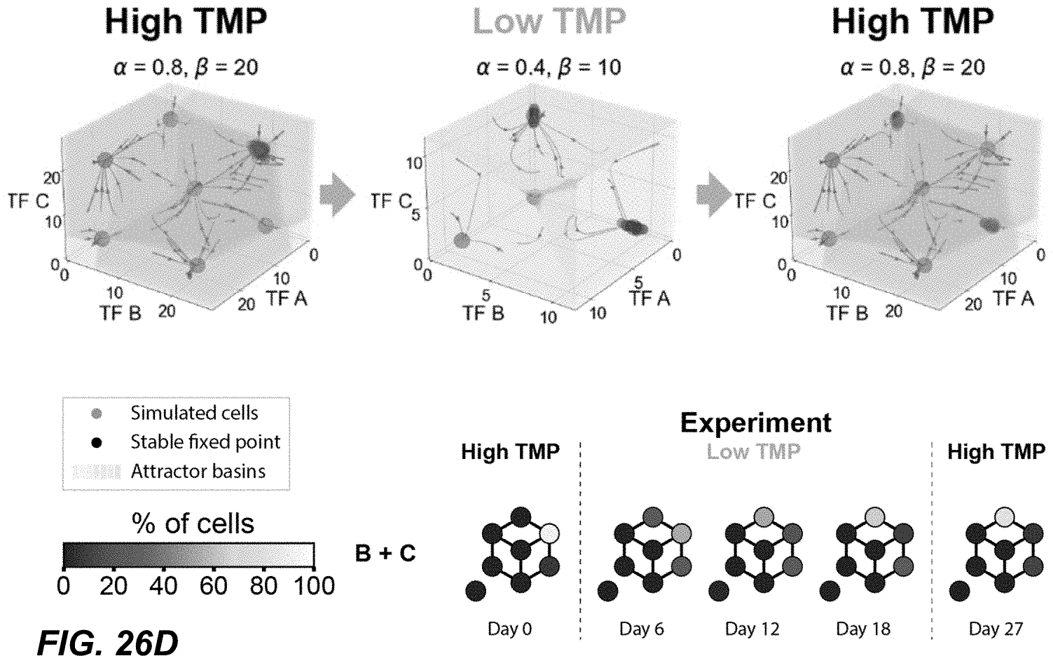

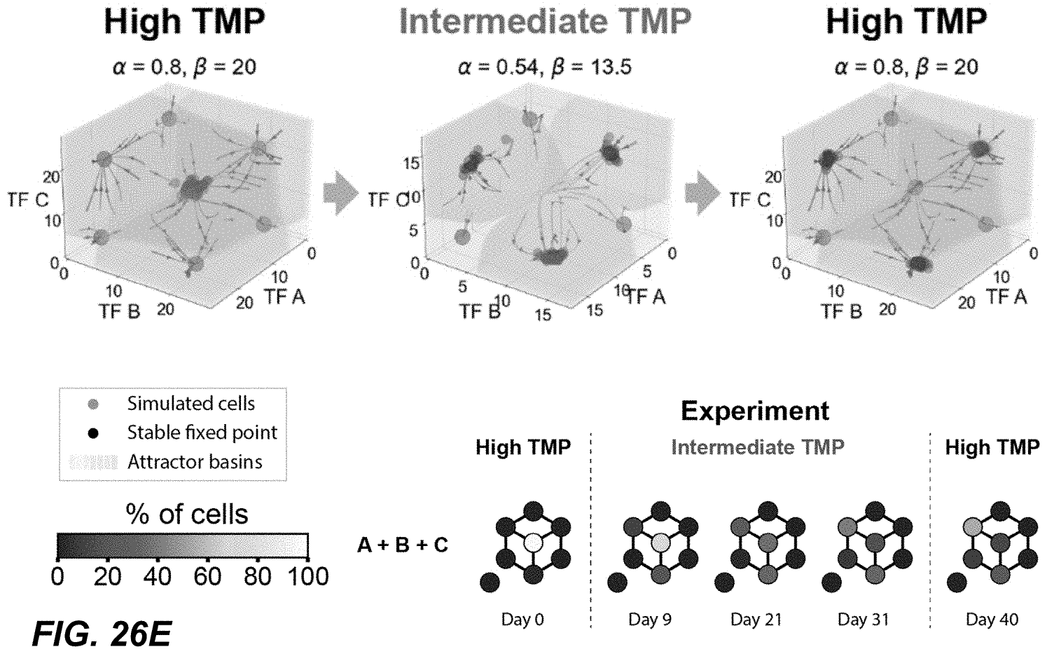

Like MultiFate-2, the number and stability of different states in MultiFate-3 can be modulated. In the model, reducing protein stability repeatedly bifurcates the system from type II septastability (7 stable states) through hexastability (6 stable states) to tristability (3 stable states) (FIG. 1D). Without being bound by any particular theory, this process resembles the progressive loss of cell fate potential during stem cell differentiation. To experimentally test this prediction, cells in each of the 7 states cultured under the High (100 nM) TMP condition (high protein stability) were transferred to similar media with Intermediate (40 nM) or Low (10 nM) TMP conditions. As predicted by the model, the Intermediate TMP condition destabilized only the A+B+C state, but not the other 6 states (FIG. 5B, Intermediate TMP columns, and FIG. 24A-FIG. 24H), whereas the Low TMP condition destabilized all multi-protein states, preserving only A-only, B-only and C-only states (FIG. 5B, Low TMP columns, and FIG. 25A-FIG. 25H). Consistent with the model, these transitions were also irreversible: restoring High TMP concentrations did not cause cells to repopulate previously destabilized states (FIG. 26A-FIG. 26E). Taken together, these results demonstrate that the MultiFate-3 circuit supports septastability, and allows controlled bifurcations to produce irreversible cell state transitions.

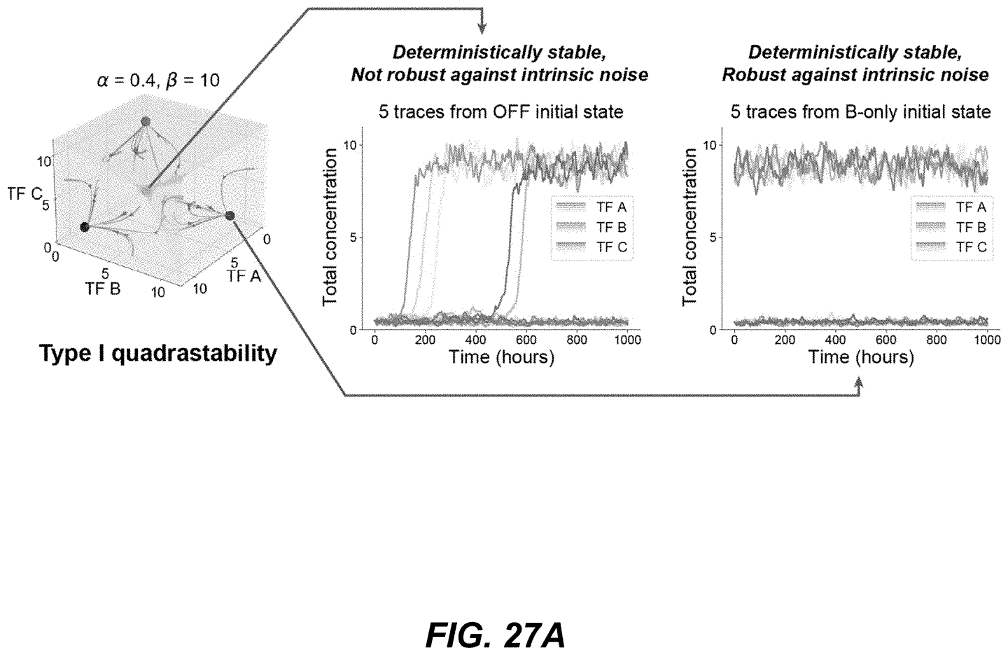

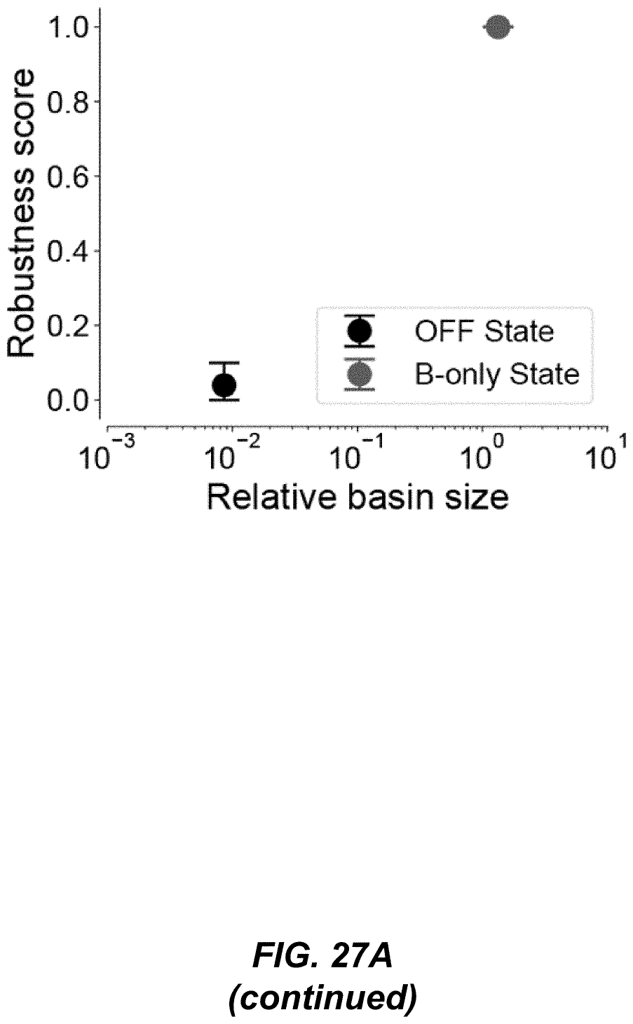

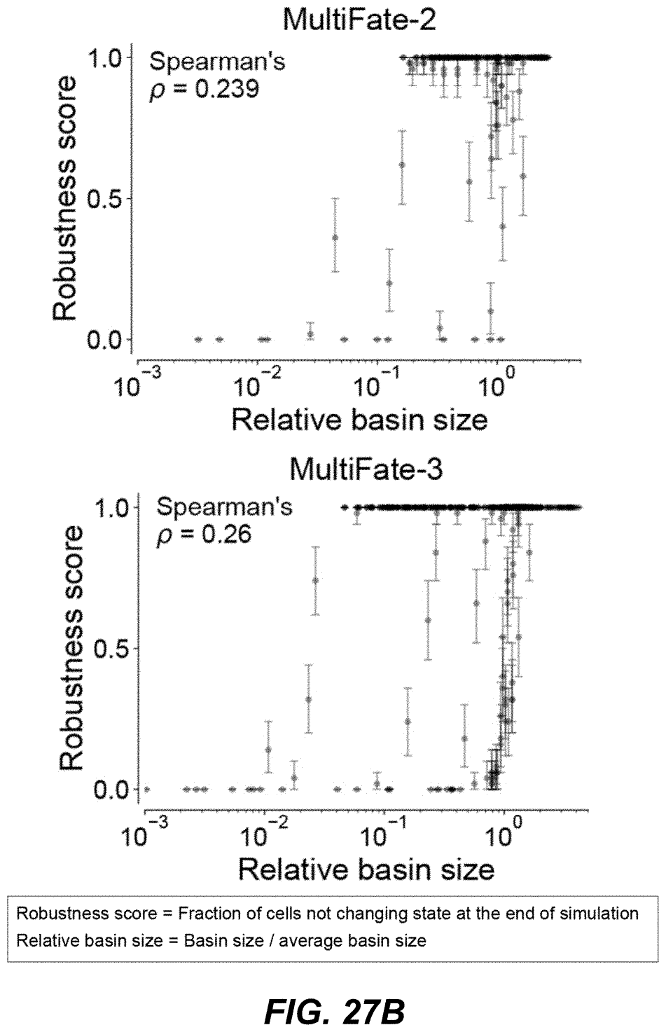

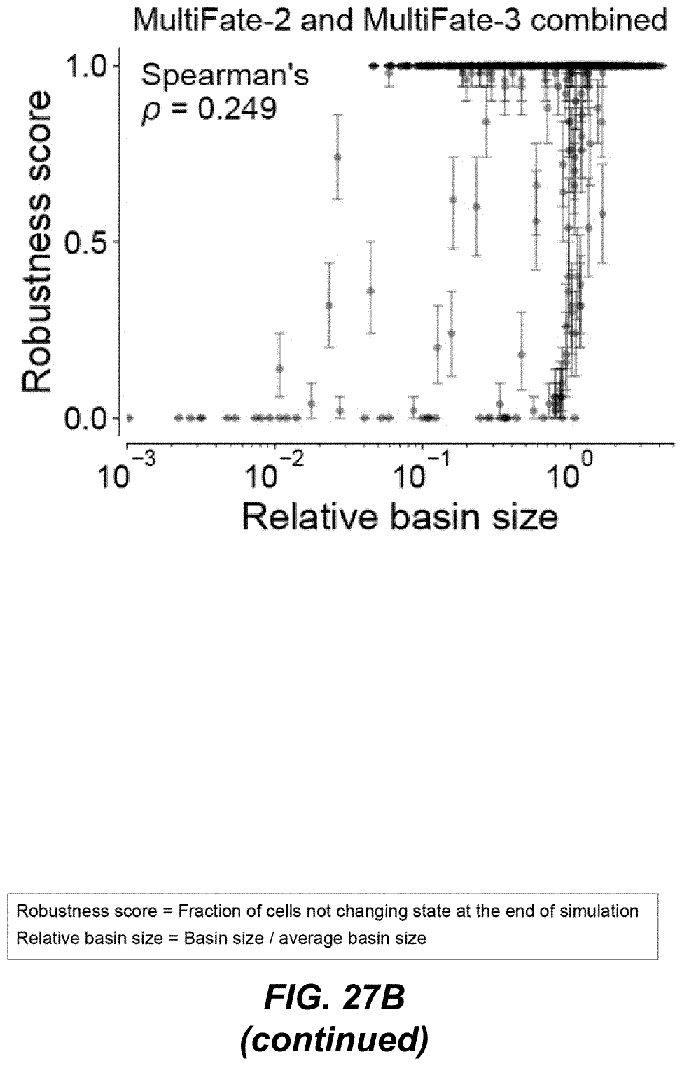

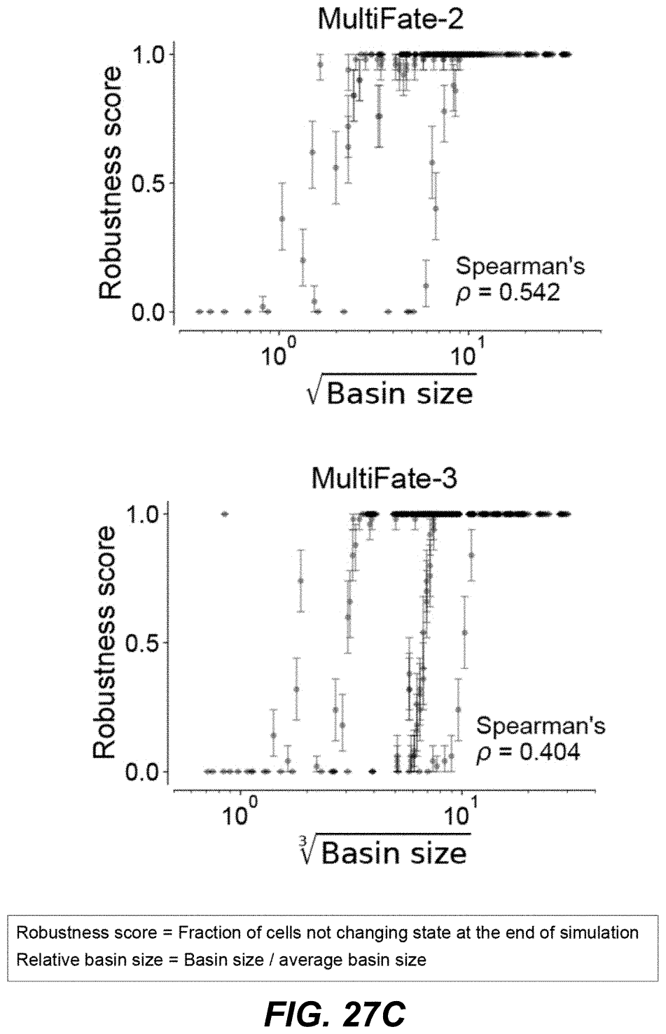

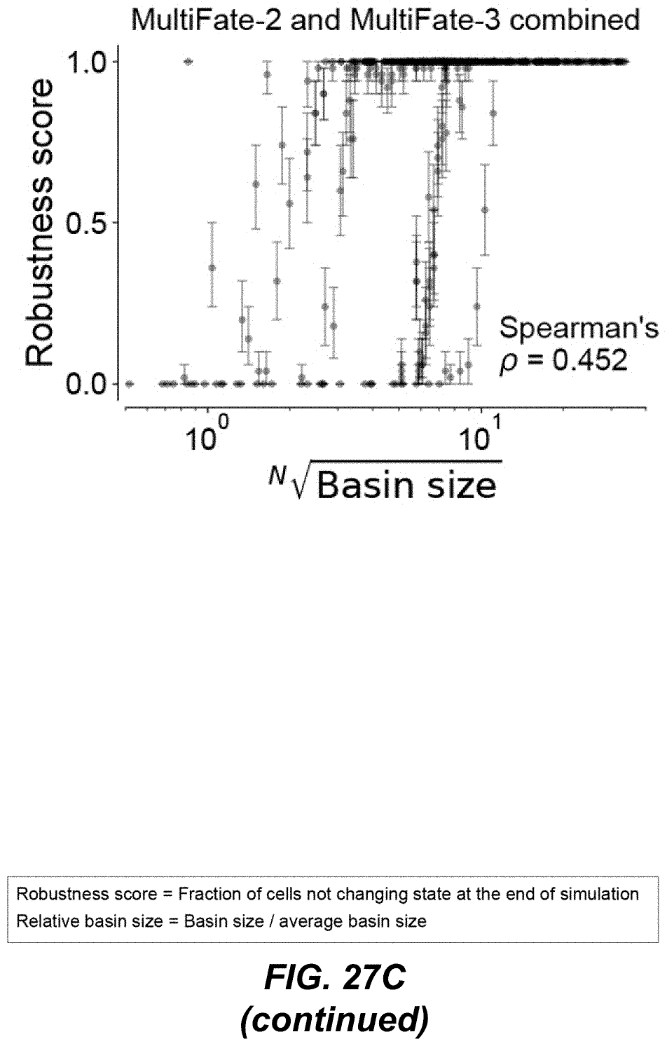

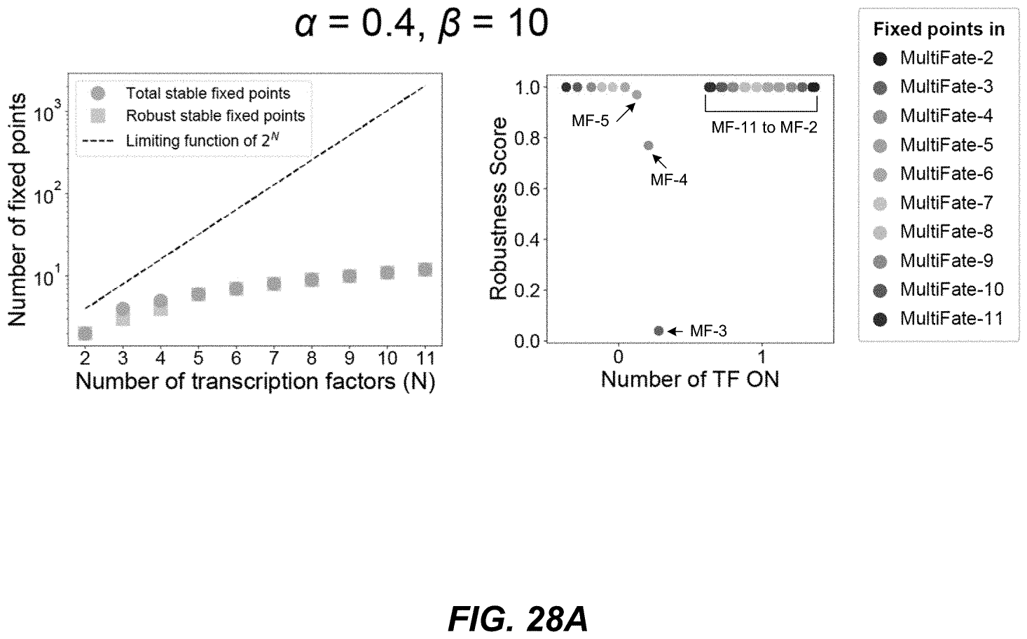

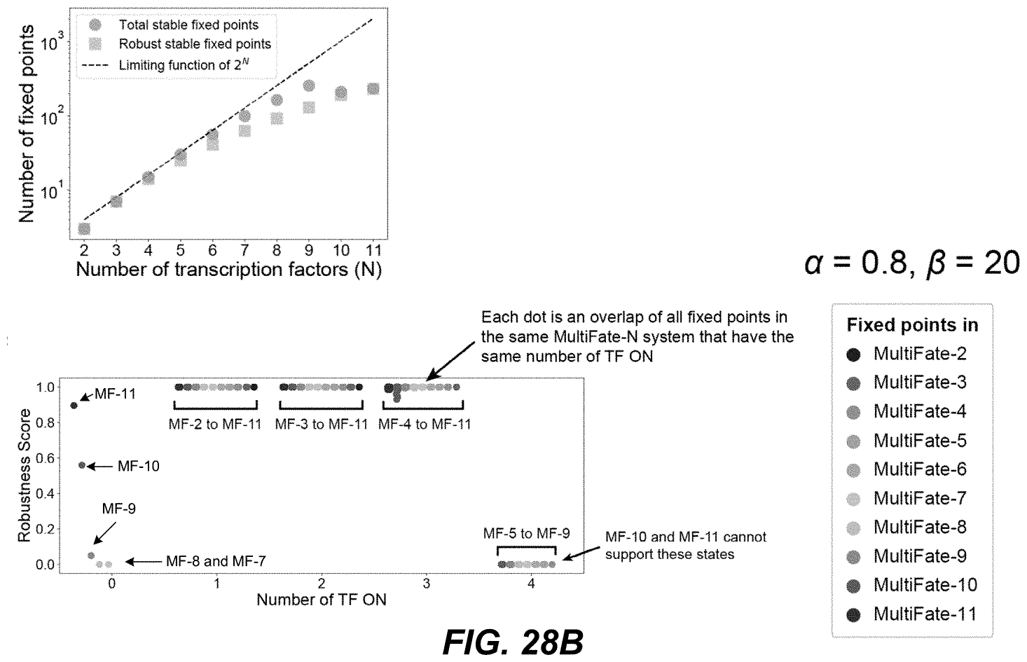

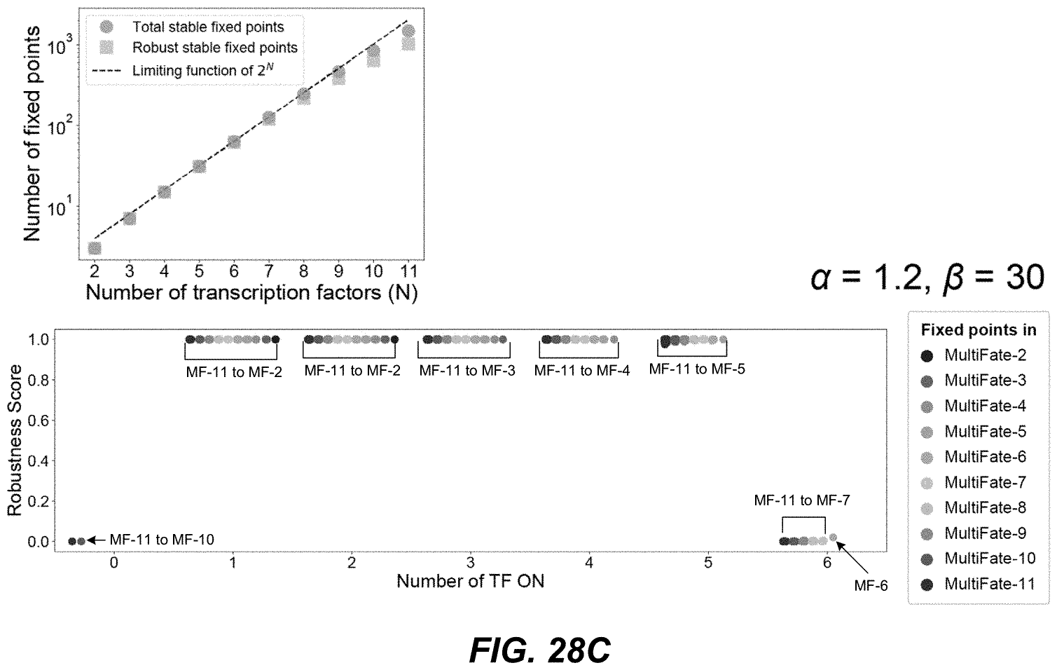

To understand higher order systems, MultiFate circuits were modeled containing up to N=11 transcription factors (See, “Additional Methods” below). Using the same parameter values established for MultiFate-2 and MultiFate-3, the number of attractors reached a maximum of 256 at N=9. Analysis of attractor escape rates in stochastic simulations revealed that most of these attractors were robust to gene expression noise (FIG. 5D and FIG. 27A-FIG. 27C) (See, “Additional Methods” below). The number of attractors grew more slowly than the theoretical limit of ˜2N because stable attractors could only sustain high levels of up to four transcription factors at a time (FIG. 28B). Without being bound by any particular theory, this limitation reflects the diminishing share of the active homodimers relative to all dimers. Similarly, the combined basal expression of all transcription factors suppressed homodimer formation, resulting in a decline in the number of attractors for systems containing more than 9 transcription factors (FIG. 5D and FIG. 28B). Finally, it is noted that the precise values of the maximum number of stable attractors can be modulated up or down by parameters that impact overall gene expression (FIG. 28A-FIG. 28C). Together, these results show that the MultiFate architecture can be expanded to generate large numbers of robust stable states.

The astonishing diversity of cell types in the human body underscores the critical importance of multistable circuits and provokes the fundamental question of how to engineer a robust, controllable, and expandable synthetic multistable system. Competitive protein-protein interactions and transcriptional autoregulation were utilized to design a synthetic multistable architecture that operates in mammalian cells. In some embodiments, the MultiFate circuits exhibit many of the hallmarks of natural cell fate control systems. In some embodiments, they may generate as many as seven molecularly distinct, mitotically heritable cell states (FIG. 3A-FIG. 3D and FIG. 5A-FIG. 5D). In some embodiments, they allow controlled switching of cells between states with transient transcription factor expression (FIG. 4B), similar to fate reprogramming. In some embodiments, they support modulation of state stability (FIG. 3A-FIG. 3D and FIG. 5A-FIG. 5D) and permit irreversible cellular transitions through externally controllable parameters such as protein stability (FIG. 4A and FIG. 26A-FIG. 26E), similar to the irreversible loss of cell fate potential during stem cell differentiation. Finally, in some embodiments, implementing cross-inhibition at the protein level makes MultiFate expandable by ‘plugging in’ additional transcription factors, without re-engineering the existing circuit, a useful feature for synthetic biology. Without being bound by any particular theory, the same design principle may play a related role in natural systems, allowing the emergence of new cell states through transcription factor duplication and sub-functionalization in a manner analogous to the stepwise expansion of MultiFate circuits demonstrated here.

A superior feature of this circuit is its close agreement with predictions from a dynamical systems model (See, “Design of the MultiFate Circuit” below). Despite a lack of precise quantitative parameter values for many molecular interactions, the qualitative behaviors enabled by this circuit design can be enumerated and explained from simple properties of the components and their interactions.

MultiFate has a relatively simple structure, requiring, in some embodiments, a small number of genes, all of the same type, yet exhibits robust memory behaviors, scalability, and predictive design. In some embodiments, MultiFate can be extended into a full-fledged synthetic cell fate control system. In some embodiments, MultiFate can be coupled to synthetic cell-cell communication systems such as synNotch, MESA, synthekines, engineered GFP, and auxin (as described in Additional Synthetic Protein Circuits) to enable navigation of cells through a series of fate choices, recapitulating cell behaviors associated with normal development. In some embodiments, MultiFate can also allow engineering of multicellular cell therapeutic programs. For example, one may engineer a stem-like state that can either self-renew or “differentiate” into other states that recognize and remember different input signals and communicate with one another to coordinate complex response programs. Such strategies will benefit from the ability of MultiFate to allow probabilistic differentiation into multiple different states in the same condition (FIG. 19A-FIG. 19D). In this way, the MultiFate architecture can provide a scalable foundation for exploring the circuit-level principles of cell fate control and enable new multicellular applications in synthetic biology.

### Design of the MultiFate Circuit

Provided herein are embodiments of the mathematical model of the MultiFate circuit and how it can be used to design the experimental system and predict its behavior. For simplicity, a symmetric MultiFate-2 circuit whose two transcription factors share identical biochemical parameters and differ only in their DNA binding site specificity is focused on first. A similar analysis of systems with more transcription factors and asymmetric parameters is presented in “Additional Methods” below.

The dynamics of protein production and degradation can be represented using ordinary differential equations (ODEs) for the total concentrations of the transcription factors A and B, denoted [Aₜₒₜ] and [Bₜₒₜ], respectively. The rate of production of each protein is assumed to follow a Hill function of the corresponding homodimer concentration, [A₂] or [B₂], with maximal rate β, Hill coefficient n, and half-maximal activation at a homodimer concentration of K_{M}. A low basal protein production rate, denoted α, is included to allow self-activation from low initial expression states. Finally, each protein can degrade and be diluted (due to cell division) at a total rate δ, regardless of its dimerization state. To simplify analysis, the model can be non-dimensionalized by rescaling time in units of δ⁻¹, concentrations in units of K_{M} (See, “Additional Methods” below), to obtain Equation 1 and Equation 2:

$$\begin{matrix}\frac{d [ {A}_{tot} ]}{d t} = α + \frac{{β [ {A}_{2} ]}^{n}}{1 + {[ {A}_{2} ]}^{n}} - [ {A}_{tot} ] & ( Equation 1 ) \\ \frac{d [ {B}_{tot} ]}{d t} = α + \frac{{β [ {B}_{2} ]}^{n}}{1 + {[ {B}_{2} ]}^{n}} - [ {B}_{tot} ] & ( Equation 2 )\end{matrix}$$

Here, Hill coefficient n only represents ultrasensitivity introduced by transcriptional activation. A more detailed discussion on additional ultrasensitivity provided by homodimerization and molecular titration is described in “Additional Methods” below.

Since dimerization dynamics occur on a faster timescale than protein production and degradation, the distribution of monomer and dimer states are assumed to remain close to their equilibrium values. This generates the following relationships between the concentrations of monomers, [A] and [B], and dimers, [A₂], [B₂], and [AB]:

[A]²=K_{d}[A₂] (Equation 3)

[B]²=K_{d}[B₂] (Equation 4)

2[A][B]=K_{d}[AB] (Equation 5)

Since the two transcription factors share the same dimerization domain, homo- and hetero-dimerization are assumed to occur with equal dissociation constants, K_{d}. Additionally, conservation of mass implies that [Aₜₒₜ]=[A]+[AB]+2[A₂] (Equation 6), with a similar relationship for B. Introducing the equilibrium equations given above into this conservation law produces expressions for the concentrations of the activating homodimers in terms of the total concentrations of A and B:

$$\begin{matrix}[ {A}_{2} ] = \frac{{2 [ {A}_{tot} ]}^{2}}{\begin{matrix}{K}_{d} + 4 ( [ {A}_{tot} ] + [ {B}_{tot} ] ) + \\ \sqrt{{K}_{d}^{2} + 8 ( [ {A}_{tot} ] + [ {B}_{tot} ] ) {K}_{d}}\end{matrix}} & ( Equation 7 ) \\ [ {B}_{2} ] = \frac{{2 [ {B}_{tot} ]}^{2}}{\begin{matrix}{K}_{d} + 4 ( [ {A}_{tot} ] + [ {B}_{tot} ] ) + \\ \sqrt{{K}_{d}^{2} + 8 ( [ {A}_{tot} ] + [ {B}_{tot} ] ) {K}_{d}}\end{matrix}} & ( Equation 8 )\end{matrix}$$

Inserting these expressions into the differential equations for [Aₜₒₜ] and [Bₜₒₜ] above, a pair of coupled ordinary differential equations are obtained with only [Aₜₒₜ] and [Bₜₒₜ] as variables.

To understand the behavior of this system in physiologically reasonable parameter regimes (Table 1), standard approaches from dynamical systems analysis were used (See, “Additional Methods” below). Based on ODEs, a phase portrait of variables [Aₜₒₜ] and [Bₜₒₜ] was first generated (labeled ‘TF A’ and ‘TF B’, which are dimensionless total TF A or B concentrations), where the linewidth of a vector (FIG. 1C, gray arrows) at any point is proportional to the speed of that point. On the phase portrait, the nullclines (FIG. 1C, solid lines) were plotted, defined by setting each of the ODEs above to zero. Fixed points at nullcline intersections were then identified, and their linear stability was determined (FIG. 1C, black and white dots). Finally, the basins of attraction for each stable fixed point were delineated (FIG. 1C, shaded regions).

Using this analysis, parameter values were identified that support type II tristability, a regime that minimally embodies the developmental concept of multilineage priming (FIG. 1C and FIG. 6B). Stronger self-activation (higher values of β) was more likely to produce type II tristability (FIG. 6B, 3 row and column). Too much leaky production (high α) allowed both transcription factors to self-activate, reducing the degree of multistability, whereas too little (low α) stabilized the undesired OFF state (FIG. 6B, α column). Strong dimerization (low K_{d}) was essential for type II tristability (FIG. 6B, K_{d} row and column). Finally, a broad range of Hill coefficients n≥1 were compatible with type II tristability. Although higher values of n led to a reduced sensitivity to other parameters and allowed the system to tolerate higher values of α, they also stabilized the OFF state (FIG. 6B, n row and column). Together, these results showed that an ideal design, in some embodiments, would maximize β, minimize K_{d}, and use intermediate values of α and n.

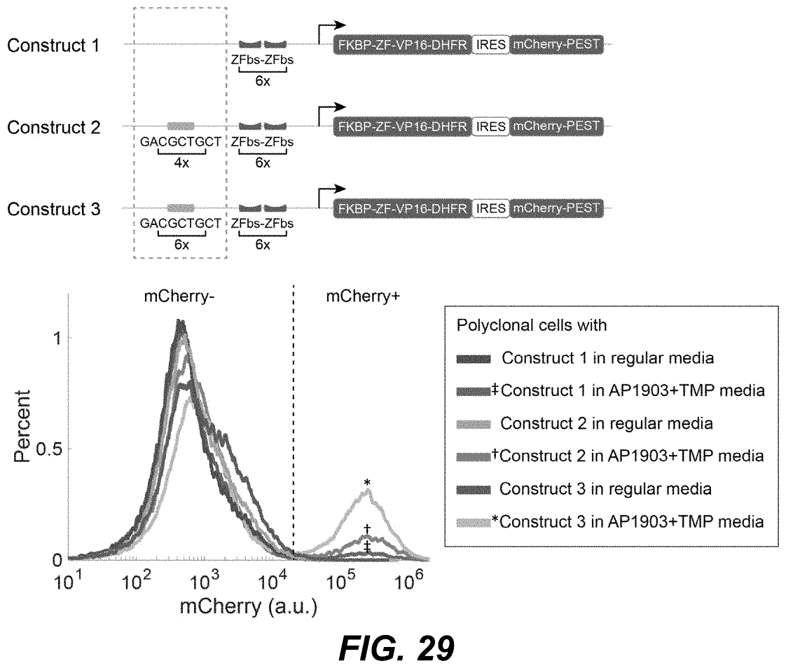

Based on these conclusions, multiple repeats of the homodimeric binding sites were incorporated to maximize β, strongly associating FKBP12F36V homodimerization domains were used to minimize K_{d}, and the promoter sequences were modified to allow some leaky expression to optimize α (FIG. 29) (See, “Additional Methods” below). Finally, although n was not directly controlled, it was expected that the repeated homodimeric binding sites should lead to some ultrasensitivity. These design choices produced the selected type II tristability in the experimental system (FIG. 3C).

A key feature of the MultiFate design, in some embodiments, is its ability to qualitatively change its multistability properties through bifurcations in response to parameter changes. The mathematical model predicts that protein stability can control the number of stable fixed points in phase space. In the non-dimensionalized model, the protein degradation rate, δ, does not appear explicitly but enters through the rescaling of α and β by (δK_{M})⁻¹ (See, “Additional Methods” below). Thus, tuning protein stability is equivalent to multiplying both α and β by a common factor, which is referred to herein as the “protein stability factor.” Reducing protein stability shifts the nullclines closer to the origin, causing the two unstable fixed points to collide with the stable A+B fixed point in a subcritical pitchfork bifurcation (FIG. 1C). The result is a bistable system with A-only and B-only stable fixed points at somewhat lower concentrations (FIG. 1C). To experimentally realize this bifurcation, the circuit was designed to allow external control of transcription factor protein stability using the drug-inducible DHFR degron (FIG. 2C). As predicted, reducing protein stability destabilized the A+B state, but preserved the A-only and B-only stable states (FIG. 3C). In this way, model-based design enabled rational engineering of tristability as well as externally controllable transitions to bistability in the experimental system.

### Methods Summary

All tissue culture experiments were performed with Chinese hamster ovary K1 cells (CHO-K1, ATCC). For flow cytometry experiments characterizing ZF transcription factors (FIG. 2A-FIG. 2B and FIG. 9A-FIG. 9C), CHO-K1 cells were co-transfected with mTagBFP2 (as co-transfection marker), reporter and ZF transcription factor (Table 2). Cells were harvested after 36 hours and cell fluorescence was measured by flow cytometry. For experiments characterizing ZF transcription factor self-activation (FIG. 2C and FIG. 10A), each self-activation construct was stably integrated (Table 2) into polyclonal Tet3G-expressing CHO-K1 cells via PiggyBac (Systems Biosciences) to make a polyclonal cell line (Table 3). The integrated self-activation cassettes in each polyclonal line were transiently activated by adding Dox (Sigma-Aldrich) for 24 hours, then Dox was washed out and cells were transferred into different combinations of AP1903 and/or TMP (Sigma-Aldrich). After another 72 hours, cells were harvested and analyzed by flow cytometry. To test inhibition through competitive dimerization (FIG. 2D and FIG. 10B-FIG. 10C), two monoclonal self-activation lines with 42ZFR2AR39AR67A DNA-binding domain and either GCN4 or FKBP dimerization domain were selected. Plasmids constitutively expressing different perturbation transcription factors were stably integrated in each monoclonal line, then transferred cells into media containing AP1903 and TMP to permit self-activation. The inhibition strength was quantified as the reduction of self-activation cell fractions.

MultiFate-2 lines were constructed by stably integrating corresponding constructs into polyclonal ERT2-Gal4-P2A-Tet3G-expressing CHO-K1 cells (Table 3). FACS was used to sort cells that have stable A+B state in media containing AP1903 and TMP as single cells into 384-well plates to obtain monoclonal MultiFate-2 lines. MultiFate-3 cells were constructed by stably integrating the TF C self-activation cassette into MultiFate-2.2 cells, and a similar sorting method was used to obtain the MultiFate-3 monoclonal cells (FIG. 12A-FIG. 12B).

For flow cytometry experiments characterizing state stability (FIG. 3C and FIG. 5B) and state-switching dynamics (FIG. 4A-FIG. 4B), cells from each state were sorted into media containing corresponding inducers. These cells were continuously cultured by trypsinizing cells and transferring 4% of cells into fresh media containing corresponding inducers every three days. The remaining 96% cells were suspended in the flow cytometry buffer and analyzed by flow cytometry. For time-lapse imaging (FIG. 3D, FIG. 5C), cells from each state were sorted, mixed with equal ratio, and the cell mixture was sparsely plated in the same well with media containing AP1903 and TMP. After 6-12 hours, the media was changed and imaging began.

Mathematical models of MultiFate circuits are summarized above and in “Additional Methods” below.

### Materials and Methods

Plasmid Construction

Constructs used in this study are listed in Table 2. Some constructs were generated using standard cloning procedures. The inserts were generated using PCR or gBlock synthesis (IDT) and were annealed by Gibson assembly with backbones that are linearized using restriction digestion. Selected constructs used to build MultiFate lines are deposited into Addgene.

Tissue Culture

Chinese hamster ovary K1 cells (CHO-K1, ATCC) were cultured at 37° C. in a humidity-controlled chamber with 5% CO₂. The growth media consisted of Alpha MEM Earle's Salts (FujiFilm Irvine Scientific) supplemented with 10% FBS, 1 U/ml penicillin, 1 μg/ml streptomycin and 1 mM L-glutamine. For experiments requiring a change of inducer conditions, cells were first washed 3 times using media with the new inducer condition. After the wash, cells were rinsed once with Dulbecco's Phosphate-Buffered Saline (DPBS, Life Technologies) and trypsinized with 0.25% Trypsin (Life Technologies) for 3 min at 37° C. Trypsinized cells were then transferred into a new well with media added with the new inducer condition.

Transient Trans Fection

24 hours before transfection, 0.05×10⁶ CHO-K1 cells were seeded per well of a 24-well plate using standard culture media. The next day, cells were transfected with plasmids using Lipofectamine LTX and PLUS Reagents (Thermo Fisher) according to manufacturer's protocol.

Cell Line Construction

Stable cell lines used in this study are listed in Table 3. Stable cell lines were generated using the PiggyBac Transposon system (System Biosciences). CHO-K1 cells in a 24-well plate were co-transfected with transgene constructs in a PiggyBac expression backbone, an EF1α-PuroR plasmid and a Super PiggyBac Transposase plasmid. Cells were transferred into a 6-well plate and selected with 10 μg/ml puromycin for 3 days to obtain a stable polyclonal population.

To identify potential MultiFate-2 clones that can operate in several multistability regimes, the MultiFate mathematical model was used. Through parameter screening it was found that when one progressively reduces protein stability starting from a value at which the state with all transcription factors expressed simultaneously (all-ON) was stable a progressive reduction of multistability can be generated (FIG. 6B).

To achieve similar behavior experimentally, MultiFate-2 monoclones that exhibit stable A+B state were selected (FIG. 12A). The expression of all transcription factors in polyclonal MultiFate-2 cells were transiently induced by Dox (Sigma-Aldrich) (and 4-OHT (Sigma-Aldrich) if the second cassette has UAS) for 36 hours, then cells were washed and replaced with media containing 100 nM AP1903 (MedChemExpress) and 10 μM TMP (Sigma-Aldrich) (each at the saturating concentration). After another 3 days, polyclonal cells with both A and B still activated were sorted by FACS as single cells into 384-well plates. The plates were checked under a microscope after 4-5 days to eliminate wells without cells or with more than one colony. For wells that only have a single colony growing, cells were expanded, and subsequent screening was performed to obtain MultiFate-2 monoclones. Using a similar method, a monoclonal line that can maintain the stability of the A+B+C state was selected as the MultiFate-3 line (FIG. 12B).

Flow Cytometry

All samples were harvested from 24-well plates. Cells were first rinsed with 500 μl DPBS, and then trypsinized with 75 μl 0.25% trypsin for 3 min at 37° C. Trypsin was neutralized by resuspending cells in 300 μl flow cytometry buffer containing Hank's Balanced Salt Solution (Life Technologies) and 2.5 mg/ml Bovine Serum Albumin. Cell samples were then filtered by 40 μm cell strainers and analyzed by a flow cytometer (CytoFLEX, Beckman Coulter). The EasyFlow Matlab-based software package developed by Yaron Antebi was used to process flow cytometry data (https://antebilab.github.io/easyflow/).

Characterization of ZF Transcription Factors

To characterize transcriptional activation of different ZF transcription factor variants (FIG. 2A-FIG. 2B and FIG. 9A-FIG. 9C), CHO-K1 cells were transfected in 24-well plates with 50 ng ZF transcription factor plasmid (Table 2, construct MF08-MF62), 50 ng reporter plasmid (Table 2, construct MF01-MF07) and 25 ng EF1α-mTagBFP2. In the “ReporterOnly” group, ZF transcription factor plasmid was replaced by an empty plasmid with only a constitutive promoter but no ZF transcription factor. In the “NoReporter” well, both ZF transcription factor plasmid and reporter plasmid were replaced by an empty plasmid. For ZF transcription factors with FKBP homodimerization domain (FIG. 2B), AP1903 was added to the transfection media. 36 hours after transfection, cells were harvested and analyzed by flow cytometry. To maximize the reporter dynamic range, highly transfected cells were selected and compared by gating cells with high levels of a BFP co-transfection marker. Median citrine fluorescence intensity of gated cells was used to calculate fold activation. To calculate fold activation, median fluorescence values of NoReporter samples, representing the cellular autofluorescence background, were first subtracted from ReporterOnly and Reporter+ZF samples. The ratio between background-subtracted Reporter+ZF value and background-subtracted ReporterOnly value is then the fold activation of that ZF transcription factor on that reporter.

Characterization of ZF Transcription Factor Self-Activation

Each self-activation construct (Table 2, construct MF63-MF69) was stably integrated into Tet3G-expressing CHO-K1 cells. After puromycin selection, polyclonal cells were transferred into media containing 500 ng/ml Dox to transiently express ZF transcription factors. After 24 hours of Dox treatment, cells were washed 3 times with regular media and transferred into media containing different concentrations of AP1903 and/or TMP to test how dimerization and/or protein stability affect self-activation. One sample of cells (Dox+ sample) continued to be cultured in 500 ng/ml Dox as the positive control. After another 72 hours, cells were harvested and analyzed by flow cytometry. Stable polyclonal cells showed a strong bimodal mCitrine distribution upon circuit activation (FIG. 2C, top graph). An empirical threshold at mCitrine=10⁴ fluorescence units was used to separate the population into mCitrine− (cells with no circuit integrated or integrated circuit cannot self-activate) and mCitrine+(cells with integrated circuit activated) subpopulations. To only consider cells with at least one stably integrated activatable circuit, the mCitrine+ fraction of each sample was normalized to the mCitrine fraction of Dox+ sample, in which high concentrations of Dox should turn on all stably integrated activatable cassettes. This normalized mCitrine+ fraction was used to compare self-activation strength across different AP1903 and TMP combinations.

Assay Showing Inhibition of Self-Activation by Competitive Dimerization

Two monoclonal self-activation stable lines (with 42ZFR2AR39AR67A DNA-binding domain and either GCN4 or FKBP as the homodimerization domains, see Table 3) were selected since they showed spontaneous and homogeneous self-activation upon the addition of 100 nM AP1903 and 10 μM TMP (FIG. 10B-FIG. 10C). To determine whether competitive dimerization inhibits self-activation (FIG. 2D and FIG. 10B-FIG. 10C), plasmids (Table 2, construct MF72-MF80) expressing different transcription factor variants and a co-translational mCherry were stably integrated in these two monoclonal lines. After puromycin selection, cells were transferred into media containing 100 nM AP1903+10 μM TMP to permit self-activation, and measured by flow cytometry after another 72 hours. The mCherry+ cell population was gated for analysis. Protein variants through stable integration were introduced, instead of transient transfection, to avoid nonspecific transcriptional interference by transient high expression of proteins during transfection, and to test inhibition of self-activation in a cellular environment better mimicking the MultiFate-2 and MultiFate-3 stable cell lines.

Fluorescence Activated Cell Sorting (FACS)

To separate MultiFate cells in distinct states for subsequent experiments (FIG. 3A-FIG. 5D), cells were harvested and resuspended in sorting buffer (BD FACS Pre-Sort Buffer) supplemented with 1 U/ml DNAse I, AP1903 and TMP. Cells were then sorted into media containing different concentrations AP1903 and TMP, according to the experiments. Cell sorting was performed by Caltech Flow Cytometry Facility.

Flow Cytometry Measurement of Long-Term State Stability of MultiFate Cells

To characterize long-term state stability of MultiFate cells in different media conditions (FIG. 3A-FIG. 5D), 4% of cells were trypsinized and transferred into fresh media with the same condition every three days. The remaining cells were resuspended in flow cytometry buffer and analyzed by a flow cytometer. The resulting two-dimensional (or three-dimensional for MultiFate-3) fluorescence intensity plots were then divided into four quadrants (or eight octants) by an empirical threshold in each of the two (or three) fluorescence channels. The exact values of empirical thresholds for different MultiFate cell lines are slightly different due to expression differences and are provided above in the description of FIG. 14A-FIG. 16B and FIG. 22A-FIG. 22H above. The percentage of cells in quadrants (or octants) were then calculated for each sample. The mean percentage of cells across three samples was plotted as a square (or a hexagon) with colored circles representing the percentages.

Time-Lapse Imaging

To visualize state stability of MultiFate cells (FIG. 3D and FIG. 5C), MultiFate cells from different states were mixed at equal ratio, and sparsely seeded in an imaging 24-well plate (μ-Plate 24 Well Black, ibidi) with media containing AP1903 and TMP. After 6 or 12 hours, media was aspirated to remove unattached cells and fresh media containing AP1903 and TMP was added. Time-lapse images were acquired on an inverted Olympus IX81 fluorescence microscope with Zero Drift Control (ZDC), an ASI 2000XY automated stage, a Photometrics 95B camera (Teledyne Photometrics) and a 20×UPlanS/Apo objective (0.75 NA, Olympus). Fluorescent proteins were excited with an X-Cite XLED1 light source (Lumen Dynamics). Images were automatically acquired every hour, controlled by MetaMorph software (Molecular Devices). Cells were kept in a custom-made environmental chamber enclosing the microscope, controlling a humidified, 37° C. and 5% CO₂ atmosphere. Media was changed every three days.

Measuring Doubling Time of MultiFate Cells in Different States

To measure the doubling time of different MultiFate lines in different states (FIG. 13A-FIG. 13E), cells were first separated from different states using cell sorting. Cells from OFF state were then cultured in regular media and cells from all other states were cultured in media containing AP1903 and TMP, so that cells do not change state during measurement. For each measurement, the same number of cells were plated in two wells in a 48-well plate. One well was counted after 24 (or 48) hours, and the other well was counted after 72 hours. Note that the wells were still sub-confluent at 72 hours. The doubling time is then

$${τ}_{D} = \frac{Δ t}{{log}_{2} ( {N}_{{t}_{2}} - {N}_{{t}_{1}} )} ,$$

where Δt is the time difference between the two timepoints, Nₜ_{₁} is the cell number at 24 (or 48) hours, and Nₜ_{₂} is the cell number at 72 hours.

Robustness Analysis of MultiFate Circuit

All the stable fixed points attract surrounding cells in the deterministic model. In some embodiments, random transcription factor concentration fluctuations arose from stochasticity of chemical reactions (intrinsic noise) such as transcription and translation may switch cells from one stable fixed point to another. The robustness of a stable fixed point is measured by how infrequent cells from that stable fixed point spontaneously switch to other fixed points due to intrinsic noise. Gillespie simulations were used to analyze the robustness of MultiFate circuit (FIG. 5D, FIG. 27A-FIG. 27C and FIG. 28A-FIG. 28C). Molecular reactions and their propensities for Gillespie simulation are listed in Table 4. To quantify the robustness of a stable fixed point, trajectories of cells starting from that stable fixed point can be simulated first. Then the robustness of that fixed point can be quantified by a robustness score, defined by the fraction of cells not switching out of that stable fixed point at the end of simulation (1000 hours). Without being bound by any particular theory, the higher the robustness score, the more robust a stable fixed point is against intrinsic noise. In FIG. 5D, robust stable fixed points have robustness scores greater than 0.9, which means less than 10% of cells spontaneously escape at the end of stochastic simulations.

Attractor Basin Analysis of MultiFate Circuit with N Transcription Factors

Without being bound by any particular theory, the attractor basin volume of a stable fixed point goes to infinity (except for the fixed point with all transcription factors OFF) if the concentration of each transcription factor has no limit. However, in some embodiments, transcription factor concentrations are bounded by their maximum expression levels. Consequently, in some embodiments, attractor basin volumes are finite, and volumes of different basins can be compared. In the non-dimensionalized model, transcription factor concentrations are confined to the interval [α, α+β], which corresponds to the equilibrium concentrations when a transcription factor is not self-activating and when it is fully self-activating, respectively. To calculate the approximate volumes of attractor basins (FIG. 27A-FIG. 27C), a N-dimensional concentration grid was initialized with k points in each dimension, spaced at equal linear intervals, for a total of k^{N} points. Using these grid points as initial conditions, the differential equations were numerically solved to compute forward trajectories to their final stable fixed point, using the expanded MultiFate-N model (see below). Each grid point was labeled based on which stable fixed point its trajectory ends at. The attractor basin volume of a stable fixed point was then approximated as

$${V}_{i} = \frac{\begin{matrix}number of grid points \\ that end at fixed point i\end{matrix} }{{k}^{N}} \times {β}^{N} \qquad ( Equation 9 )$$

where β^{N} represents the total phase space volume.

Parameter Screening of MultiFate-2 and MultiFate-3 Circuits

Each parameter dependency plot in FIG. 6B and FIG. 7B represents a field of 100×100 points. The color of each point denotes the multistability type generated through MultiFate-2 (FIG. 6B) or MultiFate-3 (FIG. 7B) with the indicated parameter combination. To identify the multistability type of each parameter set, a 2- or 3-dimensional grid was initialized in the 2- or 3-dimensional space of transcription factor concentrations, for MultiFate-2 and MultiFate-3, respectively, with eight values for each transcription factor concentration. Using these grid points as starting points, trajectories were computed using the MultiFate differential equations. The end points of these trajectories were then grouped into clusters and the centers (center of mass) of these clusters were used as estimated locations of stable fixed points. The stability of the estimated fixed points were double-checked using standard linear stability analysis type by the locations of these stable fixed points.

Asymmetry Parameter Fitting of MultiFate-2 and MultiFate-3 Lines

While symmetric MultiFate-2 and MultiFate-3 models (See, “Design of the MultiFate Circuit” above) accurately predict the number of stable states for the experimental MultiFate-2 and MultiFate-3 circuits in different protein stability regimes, they cannot, in some embodiments, explain the bias of cells towards certain stable states when they transition away from an unstable state (for example, MultiFate-2.3 cells almost exclusively transition from the unstable OFF state to the B-only state in High TMP condition). Without being bound by any particular theory, this may be because the dynamics of different transcription factors in the experimental MultiFate lines are asymmetric due to differences in the zinc fingers used, copy numbers of integrated cassettes and other factors. To explain the dynamics of different MultiFate lines, asymmetry parameters were added into the symmetric MultiFate models to construct asymmetric MultiFate models (See “Asymmetric MultiFate model” below). To find the best fitted asymmetry parameter set for MultiFate-2 lines, 16 points for each of the four asymmetry parameters were uniformly sampled (r, m, k, γ, each ranging from 0.5 to 2) and a four-dimensional parameter space was constructed consisting of 164=65536 parameter sets. Parameter sets that do not generate the expected numbers of stable states in High TMP (3 states) and Low TMP (2 states) conditions were first filtered out. For the remaining parameter sets, the Gillespie algorithm was used to simulate the exit of 200 cells from the OFF state in both High TMP and Low TMP conditions, and from the A+B state in the Low TMP condition (3 simulations total for each parameter set). Cell fractions at the end of simulations were then compared with cell fractions at the end of continuous culture (cf. FIG. 14A-FIG. 16B, FIG. 22A-FIG. 22H, FIG. 24A-FIG. 24H, FIG. 25A-FIG. 25H and other two replicates) by calculating the mean squared error (MSE). The best fitted parameter set for each MultiFate-2 line is the one that has the lowest MSE. Finally, the parameter fitting results were validated by simulating 400 cells starting from OFF, A-only, B-only and A+B state in both High TMP and Low TMP conditions (8 simulations) and the simulated cell fractions were plotted side-by-side with experimental cell fractions in FIG. 20A.

For the MultiFate-3 line, since it is constructed from MultiFate-2.2 line, around the best fitted parameter set for MultiFate-2.2 was first chosen for each of the first four asymmetry parameters (r, m, K, γ, each sampled 3 points including the best fitted parameter for MultiFate-2.2 and +/−0.1). For the four new asymmetry parameters (r₂, m₂, k₂, γ₂ for the new TF C), 16 points for each of the four new asymmetry parameters ranging from 0.5 to 2 were again uniformly sampled. This results in an 8-dimensional parameter space consisting of 3⁴×16⁴=5308416 parameter sets. Using the same method for MultiFate-2 fitting, the best fitted parameter set for the MultiFate-3 line was found, the parameter fitting results were validated by simulating 400 cells starting from 8 states in High TMP, Intermediate TMP and Low TMP conditions (24 simulations) and the simulated cell fractions were plotted side-by-side with experimental cell fractions in FIG. 20B.

### Additional Methods

Non-Dimensionalization of MultiFate Model

As provided above (See, “Design of the MultiFate Circuit”), each ordinary differential equation (ODE) for [Aₜₒₜ] and [Bₜₒₜ] consists of three terms: (i) a basal protein production rate α, (ii) a Hill function describing self-activation dynamics with maximal rate β, Hill coefficient n, and half-maximal activation at a homodimer concentration of K_{M}, and (iii) a protein removal (dilution and degradation) rate δ. One can then write:

$$\begin{matrix}\frac{d [ {A}_{tot} ]}{d t} = α + \frac{{β [ {A}_{2} ]}^{n}}{{K}_{M}^{n} + {[ {A}_{2} ]}^{n}} - δ [ {A}_{tot} ] & ( Equation 10 ) \\ \frac{d [ {B}_{tot} ]}{d t} = α + \frac{{β [ {B}_{2} ]}^{n}}{{K}_{M}^{n} + {[ {B}_{2} ]}^{n}} - δ [ {B}_{tot} ] & ( Equation 11 )\end{matrix}$$

Dimerization dynamics occur on a faster timescale than protein production and degradation. This separation of timescales permits the assumption that the distribution of monomer and dimer states remains close to equilibrium, generating the following relationships between the concentrations of monomers ([A] and [B]), and dimers ([A₂], [B₂], and [AB]) based on the law of mass action:

[A]²=K_{d}[A₂] (Equation 3)

[B]²=K_{d}[B₂] (Equation 4)

2[A][B]=K_{d}[AB] (Equation 5)

Here, because the two transcription factors share the same dimerization domain, homo- and hetero-dimerization are assumed to occur with equal dissociation constants, K_{d}. When deriving these three equations from the law of mass action, each monomer is counted twice in homodimerization reactions, and is counted once in the heterodimerization reaction, thus a factor of two is introduced in the third equation to account for this statistical difference. Additionally, conservation of mass implies that [Aₜₒₜ]=[A]+[AB]+2[A₂] (Equation 6), with a similar relationship for B.

Solving these equations produces expressions for the concentrations of the activating homodimers in terms of the total concentrations of A and B:

$$\begin{matrix}[ {A}_{2} ] = \frac{{2 [ {A}_{tot} ]}^{2}}{\begin{matrix}{K}_{d} + 4 ( [ {A}_{tot} ] + [ {B}_{tot} ] ) + \\ \sqrt{{K}_{d}^{2} + 8 ( [ {A}_{tot} ] + [ {B}_{tot} ] ) {K}_{d}}\end{matrix}} & ( Equation 7 ) \\ [ {B}_{2} ] = \frac{{2 [ {B}_{tot} ]}^{2}}{\begin{matrix}{K}_{d} + 4 ( [ {A}_{tot} ] + [ {B}_{tot} ] ) + \\ \sqrt{{K}_{d}^{2} + 8 ( [ {A}_{tot} ] + [ {B}_{tot} ] ) {K}_{d}}\end{matrix}} & ( Equation 8 )\end{matrix}$$

To non-dimensionalize the model, time can be rescaled in units of δ⁻¹, and concentrations in units of K_{M}. This gives: t←tδ, [Aₜₒₜ]←[Aₜₒₜ]/K_{M}, [Bₜₒₜ]←[Bₜₒₜ]/K_{M}, A₂]←[A₂]/K_{M}, [B₂]←[B₂]/K_{M}, α←α/(K_{M}δ), β←β/(K_{M}δ), K_{d}←K_{d}/K_{M}.

Here, the quantity to the left of the arrow is the parameter in the non-dimensionalized system. Thus, in the first assignment, the non-dimensionalized time, t, is equal to dimensionalized time multiplied by δ. The system can then be written using the non-dimensionalized quantities:

$$\begin{matrix}\frac{δ d {K}_{M} [ {A}_{tot} ]}{d t} = {K}_{M} δ α + \frac{{K}_{M} δ {β ( {K}_{M} [ {A}_{2} ] )}^{n}}{{K}_{M}^{n} + {( {K}_{M} [ {A}_{2} ] )}^{n}} - δ {K}_{M} [ {A}_{tot} ] & ( Equation 12 ) \\ \frac{δ d {K}_{M} [ {B}_{tot} ]}{d t} = {K}_{M} δ α + \frac{{K}_{M} δ {β ( {K}_{M} [ {B}_{2} ] )}^{n}}{{K}_{M}^{n} + {( {K}_{M} [ {B}_{2} ] )}^{n}} - δ {K}_{M} [ {B}_{tot} ] & ( Equation 13 ) \\ and & \\ {K}_{M} [ {A}_{2} ] = \frac{2 {{K}_{M}^{2} [ {A}_{tot} ]}^{2}}{{K}_{M} {K}_{d} + 4 {K}_{M} ( [ {A}_{tot} ] + [ {B}_{tot} ] ) + \sqrt{{K}_{M}^{2} {K}_{d}^{2} + 8 {K}_{M}^{2} ( [ {A}_{tot} ] + [ {B}_{tot} ] ) {K}_{d}}} & ( Equation 14 ) \\ {K}_{M} [ {B}_{2} ] = \frac{2 {{K}_{M}^{2} [ {B}_{tot} ]}^{2}}{{K}_{M} {K}_{d} + 4 {K}_{M} ( [ {A}_{tot} ] + [ {B}_{tot} ] ) + \sqrt{{K}_{M}^{2} {K}_{d}^{2} + 8 {K}_{M}^{2} ( [ {A}_{tot} ] + [ {B}_{tot} ] ) {K}_{d}}} & ( Equation 15 )\end{matrix}$$

After canceling δ and K_{M} from both side of equations, the non-dimensionalized MultiFate model can be obtained:

$$\begin{matrix}\frac{d [ {A}_{t o t} ]}{d t} = α + \frac{{β [ {A}_{2} ]}^{n}}{1 + {[ {A}_{2} ]}^{n}} - [ {A}_{t o t} ] & ( Equation 1 ) \\ \frac{d [ {B}_{t o t} ]}{d t} = α + \frac{{β [ {B}_{2} ]}^{n}}{1 + {[ {B}_{2} ]}^{n}} - [ {B}_{t o t} ] & ( Equation 2 ) \\ [ {A}_{2} ] = \frac{{2 [ {A}_{t o t} ]}^{2}}{\begin{matrix}{K}_{d} + 4 ( [ {A}_{t o t} ] + [ {B}_{t o t} ] ) + \\ \sqrt{{K}_{d}^{2} + 8 ( [ {A}_{t o t} ] + [ {B}_{t o t} ] ) {K}_{d}}\end{matrix}} & ( Equation 7 ) \\ [ {B}_{2} ] = \frac{{2 [ {B}_{t o t} ]}^{2}}{\begin{matrix}{K}_{d} + 4 ( [ {A}_{t o t} ] + [ {B}_{t o t} ] ) + \\ \sqrt{{K}_{d}^{2} + 8 ( [ {A}_{t o t} ] + [ {B}_{t o t} ] ) {K}_{d}}\end{matrix}} & ( Equation 8 )\end{matrix}$$

This non-dimensionalization leaves four parameters: rescaled basal protein production rate, α, rescaled maximal protein production rate in the Hill function, β, rescaled dimerization dissociation constant, K_{d}, and Hill coefficient n.

Apart from these four parameters, the mathematical model was used to predict how protein stability controls the number of stable fixed points in many parts of this example. In the non-dimensionalized model, the protein degradation rate, δ, does not appear explicitly but enters through the rescaling of α and β by (δK_{M})⁻¹ as shown above. Thus, tuning protein stability is equivalent to multiplying both α and β by a common factor, referred to herein as a “protein stability factor.”

MultiFate-2 Model Incorporating External Inputs

To allow external control of MultiFate circuit by inducers, binding sites for ERT2-Gal4 (induced by 4-OHT) and Tet3G (induced by Dox) at the promoters of TF A self-activation construct (Table 2, MF84) and TF B self-activation construct (Table 2, MF64) were designed, respectively. These two constructs were used to make the switchable MultiFate-2.3 cells (Table 3), in which the expression of TF A and TF B can be controlled by 4-OHT and Dox, respectively. Since promoters of these self-activation cassettes contain binding sites for both inducer-responsive activators (ERT2-Gal4 or Tet3G) and zinc finger transcription factor homodimers, transcriptional activation of these cassettes follows an OR logic, i.e., promoter is activated when inducer-responsive activators or zinc finger transcription factor homodimers are bound. To model OR activation logic, the Hill functions in the non-dimensionalized MultiFate model were modified by adding an ind₁ (or ind₂) term, which represents the activation strength of the ERT2-Gal4 (or Tet3G) system, to both numerator and denominator:

$$\begin{matrix}\frac{d [ {A}_{t o t} ]}{d t} = α + \frac{β ( {[ {A}_{2} ]}^{n} + i n {d}_{1} )}{1 + ( {[ {A}_{2} ]}^{n} + i n {d}_{1} )} - [ {A}_{t o t} ] & ( Equation 16 ) \\ \frac{d [ {B}_{t o t} ]}{d t} = α + \frac{β ( {[ {B}_{2} ]}^{n} + i n {d}_{2} )}{1 + ( {[ {B}_{2} ]}^{n} + i n {d}_{2} )} - [ {B}_{t o t} ] & ( Equation 17 )\end{matrix}$$

In the experiments (FIG. 4B and FIG. 21A-FIG. 21C), only saturating concentrations of 4-OHT or Dox were used to achieve a full activation of cassettes. To model this, an ind₁ (or ind₂)=100 can be chosen when inducer is added, so that the Hill function

$$\frac{β ( {[ {A}_{2} ]}^{n} + i n {d}_{1} )}{1 + ( {[ {A}_{2} ]}^{n} + i n {d}_{1} )} ( or \frac{β ( {[ {B}_{2} ]}^{n} + i n {d}_{2} )}{1 + ( {[ {B}_{2} ]}^{n} + i n {d}_{2} )} )$$

is close to its maximum 1, representing full promoter activation when a saturating concentration of inducer is added. When there is no inducer, ind₁ (or ind₂)=0. Using this modified model, the state-switching dynamics shown in FIG. 8A-FIG. 8B were simulated.

MultiFate Model Expanded to N Transcription Factors

The minimal MultiFate-2 model can be expanded to include more transcription factors. To start, the distribution of transcription factors X₁, X₂, X₃, . . . X_{N} can be considered among different dimerization states. For each transcription factor, the total concentration can be expressed as,

$$[ {X}_{tot , i} ] = [ {X}_{i} ] + {\sum }_{j \neq i} [ {X}_{i} ] [ {X}_{j} ] + 2 [ {X}_{2 , i} ] \\ for i = 1 , 2 , 3 , … N \qquad ( Equation 18 )$$

Here [Xᵢ] denotes the concentration of transcription factor Xᵢ monomers, [X_{2,i}] denote the concentration of homodimers, and [Xᵢ][Xⱼ] denote the concentration of heterodimers formed by Xᵢ and Xⱼ (i≠j). As mentioned above, homo- and hetero-dimerization can be assumed to occur with equal dissociation constants, K_{d}, reflecting the use of the same dimerization domain for both proteins. Because dimerization dynamics occur on a faster timescale than protein production and degradation, the protein dimerization states approximately follow their equilibrium values:

[Xᵢ]²=K_{d}[X_{2,i}] (Equation 19)

2[Xᵢ][Xⱼ]=K_{d}[Xᵢ][Xⱼ] (Equation 20)

for i=1, 2, 3, . . . N and i≠j

Solving these equations produces an expression for the concentrations of the activating homodimers in terms of the total concentrations of all transcription factor species:

$$[ {X}_{2 , i} ] = \frac{{2 [ {X}_{t o t , i} ]}^{2}}{{K}_{d} + 4 \sum [ {X}_{t o t , i} ] + \sqrt{{K}_{d}^{2} + 8 {K}_{d} \sum [ {X}_{t o t , i} ]}} \\ for i = 1 , 2 , 3 , … N \qquad ( Equation 21 )$$

With these expressions, protein production and degradation dynamics can be described using ODEs for [X_{tot,i}] in a similar way as provided above (See, “Design of the MultiFate Circuit”). After non-dimensionalization and adding asymmetric parameters, ODEs for protein production and degradation in the expanded MultiFate model can be obtained:

$$\frac{d [ {X}_{tot , i} ]}{d t} = {r}_{i} α + \frac{{m}_{i} {β [ {X}_{2 , i} ]}^{n}}{{κ}_{i}^{n} + {[ {X}_{2 , i} ]}^{n}} - {γ}_{i} [ {X}_{tot , i} ] \\ for i = 1 , 2 , 3 , … N \qquad ( Equation 22 )$$

where α represents the basal protein production, β represents the maximal protein production rate in the Hill function, n represents Hill coefficient and rᵢ, mᵢ, kᵢ, and γᵢ represents the asymmetric parameters for transcription factor Xᵢ.

Asymmetric MultiFate Model

While the symmetric MultiFate-2 model precisely predicts many experimental results, some asymmetric behaviors were observed in MultiFate-2 lines in some embodiments. For example, MultiFate-2.2 cells in A+B state preferentially migrated towards A-only state when transferred from the High TMP condition to the Low TMP condition (FIG. 3C). Without being bound by any particular theory, this kind of asymmetric behavior may result from several potential differences among integrated gene cassettes: (i) A, B and C used three different ZF DNA-binding domains. As shown in FIG. 9A, they may, in some embodiments, have different binding affinity to ZF binding sites, resulting in different K_{M} values, and different activated transcriptional rates, resulting in different β values. (ii) The integration number of different genes and the genomic environment of different integrated cassettes can be different in different embodiments, which may affect basal and activated promoter expression, resulting in different values of α and β. (iii) Due to sequence differences in ZF DNA-binding domains, the protein stability can be, in some embodiments, different for different genes, resulting in different values of δ.

To analyze such asymmetries, distinct values of these parameters can be allowed, indicated by subscripted A, B or C. While asymmetry in these parameters can be allowed, symmetry in others can still be assumed. Specifically, the same Hill coefficient, n, and dissociation constant for dimerization, K_{d}, can be maintained for all factors, because they share the same transcriptional activation domain and the same homodimerization domain. With these assumptions, an asymmetric dimensionalized model can then be written:

$$\begin{matrix}\frac{d [ {A}_{t o t} ]}{d t} = {α}_{A} + \frac{{{β}_{A} [ {A}_{2} ]}^{n}}{{K}_{M A}^{n} + {[ {A}_{2} ]}^{n}} - {δ}_{A} [ {A}_{t o t} ] & ( Equation 23 ) \\ \frac{d [ {B}_{t o t} ]}{d t} = {α}_{B} + \frac{{{β}_{B} [ {B}_{2} ]}^{n}}{{K}_{M B}^{n} + {[ {B}_{2} ]}^{n}} - {δ}_{B} [ {B}_{t o t} ] & ( Equation 24 ) \\ \frac{d [ {C}_{t o t} ]}{d t} = {α}_{C} + \frac{{{β}_{C} [ {C}_{2} ]}^{n}}{{K}_{M C}^{n} + {[ {C}_{2} ]}^{n}} - {δ}_{c} [ {C}_{t o t} ] & ( Equation 25 )\end{matrix}$$

As above, the model can be non-dimensionalized by rescaling time in units of δ_{A}⁻¹, and concentrations in units of K_{MA}. This provides (after canceling K_{MA} and δ_{A}⁻¹ of from both sides of the equations):

$$\begin{matrix} \frac{d [ {A}_{t o t} ]}{d t} = {α}_{A} + \frac{{{β}_{A} [ {A}_{2} ]}^{n}}{1 + {[ {A}_{2} ]}^{n}} - [ {A}_{t o t} ] & ( Equation 26 ) \\ \frac{d [ {B}_{t o t} ]}{d t} = ( {α}_{B} / {α}_{A} ) {α}_{A} + \frac{( {β}_{B} / {β}_{A} ) {{β}_{A} [ {B}_{2} ]}^{n}}{{( {K}_{M B} / {K}_{M A} )}^{n} + {[ {B}_{2} ]}^{n}} - {δ}_{B} / {δ}_{A} [ {B}_{t o t} ] & ( Equation 27 ) \\ \frac{d [ {C}_{t o t} ]}{d t} = ( {α}_{C} / {α}_{A} ) {α}_{A} + \frac{( {β}_{C} / {β}_{A} ) {{β}_{A} [ {C}_{2} ]}^{n}}{{( {K}_{M C} / {K}_{M A} )}^{n} + {[ {C}_{2} ]}^{n}} - {δ}_{C} / {δ}_{A} [ {C}_{t o t} ] & ( Equation 28 )\end{matrix}$$

To further simplify these expressions, additional parameter ratios are defined as, r=αB/α_{A}, r₂=α_{C}/α_{A}, m=β_{B}/β_{A}, m₂=β_{C}/β_{A}, k=K_{MB}/K_{MA}, k₂=K_{MC}/K_{MA}, and γ=δ_{B}/δ_{A}, γ₂=δ_{C}/δ_{A}. In some embodiments, α=α_{A}, and β=β_{A} for notational simplicity. With these definitions, the ODEs for protein production and degradation in the asymmetric MultiFate model can be obtained:

$$\begin{matrix}\frac{d [ {A}_{t o t} ]}{d t} = α + \frac{{β [ {A}_{2} ]}^{n}}{1 + {[ {A}_{2} ]}^{n}} - [ {A}_{t o t} ] & ( Equation 1 ) \\ \frac{d [ {B}_{t o t} ]}{d t} = r α + \frac{m {β [ {B}_{2} ]}^{n}}{{κ}^{n} + {[ {B}_{2} ]}^{n}} - γ [ {B}_{t o t} ] & ( Equation 29 ) \\ \frac{d [ {C}_{t o t} ]}{d t} = {r}_{2} α + \frac{{m}_{2} {β [ {C}_{2} ]}^{n}}{{κ}_{2}^{n} + {[ {C}_{2} ]}^{n}} - {γ}_{2} [ {C}_{t o t} ] & ( Equation 30 )\end{matrix}$$

with the same expressions of the [A₂], [B₂] and [C₂] in terms of [Aₜₒₜ], [Bₜₒₜ] and [Cₜₒₜ] as provided above.

Four parameters are provided that represent different types of asymmetries: r (and r₂) represents the ratio of basal TF B (and TF C) production rates to that of TF A, m (and m₂) represents the ratio of maximal TF B (and TF C) production rates by self-activation to that of TF A, K (and K₂) represents the ratio of TF B (and TF C) homodimer concentrations for half-maximal activation to that of TF A, and γ (and γ₂) represents the ratio of TF B (and TF C) degradation rates to that of TF A. The symmetric MultiFate model is then, in some embodiments, a case where all asymmetry parameters equal to 1.

MultiFate Model with mRNA and Protein Dimerization Dynamics

The treatment above lumps the processes of mRNA transcription and protein translation together into a single gene expression step and uses a steady-state approximation for dimerization dynamics. This model works accurately in some embodiments to predict the number and locations of stable fixed points. To capture the dynamics of cells during bifurcation and state-switching events, a model that includes both mRNA and protein dimerization dynamics is provided below.

To incorporate mRNA dynamics into the model, TF A, TF B and TF C mRNAs, denoted [a], [b], and [c], can be assumed to be produced at a total rate equal to their basal transcription rate k₁ plus a homodimer-dependent transcriptional activation rate, which follows a Hill function of corresponding homodimer concentration, [A₂], [B₂] and [C₂], with maximal rate k₂, Hill coefficient n, and half-maximal activation at a homodimer concentration of K_{M}. Each mRNA species can be removed at a total rate (m_{RNA}. For generality, asymmetry parameters can be allowed, with r (or r₂) representing the ratio between A and B (or C) basal transcription rates (r=k_{1,B}/k_{1,A} or r₂=k_{1,C}/k_{1,A}), m (or m₂) representing the ratio between A and B (or C) maximal rates (m=k_{2,B}/k_{2,A} or m₂=k_{2,C}/k_{2,A}), and k (or k₂) representing the ratio between A and B half-maximal homodimer concentrations (k=K_{MB}/K_{MA} or k₂=K_{MC}/K_{MA}). This provides:

$$\begin{matrix}\frac{d [ a ]}{d t} = {k}_{1} + \frac{{{k}_{2} [ {A}_{2} ]}^{n}}{{K}_{M}^{n} + {[ {A}_{2} ]}^{n}} - {δ}_{m R N A} [ a ] & ( Equation 31 ) \\ \frac{d [ b ]}{d t} = r {k}_{1} + \frac{m {{k}_{2} [ {B}_{2} ]}^{n}}{{( κ {K}_{M} )}^{n} + {[ {B}_{2} ]}^{n}} - {δ}_{m R N A} [ b ] & ( Equation 32 ) \\ \frac{d [ c ]}{d t} = {r}_{2} {k}_{1} + \frac{{m}_{2} {{k}_{2} [ {C}_{2} ]}^{n}}{{( {κ}_{2} {K}_{M} )}^{n} + {[ {C}_{2} ]}^{n}} - {δ}_{m R N A} [ c ] & ( Equation 33 )\end{matrix}$$

Where one can let k₁=k_{1,A}, k₂=k_{2,A} and K_{M}=K_{MA} for notational simplicity.

Next, the dynamics of TF A and TF B proteins can be described in different dimerization forms with ODEs. For monomers of TF A and TF B, denoted [A] and [B], each equation consists of terms describing translation, protein removal, monomer association, dimer dissociation or conversion due to degradation of one of the constituent monomers. Here k, is the translation rate, d is the protein removal rate, kₒₙ is the monomer association rate and k_{off} is the dimer dissociation rate. [AB], [AC] and [BC] denotes the concentration of AB, AC and BC heterodimers.

The asymmetry parameter represents the ratio of TF A and TF B (or TF C) removal rates (γ=δ_{C}/δ_{A} or γ₂=δ_{C}/δ_{A}), and one can let δ=δ_{A} for notational simplicity:

$$\begin{matrix}\frac{d [ A ]}{d t} = {k}_{p} [ a ] - δ [ A ] - 2 {k}_{o n} ( {[ A ]}^{2} + [ A ] [ B ] + [ A ] [ C ] ) + {k}_{o f f} ( 2 [ {A}_{2} ] + [ A B ] + [ A C ] ) + γ δ [ AB ] + {γ}_{2} δ [ A C ] & ( Equation 34 ) \\ \frac{d [ B ]}{d t} = {k}_{p} [ b ] - γ δ [ B ] - 2 {k}_{o n} ( {[ B ]}^{2} + [ B ] [ A ] + [ B ] [ C ] ) + {k}_{o f f} ( 2 [ {B}_{2} ] + [ A B ] + [ B C ] ) + δ [ AB ] + {γ}_{2} δ [ BC ] \\ & ( Equation 35 ) \\ \frac{d [ C ]}{d t} = {k}_{p} [ c ] - {γ}_{2} δ [ C ] - 2 {k}_{o n} ( {[ C ]}^{2} + [ C ] [ A ] + [ C ] [ B ] ) + {k}_{o f f} ( 2 [ {C}_{2} ] + [ A C ] + [ B C ] ) + δ [ A C ] + γ δ [ BC ] & ( Equation 36 )\end{matrix}$$

For dimers, each equation consists of terms for removal, association, and dissociation:

$$\begin{matrix} \frac{d [ {A}_{2} ]}{d t} = - δ [ {A}_{2} ] + 2 {{k}_{o n} [ A ]}^{2} - {k}_{o f f} [ {A}_{2} ] & ( Equation 37 ) \\ \frac{d [ {B}_{2} ]}{d t} = - γ δ [ {B}_{2} ] + 2 {{k}_{o n} [ B ]}^{2} - {k}_{o f f} [ {B}_{2} ] & ( Equation 38 ) \\ \frac{d [ {C}_{2} ]}{d t} = - {γ}_{2} δ [ {C}_{2} ] + 2 {{k}_{o n} [ C ]}^{2} - {k}_{o f f} [ {C}_{2} ] & ( Equation 39 ) \\ \frac{d [ A B ]}{d t} = - δ [ A B ] - γ δ [ A B ] + 2 {k}_{o n} [ A ] [ B ] - {k}_{o f f} & ( Equation 40 ) \\ \frac{d [ A C ]}{d t} = - δ [ A B ] - {γ}_{2} δ [ A C ] + 2 {k}_{o n} [ A ] [ C ] - {k}_{o f f} [ A C ] & ( Equation 41 ) \\ \frac{d [ B C ]}{d t} = - γ δ [ B C ] - {γ}_{2} δ [ B C ] + 2 {k}_{on} [ B ] [ C ] - {k}_{off} [ BC ] & ( Equation 42 )\end{matrix}$$

Stochastic Modeling of MultiFate Circuits

To simulate the dynamics of the MultiFate-2 system during state-switching (FIG. 8A-FIG. 8B) and bifurcation (FIG. 4A) events, obtain best fitted asymmetry parameters for different MultiFate lines (FIG. 20A-FIG. 20B) and test the robustness of MultiFate against intrinsic biological noise (FIG. 5D, FIG. 27A-FIG. 28C), a stochastic model based on the same reactions represented by the ODEs in the above MultiFate-2 model with mRNA and protein dimerization dynamics was constructed. Molecular reactions and their propensities for Gillespie simulation are listed in Table 4. All terms have a concentration unit of molecule number per cell (either mRNA or protein), and a time unit of hour. Gillespie simulations were performed using the biocircuits Python package (https://pypi.org/project/biocircuits/) with physiologically reasonable parameters (see below). From existing literature and experimental measurements performed in this study, the physiologically reasonable regime for each dimensionalized parameter (K_{M}, δ, α, β, K_{d}, n, k₁, k₂, δ_{mRNA}, kₚ, kₒₙ, k_{off}) was estimated. Estimated values for these parameters are summarized in Table 4.

Since some measurement data are in the unit of concentration, while others are in the unit of molecules per cell, the number of molecules equivalent to 1 nM in a CHO-K1 cell were first estimated. The diameter of a CHO cell is ˜14 μm, from which the cell volume can be calculated to be around 1.4×10⁻¹² L (assuming it to be a sphere). Then, in some embodiments, 1 nM=1 nM×1.4×10⁻¹²L×6×10²³ molecules/mol≈800 molecules per CHO cell. Below, this value was used to convert between molecules per cell and molarity.

The concentration for half-maximal activation, K_{M}, of the ZF activator homodimer, can be used to rescale all concentrations in the non-dimensionalized MultiFate model (see above). The K_{M} of a monomeric ZF activator with a 3-finger Zif268 ZF domain was estimated to be ˜600 nM (assuming the volume of yeast cells to be 40 μm³). An in vitro study showed that by linking two Zif268 ZF domain with a linker, the resulting 6-finger ZF domain could bind to a 18 bp DNA target site almost 70-fold stronger than a single Zif268 domain binding to a 9 bp target site. Therefore, an activator with a 6-finger ZF domain was estimated to have a K_{M} of 600 nM/70≈8 nM. The ZF transcription factor homodimer should bind to 18 bp target DNA site in a similar fashion with how 6-finger ZF domain does, thus the K_{M} was estimated to be comparable to, or slightly larger than the range of 8-20 nM. Based on this reasoning, a K_{M}=10 nM=8000 molecules/cell was used in the model.

Next, parameters related to protein production and removal dynamics were estimated. A protein removal rate δ=0.1 hr⁻¹ for stable proteins was used, based on an in vivo measurement of proteome half-life dynamics in living human cells. The engineered ZF transcription factors have DHFR domains at their C-terminus, whose protein removal rate is controlled by TMP concentration. The dynamic range of this regulation is at least 20 fold. Therefore, the range of 8 was estimated to be between 0.1 hr⁻¹ and 2 hr⁻¹, under saturating TMP condition and no TMP condition, respectively. In the model, δ=0.1 hr⁻¹ was used for the “High TMP” condition, and δ=0.2 hr⁻¹ for the “Low TMP” condition.

k₂, δ_{mRNA} and kₚ were next estimated, which together are critical for establishing the levels and dynamics of mRNA and protein. In mammalian cells, average transcription rates were estimated to be ˜2 mRNA/(gene·hr). A total of 50-100 gene cassettes were estimated to be integrated during the construction of MultiFate-2, with 25-50 copies integrated each, for TF A and TF B. Maximal transcription rate k₂ was then estimated to be 50-100 mRNA/(cell·hr), and an intermediate value k_{z}=80 mRNA/(cell·hr) was used. For typical mRNA half-life, different studies have provided diverse values, ranging from 50 minutes to 9 hours. This corresponds to a mRNA removal rate δ_{mRNA} ranging from ln(2)/(9 hr)−ln(2)/(50 min)≈0.077−0.83 hr⁻¹. To take mRNA dilution from cell division into consideration, a value of δ_{mRNA}=0.7 hr⁻¹ was used, closer to the upper bound of the estimated range, in the stochastic model. For protein translation rate, a value of kₚ=140 proteins/(mRNA·hr) was used.

The value of β can be estimated from the above parameters. Since the mRNA removal rate is much higher than the protein removal rate, mRNA dynamics were assumed to be approximately at steady state on the timescale of δ⁻¹. This assumption enabled estimation of the maximal protein production rate in the Hill function as β=k₂×kₚ/δ_{mRNA}=16000 proteins/(cell·hr)=20 nM/hr. In the experiment, there was a fluorescence expression difference of around 25-50 fold observed between ON and OFF states (FIG. 3B). (The estimate of the OFF level is not limited by autofluorescence). Since expression in the OFF state comes from basal transcription, the basal transcription rate k₁ was estimated to be 25-50 fold smaller than k₂, giving a range of 1.152-4.608 mRNA/hr. From this, an intermediate value of k₁=3.2 mRNA/(cell·hr) was used. Similarly, the basal protein production rate can be estimated as α=k₁×kₚ/δ_{mRNA}=640 proteins/(cell·hr)=0.8 nM/hr.

The final parameter related to protein production dynamics is the Hill coefficient n. Transcription Hill coefficients range, in some embodiments, from 1-3.6. Here, a modest Hill coefficient of n=1.5 was used.

Finally, parameters related to protein dimerization were estimated. In some embodiments, the apparent dissociation constant K_{d} of FKBP homodimerization domain may depend on AP1903 concentrations. FKBP was compared with another homodimerization domain GCN4 that was used in this example (FIG. 2A), which was shown to have a K_{d} of 10-20 nM. ZF transcription factors with FKBP (FIG. 2B) more strongly activated the reporter in 100 nM AP1903 media than ZF transcription factors with GCN4 did (FIG. 9B, BCRZF). Based on this observation, it was reasoned that in media containing 100 nM AP1903, the apparent dissociation constant K_{d}≤10 nM. An estimate of K_{d}=10 nM=8000 molecules/cell was used in the model. For monomer association rate kₒₙ, an intermediate value in the range of diffusion-limited association rates of kₒₙ=4×10⁵/(M·s)=1.8×10⁻³ cell/(protein·hr) was used. These two values together produce a dimer dissociation rate k_{off}=K_{d}×kₒₙ=14.4 hr¹.

Relationship Between Transcription Factor Concentrations and their Co-Expressed Fluorescence Proteins

The MultiFate models use the total concentrations of transcription factors as variables, whereas experimental MultiFate systems have the co-expressed fluorescent proteins as readouts. To understand the relationship between transcription factor concentrations ([Aₜₒₜ] and [Bₜₒₜ]), and their co-expressed fluorescent proteins, denoted FPA and FPB, equations describing the dynamics of immature fluorescent proteins ([FPAᵢₘ] and [FPBᵢₘ]) and mature fluorescent proteins ([FPAₘ] and [FPBₘ]) were incorporated into the MultiFate-2 model. Since fluorescent proteins are co-expressed with transcription factors, the production term of fluorescent protein has a similar form

$$α + \frac{{β [ {A}_{2} ]}^{n}}{1 + {[ {A}_{2} ]}^{n}} ,$$

scaled by translational efficiency of IRES, denoted I_{eff}. Once produced, each immature fluorescent protein matures at a rate kₘₐₜ. Finally, either immature or mature fluorescent protein degrades and is diluted at a total rate δ_{FP}. A set of ODEs can then be added to the MultiFate-2 model:

$$\begin{matrix}\frac{d [ F P {A}_{i m} ]}{d t} = {l}_{e f f} ( α + \frac{{β [ {A}_{2} ]}^{n}}{1 + {[ {A}_{2} ]}^{n}} ) - {k}_{m a t A} [ F P {A}_{i m} ] - {δ}_{F P A} [ F P {A}_{i m} ] & ( Equation 43 ) \\ \frac{d [ F P {A}_{m} ]}{d t} = {k}_{m a t A} [ F P {A}_{i m} ] - {δ}_{F P A} [ F P {A}_{m} ] & ( Equation 44 )\end{matrix}$$

with a similar set of equations for [FPBᵢₘ] and [FPBₘ].

The estimate of I_{eff} can vary, and an I_{eff}=0.5 was used in the model. Maturation rate for mCherry (k_{matA}) and mCitrine (k_{matB}) were calculated to be 1.12 hr⁻¹ and 4.62 hr⁻¹, respectively, based on their estimated maturation time. In experimental MultiFate systems, all fluorescence proteins are fused with a PEST degron, which has a half-life of 2-6.5 hours, and a δ_{FP}=0.35 hr⁻¹ based on a 2-hour half-life was used. All rates may then be rescaled by the degradation rate of transcription factors (d) to obtain a non-dimensionalized model. Fluorescent protein translation, maturation and degradation were also incorporated into the MultiFate stochastic model and their propensities for Gillespie algorithm are listed in Table 4. These models were used to simulate the dynamics of transcription factor concentrations and fluorescence readouts in the same cells.

The stochastic model was first used to simulate the relationship between transcription factors concentration and their mature fluorescent proteins for a single self-activation module (FIG. 11A). 5 different transcription factor half-lives were chosen to obtain different distributions of transcription factor concentrations at steady states. While, in some embodiments, transcription factor concentrations can vary among these 5 conditions, fluorescent readouts show a strong bimodal distribution. This shows that positive autoregulation causes each transcription factor to express in a binary (high or low) fashion: when transcription factor concentration is higher than the ‘self-activation threshold’ (defined by TF concentration where [Homodimers]=1), activating transcription factor homodimers drive gene expression to ‘high’ state. Fluorescent proteins quickly saturate in the ‘high’ state, as shown by overlapping fluorescent protein distributions in the ‘high’ expression state (FIG. 11A, top middle), whereas transcription factor concentrations are additionally affected by the protein half-life (FIG. 11A, top left). This relationship between transcription factor concentrations and fluorescent readouts is further shown in the 2D scatter plot (FIG. 11A, top right).

Since each transcription factor in a MultiFate circuit positively autoregulates itself, each transcription factor can, in some embodiments, express in a roughly binary fashion. Any stable state is thus a combination of these binary expression states, allowing one to distinguish them through fluorescence readouts.

To test this, the single-cell dynamics of the MultiFate-2 circuit were simulated in either type II tristable regime or bistable regime (FIG. 11B). In both regimes, fluorescence readouts are well separated into distinct clusters. Each cluster can be unambiguously assigned to its underlying state. Consistent with results from a single self-activation module (FIG. 11A), although the TF A concentrations in A-only state (or TF B concentrations in B-only state) differ by more than 2 folds between the tristable regime and bistable regime, the mature mCherry (or mCitrine) only differ by about 10% and are almost indistinguishable on log scale. This matches with experimental observations (FIG. 14A-FIG. 16B). Together, these simulation results show that fluorescent reporters are sufficient to unambiguously identify the underlying states.

Finally, it was asked how well the fluorescence readouts track the dynamics of cell state transitions. To test this, MultiFate-2 cells switching from A-only state to B-only state in the bistable regime (similar to FIG. 8A-FIG. 8B) with fluorescent proteins of different maturation times and half-lives were simulated. In particular, the delay in time between when transcription factor concentrations cross the state boundary (from [Aₜₒₜ]>[Bₜₒₜ] to [Aₜₒₜ]<[Bₜₒₜ]) and when mature fluorescent proteins cross the state boundary (from [FPAₘ]>[FPBₘ] to [FPAₘ]<[FPBₘ]) was measured (FIG. 11C). Longer maturation time and longer fluorescent protein half-life both increase the time delay (FIG. 11D). Based on this, three fluorescent proteins that have short maturation times (mCherry 37 minutes, mCitrine 9 minutes, mTurquoise2 34 minutes) were chosen, and fused a PEST degron to their C-terminus to shorten their half-life to 2-6.5 hours. With these modifications, the time delay between fluorescent readouts and transcription factor dynamics can be, in some embodiments, less than 6 hours. This time delay is small when compared to total switching time in the experiments (FIG. 4B), which spans several days.

Robustness of MultiFate Circuit Against Intrinsic Noise

In both flow cytometry plots (FIG. 14A-FIG. 16B) and time-lapse images (FIG. 18A-FIG. 18B), a small number of cells were found to have spontaneously escaped from their original states due to biological noise. While these cells were rare, they led to investigation of the robustness of MultiFate against biological noise, especially intrinsic noise resulting from stochasticity of chemical reactions such as transcription, translation and degradation.

The Gillespie algorithm was used to simulate MultiFate circuits with intrinsic noise. Without being bound by any particular theory, it was hypothesized that different stable steady states may have different robustness against these intrinsic noises. Cells in states with smaller attractor basins may be more likely to spontaneously switch to other states due to random concentration fluctuations introduced by intrinsic noise. To test this, a MultiFate-3 type I quadrastable regime was chosen, in which the OFF state has a much smaller attractor basin compared with other states (FIG. 27A, left). As expected, many cells from the OFF state spontaneously turn on one of the transcription factors to switch to one of the other three states (FIG. 27A, center graph), while all cells from the B-only state (and similarly for the A-only and the C-only state) remain in their original state (FIG. 27A, right graph). The robustness was quantified by the fraction of cells not changing states at the end of simulations (1000 hours), denoted as “robustness score.” For this MultiFate-3 type I quadrastable regime, the OFF state has a smaller attractor basin and a lower robustness score than the other 3 states.

The relationship between attractor basin size and robustness score for a set of MultiFate-2 and MultiFate-3 regimes was next systematically tested (FIG. 27B-FIG. 27C). Robustness score was positively correlated with attractor basin size. However, in some embodiments, there is no clear cutoff on the size of the attractor basin to separate robust stable states (robustness score=1) and non-robust stable states, suggesting, without being bound by any particular theory, that robustness against intrinsic noise may be affected by other factors, such as, e.g., promoter leakiness and ultrasensitivity. Therefore, the robustness score was used directly to determine whether a fixed point is robust against biological noise in FIG. 5D and FIG. 28A-FIG. 28C.

Source of Ultrasensitivity in MultiFate Circuit

To generate multistability, a circuit should have both positive feedback and some levels of ultrasensitivity (i.e. effective Hill exponent greater than 1). Although transcriptional activation can exhibit some ultrasensitivity in mammalian cells (represented by the Hill coefficient of n=1.5 above), parameter screening revealed that MultiFate generates multistability even when n=1 (no ultrasensitivity from transcriptional activation). Without being bound by any particular theory, two features of the MultiFate circuit can provide this additional ultrasensitivity. First, transcription factors homodimerize to self-activate, and such cooperativity has been shown to introduce ultrasensitivity. Indeed, homodimerization results in a [Aₜₒₜ]² (or [Bₜₒₜ]²) term in the numerator of the expression for [A₂] (or [B₂]) in, e.g. “Design of the MultiFate Circuit” above, which is written again here for convenience:

$$\begin{matrix}[ {A}_{2} ] = \frac{{2 [ {A}_{t o t} ]}^{2}}{\begin{matrix}{K}_{d} + 4 ( [ {A}_{t o t} ] + [ {B}_{t o t} ] ) + \\ \sqrt{{K}_{d}^{2} + 8 ( [ {A}_{t o t} ] + [ {B}_{t o t} ] ) {K}_{d}}\end{matrix}} & ( Equation 7 ) \\ [ {B}_{2} ] = \frac{{2 [ {B}_{t o t} ]}^{2}}{\begin{matrix}{K}_{d} + 4 ( [ {A}_{t o t} ] + [ {B}_{t o t} ] ) + \\ \sqrt{{K}_{d}^{2} + 8 ( [ {A}_{t o t} ] + [ {B}_{t o t} ] ) {K}_{d}}\end{matrix}} & ( Equation 8 )\end{matrix}$$

In some embodiments, contribution of homodimerization to ultrasensitivity depends on the dimerization dissociation constant K_{d}. When homodimerization is strong (small K_{d}), the [Aₜₒₜ]+[Bₜₒₜ] term dominates in the denominator, which cancels with the quadratic term [Aₜₒₜ]2 or [Bₜₒₜ]² in the numerator. This makes the expression more linear, thus reducing the ultrasensitivity by homodimerization. Conversely, when dimerization is weak (large K_{d}), the K_{d}² term dominates in the denominator, which makes the expression more quadratic and increases the ultrasensitivity by homodimerization.

In some embodiments, a second source of ultrasensitivity comes from mutual inhibition through heterodimerization, a prevalent feature in biology also known as molecular titration, which has been shown to introduce ultrasensitivity. Here, opposite to the case with homodimerization, strong heterodimerization (small K_{d}) increases the ultrasensitivity introduced through molecular titration. In some embodiments, additional ultrasensitivity comes mainly from cooperativity through homodimerization when K_{d} is large, and mainly from molecular titration through heterodimerization when K_{d} is small (note that K_{d} values for homodimerization and heterodimerization are the same, since the same dimerization domain is used for all transcription factors in this example). Without being bound by any particular theory, this explains why the MultiFate circuit generates multistability in a wide K_{d} range (FIG. 6A-FIG. 7B).

Modulating Basal Expression by Modifying Synthetic Promoter Sequences

To obtain the desired type II tristability in the MultiFate-2 circuit, parameter screening (FIG. 6B) revealed that regimes with too high basal expression, where only A+B state is stable, and regimes with too low basal expression, where OFF state is stable should, in some embodiments, be avoided. When building MultiFate self-activation modules, original promoter basal expression was found to be too low (FIG. 29, construct 1), as shown by low level of spontaneous self-activation upon the addition of AP1903+TMP. Therefore, basal promoter expression was increased by modifying promoter sequences. When characterizing different ZF transcription factors, it was found that promoters containing the 9 bp binding site GACGCTGCT (Table 5) for 42ZF have higher basal expression. Basal promoter expression thus can be modulated by introducing different numbers of GACGCTGCT motifs at promoter regions (FIG. 29), and multiple repeats of this motif were introduced into final MultiFate constructs (Table 2).

Translating MultiFate Circuits into Other Cell Types

Many components used in MultiFate circuits can work across different cell types, including transcriptional activation domains and protein dimerization domains. For example, the transcriptional activation domain VP16 has been shown to work in multiple cell lines. In some embodiments, components in MultiFate can be modified when moving into a new context. For example, some zinc finger DNA-binding domains were first developed in yeast. While the original zinc finger domains work in the example provided herein, in some embodiments, more recently optimized synthetic zinc finger domains might be desirable. In some embodiments, basal promoter expression (transcriptional leakiness) can differ among cell lines and genomic contexts. In some embodiments, modulating basal expression can help engineer MultiFate circuits in additional cell contexts.

The general strategy to engineer MultiFate described herein can be, in some embodiments, applicable to multiple cell types. The basic module of MultiFate is the dimer-dependent self-activation circuit (FIG. 2C). One should first test whether the self-activation circuit can robustly sustain its own expression, and whether the self-activation is dimer-dependent, when translating MultiFate in a new cell type. If self-activation cannot robustly sustain its own expression, it may be, in some embodiments, that protein production rate (βin the model) is not high enough in the new cell type. In that case, one should consider using stronger transcriptional activation domains such as, e.g., VP64 or p65 to boost the mRNA transcription. In some embodiments, since β in the non-dimensionalized model is rescaled by K_{M} (homodimer concentration for half-maximal activation), a smaller K_{M} results in larger rescaled β. To achieve a smaller K_{M}, one can, in some embodiments, use optimized humanized zinc finger transcription factors which can increase binding affinity of the homodimers to the DNA and thus decrease the K_{M}. If self-activation is not dimer-dependent, one can consider modifying zinc fingers using the same mutation strategy of FIG. 2A and FIG. 9B.

Once the self-activation module works, one can follow the workflow in FIG. 12A-FIG. 12B to generate MultiFate cells. To make MultiFate-2 cells, one can first stably integrate two different self-activation modules into the desired cell types, then select for cells that can maintain a stable double-positive state using FACS or other methods. This results in desired MultiFate-2 cells, since the MultiFate-2 model (FIG. 6) shows that cells with a stable double-positive state can generate diverse multistability regimes. Similarly, to make MultiFate-3 cells, one can stably integrate a third self-activation module into the existing MultiFate-2 cells, then select for cells that can maintain a stable triple-positive state.

TABLE 1
LIST OF PHYSIOLOGICALLY REASONABLE PARAMETER REGIMES.

| Parameters | Model | Estimated values |
|---|---|---|
| K_{M} | Deterministic and | 10 nM or 8000 molecules/cell |
| | stochastic | |
| δ | Deterministic and | 0.1 hr⁻¹ for “High TMP” condition; |
| | stochastic | 0.2 hr⁻¹ for “Low TMP” condition |
| n | Deterministic and | 1.5 |
| | stochastic | |
| α | Deterministic | 0.8 nM/hr |
| β | Deterministic | 20 nM/hr |
| K_{d} | Deterministic | 10 nM |
| k₁ | Stochastic | 3.2 mRNA/hr |
| k₂ | Stochastic | 80 mRNA/hr |
| δ_{mRNA} | Stochastic | 0.7 hr⁻¹ |
| kₚ | Stochastic | 140 proteins/(mRNA*hr) |
| kₒₙ | Stochastic | 1.8 × 10⁻³ cell/(protein*hr) |
| k_{off} | Stochastic | 14.4 hr⁻¹ |
| I_{eff} | Deterministic and | 0.5 |
| | stochastic | |
| kₘₐₜ | Deterministic and | 1.12 hr⁻¹ for mCherry; 4.62 hr⁻¹ for |
| | stochastic | mCitrine |
| δ_{FP} | Deterministic and | 0.35 hr⁻¹ |
| | stochastic | |

Note:
1 nm is equivalent to 800 molecules per CHO cell.

TABLE 2
LIST OF PLASMIDS USED IN THIS STUDY AND THEIR USE IN THE FIGS.

| | | Usage in | FIG. or |
|---|---|---|---|
| Index | Construct name | this study | MultiFate lines |
| MF01 | PB-2x(ErbB2bs_ErbB2bs)-TATA-3xNLS-Citrine- | Reporter | FIG. 2A, FIG. |
| | BGHpA | | 9A, FIG. 9B, |
| | | | FIG. 9C |
| MF02 | PB-2x(37bs_37bs)-TATA-3xNLS-Citrine-BGHpA | Reporter | FIG. 2A, FIG. |
| | | | 9A, FIG. 9B, |
| | | | FIG. 9C |
| MF03 | PB-2x(42bs_42bs)-TATA-3xNLS-Citrine-BGHpA | Reporter | FIG. 2A, FIG. |
| | | | 9A, FIG. 9B, |
| | | | FIG. 9C |
| MF04 | PB-2x(92bs_92bs)-TATA-3xNLS-Citrine-BGHpA | Reporter | FIG. 2A, FIG. |
| | | | 9A, FIG. 9B |
| MF05 | PB-2x(97bs_97bs)-TATA-3xNLS-Citrine-BGHpA | Reporter | FIG. 2A, FIG. |
| | | | 9A, FIG. 9B |
| MF06 | PB-2x(BCRbs_BCRbs)-TATA-3xNLS-Citrine-BGHpA | Reporter | FIG. 2A, FIG. |
| | | | 9A, FIG. 9B, |
| | | | FIG. 9C |
| MF07 | PB-2x(HIVbs_HIVbs)-TATA-3xNLS-Citrine-BGHpA | Reporter | FIG. 2A, FIG. |
| | | | 9A, FIG. 9B |
| MF08 | PB-CAG-ErbB2ZFWT-VP48-mCherry-BGHpA | Transcription | FIG. 2A, FIG. |
| | | factors | 9A |
| MF09 | PB-CAG-ErbB2ZFWT-GCN4-VP48-mCherry- | Transcription | FIG. 2A, FIG. |
| | BGHpA | factors | 9A |
| MF10 | PB-CAG-ErbB2ZFR39A-VP48-mCherry-BGHpA | Transcription | FIG. 2A, FIG. |
| | | factors | 9A |
| MF11 | PB-CAG-ErbB2ZFR39A-GCN4-VP48-mCherry- | Transcription | FIG. 2A, FIG. |
| | BGHpA | factors | 9A |
| MF12 | PB-CAG-ErbB2ZFR2AR39A-VP48-mCherry-BGHpA | Transcription | FIG. 2A, FIG. |
| | | factors | 9A |
| MF13 | PB-CAG-ErbB2ZFR2AR39A-GCN4-VP48-mCherry- | Transcription | FIG. 2A, FIG. |
| | BGHpA | factors | 9A, FIG. 9C |
| MF14 | PB-CAG-ErbB2ZFR2AR39AR67A-VP48-mCherry- | Transcription | FIG. 2A, FIG. |
| | BGHpA | factors | 9A |
| MF15 | PB-CAG-ErbB2ZFR2AR39AR67A-GCN4-VP48- | Transcription | FIG. 2A, FIG. |
| | mCherry-BGHpA | factors | 9A |
| MF16 | PB-CAG-FKBP12F36V-BCRZFR39A-VP48- | Transcription | FIG. 2B |
| | mCherry-BGHpA | factors | |
| MF17 | PB-CAG-37ZFWT-VP48-mCherry-BGHpA | Transcription | FIG. 9B |
| | | factors | |
| MF18 | PB-CAG-37ZFWT-GCN4-VP48-mCherry-BGHpA | Transcription | FIG. 9B |
| | | factors | |
| MF19 | PB-CAG-37ZFR39A-VP48-mCherry-BGHpA | Transcription | FIG. 9B |
| | | factors | |
| MF20 | PB-CAG-37ZFR39A-GCN4-VP48-mCherry-BGHpA | Transcription | FIG. 9B |
| | | factors | |
| MF21 | PB-CAG-37ZFR2AR39A-VP48-mCherry-BGHpA | Transcription | FIG. 9B |
| | | factors | |
| MF22 | PB-CAG-37ZFR2AR39A-GCN4-VP48-mCherry- | Transcription | FIG. 9B |
| | BGHpA | factors | |
| MF23 | PB-CAG-37ZFR2AR39AR67A-VP48-mCherry- | Transcription | FIG. 9B |
| | BGHpA | factors | |
| MF24 | PB-CAG-37ZFR2AR39AR67A-GCN4-VP48- | Transcription | FIG. 9B |
| | mCherry-BGHpA | factors | |
| MF25 | PB-CAG-37ZFR2AR11AR39AR67A-VP48-mCherry- | Transcription | FIG. 9B |
| | BGHpA | factors | |
| MF26 | PB-CAG-37ZFR2AR11AR39AR67A-GCN4-VP48- | Transcription | FIG. 9B, FIG. |
| | mCherry-BGHpA | factors | 9C |
| MF27 | PB-CAG-42ZFR2AR39AR67A-VP48-mCherry- | Transcription | FIG. 9B |
| | BGHpA | factors | |
| MF28 | PB-CAG-42ZFR2AR39AR67A-GCN4-VP48- | Transcription | FIG. 9B, FIG. |
| | mCherry-BGHpA | factors | 9C |
| MF29 | PB-CAG-92ZFWT-VP48-mCherry-BGHpA | Transcription | FIG. 9B |
| | | factors | |
| MF30 | PB-CAG-92ZFWT-GCN4-VP48-mCherry-BGHpA | Transcription | FIG. 9B |
| | | factors | |
| MF31 | PB-CAG-92ZFR39A-VP48-mCherry-BGHpA | Transcription | FIG. 9B |
| | | factors | |
| MF32 | PB-CAG-92ZFR39A-GCN4-VP48-mCherry-BGHpA | Transcription | FIG. 9B |
| | | factors | |
| MF33 | PB-CAG-92ZFR2AR39A-VP48-mCherry-BGHpA | Transcription | FIG. 9B |
| | | factors | |
| MF34 | PB-CAG-92ZFR2AR39A-GCN4-VP48-mCherry- | Transcription | FIG. 9B |
| | BGHpA | factors | |
| MF35 | PB-CAG-92ZFR2AR39AR67A-VP48-mCherry- | Transcription | FIG. 9B |
| | BGHpA | factors | |
| MF36 | PB-CAG-92ZFR2AR39AR67A-GCN4-VP48- | Transcription | FIG. 9B |
| | mCherry-BGHpA | factors | |
| MF37 | PB-CAG-97ZFWT-VP48-mCherry-BGHpA | Transcription | FIG. 9B |
| | | factors | |
| MF38 | PB-CAG-97ZFWT-GCN4-VP48-mCherry-BGHpA | Transcription | FIG. 9B |
| | | factors | |
| MF39 | PB-CAG-97ZFR39A-VP48-mCherry-BGHpA | Transcription | FIG. 9B |
| | | factors | |
| MF40 | PB-CAG-97ZFR39A-GCN4-VP48-mCherry-BGHpA | Transcription | FIG. 9B |
| | | factors | |
| MF41 | PB-CAG-97ZFR2AR39A-VP48-mCherry-BGHpA | Transcription | FIG. 9B |
| | | factors | |
| MF42 | PB-CAG-97ZFR2AR39A-GCN4-VP48-mCherry- | Transcription | FIG. 9B |
| | BGHpA | factors | |
| MF43 | PB-CAG-BCRZF-VP48-mCherry-BGHpA | Transcription | FIG. 9B |
| | | factors | |
| MF44 | PB-CAG-BCRZF-GCN4-VP48-mCherry-BGHpA | Transcription | FIG. 9B |
| | | factors | |
| MF45 | PB-CAG-BCRZFR39A-VP48-mCherry-BGHpA | Transcription | FIG. 9B |
| | | factors | |
| MF46 | PB-CAG-BCRZFR39A-GCN4-VP48-mCherry- | Transcription | FIG. 9B, FIG. |
| | BGHpA | factors | 9C |
| MF47 | PB-CAG-HIV1ZFWT-VP48-mCherry-BGHpA | Transcription | FIG. 9B |
| | | factors | |
| MF48 | PB-CAG-HIV1ZFWT-GCN4-VP48-mCherry-BGHpA | Transcription | FIG. 9B |
| | | factors | |
| MF49 | PB-CAG-HIV1ZFR39A-VP48-mCherry-BGHpA | Transcription | FIG. 9B |
| | | factors | |
| MF50 | PB-CAG-HIV1ZFR39A-GCN4-VP48-mCherry- | Transcription | FIG. 9B |
| | BGHpA | factors | |
| MF51 | PB-CAG-HIV1ZFR2AR39A-VP48-mCherry-BGHpA | Transcription | FIG. 9B |
| | | factors | |
| MF52 | PB-CAG-HIV1ZFR2AR39A-GCN4-VP48-mCherry- | Transcription | FIG. 9B |
| | BGHpA | factors | |
| MF53 | PB-CAG-HIV1ZFR2AR39AR67A-VP48-mCherry- | Transcription | FIG. 9B |
| | BGHpA | factors | |
| MF54 | PB-CAG-HIV1ZFR2AR39AR67A-GCN4-VP48- | Transcription | FIG. 9B |
| | mCherry-BGHpA | factors | |
| MF55 | PB-CAG-HIV2ZFWT-VP48-mCherry-BGHpA | Transcription | FIG. 9B |
| | | factors | |
| MF56 | PB-CAG-HIV2ZFWT-GCN4-VP48-mCherry-BGHpA | Transcription | FIG. 9B |
| | | factors | |
| MF57 | PB-CAG-HIV2ZFR39A-VP48-mCherry-BGHpA | Transcription | FIG. 9B |
| | | factors | |
| MF58 | PB-CAG-HIV2ZFR39A-GCN4-VP48-mCherry- | Transcription | FIG. 9B |
| | BGHpA | factors | |
| MF59 | PB-CAG-HIV2ZFR2AR39A-VP48-mCherry-BGHpA | Transcription | FIG. 9B |
| | | factors | |
| MF60 | PB-CAG-HIV2ZFR2AR39A-GCN4-VP48-mCherry- | Transcription | FIG. 9B |
| | BGHpA | factors | |
| MF61 | PB-CAG-HIV2ZFR2AR39AR67A-VP48-mCherry- | Transcription | FIG. 9B |
| | BGHpA | factors | |
| MF62 | PB-CAG-HIV2ZFR2AR39AR67A-GCN4-VP48- | Transcription | FIG. 9B |
| | mCherry-BGHpA | factors | |
| MF63 | PB-TRE3G-6x42bs-6x(BCRbs_BCRbs)-miniCMV- | Self-activation | FIG. 2C |
| | NLS-FKBP12F36V-BCRZFR39A-VP16-NLS-DHFR- | construct | |
| | IRES-mCitrine-PEST-BGHpA | | |
| MF64 | PB-TRE3G-6x42bs-6x(37bs_37bs)-miniCMV-NLS- | Self-activation | FIG. 10A, |
| | FKBP12F36V-37ZFR2AR11AR39AR67A-VP16- | construct | MultiFate-2.1, |
| | NLS-DHFR-IRES-mCitrine-PEST-BGHpA | | MultiFate-2.3 |
| MF65 | PB-TRE3G-6x42bs-6x(92bs_92bs)-miniCMV-NLS- | Self-activation | FIG. 10A |
| | FKBP12F36V-92ZFR2AR39AR67A-VP16-NLS- | construct | |
| | DHFR-IRES-mCitrine-PEST-BGHpA | | |
| MF66 | PB-TRE3G-6x42bs-6x(97bs_97bs)-miniCMV-NLS- | Self-activation | FIG. 10A |
| | FKBP12F36V-97ZFR39A-VP16-NLS-DHFR-IRES- | construct | |
| | mCitrine-PEST-BGHpA | | |
| MF67 | PB-TRE3G-6x42bs-6x(ErbB2bs_ErbB2bs)-miniCMV- | Self-activation | FIG. 10A |
| | NLS-FKBP12F36V-ErbB2ZFR2AR39A-VP16-NLS- | construct | |
| | DHFR-IRES-mCitrine-PEST-BGHpA | | |
| MF68 | PB-TRE3G-6x42bs-6x(HIVbs_HIVbs)-miniCMV- | Self-activation | FIG. 10A |
| | NLS-FKBP12F36V-HIV1ZFR2AR39A-VP16-NLS- | construct | |
| | DHFR-IRES-mCitrine-PEST-BGHpA | | |
| MF69 | PB-TRE3G-6x42bs-6x(HIVbs_HIVbs)-miniCMV- | Self-activation | FIG. 10A |
| | NLS-FKBP12F36V-HIV2ZFR2AR39AR67A-VP16- | construct | |
| | NLS-DHFR-IRES-mCitrine-PEST-BGHpA | | |
| MF70 | PB-TRE3G-6x(42bs_42bs)-miniPromo- | Self-activation | FIG. 2D, FIG. |
| | 42ZFR2AR39AR67A-GCN4-VP48-DHFR-IRES- | construct | 10B-FIG. 10C |
| | mCitrine-PEST-BGHpA | | |
| MF71 | PB-TRE3G-6x(42bs_42bs)-miniPromo-FKBP12F36V- | Self-activation | FIG. 2D, FIG. |
| | 42ZFR2AR39AR67A-VP48-DHFR-IRES-mCitrine- | construct | 10B-FIG. 10C |
| | PEST-BGHpA | | |
| MF72 | PB-CAG-IRES-mCherry-PEST-BGHpA (Control) | Protein | FIG. 2D, FIG. |
| | | perturbations | 10B-FIG. 10C |
| MF73 | PB-CAG-BCRZFR39A-GCN4-VP48-IRES-mCherry- | Protein | FIG. 2D, FIG. |
| | PEST-BGHpA | perturbations | FIG. 10B-FIG. |
| | | | 10C |
| MF74 | PB-CAG-FKBP12F36V-BCRZFR39A-VP48-IRES- | Protein | FIG. 2D, FIG. |
| | mCherry-PEST-BGHpA | perturbations | 10B-FIG. 10C |
| MF75 | PB-CAG-BCRZFR39A-GCN4-IRES-mCherry-PEST- | Protein | FIG. 10B-FIG. |
| | BGHpA | perturbations | 10C |
| MF76 | PB-CAG-FKBP12F36V-IRES-mCherry-PEST- | Protein | FIG. 10B-FIG. |
| | BGHpA | perturbations | 10C |
| MF77 | PB-CAG-BCRZFR39A-VP48-IRES-mCherry-PEST- | Protein | FIG. 2D, FIG. |
| | BGHpA | perturbations | 10B-FIG. 10C |
| MF78 | PB-CAG-BCRZFR39A-IRES-mCherry-PEST-BGHpA | Protein | FIG. 10B-FIG. |
| | | perturbations | 10C |
| MF79 | PB-CAG-GCN4-IRES-mCherry-PEST-BGHpA | Protein | FIG. 10B-FIG. |
| | | perturbations | 10C |
| MF80 | PB-CAG-VP48-IRES-mCherry-PEST-BGHpA | Protein | FIG. 10B-FIG. |
| | | perturbations | 10C |
| MF81 | PB-TRE3G-12x42bs-6x(BCRbs_BCRbs)-miniCMV- | Self-activation | MultiFate-2.1 |
| | NLS-FKBP12F36V-BCRZFR39A-VP16-NLS-DHFR- | construct | |
| | IRES-mCherry-PEST-BGHpA | | |
| MF82 | PB-TRE3G-12x42bs-10x(BCRbs_BCRbs)-miniCMV- | Self-activation | MultiFate-2.2, |
| | NLS-FKBP12F36V-BCRZFR39A-VP16-NLS-DHFR- | construct | MultiFate-3 |
| | IRES-mCherry-PEST-BGHpA | | |
| MF83 | PB-TRE3G-6x42bs-10x(37bs_37bs)-miniCMV-NLS- | Self-activation | MultiFate-2.2, |
| | FKBP12F36V-37ZFR2AR11AR39AR67A-VP16- | construct | MultiFate-3 |
| | NLS-DHFR-IRES-mCitrine-PEST-BGHpA | | |
| MF84 | PB-14xUAS-6x42bs-6x(BCRbs_BCRbs)-miniCMV- | Self-activation | MultiFate-2.3 |
| | NLS-FKBP12F36V-BCRZFR39A-VP16-NLS-DHFR- | construct | |
| | IRES-mCherry-PEST-BGHpA | | |
| MF85 | PB-14xUAS-12x42bs-10x(ErbB2bs_ErbB2bs)- | Self-activation | MultiFate-3 |
| | miniCMV-NLS-FKBP12F36V-ErbB2ZFR2AR39A- | construct | |
| | VP16-NLS-DHFR-IRES-mTurquoise2-PEST-BGHpA | | |
| MF86 | PB-EF1α-Tet3G-BGHpA | Inducible system | FIG. 2C, FIG. |
| | | | 2D, FIG. 10A- |
| | | | FIG. 10C |
| MF87 | PB-EF1α-Tet3G-P2A-ERT2-Gal4-BGHpA | Inducible system | All MultiFate |
| | | | cells |
| MF88 | PB-TRE3G-6x(BCRbs_BCRbs)-miniCMV-NLS- | Self-activation | FIG. 29 |
| | FKBP12F36V-BCRZFR39A-VP16-NLS-FLAG- | construct | |
| | DHFR-IRES-mCherry-PEST-BGHpA | | |
| MF89 | PB-TRE3G-4x42bs-6x(BCRbs_BCRbs)-miniCMV- | Self-activation | FIG. 29 |
| | NLS-FKBP12F36V-BCRZFR39A-VP16-NLS-FLAG- | construct | |
| | DHFR-IRES-mCherry-PEST-BGHpA | | |
| MF90 | PB-TRE3G-6x42bs-6x(BCRbs_BCRbs)-miniCMV- | Self-activation | FIG. 29 |
| | NLS-FKBP12F36V-BCRZFR39A-VP16-NLS-FLAG- | construct | |
| | DHFR-IRES-mCherry-PEST-BGHpA | | |

Note:
PB = PiggyBac backbone;
TRE3G = Tet3G binding site;
UAS = ERT2-Gal4 binding site;
TATA, miniCMV, miniPromo are three different minimal promoters;
VP48, VP16 are two different transcriptional activation domains;
CAG = the constitutive CAG promoter (52);
EF1α = the constitutive EF1α promoter;
NLS = nuclear localization sequence;
IRES = internal ribosome entry site;
BGHpA = bovine growth hormone polyadenylation signal;
PEST = constitutive signal peptide for protein degradation (53);
42bs = both the 42ZF binding site and 9bp motif that increase promoter leakiness;
ZFbs_ZFbs = 18bp tandem ZF binding site pairs.

TABLE 3
LIST OF STABLE CELL LINES CONSTRUCTED FOR THIS STUDY AND THEIR USE IN THE FIGS.

| | Parental | Poly- or | Integrated | | Additional procedures to |
|---|---|---|---|---|---|
| Cell lines | cells | monoclonal | constructs | FIG. | screen monoclones |
| Tet3G- | CHO-K1 | Polyclonal | MF86 | FIG. 2C-2D, FIG. | N/A |
| expressing | | | | 10A-FIG. 10C | |
| CHO-K1 | | | | | |
| ERT2-Gal4- | CHO-K1 | Polyclonal | MF87 | FIG. 3A-FIG. 5D, | N/A |
| T2A-Tet3G | | | | FIG. 11A-FIG. 22H | |
| expressing | | | | | |
| CHO-K1 | | | | | |
| FKBP- | Tet3G- | Polyclonal | MF63 | FIG. 2C | N/A |
| BCRZFR39A- | expressing | | | | |
| VP48-DHFR | CHO-K1 | | | | |
| self-activation | | | | | |
| FKBP- | Tet3G- | Polyclonal | MF64 | FIG. 10A | N/A |
| 37ZFR2AR11 | expressing | | | | |
| AR39AR67A- | CHO-K1 | | | | |
| VP48-DHFR | | | | | |
| self-activation | | | | | |
| FKBP- | Tet3G- | Polyclonal | MF65 | FIG. 10A | N/A |
| 92ZFR2AR39 | expressing | | | | |
| AR67A-VP48- | CHO-K1 | | | | |
| DHFR self- | | | | | |
| activation | | | | | |
| FKBP- | Tet3G- | Polyclonal | MF66 | FIG. 10A | N/A |
| 97ZFR39A- | expressing | | | | |
| VP48-DHFR | CHO-K1 | | | | |
| self-activation | | | | | |
| FKBP- | Tet3G- | Polyclonal | MF67 | FIG. 10A | N/A |
| ErbB2ZFR2AR | expressing | | | | |
| 39A-VP48- | CHO-K1 | | | | |
| DHFR self- | | | | | |
| activation | | | | | |
| FKBP- | Tet3G- | Polyclonal | MF68 | FIG. 10A | N/A |
| HIV1ZFR2AR | expressing | | | | |
| 39A-VP48- | CHO-K1 | | | | |
| DHFR self- | | | | | |
| activation | | | | | |
| FKBP- | Tet3G- | Polyclonal | MF69 | FIG. 10A | N/A |
| HIV2ZFR2AR | expressing | | | | |
| 39AR67A- | CHO-K1 | | | | |
| VP48-DHFR | | | | | |
| self-activation | | | | | |
| FKBP- | CHO-K1 | Polyclonal | MF88 | FIG. 29 | N/A |
| BCRZFR39A- | | | | | |
| VP16-DHFR | | | | | |
| self-activation | | | | | |
| (with no 42bs | | | | | |
| in promoter) | | | | | |
| FKBP- | CHO-K1 | Polyclonal | MF89 | FIG. 29 | N/A |
| BCRZFR39A- | | | | | |
| VP16-DHFR | | | | | |
| self-activation | | | | | |
| (with 4x 42bs | | | | | |
| in promoter) | | | | | |
| FKBP- | CHO-K1 | Polyclonal | MF90 | FIG. 29 | N/A |
| BCRZFR39A- | | | | | |
| VP16-DHFR | | | | | |
| self-activation | | | | | |
| (with 6x 42bs | | | | | |
| in promoter) | | | | | |
| 42ZFR2AR39 | Tet3G- | Monoclonal | MF70 | FIG. 2D, FIG. 10B- | Obtained monoclone |
| AR67A- | expressing | | | FIG. 10C | candidates by limiting |
| GCN4-VP48- | CHO-K1 | | | | dilution, induced candidates |
| DHFR self- | | | | | with 10 μM TMP and |
| activation | | | | | selected the monoclone that |
| | | | | | spontaneously and |
| | | | | | homogenously self-activate |
| FKBP- | Tet3G- | Monoclonal | MF71 | FIG. 2D, FIG. 10B- | Obtained monoclone |
| 42ZFR2AR39 | expressing | | | FIG. 10C | candidates by limiting |
| AR67A-VP48- | CHO-K1 | | | | dilution, induced candidates |
| DHFR self- | | | | | with 100 nM AP1903 + 10 |
| activation | | | | | μM TMP and selected the |
| | | | | | monoclone that spontaneously |
| | | | | | and homogenously self- |
| | | | | | activate |
| MultiFate-2.1 | ERT2-Gal4- | Monoclonal | MF64, | FIG. 3C, FIG. 4B, | Induced the polyclonal |
| | T2A-Tet3G | | MF81 | FIG. 11A-FIG. 11D | population with 500 ng/ml |
| | expressing | | | | Dox for 12 hours, then washed |
| | CHO-K1 | | | | out Dox and changed to 100 |
| | | | | | nM AP1903 + 10 μM TMP for |
| | | | | | 3 days, FACS sorted |
| | | | | | monoclones that were |
| | | | | | mCherry+ and mCitrine+ |
| MultiFate-2.2 | ERT2-Gal4- | Monoclonal | MF82, | FIG. 3C, FIG. 12A- | Induced the polyclonal |
| | T2A-Tet3G | | MF83 | FIG. 12B | population with 500 ng/ml |
| | expressing | | | | Dox for 12 hours, then washed |
| | CHO-K1 | | | | out Dox and changed to 100 |
| | | | | | nM AP1903 + 10 μM TMP for |
| | | | | | 3 days, FACS sorted |
| | | | | | monoclones that were |
| | | | | | mCherry+ and mCitrine+ |
| MultiFate-2.3 | ERT2-Gal4- | Monoclonal | MF64, | FIG. 3C, FIG. 3D, | Induced the polyclonal |
| | T2A-Tet3G | | MF84 | FIG. 4A, FIG.13A- | population with 500 ng/ml |
| | expressing | | | FIG. 14C, FIG. 20A- | Dox and 75 nM 4-OHT for 12 |
| | CHO-K1 | | | FIG. 20B | hours, then washed out Dox |
| | | | | | and 4-OHT and changed to 100 |
| | | | | | nM AP1903 + 10 μM TMP for |
| | | | | | 3 days, FACS sorted |
| | | | | | monoclones that were |
| | | | | | mCherry+ and mCitrine+ |
| MultiFate-3 | MultiFate-2.2 | Monoclonal | MF85 | FIG. 5B-FIG. 5C, | Induced the polyclonal |
| | | | | FIG. 15A-FIG. 20B | population with 500 ng/ml Dox |
| | | | | | and 75 nM 4-OHT for 12 hours, |
| | | | | | then washed out Dox and |
| | | | | | 4-OHT and changed to 100 nM |
| | | | | | AP1903 + 10 μM TMP for 3 |
| | | | | | days, FACS sorted monoclones |
| | | | | | that were mCherry+, mCitrine+ |
| | | | | | and mTurquoise2+ |

Promoter structures of different MultiFate lines:
MultiFate-2.1
TF A promoter has Tet3G binding sites, 6x(BCRbs_BCRbs);
TF B promoter has Tet3G binding sites, 6x(37bs_37bs);
MultiFate-2.2
TF A promoter has Tet3G binding sites, 10x(BCRbs_BCRbs);
TF B promoter has Tet3G binding sites, 10x(37bs_37bs);
MultiFate-2.3
TF A promoter has ERT2-Gal4 binding sites, 6x(BCRbs_BCRbs);
TF B promoter has Tet3G binding sites, 6x(37bs_37bs);
MultiFate-3
TF A promoter has Tet3G binding sites, 10x(BCRbs_BCRbs);
TF B promoter has Tet3G binding sites, 10x(37bs_37bs);
TF C promoter has ERT2-Gal4 binding sites, 10x(ErbB2bs ErbB2bs);

TABLE 4
LIST OF MOLECULAR REACTIONS AND THEIR
PROPENSITIES FOR GILLESPIE SIMULATION.

| Reactions | Molecule update | Propensity |
|---|---|---|
| TF A mRNA transcription | a → a + 1 | ${k}_{1} + {k}_{2} \frac{{[ {A}_{2} ]}^{n}}{{K}_{M}^{n} + {[ {A}_{2} ]}^{n}}$ |
| TF A mRNA removal | a → a − 1 | δ_{mRNA}[a] |
| TF B mRNA transcription | b → b + 1 | ${rk}_{1} + {mk}_{2} \frac{{[ {B}_{2} ]}^{2}}{{( κ {K}_{M} )}^{n} + {[ {B}_{2} ]}^{n}}$ |
| TF B mRNA removal | b → b − 1 | δ_{mRNA}[b] |
| TF C mRNA transcription | c → c + 1 | ${r}_{2} {k}_{1} + {m}_{2} {k}_{2} \frac{{[ {C}_{2} ]}^{n}}{{( {κ}_{2} {K}_{M} )}^{n} + {[ {C}_{2} ]}^{n}}$ |
| TF C mRNA removal | c → c − 1 | δ_{mRNA}[c] |
| TF A protein translation | A → A + 1 | kₚ[a] |
| TF A protein removal | A → A − 1 | δ[A] |
| TF B protein translation | B → B + 1 | kₚ[b] |
| TF B protein removal | B → B − 1 | γδ[B] |
| TF C protein translation | C → C + 1 | kₚ[b] |
| TF C protein removal | C → C − 1 | γ₂δ[B] |
| TF A homodimerization | A → A − 2, A₂ → A₂ + 1 | kₒₙ[A]² × ([A] ≥ 2) |
| AA homodimer dissociation | A₂ → A₂ − 1, A → A + 2 | k_{off}[A₂] |
| TF B homodimerization | B → B − 2, B₂ → B₂ + 1 | kₒₙ[B]² × ([B] ≥ 2) |
| BB homodimer dissociation | B₂ → B₂ − 1, B → B + 2 | k_{off}[B₂] |
| TF C homodimerization | C → C − 2, C₂ → C₂ + 1 | kₒₙ[C]² × ([C] ≥ 2) |
| CC homodimer dissociation | C₂ → C₂ − 1, C → C + 2 | k_{off}[C₂] |
| AB heterodimerization | A → A − 1, B → B − 1, AB → AB + 1 | 2kₒₙ[A][B] |
| AB heterodimer dissociation | AB → AB − 1, A → A + 1, B → B + 1 | k_{off}[AB] |
| AC heterodimerization | A → A − 1, C → C − 1, AC → AC + 1 | 2kₒₙ[A][C] |
| AC heterodimer dissociation | AC → AC − 1, A → A + 1, C → C + 1 | k_{off}[AC] |
| BC heterodimerization | B → B − 1, C → C − 1, BC → BC + 1 | 2kₒₙ[B][C] |
| BC heterodimer dissociation | BC → BC − 1, B → B + 1, C → C + 1 | k_{off}[BC] |
| AA homodimer removal | A₂ → A₂ − 1 | δ[A₂] |
| BB homodimer removal | B₂ → B₂ − 1 | γδ[B₂] |
| CC homodimer removal | C₂ → C₂ − 1 | γ₂δ[C₂] |
| AB -> B due to A removal | AB → AB − 1, B → B + 1 | δ[AB] |
| AB -> A due to B removal | AB → AB − 1, A → A + 1 | γδ[AB] |
| AC -> C due to A removal | AC → AC − 1, C → C + 1 | δ[AC] |
| AC -> A due to C removal | AC → AC − 1, A → A + 1 | γ₂δ[AC] |
| BC -> C due to B removal | BC → BC − 1, C → C + 1 | γδ[BC] |
| BC -> B due to C removal | BC → BC − 1, B → B + 1 | γ₂δ[BC] |
| FPAᵢₘ protein translation | FPAᵢₘ → FPAᵢₘ + 1 | I_{eff}kₚ[a] |
| FPBᵢₘ protein translation | FPBᵢₘ → FPBᵢₘ + 1 | I_{eff}kₚ[b] |
| FPAᵢₘ protein maturation | FPAᵢₘ → FPAᵢₘ − 1, FPAₘ → FPAₘ + 1 | k_{matA}[FPAᵢₘ] |
| FPBᵢₘ protein maturation | FPBᵢₘ → FPBᵢₘ − 1, FPBₘ → FPBₘ + 1 | k_{matB}[FPBᵢₘ] |
| FPAᵢₘ protein removal | FPAᵢₘ → FPAᵢₘ − 1 | δ_{FPA}[FPAᵢₘ] |
| FPBᵢₘ protein removal | FPBᵢₘ → FPBᵢₘ + 1 | δ_{FPB}[FPBᵢₘ] |
| FPAₘ protein removal | FPAₘ → FPAₘ − 1 | δ_{FPA}[FPAₘ] |
| FPBₘ protein removal | FPAₘ → FPAₘ + 1 | δ_{FPB}[FPBₘ] |

Note:
A, B, C, A₂, B₂, C₂, AB, AC, BC represent proteins of monomer A, monomer B, monomer C, homodimer AA, homodimer BB, homodimer CC, heterodimer AB, heterodimer AC, heterodimer BC, respectively.
a, b, c represents mRNAs of A, B, C, respectively.
FPAᵢₘ, FPBᵢₘ, FPAₘ, FPBₘ, represents immature fluorescent protein A, immature fluorescent protein B, mature fluorescent protein A, mature fluorescent protein B, respectively.

TABLE 5
PROMOTER BINDING SITE AND ZINC FINGER
DOMAIN SEQUENCES.

| | SEQ ID | |
|---|---|---|
| NAME | NO | SEQUENCE |
| Basal | | GACGCTGCT |
| Expression | | |
| motif (42bs) | | |
| 42bs 42bs | SEQ ID | GACGCTGCTGACGCTGCT |
| | NO: 1 | |
| 37bs 37bs | SEQ ID | TGAGGACGTGTTGAGGACGT |
| | NO: 2 | GT |
| BCRbs BCRbs | SEQ ID | GCAGAAGCCGCAGAAGCC |
| | NO: 3 | |
| ErbB2bs ErbB2bs | SEQ ID | GCCGCAGTGGCCGCAGTG |
| | NO: 4 | |
| FKBP12F36V | SEQ ID | GVQVETISPGDGRTFPKRGQ |
| | NO: 5 | TCVVHYTGMLEDGKKVDSSR |
| | | DRNKPFKFMLGKQEVIRGWE |
| | | EGVAQMSVGQRAKLTISPDY |
| | | AYGATGHPGIIPPHATLVFD |
| | | VELLKLE |
| ErbB2ZFWT | SEQ ID | ERPFQCRICMRNFSRSDVLA |
| | NO: 6 | NHTRTHTGEKPFQCRICMRN |
| | | FSQSSTLTRHLRTHTGEKPF |
| | | QCRICMRNFSERQGLKRHLK |
| | | THTGEKG |
| ErbB2ZFR39A | SEQ ID | ERPFQCRICMRNFSRSDVLA |
| | NO: 7 | NHTRTHTGEKPFQCRICMAN |
| | | FSQSSTLTRHLRTHTGEKPF |
| | | QCRICMRNFSERQGLKRHLK |
| | | THTGEKG |
| ErbB2ZFR2AR39A | SEQ ID | EAPFQCRICMRNFSRSDVLA |
| | NO: 8 | NHTRTHTGEKPFQCRICMAN |
| | | FSQSSTLTRHLRTHTGEKPF |
| | | QCRICMRNFSERQGLKRHLK |
| | | THTGEKG |
| ErbB2ZFR2AR39AR67A | SEQ ID | EAPFQCRICMRNFSRSDVLA |
| | NO: 9 | NHTRTHTGEKPFQCRICMAN |
| | | FSQSSTLTRHLRTHTGEKPF |
| | | QCRICMANFSERQGLKRHLK |
| | | THTGEKG |
| 37ZFWT | SEQ ID | ERPFQCRICMRNFSRNFILQ |
| | NO: 10 | RHIRTHTGEKPFQCRICMRN |
| | | FSDRANLRRHIRTHTGEKPF |
| | | QCRICMRNFSRHDQLTRHIR |
| | | THTGLR |
| 37ZFR39A | SEQ ID | ERPFQCRICMRNFSRNFILQ |
| | NO: 11 | RHIRTHTGEKPFQCRICMAN |
| | | FSDRANLRRHIRTHTGEKPF |
| | | QCRICMRNFSRHDQLTRHIR |
| | | THTGLR |
| 37ZFR2AR39A | SEQ ID | EAPFQCRICMRNFSRNFILQ |
| | NO: 12 | RHIRTHTGEKPFQCRICMAN |
| | | FSDRANLRRHIRTHTGEKPF |
| | | QCRICMRNFSRHDQLTRHIR |
| | | THTGLR |
| 37ZFR2AR39AR67A | SEQ ID | EAPFQCRICMRNFSRNFILQ |
| | NO: 13 | RHIRTHTGEKPFQC |
| | | RICMANFSDRANLRRHIRTH |
| | | TGEKPFQCRICMANFSRHDQ |
| | | LTRHIRTHTGLR |
| 42ZFR2AR39AR67A | SEQ ID | EAPFQCRICMRNFSTGQILD |
| | NO: 14 | RHIRTHTGEKPFQCRICMAN |
| | | FSVAHSLKRHIRTHTGEKPF |
| | | QCRICMANFSDPSNLRRHIR |
| | | THTGLR |
| 92ZFVVT | SEQ ID | ERPFQCRICMRNFSDSPTLR |
| | NO: 15 | RHIRTHTGEKPFQCRICMRN |
| | | FSQRSSLVRHIRTHTGEKPF |
| | | QCRICMRNFSERGNLTRHIR |
| | | THTGLR |
| 92ZFR39A | SEQ ID | ERPFQCRICMRNFSDSPTLR |
| | NO: 16 | RHIRTHTGEKPFQCRICMAN |
| | | FSQRSSLVRHIRTHTGEKPF |
| | | QCRICMRNFSERGNLTRHIR |
| | | THTGLR |
| 92ZFR2AR39A | SEQ ID | EAPFQCRICMRNFSDSPTLR |
| | NO: 17 | RHIRTHTGEKPFQCRICMAN |
| | | FSQRSSLVRHIRTHTGEKPF |
| | | QCRICMRNFSERGNLTRHIR |
| | | THTGLR |
| 92ZFR2AR39AR67A | SEQ ID | EAPFQCRICMRNFSDSPTLR |
| | NO: 18 | RHTRTHTGEKPFQCRICMAN |
| | | FSQRSSLVRHLRTHTGEKPF |
| | | QCRICMANFSERGNLTRHLK |
| | | THTGEKG |
| 97ZFWT | SEQ ID | ERPFQCRICMRNFSRQSNLS |
| | NO: 19 | RHIRTHTGEKPFQCRICMRN |
| | | FSRNEHLVLHIRTHTGEKPF |
| | | QCRICMRNFSQKTGLRVHIR |
| | | THTGLR |
| 97ZFR39A | SEQ ID | ERPFQCRICMRNFSRQSNLS |
| | NO: 20 | RHIRTHTGEKPFQCRICMAN |
| | | FSRNEHLVLHIRTHTGEKPF |
| | | QCRICMRNFSQKTGLRVHIR |
| | | THTGLR |
| 97ZFR2AR39A | SEQ ID | EAPFQCRICMRNFSRQSNLS |
| | NO: 21 | RHIRTHTGEKPFQCRICMAN |
| | | FSRNEHLVLHIRTHTGEKPF |
| | | QCRICMRNFSQKTGLRVHIR |
| | | THTGLR |
| BCRZF | SEQ ID | ERPFQCRICMRNFSDSPTLR |
| | NO: 22 | RHTRTHTGEKPFQCRICMRN |
| | | FSQGANLRRHLRTHTGEKPF |
| | | QCRICMRNFSQANTLQRHLK |
| | | THTGEKG |
| BCRZFR39A | SEQ ID | ERPFQCRICMRNFSDSPTLR |
| | NO: 23 | RHTRTHTGEKPFQCRICMAN |
| | | FSQGANLRRHLRTHTGEKPF |
| | | QCRICMRNFSQANTLQRHLK |
| | | THTGEKG |
| HIV1ZFWT | SEQ ID | ERPFQCRICMRNFSLRTDLD |
| | NO: 24 | RHTRTHTGEKPFQCRICMRN |
| | | FSLSQTLRRHLRTHTGEKPF |
| | | QCRICMRNFSLRSNLGRHLK |
| | | THTGEKG |
| HIV1ZFR39A | SEQ ID | ERPFQCRICMRNFSLRTDLD |
| | NO: 25 | RHTRTHTGEKPFQCRICMAN |
| | | FSLSQTLRRHLRTHTGEKPF |
| | | QCRICMRNFSLRSNLGRHLK |
| | | THTGEKG |
| HIV1ZFR2AR39A | SEQ ID | EAPFQCRICMRNFSLRTDLD |
| | NO: 26 | RHTRTHTGEKPFQCRICMAN |
| | | FSLSQTLRRHLRTHTGEKPF |
| | | QCRICMRNFSLRSNLGRHLK |
| | | THTGEKG |
| HIV1ZFR2AR39AR67A | SEQ ID | EAPFQCRICMRNFSLRTDLD |
| | NO: 27 | RHTRTHTGEKPFQCRICMAN |
| | | FSLSQTLRRHLRTHTGEKPF |
| | | QCRICMANFSLRSNLGRHLK |
| | | THTGEKG |
| HIV2ZFWT | SEQ ID | ERPFQCRICMRNFSNNAMLV |
| | NO: 28 | RHTRTHTGEKPFQCRICMRN |
| | | FSLSQTLQRHLRTHTGEKPF |
| | | QCRICMRNFSMQGNLSRHLK |
| | | THTGEKG |
| HIV2ZFR39A | SEQ ID | ERPFQCRICMRNFSNNAMLV |
| | NO: 29 | RHTRTHTGEKPFQCRICMAN |
| | | FSLSQTLQRHLRTHTGEKPF |
| | | QCRICMRNFSMQGNLSRHLK |
| | | THTGEKG |
| HIV2ZFR2AR39A | SEQ ID | EAPFQCRICMRNFSNNAMLV |
| | NO: 30 | RHTRTHTGEKPFQCRICMAN |
| | | FSLSQTLQRHLRTHTGEKPF |
| | | QCRICMRNFSMQGNLSRHLK |
| | | THTGEKG |
| H1V2ZFR2AR39AR67A | SEQ ID | EAPFQCRICMRNFSNNAMLV |
| | NO: 31 | RHTRTHTGEKPFQCRICMAN |
| | | FSLSQTLQRHLRTHTGEKPF |
| | | QCRICMANFSMQGNLSRHLK |
| | | THTGEKG |
| NLS-FKBP12F36V- | SEQ ID | PKKKRKVSGVQVETISPGDG |
| 37ZFR2AR11AR39AR67A- | NO: 32 | RTFPKRGQTCVVHYTGMLED |
| VP16-NLS-DHFR | | GKKVDSSRDRNKPFKFMLGK |
| | | QEVIRGWEEGVAQMSVGQRA |
| | | KLTISPDYAYGATGHPGIIP |
| | | PHATLVFDVELLKLEGSEAP |
| | | FQCRICMANFSRNFILQRHT |
| | | RTHTGEKPFQCRICMANFSD |
| | | RANLRRHLRTHTGEKPFQCR |
| | | ICMANFSRHDQLTRHLKTHT |
| | | GEKGGGSSGAPPTDVSLGDE |
| | | LHLDGEDVAMAHADALDDFD |
| | | LDMLGDGDSPGPGFTPHDSA |
| | | PYGALDMADFEFEQMFTDAL |
| | | GIDEYGGGSPKKKRKVGSSD |
| | | YKDDDDKSSISLIAALAVDY |
| | | VIGMENAMPWNLPADLAWFK |
| | | RNTLNKPVIMGRHTWESIGR |
| | | PLPGRKNIILSSQPSTDDRV |
| | | TWVKSVDEAIAACGDVPEIM |
| | | VIGGGRVIEQFLPKAQKLYL |
| | | THIDAEVEGDTHFPDYEPDD |
| | | WESVFSEFHDADAQNSHSYC |
| | | FEILERR |
| NLS-FKBP12F36V- | SEQ ID | PKKKRKVSGVQVETISPGDG |
| BCRZFR39A- | NO: 33 | RTFPKRGQTCVVHYTGMLED |
| VP16-NLS- | | GKKVDSSRDRNKPFKFMLGK |
| DHFR | | QEVIRGWEEGVAQMSVGQRA |
| | | KLTISPDYAYGATGHPGIIP |
| | | PHATLVFDVELLKLEGSERP |
| | | FQCRICMRNFSDSPTLRRHT |
| | | RTHTGEKPFQCRICMANFSQ |
| | | GANLRRHLRTHTGEKPFQCR |
| | | ICMRNFSQANTLQRHLKTHT |
| | | GEKGGGSSGAPPTDVSLGDE |
| | | LHLDGEDVAMAHADALDDFD |
| | | LDMLGDGDSPGPGFTPHDSA |
| | | PYGALDMADFEFEQMFTDAL |
| | | GIDEYGGGSPKKKRKVGSSD |
| | | YKDDDDKSSISLIAALAVDY |
| | | VIGMENAMPWNLPADLAWFK |
| | | RNTLNKPVIMGRHTWESIGR |
| | | PLPGRKNIILSSQPSTDDRV |
| | | TWVKSVDEAIAACGDVPEIM |
| | | VIGGGRVIEQFLPKAQKLYL |
| | | THIDAEVEGDTHFPDYEPDD |
| | | WESVFSEFHDADAQNSHSYC |
| | | FEILERR |
| NLS-FKBP 12F36V- | SEQ ID | PKKKRKVSGVQVETISPGDG |
| ErbB2ZFR2AR39A- | NO: 34 | RTFPKRGQTCVVHYTGMLED |
| VP16-NLS-DHFR | | GKKVDSSRDRNKPFKFMLGK |
| | | QEVIRGWEEGVAQMSVGQRA |
| | | KLTISPDYAYGATGHPGIIP |
| | | PHATLVFDVELLKLEGSEAP |
| | | FQCRICMRNFSRSDVLANHT |
| | | RTHTGEKPFQCRICMANFSQ |
| | | SSTLTRHLRTHTGEKPFQCR |
| | | ICMRNFSERQGLKRHLKTHT |
| | | GEKGGGSSGAPPTDVSLGDE |
| | | LHLDGEDVAMAHADALDDFD |
| | | LDMLGDGDSPGPGFTPHDSA |
| | | PYGALDMADFEFEQMFTDAL |
| | | GIDEYGGGSPKKKRKVGSSD |
| | | YKDDDDKSSISLIAALAVDY |
| | | VIGMENAMPWNLPADLAWFK |
| | | RNTLNKPVIMGRHTWESIGR |
| | | PLPGRKNIILSSQPSTDDRV |
| | | TWVKSVDEAIAACGDVPEIM |
| | | VIGGGRVIEQFLPKAQKLYL |
| | | THIDAEVEGDTHFPDYEPDD |
| | | WESVFSEFHDADAQNSHSYC |
| | | FEILERR |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “of” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A nucleic acid composition, comprising:
a first promoter operably linked to a first polynucleotide encoding a first transcription factor (TF) and to a second polynucleotide encoding one or more first payloads,
wherein the first promoter comprises one or more pairs of first TF binding sites, and
wherein the first TF comprises a first DNA-binding domain capable of binding a first TF binding site;
a second promoter operably linked to a third polynucleotide encoding a second transcription factor (TF) and to a fourth polynucleotide encoding one or more second payloads,
wherein the second promoter comprises one or more pairs of second TF binding sites, and
wherein the second TF comprises a second DNA-binding domain capable of binding a second TF binding site; and
a third promoter operably linked to a fifth polynucleotide encoding a third transcription factor (TF) and to a sixth polynucleotide encoding one or more third payloads,
wherein the third promoter comprises one or more pairs of third TF binding sites, and
wherein the third TF comprises a third DNA-binding domain capable of binding a third TF binding site.

2. The nucleic acid composition of claim 1,
wherein the first TF comprises a dimerization domain,
wherein the dimerization domain of two first TF are capable of associating to generate a first TF homodimer, wherein a first TF homodimer is capable of binding the pair of first TF binding sites, wherein the dimerization domain of each of two first TF are capable of associating to generate the first TF homodimer in the presence of a dimerization ligand;
wherein the second TF comprises a dimerization domain,
wherein the dimerization domain of two second TF are capable of associating to generate a second TF homodimer, wherein a second TF homodimer is capable of binding the pair of second TF binding sites, wherein the dimerization domain of each of two second TF are capable of associating to generate the second TF homodimer in the presence of a dimerization ligand; and/or
wherein the third TF comprises a dimerization domain,
wherein the dimerization domain of two third TF are capable of associating to generate a third TF homodimer, wherein a third TF homodimer is capable of binding the pair of third TF binding sites, wherein the dimerization domain of each of two third TF are capable of associating to generate the third TF homodimer in the presence of a dimerization ligand.

3. The nucleic acid composition of claim 1,
wherein the first TF further comprises a degron capable of binding a degron stabilizing molecule, and wherein the first TF changes from a destabilized state to a stabilized state when the degron binds to the degron stabilizing molecule;
wherein the second TF further comprises a degron capable of binding a degron stabilizing molecule, and wherein the second TF changes from a destabilized state to a stabilized state when the degron binds to the degron stabilizing molecule; and/or
wherein the third TF further comprises a degron capable of binding a degron stabilizing molecule, and wherein the third TF changes from a destabilized state to a stabilized state when the degron binds to the degron stabilizing molecule.

4. The nucleic acid composition of claim 1,
wherein the one or more first payloads comprise one or more first payload proteins and/or one or more first payload RNA agents,
wherein, upon the first TF homodimer binding a pair of first TF binding sites, the first promoter is capable of inducing transcription of the first polynucleotide and the second polynucleotide to generate a first polycistronic transcript,
wherein the first polynucleotide and the second polynucleotide are operably linked to a tandem gene expression element, and
wherein the first polycistronic transcript is capable of being translated to generate the first TF and the one or more first payloads;
wherein the one or more second payloads comprise one or more second payload proteins and/or one or more second payload RNA agents,
wherein, upon the second TF homodimer binding a pair of second TF binding sites, the second promoter is capable of inducing transcription of the third polynucleotide and the fourth polynucleotide to generate a second polycistronic transcript,
wherein the third polynucleotide and the fourth polynucleotide are operably linked to a tandem gene expression element, and
wherein the second polycistronic transcript is capable of being translated to generate the second TF and the one or more second payloads; and/or
wherein the one or more third payloads comprise one or more third payload proteins and/or one or more third payload RNA agents,
wherein, upon the third TF homodimer binding a pair of third TF binding sites, the third promoter is capable of inducing transcription of the fifth polynucleotide and the sixth polynucleotide to generate a third polycistronic transcript,
wherein the fifth polynucleotide and the sixth polynucleotide are operably linked to a tandem gene expression element, and
wherein the third polycistronic transcript is capable of being translated to generate the third TF and the one or more third payloads.

5. The nucleic acid composition of claim 1,
wherein the first promoter further comprises:
one or more copies of a transactivator recognition sequence that a transactivator is capable of binding, and wherein, in the presence of the transactivator and a transactivator-binding compound, the first promoter is capable of inducing transcription of the first polynucleotide and the second polynucleotide to generate the first polycistronic transcript, and/or
one or more copies of a basal expression motif capable of inducing transcription of the first polynucleotide and the second polynucleotide to generate the first polycistronic transcript;
wherein the second promoter further comprises:
one or more copies of a transactivator recognition sequence that a transactivator is capable of binding, and wherein, in the presence of the transactivator and a transactivator-binding compound, the second promoter is capable of inducing transcription of the third polynucleotide and the fourth polynucleotide to generate the second polycistronic transcript, and/or
one or more copies of a basal expression motif capable of inducing transcription of the third polynucleotide and the fourth polynucleotide to generate the second polycistronic transcript; and/or
wherein the third promoter further comprises:
one or more copies of a transactivator recognition sequence that a transactivator is capable of binding, and wherein, in the presence of the transactivator and a transactivator-binding compound, the third promoter is capable of inducing transcription of the fifth polynucleotide and the sixth polynucleotide to generate the third polycistronic transcript, and/or
one or more copies of a basal expression motif capable of inducing transcription of the fifth polynucleotide and the sixth polynucleotide to generate the third polycistronic transcript.

6. The nucleic acid composition of claim 2, wherein the dimerization domain:
(i) comprises or is derived from GCN4, FKBP, cyclophilin, steroid binding protein, estrogen binding protein, glucocorticoid binding protein, vitamin D binding protein, tetracycline binding protein, extracellular domain of a cytokine receptor, a receptor tyrosine kinase, a TNFR-family receptor, an immune co-receptor, or any combination thereof;
(ii) comprises an amino acid sequence at least 70 percent identical to FKBP12F36V (SEQ ID NO: 5); and/or
(iii) comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof.

7. The nucleic acid composition of claim 2, wherein the dimerization ligand comprises or is derived from AP1903, AP20187, dimeric FK506, a dimeric FK506-like analog, derivatives thereof, or any combination thereof.

8. The nucleic acid composition of claim 2, wherein the dimerization domain of the first TF, the second TF, and/or the third TF are the same.

9. The nucleic acid composition of claim 2, wherein:
the dimerization domains of
(i) a first TF and a second TF,
(ii) a first TF and a third TF, and/or
(iii) a second TF and a third TF,
are capable of associating to generate a TF heterodimer in the presence of a dimerization ligand.

10. The nucleic acid composition of claim 9, wherein a TF heterodimer has at least about 1.1-fold less binding affinity for a pair of TF binding sites as compared to a TF homodimer.

11. The nucleic acid composition of claim 2, wherein a TF monomer has at least about 1.1-fold less binding affinity for a pair of TF binding sites as compared to a TF homodimer.

12. The nucleic acid composition of claim 1, wherein the first DNA-binding domain, the second DNA-binding domain, and/or the third DNA-binding domain:
(i) comprises or is derived from a TALE DNA binding domain 2, catalytically dead CRISPR/Cas9 (dCas9) 3-5, Gal4, hypoxia inducible factor (HIF), HIF1a, cyclic AMP response element binding (CREB) protein, LexA, rtTA, an endonuclease, a zinc finger (ZF) binding domain, a transcription factor, portions thereof, or any combination thereof;
(ii) is a synthetic DNA-binding domain configured to decrease monomeric TF activity without reducing TF homodimer activity; and/or
(iii) comprises or is derived from a zinc finger DNA-binding domain, wherein the zinc finger (ZF) DNA-binding domain comprises or is derived from ErbB2 ZF, BCRZF, HIV1ZF, HIV2ZF, 37ZF (37-12 array), 42ZF (42-10 array), 43ZF (43-8 array), 92ZF (92-1 array), and/or 97ZF (97-4 array).

13. The nucleic acid composition of claim 1, wherein the first DNA-binding domain, the second DNA-binding domain, and/or the third DNA-binding domain comprises an amino acid sequence at least 70 percent identical to ErbB2ZFWT (SEQ ID NO: 6), ErbB2ZFR39A (SEQ ID NO: 7), ErbB2ZFR2AR39A (SEQ ID NO: 8), ErbB2ZFR2AR39AR67A (SEQ ID NO: 9), 37ZFWT (SEQ ID NO: 10), 37ZFR39A (SEQ ID NO: 11), 37ZFR2AR39A (SEQ ID NO: 12), 37ZFR2AR39AR67A (SEQ ID NO: 13), 42ZFR2AR39AR67A (SEQ ID NO: 14), 92ZFWT (SEQ ID NO: 15), 92ZFR39A (SEQ ID NO: 16), 92ZFR2AR39A (SEQ ID NO: 17), 92ZFR2AR39AR67A (SEQ ID NO: 18), 97ZFWT (SEQ ID NO: 19), 97ZFR39A (SEQ ID NO: 20), 97ZFR2AR39A (SEQ ID NO: 21), BCRZF (SEQ ID NO: 22), BCRZFR39A (SEQ ID NO: 23), HIV1ZFWT (SEQ ID NO: 24), HIV1ZFR39A (SEQ ID NO: 25), HIV1ZFR2AR39A (SEQ ID NO: 26), HIV1ZFR2AR39AR67A (SEQ ID NO: 27), HIV2ZFWT (SEQ ID NO: 28), HIV2ZFR39A (SEQ ID NO: 29), HIV2ZFR2AR39A (SEQ ID NO: 30), and/or HIV2ZFR2AR39AR67A (SEQ ID NO: 31).

14. The nucleic acid composition of claim 1, wherein the first TF, second TF, and/or third TF comprises a transactivation domain, and wherein a transactivation domain comprises or is derived from VP16, TA2, VP64 (a tetrameric repeat of the minimal activation domain of VP16), VP48 (a trimeric repeat of the minimal activation domain of VP16), signal transducer and activator of transcription 6 (STAT6), reticuloendotheliosis virus A oncogene (relA), TATA binding protein associated factor-1 (TAF-1), TATA binding protein associated factor-2 (TAF-2), glucocorticoid receptor TAU-1, or glucocorticoid receptor TAU-2, a steroid/thyroid hormone nuclear receptor transactivation domain, a polyglutamine transactivation domain, a basic or acidic amino acid transactivation domain, a GAL4 transactivation domain, an NF-κB transactivation domain, a p65 transactivation domain, a BP42 transactivation domain, HSF1, VP16, VP64, p65, MyoD1, RTA, SET7/9, VPR, histone acetyltransferase p300, an hydroxylase catalytic domain of a TET family protein (e.g., TETl hydroxylase catalytic domain), LSD1, CIB1, AD2, CR3, EKLF1, GATA4, PRVIE, p53, SP1, MEF2C, TAX, and PPARγ, Gal4, Gcn4, MLL, Rtg3, Gln3, Oaf1, Pip2, Pdr1, Pdr3, Pho4, Leu3, portions thereof having transcription activating activity, or any combination thereof.

15. The nucleic acid composition of claim 5, wherein:
a transactivator recognition sequence comprises a Tet3G binding site (TRE3G) or a ERT2-Gal4 binding site (UAS);
a transactivator recognition sequence comprises a element of an inducible promoter, wherein the inducible promoter is selected from the group comprising a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, an RU-486 responsive promoter, or any combination thereof, and/or
the transactivator-binding compound comprises 4-hydroxy-tamoxifen (4-OHT), Dox, derivatives thereof, or any combination thereof.

16. The nucleic acid composition of claim 3, wherein,
the degron comprises a dihydrofolate reductase (DHFR) degron, a FKB protein (FKBP) degron, derivatives thereof, or any combination thereof, and/or
the degron stabilizing molecule comprises trimethoprim (TMP), Shield-1, derivatives thereof, or any combination thereof.

17. The nucleic acid composition of claim 4, wherein one or more first payload proteins, second payload proteins, and/or third payload proteins comprise:
(i) fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof;
(ii) nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof;
(iii) a CRE recombinase, GCaMP, a cell therapy component, a knock-down gene therapy component, a cell-surface exposed epitope, or any combination thereof;
(iv) a diagnostic agent selected from the group comprising green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCitrine, mCherry, mruby3, rsCherry, rsCherryRev, derivatives thereof, or any combination thereof;
(v) a bispecific T cell engager (BiTE);
(vi) a cytokine selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof;
(vii) a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5;
(viii) a member of the TNF family of cytokines selected from the group consisting of TNF-alpha, TNF-beta, LT-beta, CD40 ligand, Fas ligand, CD 27 ligand, CD 30 ligand, and 4-1 BBL;
(ix) a member of the immunoglobulin superfamily of cytokines selected from the group consisting of B7.1 (CD80) and B7.2 (B70);
(x) an interferon selected from the group comprising interferon alpha, interferon beta, interferon gamma, or any combination thereof;
(xi) a chemokine selected from the group comprising CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, CXCL15, or any combination thereof;
(xii) a interleukin selected from IL-10 IL-12, IL-1, IL-6, IL-7, IL-15, IL-2, IL-18, IL-21, or any combination thereof;
(xiii) a tumor necrosis factor (TNF) selected from TNF-alpha, TNF-beta, TNF-gamma, CD252, CD154, CD178, CD70, CD153, 4-1BBL, or any combination thereof;
(xv) a component of a synthetic protein circuit;
(xv) a factor locally down-regulating the activity of endogenous immune cells; and/or
(xvi) a chimeric antigen receptor (CAR) or T-cell receptor (TCR).

18. The nucleic acid composition of claim 1, wherein the first polynucleotide, the second polynucleotide, the third polynucleotide, the fourth polynucleotide, the fifth polynucleotide, and/or the sixth polynucleotide:
(i) is operably linked to a tandem gene expression element, wherein the tandem gene expression element is an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof, and/or
(ii) further comprises a transcript stabilization element, wherein the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

19. A cell population comprising a plurality of cells, each cell comprising:
the nucleic acid composition of claim 1.

20. A method of treating a disease or disorder in a subject, the method comprising:
administering to the subject an effective amount of the cell population of claim 19.
